SEQUENCE LISTING (417 sequences)

SEQ ID NO: 1 - protein, 26 aa, synthetic construct
EVQLVESGGGLVQPGGSLRLSCSASG

SEQ ID NO: 2 - protein, 9 aa, synthetic construct
FTFSRFAMH

SEQ ID NO: 3 - protein, 14 aa, synthetic construct
WVRQAPGKGLEYVS

SEQ ID NO: 4 - protein, 17 aa, synthetic construct
AINLNGDSKYYTDSVRG

SEQ ID NO: 5 - protein, 30 aa, synthetic construct
RFTISRDNSKNTLYLQMSSVRPDDTAFYYC

SEQ ID NO: 6 - protein, 14 aa, synthetic construct
VKDGGYYDSSGPGH

SEQ ID NO: 7 - protein, 11 aa, synthetic construct
WGQGTLVTVSS

SEQ ID NO: 8 - protein, 121 aa, synthetic construct
EVQLVESGGGLVQPGGSLRLSCSASGFTFSRFAMHWVRQAPGKGLEYVSAINLNGDSKYYTDSVRGRFTISRDNSKNTLYLQMSSVRPDDTAFYYCVKDGGYYDSSGPGHWGQGTLVTVSS

SEQ ID NO: 9 - DNA, 363 bp, synthetic construct
  source 1..363: mol_type = other DNA
gaggtgcagctggtggagtctgggggaggcttggtccagcctggggggtccctgagactttcctgttcagcctctggattcaccttcagtaggtttgctatgcactgggtcaggcaggctccagggaagggactggaatatgtttcagctattaatctgaatggggacagcaaatactacaccgactccgtgaggggcagattcaccatctccagagacaattccaagaacacgctgtatcttcaaatgagcagtgtgagacccgacgacacggctttctattactgtgtaaaagacgggggttactacgattcctccggtcctgggcactgggggcaagggaccttggtcaccgtctcctca

SEQ ID NO: 10 - protein, 451 aa, synthetic construct
EVQLVESGGGLVQPGGSLRLSCSASGFTFSRFAMHWVRQAPGKGLEYVSAINLNGDSKYYTDSVRGRFTISRDNSKNTLYLQMSSVRPDDTAFYYCVKDGGYYDSSGPGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 26 aa, synthetic construct
EVQLVESGGGLVQPGGSLRLSCSASG

SEQ ID NO: 12 - protein, 9 aa, synthetic construct
FTFSRFAMH

SEQ ID NO: 13 - protein, 14 aa, synthetic construct
WVRQAPGKGLEYVS

SEQ ID NO: 14 - protein, 17 aa, synthetic construct
AINLNGDSKYYTDSVRG

SEQ ID NO: 15 - protein, 30 aa, synthetic construct; identical to SEQ ID NO: 5

SEQ ID NO: 16 - protein, 14 aa, synthetic construct
VKDGGYYDSSGPGH

SEQ ID NO: 17 - protein, 11 aa, synthetic construct
WGQGTLVTVSS

SEQ ID NO: 18 - protein, 121 aa, synthetic construct; identical to SEQ ID NO: 8

SEQ ID NO: 19 - DNA, 363 bp, synthetic construct
  source 1..363: mol_type = other DNA
gaggtgcagctggtggagtctgggggaggcttggtccagcctggggggtccctgagattgtcctgttcagcctctggattcaccttcagtaggtttgctatgcactgggtcaggcaggctccagggaagggactggaatatgtttcagctattaatctgaatggggatagcaagtactacacagactccgtgagaggcagattcaccatctccagagacaattccaagaacacgctgtatcttcaaatgagcagtgtaagaccagacgacacggctttctattactgtgttaaggatggcggctactacgatagttccggtcctggtcactgggggcaagggacccttgtcaccgtctcctca

SEQ ID NO: 20 - protein, 451 aa, synthetic construct
EVQLVESGGGLVQPGGSLRLSCSASGFTFSRFAMHWVRQAPGKGLEYVSAINLNGDSKYYTDSVRGRFTISRDNSKNTLYLQMSSVRPDDTAFYYCVKDGGYYDSSGPGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 26 aa, synthetic construct
EVQLVESGGGLVQPGGSLRLSCSASG

SEQ ID NO: 22 - protein, 9 aa, synthetic construct
FTFSRFAMH

SEQ ID NO: 23 - protein, 14 aa, synthetic construct
WVRQAPGKGLEYVS

SEQ ID NO: 24 - protein, 17 aa, synthetic construct
AINLNGDSKYYTDSVRG

SEQ ID NO: 25 - protein, 30 aa, synthetic construct; identical to SEQ ID NO: 5

SEQ ID NO: 26 - protein, 14 aa, synthetic construct
VKDGGYYDSSGPGH

SEQ ID NO: 27 - protein, 11 aa, synthetic construct
WGQGTLVTVSS

SEQ ID NO: 28 - protein, 121 aa, synthetic construct; identical to SEQ ID NO: 8

SEQ ID NO: 29 - DNA, 363 bp, synthetic construct
  source 1..363: mol_type = other DNA
gaggtgcagctggtggagtctgggggaggcttggtccagcctggggggtccctgagactttcctgttcagcctctggattcaccttcagtagattcgctatgcactgggtcaggcaggctccagggaagggactggaatatgtttcagctattaatcttaatggggacagcaagtactacaccgactccgtgaggggcagattcaccatctccagagacaattccaagaacacgctgtatcttcaaatgagcagtgttagacctgacgacacggctttctattactgtgtaaaagacggaggatactacgacagttccggtcccggacactgggggcaagggacccttgtcaccgtctcctca

SEQ ID NO: 30 - protein, 451 aa, synthetic construct
EVQLVESGGGLVQPGGSLRLSCSASGFTFSRFAMHWVRQAPGKGLEYVSAINLNGDSKYYTDSVRGRFTISRDNSKNTLYLQMSSVRPDDTAFYYCVKDGGYYDSSGPGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 31 - protein, 26 aa, synthetic construct
EVQLVESGGGLVQPGGSLRLSCSASG

SEQ ID NO: 32 - protein, 9 aa, synthetic construct
FTFSRFAMH

SEQ ID NO: 33 - protein, 14 aa, synthetic construct
WVRQAPGKGLEYVS

SEQ ID NO: 34 - protein, 17 aa, synthetic construct
AINLNGDSKYYTDSVRG

SEQ ID NO: 35 - protein, 30 aa, synthetic construct; identical to SEQ ID NO: 5

SEQ ID NO: 36 - protein, 14 aa, synthetic construct
VKDGGYYDSSGPGH

SEQ ID NO: 37 - protein, 11 aa, synthetic construct
WGQGTLVTVSS

SEQ ID NO: 38 - protein, 121 aa, synthetic construct; identical to SEQ ID NO: 8

SEQ ID NO: 39 - DNA, 363 bp, synthetic construct
  source 1..363: mol_type = other DNA
gaggtgcagctggtggagtctgggggaggcttggtccagcctggggggtccctgagattgtcctgttcagcctctggattcaccttcagtagatttgctatgcactgggtcaggcaggctccagggaagggactggaatatgtttcagctattaacttgaatggggatagcaaatactacacggactccgtgagaggcagattcaccatctccagagacaattccaagaacacgctgtatcttcaaatgagcagtgttagaccggacgacacggctttctattactgtgtcaaagacgggggatactacgatagtagtggtcctggccactgggggcaagggacccttgtcaccgtctcctca

SEQ ID NO: 40 - protein, 451 aa, synthetic construct
EVQLVESGGGLVQPGGSLRLSCSASGFTFSRFAMHWVRQAPGKGLEYVSAINLNGDSKYYTDSVRGRFTISRDNSKNTLYLQMSSVRPDDTAFYYCVKDGGYYDSSGPGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 41 - protein, 26 aa, synthetic construct
EVQLVESGGGLVKPGGSLRLSCAASG

SEQ ID NO: 42 - protein, 9 aa, synthetic construct
FTFSSYYMN

SEQ ID NO: 43 - protein, 14 aa, synthetic construct
WVRQAPGKGLEWVS

SEQ ID NO: 44 - protein, 17 aa, synthetic construct
SISEDGYSTYYPDSLKG

SEQ ID NO: 45 - protein, 30 aa, synthetic construct
RFTISRDSAKNSLYLQMNSLRADDTAVYYC

SEQ ID NO: 46 - protein, 17 aa, synthetic construct
ARDFSGHTAWAGTGFEY

SEQ ID NO: 47 - protein, 11 aa, synthetic construct
WGQGTLVTVSS

SEQ ID NO: 48 - protein, 124 aa, synthetic construct
EVQLVESGGGLVKPGGSLRLSCAASGFTFSSYYMNWVRQAPGKGLEWVSSISEDGYSTYYPDSLKGRFTISRDSAKNSLYLQMNSLRADDTAVYYCARDFSGHTAWAGTGFEYWGQGTLVTVSS

SEQ ID NO: 49 - DNA, 372 bp, synthetic construct
  source 1..372: mol_type = other DNA
gaggtgcagctggtggagtctgggggaggcctggtcaagcctggggggtccctgagactttcctgtgcagcctctggattcaccttcagtagctattatatgaactgggtcagacaggctccagggaaggggctggagtgggtctcatccattagtgaggatggatacagtacatactaccctgactcacttaagggcagattcaccatctccagagacagtgccaagaactcactgtatctgcaaatgaacagcctgagagccgacgacacggctgtgtattactgtgccagagatttcagtggtcacaccgcttgggccggtacgggatttgaatactggggccaaggaaccctggtcaccgtctcctca

SEQ ID NO: 50 - protein, 454 aa, synthetic construct
EVQLVESGGGLVKPGGSLRLSCAASGFTFSSYYMNWVRQAPGKGLEWVSSISEDGYSTYYPDSLKGRFTISRDSAKNSLYLQMNSLRADDTAVYYCARDFSGHTAWAGTGFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 26 aa, synthetic construct
EVQLVESGGGLVKPGGSLRLSCAASG

SEQ ID NO: 52 - protein, 9 aa, synthetic construct
FTFSSYYMN

SEQ ID NO: 53 - protein, 14 aa, synthetic construct
WVRQAPGKGLEWVS

SEQ ID NO: 54 - protein, 17 aa, synthetic construct
SISEDGYSTYYPDSLKG

SEQ ID NO: 55 - protein, 30 aa, synthetic construct; identical to SEQ ID NO: 45

SEQ ID NO: 56 - protein, 17 aa, synthetic construct
ARDFSGHTAWAGTGFEY

SEQ ID NO: 57 - protein, 11 aa, synthetic construct
WGQGTLVTVSS

SEQ ID NO: 58 - protein, 124 aa, synthetic construct; identical to SEQ ID NO: 48

SEQ ID NO: 59 - DNA, 372 bp, synthetic construct
  source 1..372: mol_type = other DNA
gaggtgcagctggtggagtctgggggaggcctggtcaagcctggggggtccctgagactgtcctgtgcagcctctggattcaccttcagtagctattatatgaactgggtcaggcaggctccagggaaggggctggagtgggtctcatccattagtgaggacggatacagcacctactaccccgactcactgaagggcagattcaccatctccagagacagcgccaagaactcactgtatctgcaaatgaacagcctgagagccgatgacacggctgtgtattactgtgccagagatttttcaggccacacggcgtgggcaggaacgggatttgagtactggggccaaggaaccctggtcaccgtctcctca

SEQ ID NO: 60 - protein, 454 aa, synthetic construct
EVQLVESGGGLVKPGGSLRLSCAASGFTFSSYYMNWVRQAPGKGLEWVSSISEDGYSTYYPDSLKGRFTISRDSAKNSLYLQMNSLRADDTAVYYCARDFSGHTAWAGTGFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 61 - protein, 26 aa, synthetic construct
EVQLVESGGGLIQPGGSLRLSCAASG

SEQ ID NO: 62 - protein, 9 aa, synthetic construct
FPFKGTYMT

SEQ ID NO: 63 - protein, 14 aa, synthetic construct
WVRQAPGKGLEWVS

SEQ ID NO: 64 - protein, 16 aa, synthetic construct
IIYSGGDTYYADSVKG

SEQ ID NO: 65 - protein, 30 aa, synthetic construct
RFTISRDNSKNTLFLQMNSLRVEDTAMYYC

SEQ ID NO: 66 - protein, 18 aa, synthetic construct
ARDREMAIITERTYGLDV

SEQ ID NO: 67 - protein, 11 aa, synthetic construct
WGQGTTVTVSS

SEQ ID NO: 68 - protein, 124 aa, synthetic construct
EVQLVESGGGLIQPGGSLRLSCAASGFPFKGTYMTWVRQAPGKGLEWVSIIYSGGDTYYADSVKGRFTISRDNSKNTLFLQMNSLRVEDTAMYYCARDREMAIITERTYGLDVWGQGTTVTVSS

SEQ ID NO: 69 - DNA, 372 bp, synthetic construct
  source 1..372: mol_type = other DNA
gaggtgcagctggtggagtctggaggaggcttgatccagcctggggggtccctgagactgtcctgtgcagcctctgggttcccgtttaagggcacctacatgacctgggtcagacaggctccagggaaggggctggagtgggtctcaatcatttatagcggtggtgatacatactacgcagactccgtgaagggcagattcaccatctccagagacaattccaagaacacgctgttccttcaaatgaacagcctgagagtggaggacacggccatgtattactgtgccagggatagagagatggcgataatcacggaaaggacttacggtttggacgtctgggggcaagggaccacggtcaccgtctcctca

SEQ ID NO: 70 - protein, 454 aa, synthetic construct
EVQLVESGGGLIQPGGSLRLSCAASGFPFKGTYMTWVRQAPGKGLEWVSIIYSGGDTYYADSVKGRFTISRDNSKNTLFLQMNSLRVEDTAMYYCARDREMAIITERTYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 26 aa, synthetic construct
EVQLVESGGGLIQPGGSLRLSCAASG

SEQ ID NO: 72 - protein, 9 aa, synthetic construct
FPFKGTYMT

SEQ ID NO: 73 - protein, 14 aa, synthetic construct
WVRQAPGKGLEWVS

SEQ ID NO: 74 - protein, 16 aa, synthetic construct
IIYSGGDTYYADSVKG

SEQ ID NO: 75 - protein, 30 aa, synthetic construct; identical to SEQ ID NO: 65

SEQ ID NO: 76 - protein, 18 aa, synthetic construct
ARDREMAIITERTYGLDV

SEQ ID NO: 77 - protein, 11 aa, synthetic construct
WGQGTTVTVSS

SEQ ID NO: 78 - protein, 124 aa, synthetic construct; identical to SEQ ID NO: 68

SEQ ID NO: 79 - DNA, 372 bp, synthetic construct
  source 1..372: mol_type = other DNA
gaggtgcagctggtggagtctggaggaggcttgatccagcctggggggtccctgagactgtcctgtgcagcctctgggttccccttcaagggaacttacatgacatgggtcaggcaggctccagggaaggggctggagtgggtctcaatcatttatagcggtggtgatacatactacgcagactccgtgaagggcagattcaccatctccagagacaattccaagaacacgctgttccttcaaatgaacagcctgagagtcgaggacacggccatgtattactgtgccagagatagggagatggcgatcattaccgagaggacgtacggtcttgacgtctgggggcaagggaccacggtcaccgtctcctca

SEQ ID NO: 80 - protein, 454 aa, synthetic construct
EVQLVESGGGLIQPGGSLRLSCAASGFPFKGTYMTWVRQAPGKGLEWVSIIYSGGDTYYADSVKGRFTISRDNSKNTLFLQMNSLRVEDTAMYYCARDREMAIITERTYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 81 - protein, 26 aa, synthetic construct
QVQLVQSGAEVKKPGSSVKVSCKASG

SEQ ID NO: 82 - protein, 9 aa, synthetic construct
GTFSSYAIS

SEQ ID NO: 83 - protein, 14 aa, synthetic construct
WVRQAPGQGLEWMG

SEQ ID NO: 84 - protein, 17 aa, synthetic construct
GIIPIFVTANYAQKFQG

SEQ ID NO: 85 - protein, 30 aa, synthetic construct
RVTITADKSTTTVYMELSSLRSEDTAVYYC

SEQ ID NO: 86 - protein, 22 aa, synthetic construct
ARGRMATIRGGQNYYYYYGMDV

SEQ ID NO: 87 - protein, 11 aa, synthetic construct
WGQGTTVTVSS

SEQ ID NO: 88 - protein, 129 aa, synthetic construct
QVQLVQSGAEVKKPGSSVKVSCKASGGTFSSYAISWVRQAPGQGLEWMGGIIPIFVTANYAQKFQGRVTITADKSTTTVYMELSSLRSEDTAVYYCARGRMATIRGGQNYYYYYGMDVWGQGTTVTVSS

SEQ ID NO: 89 - DNA, 387 bp, synthetic construct
  source 1..387: mol_type = other DNA
caggtgcagctggtgcagtctggggctgaggtgaagaagcctgggtccagcgtgaaggtctcctgcaaggcttctggaggcaccttcagcagctatgctatcagctgggtgaggcaggcccctggacaagggcttgagtggatgggagggatcatccctatctttgttacagcaaactacgcacagaagttccagggcagagtcacgattaccgcggacaaatccacgacaacagtatacatggagctgagcagcctgagatctgaggacacggccgtgtattactgtgctagaggaaggatggccacaataagaggcgggcagaactactactactactacggtatggacgtctgggggcaagggaccacggtcaccgtctcctca

SEQ ID NO: 90 - protein, 459 aa, synthetic construct
QVQLVQSGAEVKKPGSSVKVSCKASGGTFSSYAISWVRQAPGQGLEWMGGIIPIFVTANYAQKFQGRVTITADKSTTTVYMELSSLRSEDTAVYYCARGRMATIRGGQNYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 26 aa, synthetic construct
QVQLVQSGAEVKKPGSSVKVSCKASG

SEQ ID NO: 92 - protein, 9 aa, synthetic construct
GTFSSYAIS

SEQ ID NO: 93 - protein, 14 aa, synthetic construct
WVRQAPGQGLEWMG

SEQ ID NO: 94 - protein, 17 aa, synthetic construct
GIIPIFVTANYAQKFQG

SEQ ID NO: 95 - protein, 30 aa, synthetic construct; identical to SEQ ID NO: 85

SEQ ID NO: 96 - protein, 22 aa, synthetic construct
ARGRMATIRGGQNYYYYYGMDV

SEQ ID NO: 97 - protein, 11 aa, synthetic construct
WGQGTTVTVSS

SEQ ID NO: 98 - protein, 129 aa, synthetic construct; identical to SEQ ID NO: 88

SEQ ID NO: 99 - DNA, 387 bp, synthetic construct
  source 1..387: mol_type = other DNA
caggtgcagctggtgcagtctggggctgaggtgaagaagcctgggtcctcagtgaaggtctcctgcaaggcttctggaggcaccttcagcagctatgctatcagctgggtgagacaggcccctggacaagggcttgagtggatgggagggatcatccctatctttgtaacagcaaactacgcacagaagttccagggcagagtcacgattaccgcggacaaatccacgacaacagtttacatggagctgagcagcctgagatctgaggacacggccgtgtattactgtgccagaggtaggatggccacgattaggggtggacagaattactactactactacggtatggacgtctgggggcaagggaccacggtcaccgtctcctca

SEQ ID NO: 100 - protein, 459 aa, synthetic construct
QVQLVQSGAEVKKPGSSVKVSCKASGGTFSSYAISWVRQAPGQGLEWMGGIIPIFVTANYAQKFQGRVTITADKSTTTVYMELSSLRSEDTAVYYCARGRMATIRGGQNYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 101 - protein, 26 aa, synthetic construct
EVQLVESGGGLVQPGRSLRLSCAASG

SEQ ID NO: 102 - protein, 9 aa, synthetic construct
FTFDDYAMH

SEQ ID NO: 103 - protein, 14 aa, synthetic construct
WVRQAPGKGLEWVS

SEQ ID NO: 104 - protein, 17 aa, synthetic construct
GISWNSGSINYADSVKG

SEQ ID NO: 105 - protein, 30 aa, synthetic construct
RFTISRDNAKNSLYLQMNSLRAEDTALYYC

SEQ ID NO: 106 - protein, 17 aa, synthetic construct
ASDSNYRDYYYHYGMDV

SEQ ID NO: 107 - protein, 11 aa, synthetic construct
WGQGTTVTVSS

SEQ ID NO: 108 - protein, 124 aa, synthetic construct
EVQLVESGGGLVQPGRSLRLSCAASGFTFDDYAMHWVRQAPGKGLEWVSGISWNSGSINYADSVKGRFTISRDNAKNSLYLQMNSLRAEDTALYYCASDSNYRDYYYHYGMDVWGQGTTVTVSS

SEQ ID NO: 109 - DNA, 372 bp, synthetic construct
  source 1..372: mol_type = other DNA
gaagtgcagctggtggagtctgggggaggcttggtacagcctggcaggtccctgagactgtcctgtgcagcctctggattcacctttgatgattatgccatgcactgggtcagacaagctccagggaagggcctggagtgggtctcaggtattagttggaatagtggtagcataaattatgcggactctgtgaagggcaggttcaccatctccagagacaacgccaagaactccctgtatctgcaaatgaacagtctgagagctgaggacacggccttgtattactgtgcgtccgatagtaactatagggactactactaccattacggtatggacgtctgggggcaagggaccacggtcaccgtctcctca

SEQ ID NO: 110 - protein, 454 aa, synthetic construct
EVQLVESGGGLVQPGRSLRLSCAASGFTFDDYAMHWVRQAPGKGLEWVSGISWNSGSINYADSVKGRFTISRDNAKNSLYLQMNSLRAEDTALYYCASDSNYRDYYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 26 aa, synthetic construct
EVQLVESGGGLVQPGRSLRLSCAASG

SEQ ID NO: 112 - protein, 9 aa, synthetic construct
FTFDDYAMH

SEQ ID NO: 113 - protein, 14 aa, synthetic construct
WVRQAPGKGLEWVS

SEQ ID NO: 114 - protein, 17 aa, synthetic construct
GISWNSGSINYADSVKG

SEQ ID NO: 115 - protein, 30 aa, synthetic construct; identical to SEQ ID NO: 105

SEQ ID NO: 116 - protein, 17 aa, synthetic construct
ASDSNYRDYYYHYGMDV

SEQ ID NO: 117 - protein, 11 aa, synthetic construct
WGQGTTVTVSS

SEQ ID NO: 118 - protein, 124 aa, synthetic construct; identical to SEQ ID NO: 108

SEQ ID NO: 119 - DNA, 372 bp, synthetic construct
  source 1..372: mol_type = other DNA
gaagtgcagctggtggagtctgggggaggcttggtacagcctggcaggtccctgagattgtcctgtgcagcctctggattcacctttgatgattatgccatgcactgggtcaggcaagctccagggaagggcctggagtgggtctcaggtattagttggaatagtggtagcataaactatgcggactctgtgaagggcagattcaccatctccagagacaacgccaagaactccctgtatctgcaaatgaacagtctgagagctgaggacacggccttgtattactgtgcttctgatagtaattatagggactactactaccattacggtatggacgtctgggggcaagggaccacggtcaccgtctcctca

SEQ ID NO: 120 - protein, 454 aa, synthetic construct
EVQLVESGGGLVQPGRSLRLSCAASGFTFDDYAMHWVRQAPGKGLEWVSGISWNSGSINYADSVKGRFTISRDNAKNSLYLQMNSLRAEDTALYYCASDSNYRDYYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 121-200 - skipped (80 entries)

SEQ ID NO: 201 - protein, 23 aa, synthetic construct
EIVLTQSPATLSLSPGERATLSC

SEQ ID NO: 202 - protein, 11 aa, synthetic construct
RASENILHYLA

SEQ ID NO: 203 - protein, 15 aa, synthetic construct
WYQQKPGQAPRLVIY

SEQ ID NO: 204 - protein, 7 aa, synthetic construct
DASRRAT

SEQ ID NO: 205 - protein, 32 aa, synthetic construct
GIPGRFSGSGAGTDFTLTINSLEPEDFAVYYC

SEQ ID NO: 206 - protein, 9 aa, synthetic construct
QQRANWPQN

SEQ ID NO: 207 - protein, 10 aa, synthetic construct
FGGGTKVEIK

SEQ ID NO: 208 - protein, 107 aa, synthetic construct
EIVLTQSPATLSLSPGERATLSCRASENILHYLAWYQQKPGQAPRLVIYDASRRATGIPGRFSGSGAGTDFTLTINSLEPEDFAVYYCQQRANWPQNFGGGTKVEIK

SEQ ID NO: 209 - DNA, 321 bp, synthetic construct
  source 1..321: mol_type = other DNA
gaaattgtgttgacacagtctccagccaccctgtctttgtctccaggggaaagagccaccctgtcctgcagggccagtgaaaatatattgcactacttggcctggtaccaacagaaacctggccaggctcccaggcttgtcatctatgatgcatccaggagggccactggcatcccaggaaggttcagtggcagtggggcggggacagacttcactctgaccatcaatagcttggagcctgaagattttgcagtttattactgtcaacaaagagccaattggccccaaaacttcggcggagggaccaaggtggagatcaaa

SEQ ID NO: 210 - protein, 214 aa, synthetic construct
EIVLTQSPATLSLSPGERATLSCRASENILHYLAWYQQKPGQAPRLVIYDASRRATGIPGRFSGSGAGTDFTLTINSLEPEDFAVYYCQQRANWPQNFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 23 aa, synthetic construct
EIVLTQSPATLSLSPGERATLSC

SEQ ID NO: 212 - protein, 11 aa, synthetic construct
RASENILHYLA

SEQ ID NO: 213 - protein, 15 aa, synthetic construct
WYQQKPGQAPRLVIY

SEQ ID NO: 214 - protein, 7 aa, synthetic construct
DASRRAT

SEQ ID NO: 215 - protein, 32 aa, synthetic construct; identical to SEQ ID NO: 205

SEQ ID NO: 216 - protein, 9 aa, synthetic construct
QQRANWPQN

SEQ ID NO: 217 - protein, 10 aa, synthetic construct
FGGGTKVEIK

SEQ ID NO: 218 - protein, 107 aa, synthetic construct; identical to SEQ ID NO: 208

SEQ ID NO: 219 - DNA, 321 bp, synthetic construct
  source 1..321: mol_type = other DNA
gaaattgtgttgacacagtctccagccaccctgtctttgtctccaggggaaagagccaccctttcctgcagggccagtgagaacatcctgcactacttggcctggtaccaacagaaacctggccaggctcccaggttggtaatctatgatgcatccaggagggccactggcatcccaggtaggttcagtggcagtggggcggggacagacttcactctgaccatcaacagcctggagcctgaagattttgcagtttattactgtcaacaaagagccaattggccacagaatttcggcggagggaccaaggtggagatcaaa

SEQ ID NO: 220 - protein, 214 aa, synthetic construct; identical to SEQ ID NO: 210

SEQ ID NO: 221 - protein, 23 aa, synthetic construct
EIVLTQSPATLSLSPGERATLSC

SEQ ID NO: 222 - protein, 11 aa, synthetic construct
RASENILHYLA

SEQ ID NO: 223 - protein, 15 aa, synthetic construct
WYQQKPGQAPRLVIY

SEQ ID NO: 224 - protein, 7 aa, synthetic construct
DASRRAT

SEQ ID NO: 225 - protein, 32 aa, synthetic construct; identical to SEQ ID NO: 205

SEQ ID NO: 226 - protein, 9 aa, synthetic construct
QQRANWPQN

SEQ ID NO: 227 - protein, 10 aa, synthetic construct
FGGGTKVEIK

SEQ ID NO: 228 - protein, 107 aa, synthetic construct; identical to SEQ ID NO: 208

SEQ ID NO: 229 - DNA, 321 bp, synthetic construct
  source 1..321: mol_type = other DNA
gaaattgtgttgacacagtctccagccaccctgtctttgtctccaggggaaagagccaccctttcctgcagggccagtgagaatattttgcactacttggcctggtaccaacagaaacctggccaggctcccaggctggttatctatgatgcatccagaagggccactggcatcccagggaggttcagtggcagtggggccgggacagacttcactctgaccatcaatagcctggagcctgaagattttgcagtttattactgtcaacagagggcgaactggcctcaaaacttcggcggagggaccaaggtggagatcaaa

SEQ ID NO: 230 - protein, 214 aa, synthetic construct; identical to SEQ ID NO: 210

SEQ ID NO: 231 - protein, 23 aa, synthetic construct
EIVLTQSPATLSLSPGERATLSC

SEQ ID NO: 232 - protein, 11 aa, synthetic construct
RASENILHYLA

SEQ ID NO: 233 - protein, 15 aa, synthetic construct
WYQQKPGQAPRLVIY

SEQ ID NO: 234 - protein, 7 aa, synthetic construct
DASRRAT

SEQ ID NO: 235 - protein, 32 aa, synthetic construct; identical to SEQ ID NO: 205

SEQ ID NO: 236 - protein, 9 aa, synthetic construct
QQRANWPQN

SEQ ID NO: 237 - protein, 10 aa, synthetic construct
FGGGTKVEIK

SEQ ID NO: 238 - protein, 107 aa, synthetic construct; identical to SEQ ID NO: 208

SEQ ID NO: 239 - DNA, 321 bp, synthetic construct
  source 1..321: mol_type = other DNA
gaaattgtgttgacacagtctccagccaccctgtctttgtctccaggggaaagagccaccctttcctgcagggccagtgaaaatatcttgcattacttggcctggtaccaacagaaacctggccaggctcccaggctggtgatctatgatgcatccaggagggccactggcatcccaggcaggttcagtggcagtggggcagggacagacttcactttgaccatcaacagccttgagcctgaagattttgcagtttattactgtcaacagagagcaaattggcctcaaaatttcggcggagggaccaaggtggagatcaaa

SEQ ID NO: 240 - protein, 214 aa, synthetic construct; identical to SEQ ID NO: 210

SEQ ID NO: 241 - protein, 22 aa, synthetic construct
QSVLTQPPSVSGAPGQRITISC

SEQ ID NO: 242 - protein, 14 aa, synthetic construct
TGSSSNIGAGYDVH

SEQ ID NO: 243 - protein, 15 aa, synthetic construct
WYQQLPGTAPKLLIY

SEQ ID NO: 244 - protein, 7 aa, synthetic construct
GSSSRNS

SEQ ID NO: 245 - protein, 32 aa, synthetic construct
GVPDRFSGSKSGTSASLAITGLQAEDEADYYC

SEQ ID NO: 246 - protein, 12 aa, synthetic construct
QSYDSSLSVLYT

SEQ ID NO: 247 - protein, 10 aa, synthetic construct
FGTGTKVTVL

SEQ ID NO: 248 - protein, 112 aa, synthetic construct
QSVLTQPPSVSGAPGQRITISCTGSSSNIGAGYDVHWYQQLPGTAPKLLIYGSSSRNSGVPDRFSGSKSGTSASLAITGLQAEDEADYYCQSYDSSLSVLYTFGTGTKVTVL

SEQ ID NO: 249 - DNA, 336 bp, synthetic construct
  source 1..336: mol_type = other DNA
cagtctgtgctgacgcagccgccctcagtgtctggggccccagggcagaggattaccatctcctgcactgggagcagctccaacatcggggcaggttatgatgtacactggtaccagcagcttccaggaacagcccccaaactgttgatctatggtagtagctccaggaattcaggggtccctgacagattctctggctccaagtctggcacctcagcctccctggccatcactgggttgcaggctgaggatgaggctgattattactgccaatcatacgacagctcactgtccgtgctttatacgttcggaactgggaccaaggtcaccgtcctg

SEQ ID NO: 250 - protein, 218 aa, synthetic construct
QSVLTQPPSVSGAPGQRITISCTGSSSNIGAGYDVHWYQQLPGTAPKLLIYGSSSRNSGVPDRFSGSKSGTSASLAITGLQAEDEADYYCQSYDSSLSVLYTFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 251 - protein, 22 aa, synthetic construct
QSVLTQPPSVSGAPGQRITISC

SEQ ID NO: 252 - protein, 14 aa, synthetic construct
TGSSSNIGAGYDVH

SEQ ID NO: 253 - protein, 15 aa, synthetic construct
WYQQLPGTAPKLLIY

SEQ ID NO: 254 - protein, 7 aa, synthetic construct
GSSSRNS

SEQ ID NO: 255 - protein, 32 aa, synthetic construct; identical to SEQ ID NO: 245

SEQ ID NO: 256 - protein, 12 aa, synthetic construct
QSYDSSLSVLYT

SEQ ID NO: 257 - protein, 10 aa, synthetic construct
FGTGTKVTVL

SEQ ID NO: 258 - protein, 112 aa, synthetic construct; identical to SEQ ID NO: 248

SEQ ID NO: 259 - DNA, 336 bp, synthetic construct
  source 1..336: mol_type = other DNA
cagtctgtgctgacgcagccgccctcagtgtctggggccccagggcagaggatcaccatctcctgcactgggagcagctccaacatcggggcaggttatgatgtacactggtaccagcagcttccaggaacagcccccaaacttctgatctatggttctagctccaggaactcaggggtccctgacaggttctctggctccaagtctggcacctcagcctccctggccatcactgggcttcaggctgaggatgaggctgattattactgccaaagttatgactctagtctgtccgtcttgtatacgttcggaactgggaccaaggtcaccgtcctg

SEQ ID NO: 260 - protein, 218 aa, synthetic construct; identical to SEQ ID NO: 250

SEQ ID NO: 261 - protein, 22 aa, synthetic construct
QSVLTQPPSASGTPGQRVTISC

SEQ ID NO: 262 - protein, 13 aa, synthetic construct
SGGSSNIGSNSVN

SEQ ID NO: 263 - protein, 15 aa, synthetic construct
WYQQLPGTAPKLLIY

SEQ ID NO: 264 - protein, 7 aa, synthetic construct
SNSQRPS

SEQ ID NO: 265 - protein, 32 aa, synthetic construct
GVPDRFSGSKSGTSASLAISGLQSEDEADYYC

SEQ ID NO: 266 - protein, 13 aa, synthetic construct
AAWDDSLNTFRYV

SEQ ID NO: 267 - protein, 10 aa, synthetic construct
FGTGTKVTVL

SEQ ID NO: 268 - protein, 112 aa, synthetic construct
QSVLTQPPSASGTPGQRVTISCSGGSSNIGSNSVNWYQQLPGTAPKLLIYSNSQRPSGVPDRFSGSKSGTSASLAISGLQSEDEADYYCAAWDDSLNTFRYVFGTGTKVTVL

SEQ ID NO: 269 - DNA, 336 bp, synthetic construct
  source 1..336: mol_type = other DNA
cagtctgtgctgactcagccaccctcagcgtctgggacccccgggcagagggtcaccatctcttgttctggaggtagctccaacatcggaagtaattcagtaaactggtaccagcagttgccaggaacggcccccaaacttcttatctatagtaattctcagagaccctcaggggtccctgacaggttctctggctccaagtctggcacctcagcctccctggccatcagtgggttgcagtctgaggatgaggctgattattactgtgcagcatgggacgattccttgaatactttcagatatgtcttcggaactgggaccaaggtcaccgtcctg

SEQ ID NO: 270 - protein, 218 aa, synthetic construct
QSVLTQPPSASGTPGQRVTISCSGGSSNIGSNSVNWYQQLPGTAPKLLIYSNSQRPSGVPDRFSGSKSGTSASLAISGLQSEDEADYYCAAWDDSLNTFRYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 271 - protein, 22 aa, synthetic construct
QSVLTQPPSASGTPGQRVTISC

SEQ ID NO: 272 - protein, 13 aa, synthetic construct
SGGSSNIGSNSVN

SEQ ID NO: 273 - protein, 15 aa, synthetic construct
WYQQLPGTAPKLLIY

SEQ ID NO: 274 - protein, 7 aa, synthetic construct
SNSQRPS

SEQ ID NO: 275 - protein, 32 aa, synthetic construct; identical to SEQ ID NO: 265

SEQ ID NO: 276 - protein, 13 aa, synthetic construct
AAWDDSLNTFRYV

SEQ ID NO: 277 - protein, 10 aa, synthetic construct
FGTGTKVTVL

SEQ ID NO: 278 - protein, 112 aa, synthetic construct; identical to SEQ ID NO: 268

SEQ ID NO: 279 - DNA, 336 bp, synthetic construct
  source 1..336: mol_type = other DNA
cagtctgtgctgactcagccaccctcagcgtctgggacccccgggcagagggtcaccatctcttgttctggaggcagctccaacatcggaagtaatagcgtaaactggtaccagcagttgccaggaacggcccccaaattgttgatctatagtaatagtcagaggccctcaggggtccctgacagattctctggctccaagtctggcacctcagcctccctggccatcagtgggcttcagtctgaggatgaggctgattattactgtgctgcatgggacgattctttgaacactttcaggtatgtcttcggaactgggaccaaggtcaccgtcctt

SEQ ID NO: 280 - protein, 218 aa, synthetic construct; identical to SEQ ID NO: 270

SEQ ID NO: 281 - protein, 23 aa, synthetic construct
DIQMTQSPSSLSASVGDRVTITC

SEQ ID NO: 282 - protein, 11 aa, synthetic construct
QASQDISNYLN

SEQ ID NO: 283 - protein, 15 aa, synthetic construct
WYQQKPGKAPKLLIY

SEQ ID NO: 284 - protein, 7 aa, synthetic construct
DASNLET

SEQ ID NO: 285 - protein, 32 aa, synthetic construct
GVPSRFSGSGSGTDFTFTISSLQPEDIATYYC

SEQ ID NO: 286 - protein, 9 aa, synthetic construct
QQYDNLPLT

SEQ ID NO: 287 - protein, 10 aa, synthetic construct
FGPGTKVDIK

SEQ ID NO: 288 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGPGTKVDIK

SEQ ID NO: 289 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatcttaattggtatcagcagaaaccagggaaagcccctaagttgctgatctacgatgcatccaatttggaaacaggggtcccatcaaggttcagtggaagtggatctgggacagattttactttcaccatcagcagcctgcagcctgaagatattgcaacatattactgtcaacaatacgataatctgcccttgactttcggccctgggaccaaagtggatatcaaa

SEQ ID NO: 290 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - protein, 23 aa, synthetic construct
DIQMTQSPSSLSASVGDRVTITC

SEQ ID NO: 292 - protein, 11 aa, synthetic construct
QASQDISNYLN

SEQ ID NO: 293 - protein, 15 aa, synthetic construct
WYQQKPGKAPKLLIY

SEQ ID NO: 294 - protein, 7 aa, synthetic construct
DASNLET

SEQ ID NO: 295 - protein, 32 aa, synthetic construct; identical to SEQ ID NO: 285

SEQ ID NO: 296 - protein, 9 aa, synthetic construct
QQYDNLPLT

SEQ ID NO: 297 - protein, 10 aa, synthetic construct
FGPGTKVDIK

SEQ ID NO: 298 - protein, 107 aa, synthetic construct; identical to SEQ ID NO: 288

SEQ ID NO: 299 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttgaattggtatcagcagaaaccagggaaagcccctaagctgctgatctacgatgcatccaatttggaaacaggggtcccatcaaggttcagtggaagtggatctgggacagattttactttcaccatcagcagcctgcagcctgaagatattgcaacatattactgtcaacagtatgataacctgccgcttactttcggccctgggaccaaagtggatatcaaa

SEQ ID NO: 300 - protein, 214 aa, synthetic construct; identical to SEQ ID NO: 290

SEQ ID NO: 301 - protein, 23 aa, synthetic construct
DIVMTQSPLSLPVTPGEPASISC

SEQ ID NO: 302 - protein, 16 aa, synthetic construct
RSSQSLLHSNGYNYLD

SEQ ID NO: 303 - protein, 15 aa, synthetic construct
WYLQKPGQSPQLLIY

SEQ ID NO: 304 - protein, 7 aa, synthetic construct
LGSNRAS

SEQ ID NO: 305 - protein, 32 aa, synthetic construct
GVPDRFSGSGSGTDFTLKISRVEAEDVGVYYC

SEQ ID NO: 306 - protein, 9 aa, synthetic construct
MQALQTPRT

SEQ ID NO: 307 - protein, 10 aa, synthetic construct
FGQGTKVEIK

SEQ ID NO: 308 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 309 - DNA, 336 bp, synthetic construct
  source 1..336: mol_type = other DNA
gatattgtgatgactcagtctccactttccctgcccgtcacccctggagagccggcctccatctcctgcaggtctagtcagagcctgctgcatagtaatggatacaactatttggattggtacctgcagaagccagggcagtctccacagcttctgatctatttgggttctaatagggcctccggggtccctgacaggttcagtggcagtggatcaggcacagattttacactgaaaatcagcagagtggaggctgaggatgttggggtttattactgcatgcaagccctgcagactccaaggacgttcggccaagggaccaaggtggaaatcaaa

SEQ ID NO: 310 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 23 aa, synthetic construct
DIVMTQSPLSLPVTPGEPASISC

SEQ ID NO: 312 - protein, 16 aa, synthetic construct
RSSQSLLHSNGYNYLD

SEQ ID NO: 313 - protein, 15 aa, synthetic construct
WYLQKPGQSPQLLIY

SEQ ID NO: 314 - protein, 7 aa, synthetic construct
LGSNRAS

SEQ ID NO: 315 - protein, 32 aa, synthetic construct; identical to SEQ ID NO: 305

SEQ ID NO: 316 - protein, 9 aa, synthetic construct
MQALQTPRT

SEQ ID NO: 317 - protein, 10 aa, synthetic construct
FGQGTKVEIK

SEQ ID NO: 318 - protein, 112 aa, synthetic construct; identical to SEQ ID NO: 308

SEQ ID NO: 319 - DNA, 336 bp, synthetic construct
  source 1..336: mol_type = other DNA
gatattgtgatgactcagtctccactgtccctgcccgtcacccctggagagccggcctccatctcctgcaggtctagtcagagcctgctgcatagtaatggatacaactatttggattggtacctgcagaagccagggcagtctccacagcttctgatctatttgggttctaatagagcctccggggtccctgacaggttcagtggcagtggatcaggcacagattttacactgaaaatcagcagagtggaggctgaggatgttggggtttattactgcatgcaggctttgcagacacctagaacgttcggccaagggaccaaggtggaaatcaaa

SEQ ID NO: 320 - protein, 219 aa, synthetic construct; identical to SEQ ID NO: 310

SEQ ID NO: 321-400 - skipped (80 entries)

SEQ ID NO: 401 - protein, 1288 aa, Severe acute respiratory syndrome coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 402 - DNA, 3864 bp, Severe acute respiratory syndrome coronavirus
  source 1..3864: mol_type = genomic DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaag

SEQ ID NO: 403 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 404 - DNA, 3822 bp, Severe acute respiratory syndrome coronavirus 2
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 405 - RNA, 2000 nt, synthetic construct
  source 1..2000: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 406-410 - skipped (5 entries)

SEQ ID NO: 411 - protein, 220 aa, synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 412 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 413 - protein, 220 aa, synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 414 - protein, 220 aa, synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 415 - protein, 220 aa, synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSPG

SEQ ID NO: 416 - protein, 220 aa, synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 417 - protein, 220 aa, synthetic construct
PPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0425567 A1
(43) Pub. Date: Dec. 26, 2024

(54) PHARMACEUTICAL FORMULATIONS COMPRISING ANTI-CORONAVIRUS S PROTEIN ANTIBODIES AND USES THEREOF

(71) Applicant: Invivyd, Inc., Waltham, MA (US)

(72) Inventors: Rebecca Dabora, Bethlehem, PA (US); Gayathri Ratnaswamy, Encino, CA (US); Sheryl Martin-Moe, Oakland, CA (US); Mark Cornell Manning, Johnstown, CO (US); Ryan Holcolm, Fort Collins, CO (US)

(21) Appl. No.: 18/699,101

(22) PCT Filed: Oct. 7, 2022
(86) PCT No.: PCT/US2022/046089

§ 371 Date: Apr. 5, 2024

Related U.S. Application Data
(60) Provisional application No. 63/253,807, filed on Oct. 8, 2021.

(51) Int. Cl. C07K 16/10

(52) U.S. Cl. CPC C07K 16/1003

(57) ABSTRACT

The present disclosure is directed to pharmaceutical formulations comprising antibodies, and antigen binding fragments thereof, having binding specificity for the S protein of coronaviruses (CoV-S). Other embodiments of the disclosure include the use of pharmaceutical formulations for the diagnosis, assessment, and treatment of diseases and disorders associated with coronaviruses, or the S protein thereof, and conditions where neutralization or inhibition of coronaviruses, or the S protein thereof, would be therapeutically and/or prophylactically beneficial.

### RELATED APPLICATION

The present application claims the benefit of priority to U.S. Provisional Application No. 63/253,807, filed on Oct. 8, 2021. The entire contents of the foregoing application are incorporated herein by reference.

### BACKGROUND

Coronaviruses (“CoV”) are genetically classified into four major genera: the Alphacoronavirus genus (ACoV genus); the Betacoronavirus genus (BCoV genus); the Gammacoronavirus genus (CCoV genus); and Deltacoronavirus genus (DCoV genus), and while ACoV and BCOV primarily infect mammals CCOV and DCOV predominantly infect birds (Wu A, et al., Cell Host Microbe. 2020 Mar. 11; 27 (3): 325-328). Coronaviruses that infect humans were first identified in the mid-1960s, and currently, seven confirmed CoV species are known as human pathogens. Four CoV species, the HCoV-HKU1 and HCoV-OC43 from the BCOV genus and the HCoV-229E and HCoV-NL63 from the ACoV genus, are endemic species in humans and cause mild respiratory symptoms, mostly in pediatric patients (Brielle E. S., et al., BioRxiv reprint, doi: https://doi.org/10.1101/2020.03.10.986398). The other three human CoV species, the SARS-COV, the MERS-COV, and the SARS-COV-2 (also known as “2019-nCOV”), all of which are from the BCoV genus, have caused severe outbreaks, including the Severe Acute Respiratory Syndrome (SARS) outbreak in 2002-2003, the Middle East Respiratory Syndrome (MERS) outbreak in 2012-2013, and the current (2019-) pandemic of the coronavirus disease of 2019 (“COVID-19”).

The genome of coronaviruses, whose size ranges between approximately 26,000 and 32,000 bases, includes a variable number (from 6 to 11) of open reading frames (“ORFs”) (Wu A, et al., Cell Host Microbe. 2020 Mar. 11; 27 (3): 325-328). The first ORF encodes 16 non-structural proteins (“nsps”), and the remaining ORFs encode accessory proteins and structural proteins. The four major structural proteins are the spike surface glycoprotein (“S protein” or “S” or “spike protein”), small envelope protein (“E protein” or “E”), matrix protein (“M protein” or “M”), and nucleocapsid protein (“N protein”, or “N”).

The S protein, which plays an essential role in binding to receptors on the host cell and determines host tropism (Zhu Z, et al., Infect Genet Evol. 2018 July; 61:183-184), forms homotrimers protruding from the viral surface (Li F. Annu Rev Virol. 2016 Sep. 29; 3 (1): 237-261). The S protein is processed into two non-covalently associated subunits, S1 and S2, and each monomer in the trimeric S assembly is a heterodimer of S1 and S2 subunits. Cryo-EM studies have revealed that the S1 subunit is comprised of four domains: an N-terminal domain (NTD), a C-terminal domain (CTD), and two subdomains (Walls A. C, et al., Nature 531, 114-117 (2016).; Tortorici M. A, and Veesler D., Adv Virus Res. 2019; 105:93-116; Wrapp D, et al., Science 367, 1260-1263 (2020)). The CTD functions as the receptor-binding domain (RBD) for both SARS-COV and SARS-COV-2 (Li F. J Virol. 2015 February; 89 (4): 1954-64). The S2 subunit contains the fusion peptide, heptad repeat 1 and 2, and a transmembrane domain, all of which are required to mediate fusion of the viral and host cell membranes.

SARS-COV and SARS-COV-2 bind to and use angiotensin-converting enzyme 2 (ACE2) of a host cell as a receptor to enter the host cells (Ge X. Y, et al., Nature. 2013 Nov. 28; 503 (7477): 535-8; Hoffmann M, et al., Cell. 2020 Mar. 4, pii: S0092-8674 (20) 30229-4). The motif within the RBD that particularly binds to RCE2 is often referred to as the “ACE2-binding motif”. SARS-COV can also use CD209L (also known as L-SIGN) as an alternative receptor (Jeffers S. A, et al., Proc Natl Acad Sci USA. 2004 Nov. 2; 101 (44): 15748-53). In contrast, MERS-COV binds dipeptidyl peptidase 4 (“DPP4”, also known as CD26) of the host cell via a different RBD of the S protein.

Cell entry of coronaviruses often depends also on priming of the S protein by host cell proteases. Recently, SARS-COV-2 was found to use the serine protease TMPRSS2 for S protein priming and ACE2 for entry (Wu A, et al., Cell Host Microbe. 2020 Mar. 11; 27 (3): 325-328; Hoffmann M, et al., Cell. 2020 Mar. 4, pii: S0092-8674 (20) 30229-4).

The genome of SARS-COV-2 is about 29.8 kb nucleotides and encodes 15 nsps, four structural proteins (S. E. M, and N) and eight accessory proteins (3a, 3b, p6, 7a, 7b, 8b, 9b, and orf14) (Wu A, et al., Cell Host Microbe. 2020 Mar. 11; 27 (3): 325-328). While SARS-COV-2 is genetically close to a SARS-like bat CoV and also to SARS-COV, a number of sequence differences have been identified. When SARS-COV-2 is compared to SARS-COV or SARS-like bat CoV, 380 amino acid differences or substitutions were found, 27 of which are in the S protein, including 6 substitutions in the RBD at amino acid region 357-528 (but not in the receptor-binding motifs that directly interact with ACE2) and 6 substitutions in the underpinning subdomain (SD) at amino acid region 569-655.

One of the few drugs approved by the U.S. Food and Drug Administration (“FDA”) for use in treating COVID-19 is the viral replication inhibitor remdesivir. The Emergency Use Authorization allows for remdesivir to be administered intravenously in adults and pediatric patients 12 years of age and greater for the treatment of COVID-19 requiring hospitalization. Clinical trials demonstrated that remdesivir shortens the time to recovery in hospitalized patients, but more effective therapy is in great need. Convalescent plasma received the emergency use authorization status by the FDA. Other treatments given to COVID-19 patients include anti-inflammatoireuch as corticosteroids and other treatments for managing symptoms such as supplemental oxygen and mechanical ventilatory support. Several drugs, particularly those that have been approved for preventing or treating other infectious disease, are currently being tested in the clinic, which includes e.g., lopinavir-ritonavir (HIV protease inhibitor), ABX464 (viral RNA splicer), favilavir (RNA-dependent RNA polymerase inhibitor used for influenza virus infection), niclosamide and ivermectin (antihelmintic), and BCG vaccine (vaccine for tuberculosis). Also, other ongoing clinical trials reportedly are using IL-6 receptor antagonist antibodies, an anti-GM-CSF or anti-GM-CSF receptor antibody, an anti-TNF antibody, an anti-IL-1beta antibody, or an anti-complement component 5 antibody, in an effort to inhibit inflammation and thereby potentially inhibit cytokine storm and sepsis which can manifest in some SARS-COV-2-infected patients and may cause death. However, more effective therapeutic and prophylactic treatments are needed.

### SUMMARY

The applicant has recently discovered compounds specific to coronavirus S protein, such as antibodies and antigen-binding fragments thereof, and therapeutic and prophylactic uses thereof. For example, see PCT/US2021/026574, filed on Apr. 9, 2021, the entire contents of which are expressly incorporated herein by reference. However, providing stable, high concentration antibody formulations with acceptable storage conditions, such as temperature and duration, is challenging (see, e.g., Wang et al., J of Pharmaceutical Sciences, Vol. 96, No. 1, January 2007; and Daugherty et al. (2006) Advanced Drug Delivery Reviews, 58, 686-706; Éva Kollár, et al., Drug Discovery Today: Technologies 2020, 8; Steven. J. Shire et al., Book Chapter in ‘Formulation and Process Development Strategies for Manufacturing Biopharmaceuticals’ (ISBN: 978-0-470-11812-2), Chapter 15: High Concentration antibody formulations). There are a number of critical factors to consider when formulating antibody products, including protein concentration, effect of formulation pH, effect of buffering agents, effect of formulation excipients, effect of shaking, effect of preservatives, effect of processing equipment, and effect of storage containers and storage time. In particular, one challenge associated with high concentration formulations is increased electrostatic interaction between proteins and excipients and is a result of increased protein-charge density at high-protein concentrations. Such interactions can create an offset between excipient levels in final products and diafiltration buffers during the ultrafiltration process.

The inventors of the present disclosure, however, have surprisingly and unexpectedly overcome these challenges and have achieved a final formulation having a high concentration e.g., greater than 100 mg/mL, of anti-coronavirus S protein antibody, or antigen-binding fragment thereof, with optimal characteristics, e.g., 150 mg/mL, or 200 mg/mL; a viscosity of under 20 cPoise; a physiological isotonicity, e.g., between 270-400 mOsm/kg; an opalescence less than about 60 NTU opalescence; and/or a high level of stability at about 2-8° C., e.g., a shelf-life of at least 2 years, and at about 25° C., e.g., a shelf-life of at least 2 weeks. The high concentration formulation surprisingly enables intramuscular (IM) administration. In addition, the high concentration formulation of the present disclosure is also stable upon freezing and thawing, thereby enabling the drug substance (i.e., stored frozen) to have the same formulation and product concentration as the drug product.

Accordingly, in one aspect, the present disclosure provides a pharmaceutical formulation comprising (i) an isolated antibody, or antigen-binding fragment thereof, which binds to the spike protein of a coronavirus (“CoV-S”); (ii) one or more buffers selected from the group consisting of an acetate buffer, a succinate buffer, a citrate buffer, a histidine buffer and a phosphate buffer having a pH of about 4.5-7.0; (iii) one or more pharmaceutically acceptable excipients selected from the group consisting of sucrose, mannitol, glycine, proline, sodium chloride, arginine hydrochloride, arginine-glutamate, and sorbitol; and/or (iv) a surfactant.

In some embodiments, the buffer has a concentration of about 1-50 mM, about 1-30 mM, about 1-20 mM, about 1-10 mM, or about 5-15 mM.

In some embodiments, the buffer has a concentration of about 1 mM, about 10 mM, about 20 mM, about 30 mM, about 40 Mm, or about 50 mM.

In some embodiments, the buffer is a histidine buffer.

In some embodiments, the buffer is an acetate buffer.

In some embodiments, the pharmaceutically acceptable excipient is sucrose. In some embodiments, the sucrose concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the sucrose concentration is about 1 mM, about 50 mM, about 60 mM, about 75 mM, about 80 mM, about 90 mM, about 100 mM, about 110 mM, about 125 mM, about 145 mM, about 150 mM, about 200 mM, about 235 mM, about 250 mM, or about 300 mM.

In some embodiments, the pharmaceutically acceptable excipient is mannitol. In some embodiments, the mannitol concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the mannitol concentration is about 1 mM, about 50 mM, about 60 mM, about 75 mM, about 80 mM, about 90 mM, about 100 mM, about 110 mM, about 125 mM, about 145 mM, about 150 mM, about 200 mM, about 235 mM, about 250 mM, or about 300 mM.

In some embodiments, the pharmaceutically acceptable excipient is arginine hydrochloride. In some embodiments, the arginine hydrochloride concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the arginine hydrochloride concentration is about 1 mM, about 50 mM, about 75 mM, about 80 mM, about 90 mM, about 100 mM, about 110 mM, about 125 mM, about 150 mM, about 200 mM, about 250 mM, about 270 mM, or about 300 mM.

In some embodiments, the pharmaceutically acceptable excipient is glycine. In some embodiments, the glycine concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the glycine concentration is about 1 mM, about 50 mM, about 75 mM, about 80 mM, about 90 mM, about 100 mM, about 110 mM, about 125 mM, about 150 mM, about 200 mM, about 250 mM, about 270 mM, or about 300 mM.

In some embodiments, the pharmaceutically acceptable excipient is proline. In some embodiments, the proline concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the proline concentration is about 1 mM, about 50 mM, about 75 mM, about 80 mM, about 90 mM, about 100 mM, about 110 mM, about 125 mM, about 150 mM, about 200 mM, about 250 mM, about 270 mM, or about 300 mM.

In some embodiments, the pharmaceutically acceptable excipient is sodium chloride. In some embodiments, the sodium chloride concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the sodium chloride concentration is about 1 mM, about 50 mM, about 75 mM, about 80 mM, about 90 mM, about 100 mM, about 110 mM, about 125 mM, about 140 mM, about 150 mM, about 200 mM, about 250 mM, about 270 mM, or about 300 mM.

In some embodiments, the pharmaceutically acceptable excipient is arginine glutamate. In some embodiments, the arginine glutamate concentration is about 1-300 mM, about 1-250 mM, about 1-150 mM, about 50-150 mM, about 50-100 mM, about 75-125 mM, about 50-200 mM, about 100-300 mM, or about 150-300 mM. In some embodiments, the arginine glutamate concentration is about 1 mM, about 50 mM, about 75 mM, about 80 mM, about 90 mM, about 100 mM, about 110 mM, about 125 mM, about 140 mM, about 150 mM, about 200 mM, about 250 mM, about 270 mM, or about 300 mM.

In some embodiments, the pharmaceutically acceptable excipient is sorbitol. In some embodiments, the sorbitol concentration is about 1-300 mM, about 1-260 mM, about 1-150 mM, about 50-150 mM, about 50-100 mM, about 75-125 mM, about 50-200 mM, about 100-300 mM, or about 150-300 mM. In some embodiments, the sorbitol concentration is about 1 mM, about 50 mM, about 75 mM, about 80 mM, about 90 mM, about 100 mM, about 110 mM, about 125 mM, about 140 mM, about 150 mM, about 180 mM, about 200 mM, about 250 mM, about 260 mM, about 270 mM, or about 300 mM.

In some embodiments, the surfactant is Polysorbate 80 (PS80). In some embodiments, the PS80 is present at about 0.01-1.0% w/v, about 0.01-0.06% w/v, about 0.01-0.05% w/v, about 0.015-0.045% w/v, or about 0.02-0.04% w/v. In some embodiments, the PS80 is present at about 0.01% w/v, about 0.015% w/v, about 0.02% w/v, about 0.03% w/v, about 0.04% w/v, about 0.045% w/v, about 0.05% w/v, about 0.06% w/v, or about 0.07% w/v.

In some embodiments, the formulation has a pH of about 5.0-7.0, about 5.0-6.0, about 5.2-6.0, about 5.2-5.8, about 5.4-5.7, or about 5.4-5.6.

In some embodiments, the formulation has a pH of about 4.0, about 4.5, about 5.0, about 5.1, about 5.2, about 5.3, about 5.4, about 5.5, about 5.6, about 5.7, about 5.8, about 5.9, about 6.0, about 6.5, or about 7.0.

In some embodiments, the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO: 254, and a VL CDR3 comprising SEQ ID NO:256.

In some embodiments, the antibody, or antigen-binding fragment thereof, comprises a VH having at least 95% identity to SEQ ID NO:58 and a VL having at least 95% identity to SEQ ID NO: 258.

In some embodiments, the antibody, or antigen-binding fragment thereof, comprises a VH comprising of SEQ ID NO:58 and a VL comprising of SEQ ID NO:258.

In some embodiments, the antibody, or antigen-binding fragment thereof, comprises a heavy chain variable region (VH) comprising a VH CDR1 comprising SEQ ID NO:22, a VH CDR2 comprising SEQ ID NO:24, and a VH CDR3 comprising SEQ ID NO:26, and a light chain variable region (VL) comprising a VL CDR1 comprising SEQ ID NO:222, a VL CDR2 comprising SEQ ID NO: 224, and a VL CDR3 comprising SEQ ID NO:226.

In some embodiments, the antibody, or antigen-binding fragment thereof, comprises a VH having at least 95% identity to SEQ ID NO:28 and a VL having at least 95% identity to SEQ ID NO: 228.

In some embodiments, the antibody, or antigen-binding fragment thereof, comprises a VH comprising of SEQ ID NO:28 and a VL comprising of SEQ ID NO:228.

In some embodiments, the antibody, or antigen-binding fragment thereof, has a concentration of about 10-500 mg/mL, about 50-400 mg/mL, about 100-300 mg/mL, about 100-200 mg/mL, or about 125-175 mg/mL, or about 130-170 mg/mL.

In some embodiments, the antibody, or antigen-binding fragment thereof, has a concentration of about 10 mg/mL, about 50 mg/mL, about 100 mg/mL, about 125 mg/mL, about 130 mg/mL about 150 mg/mL, about 170 mg/mL, about 175 mg/mL, about 200 mg/mL, about 250 mg/mL, about 300 mg/mL, about 400 mg/mL, or about 500 mg/mL.

In another aspect, provided herein is a pharmaceutical formulation comprising (i) an isolated antibody, or antigen-binding fragment thereof, which binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) about 1-20 mM histidine or acetate buffer having a pH of about 5.0-6.0; (iii) about 50-150 mM sucrose or mannitol; (iv) about 50-100 mM arginine hydrochloride; and (v) about 0.01-0.05% w/v polysorbate 80.

In some embodiments, the antibody, or antigen-binding fragment thereof, has a concentration of about 10-500 mg/mL, about 50-400 mg/mL, about 100-300 mg/mL, about 100-200 mg/mL, or about 125-175 mg/mL, or about 130-170 mg/mL.

In some embodiments, the antibody, or antigen-binding fragment thereof, has a concentration of about 10 mg/mL, about 50 mg/mL, about 100 mg/mL, about 125 mg/mL, about 130 mg/mL about 150 mg/mL, about 170 mg/mL, about 175 mg/mL, about 200 mg/mL, about 250 mg/mL, about 300 mg/mL, about 400 mg/mL, or about 500 mg/mL.

In some embodiments, the concentration of the antibody, or antigen-binding fragment thereof, is about 150 mg/mL.

In some embodiments, the pharmaceutical formulation comprises (i) about 150 mg/mL of the isolated antibody, or antigen-binding fragment thereof, (ii) about 10 mM histidine buffer having a pH of about 5.2-6.0; (iii) about 100 mM sucrose; (iv) about 75 mM arginine hydrochloride; and (v) about 0.03% w/v polysorbate 80.

In some embodiments, the pharmaceutical formulation comprises (i) about 150 mg/mL of the isolated antibody, or antigen-binding fragment thereof, (ii) about 10 mM histidine buffer having a pH of about 5.2-5.8; (iii) about 100 mM sucrose; (iv) about 75 mM arginine hydrochloride; and (v) about 0.03% w/v polysorbate 80.

In some embodiments, the pharmaceutical formulation comprises (i) about 150 mg/mL of the isolated antibody, or antigen-binding fragment thereof, (ii) about 10 mM histidine buffer having a pH of about 5.4-5.6; (iii) about 100 mM sucrose; (iv) about 75 mM arginine hydrochloride; and (v) about 0.03% w/v polysorbate 80.

In some embodiments, the pharmaceutical formulation comprises (i) about 150 mg/mL of the isolated antibody, or antigen-binding fragment thereof, (ii) about 10 mM histidine buffer having a pH of about 5.4; (iii) about 100 mM sucrose; (iv) about 75 mM arginine hydrochloride; and (v) about 0.03% w/v polysorbate 80.

In some embodiments, the pharmaceutical formulation comprises (i) about 150 mg/mL of the isolated antibody, or antigen-binding fragment thereof, (ii) about 10 mM histidine buffer having a pH of about 5.5; (iii) about 100 mM sucrose; (iv) about 75 mM arginine hydrochloride; and (v) about 0.03% w/v polysorbate 80.

In some embodiments, the pharmaceutical formulation comprises (i) about 150 mg/mL of the isolated antibody, or antigen-binding fragment thereof, (ii) about 10 mM histidine buffer having a pH of about 5.6; (iii) about 100 mM sucrose; (iv) about 75 mM arginine hydrochloride; and (v) about 0.03% w/v polysorbate 80.

In some embodiments, the formulation is stable at about 2-8° C. for at least 1, 2 or 3 years.

In some embodiments, the formulation is stable at about 25° C., or about 40° C. for at least one, two, or four weeks.

In some embodiments, the formulation is stable at about ≤−30° C. for at least 1, 2, 3, 4 or 5 years.

In one aspect, provided herein is a method of inducing an immune response against SARS-CoV. SARS-COV-2, or another coronavirus selected from the group consisting of MERS-COV. HCoV-HKU1, HCoV-OC43, HCOV-229E, and HCoV-NL63 in a subject in need thereof, the method comprising administering a pharmaceutical formulation disclosed herein.

In another aspect, provided herein is a method of preventing infection of susceptible cells by SARS-COV. SARS-COV-2, or another coronavirus selected from the group consisting of MERS-COV, HCoV-HKU1, HCoV-OC43, HCOV-229E, and HCoV-NL63 in a subject in need thereof, the method comprising administering a pharmaceutical formulation disclosed herein.

In one aspect, provided herein is a method of treating a coronavirus infection of a subject by SARS-COV. SARS-COV-2, or another coronavirus selected from the group consisting of MERS-COV, HCoV-HKU1, HCoV-OC43, HCOV-229E, and HCoV-NL63 in a subject in need thereof, the method comprising administering a pharmaceutical formulation disclosed herein.

In another aspect, provided herein is a method of treating a symptom of an infection of a subject by SARS-COV, SARS-COV-2, or another coronavirus selected from the group consisting of MERS-COV, HCoV-HKU1, HCoV-OC43, HCOV-229E, and HCoV-NL63 in a subject in need thereof, the method comprising administering a pharmaceutical formulation disclosed herein.

In some embodiments, the symptom comprises at least one of bronchitis, pneumonia, respiratory failure, acute respiratory failure, organ failure, multi-organ system failure, pediatric inflammatory multisystem syndrome, acute respiratory distress syndrome, blood clot, a cardiac condition, myocardial injury, myocarditis, heart failure, cardiac arrest, acute myocardial infarction, dysrhythmia, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, a gastrointestinal complication, aspergillosis, increased susceptibility to infection with another virus or bacteria, and/or a pregnancy-related complication.

In one aspect, provided herein is a method of decreasing the risk of mortality, hospitalization, mechanical ventilation, or a combination thereof, in a subject infected by SARS-COV, SARS-COV-2, or another coronavirus selected from the group consisting of MERS-COV, HCoV-HKU1, HCOV-OC43, HCoV-229E, and HCoV-NL63, the method comprising administering a pharmaceutical formulation disclosed herein.

In some embodiments, the subject is a human subject.

In some embodiments, the subject has at least one risk factor which renders them more prone to a poor clinical outcome.

In some embodiments, the at least one risk factor is selected from the group consisting of: an old age selected from the group consisting of over 55, over 60 or over 65 years old; diabetes, a chronic respiratory condition, obesity, hypertension, a cardiac or cardiovascular condition, a chronic inflammatory or autoimmune condition, and an immune compromised status.

In some embodiments, the pharmaceutical formulation is administered intramuscularly or intravenously.

In some embodiments, the pharmaceutical formulation is administered once or is administered yearly, monthly, or weekly.

In one aspect, the present disclosure relates to a compound which binds to coronavirus (CoV) or the spike protein (S protein) of a CoV (“CoV-S”). In some embodiments, the compound may be an isolated antibody or antigen-binding antibody fragment which binds to a CoV-S. In some embodiments, the antibody or antigen-binding antibody fragment may comprise a heavy chain variable region (VH), or fragments thereof, and/or a light chain variable region (VL), or fragments thereof. In certain embodiments, the VH or fragment thereof may comprise a complementarity-determining region 1 (CDR1), a complementarity-determining region 2 (CDR2), and a complementarity-determining region 3 (CDR3), which may also be referred to as VH CDR1, VH CDR2, and VH CDR3, respectively. In certain embodiments, the VL or fragment thereof may comprise a CDR1, a CDR2, and a CDR3, which may also be referred to as VL CDR1, VL CDR2, and VL CDR3, respectively. In some embodiments, the antibody, or antigen-binding antibody fragment thereof, may comprise a heavy chain CDR1, a heavy chain CDR2, a heavy chain CDR3, a light chain CDR1, a light chain CDR2, and a light chain CDR3.

In some embodiments, the antibody or antigen-binding antibody fragment may comprise an antibody or antigen-binding antibody fragment thereof, or an affinity-matured variant of an anti-CoV-S antibody or antigen-binding antibody fragment thereof; selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, optionally wherein the CoV-S is SARS-COV—S or SARS-COV-2-S.

In some embodiments, the antibody, or antigen-binding antibody fragment thereof, may comprise a VH and/or VL. In certain embodiments, the VH may comprise a CDR3 having an amino acid sequence identical to the VH CDR3 of any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, and optionally, the VL CDR3 may comprise a CDR3 having an amino acid sequence identical to the VL CDR3 of the same anti-CoV-S antibody that the VH CDR3 is derived from, and the anti-CoV-S antibody may be selected from any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131. Here, the CoV-S may be the spike protein (“S protein”) of Severe Acute Respiratory Syndrome (SARS) coronavirus (“SARS-COV”), which may be referred to as “SARS—CoV-S”, or the S protein of SARS-COV-2 (also known as “n2019-nCOV”), which may be referred to as “SARS-COV-2-S”. Optionally, the CoV-S may comprise a sequence having at least 90%, 91%, 92%, 93%, 94%, 95%, 96%, 97%, 98%, 99% identity to, comprising, or consisting of the amino acid sequence of SEQ ID NO: 401 (SARS-COV—S, 1288 amino acids, Accession #PDB: 6VSB_B) or having at least 90%, 91%, 92%, 93%, 94%, 95%, 96%, 97%, 98%, 99% identity to, comprising, or consisting of SEQ ID NO: 403 (SARS-COV-2-S, 1273 amino acids, GenBank: QHD43416.1).

In some embodiments, the SARS-COV-2-S is a B.1.1.7 variant, a B. 1.351 variant, a B.1.1.28 variant, a B. 1.429 variant, a P.1 variant, a B.1617 variant (e.g., B.1.617.1 and B.1.672.2), a C.37 variant, a 1.621 variant, an AY.1 variant, or a D614G variant of SEQ ID NO: 403.

In some embodiments, the antibody or antigen-binding antibody fragment, optionally an affinity-matured variant of any of the anti-CoV-S antibodies disclosed herein, may comprise at least 1, 2, 3, 4, 5 or all 6 complementarity-determining regions (CDRs) of any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, optionally wherein the CoV-S is SARS-COV—S or SARS-COV-2-S. Optionally, the CoV-S may comprise the amino acid sequence of SEQ ID NO: 401 (SARS-COV—S, 1288 amino acids, Accession #PDB: 6VSB_B) or SEQ ID NO: 403 (SARS-COV-2-S, 1273 amino acids, GenBank: QHD43416.1).

In some embodiments, the isolated antibody or antigen-binding antibody fragment, optionally an affinity-matured variant of any of the anti-CoV-S antibodies disclosed herein, may comprise: (a) a VH CDR1 polypeptide; (b) a VH CDR2 polypeptide; (c) a VH CDR3 polypeptide; (d) a VL CDR1 polypeptide; (e) a VL CDR2 polypeptide; and (f) a VL CDR3 polypeptide. The amino acid sequences of the VH CDR1, the VH CDR2, the VH CDR3, the VL CDR1, the VL CDR2, and the VL CDR3 may be identical to the amino acid sequences of the VH CDR1, VH CDR2, VH CDR3, VL CDR1, VL CDR2, and VL CDR3, respectively, of any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131. Optionally, the CoV-S may be SARS—CoV—S or of “SARS-COV-2-S”. Further optionally, the CoV-S may comprise the amino acid sequence of SEQ ID NO: 401 (SARS-COV—S, 1288 amino acids, Accession #PDB: 6VSB_B) or SEQ ID NO: 403 (SARS-COV-2-S, 1273 amino acids, GenBank: QHD43416.1).

In certain embodiments, the isolated antibody or antigen-binding antibody fragment, optionally an affinity-matured variant of any of the anti-CoV-S antibodies disclosed herein, which specifically binds to CoV-S, may comprise: (a) a VH comprising a VH CDR1, VH CDR2, and VH CDR3; and (b) a VL comprising a VL CDR1, VL CDR2, and VL CDR3.

In some exemplary embodiments, the amino acid sequences of the VH CDR1, the VH CDR2, the VH CDR3, the VL CDR1, the VL CDR2, and the VL CDR3 may be identical to the amino acid sequences of:
(1) SEQ ID NOS: 2, 4, 6, 202, 204, and 206, respectively;
(2) SEQ ID NOS: 12, 14, 16, 212, 214, and 216, respectively;
(3) SEQ ID NOS: 22, 24, 26, 222, 224, and 226, respectively;
(4) SEQ ID NOS: 32, 34, 36, 232, 234, and 236, respectively;
(5) SEQ ID NOS: 42, 44, 46, 242, 244, and 246, respectively;
(6) SEQ ID NOS: 52, 54, 56, 252, 254, and 256, respectively;
(7) SEQ ID NOS: 62, 64, 66, 262, 264, and 66, respectively;
(8) SEQ ID NOS: 72, 74, 76, 272, 274, and 276, respectively;
(9) SEQ ID NOS: 82, 84, 86, 282, 284, and 286, respectively;
(10) SEQ ID NOS: 92, 94, 96, 292, 294, and 296, respectively;
(11) SEQ ID NOS: 102, 104, 106, 302, 304, and 306, respectively; or
(12) SEQ ID NOS: 112, 114, 116, 312, 314, and 316, respectively.

In other words, the amino acid sequences of the VH CDR1, the VH CDR2, the VH CDR3, the VL CDR1, the VL CDR2, and the VL CDR3 may be identical to the VH CDR1, the VH CDR2, the VH CDR3, the VL CDR1, the VL CDR2, and the VL CDR3 and VL amino acid sequences of any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131.

In some embodiments, the isolated antibody or antigen-binding antibody fragment, optionally an affinity-matured variant of any of the anti-CoV-S antibodies disclosed herein, may possess one of the following structural features:
(1) (a) the VH may comprise an amino acid sequence with at least 90, 91, 92, 93, 94, 95, 96, 97, 98, 99, or 100% sequence identity to the amino acid sequence of SEQ ID NO: 8, and (b) the VL may comprise an amino acid sequence with at least 90, 91, 92, 93, 94, 95, 96, 97, 98, 99, or 100% sequence identity to the amino acid sequence of SEQ ID NO: 208;
(2) (a) the VH may comprise an amino acid sequence with at least 90, 91, 92, 93, 94, 95, 96, 97, 98, 99, or 100% sequence identity to the amino acid sequence of SEQ ID NO: 18, and (b) the VL may comprise an amino acid sequence with at least 90, 91, 92, 93, 94, 95, 96, 97, 98, 99, or 100% sequence identity to the amino acid sequence of SEQ ID NO: 218;
(3) (a) the VH may comprise an amino acid sequence with at least 90, 91, 92, 93, 94, 95, 96, 97, 98, 99, or 100% sequence identity to the amino acid sequence of SEQ ID NO: 28, and (b) the VL may comprise an amino acid sequence with at least 90, 91, 92, 93, 94, 95, 96, 97, 98, 99, or 100% sequence identity to the amino acid sequence of SEQ ID NO: 228;
(4) (a) the VH may comprise an amino acid sequence with at least 90, 91, 92, 93, 94, 95, 96, 97, 98, 99, or 100% sequence identity to the amino acid sequence of SEQ ID NO: 38, and (b) the VL may comprise an amino acid sequence with at least 90, 91, 92, 93, 94, 95, 96, 97, 98, 99, or 100% sequence identity to the amino acid sequence of SEQ ID NO: 238;
(5) (a) the VH may comprise an amino acid sequence with at least 90, 91, 92, 93, 94, 95, 96, 97, 98, 99, or 100% sequence identity to the amino acid sequence of SEQ ID NO: 48, and (b) the VL may comprise an amino acid sequence with at least 90, 91, 92, 93, 94, 95, 96, 97, 98, 99, or 100% sequence identity to the amino acid sequence of SEQ ID NO: 248;
(6) (a) the VH may comprise an amino acid sequence with at least 90, 91, 92, 93, 94, 95, 96, 97, 98, 99, or 100% sequence identity to the amino acid sequence of SEQ ID NO: 58, and (b) the VL may comprise an amino acid sequence with at least 90, 91, 92, 93, 94, 95, 96, 97, 98, 99, or 100% sequence identity to the amino acid sequence of SEQ ID NO: 258;
(7) (a) the VH may comprise an amino acid sequence with at least 90, 91, 92, 93, 94, 95, 96, 97, 98, 99, or 100% sequence identity to the amino acid sequence of SEQ ID NO: 68, and (b) the VL may comprise an amino acid sequence with at least 90, 91, 92, 93, 94, 95, 96, 97, 98, 99, or 100% sequence identity to the amino acid sequence of SEQ ID NO: 268;
(8) (a) the VH may comprise an amino acid sequence with at least 90, 91, 92, 93, 94, 95, 96, 97, 98, 99, or 100% sequence identity to the amino acid sequence of SEQ ID NO: 78, and (b) the VL may comprise an amino acid sequence with at least 90, 91, 92, 93, 94, 95, 96, 97, 98, 99, or 100% sequence identity to the amino acid sequence of SEQ ID NO: 278;
(9) (a) the VH may comprise an amino acid sequence with at least 90, 91, 92, 93, 94, 95, 96, 97, 98, 99, or 100% sequence identity to the amino acid sequence of SEQ ID NO: 88, and (b) the VL may comprise an amino acid sequence with at least 90, 91, 92, 93, 94, 95, 96, 97, 98, 99, or 100% sequence identity to the amino acid sequence of SEQ ID NO: 288;
(10) (a) the VH may comprise an amino acid sequence with at least 90, 91, 92, 93, 94, 95, 96, 97, 98, 99, or 100% sequence identity to the amino acid sequence of SEQ ID NO: 98, and (b) the VL may comprise an amino acid sequence with at least 90, 91, 92, 93, 94, 95, 96, 97, 98, 99, or 100% sequence identity to the amino acid sequence of SEQ ID NO: 298;
(11) (a) the VH may comprise an amino acid sequence with at least 90, 91, 92, 93, 94, 95, 96, 97, 98, 99, or 100% sequence identity to the amino acid sequence of SEQ ID NO: 108, and (b) the VL may comprise an amino acid sequence with at least 90, 91, 92, 93, 94, 95, 96, 97, 98, 99, or 100% sequence identity to the amino acid sequence of SEQ ID NO: 308; or
(12) (a) the VH may comprise an amino acid sequence with at least 90, 91, 92, 93, 94, 95, 96, 97, 98, 99, or 100% sequence identity to the amino acid sequence of SEQ ID NO: 118, and (b) the VL may comprise an amino acid sequence with at least 90, 91, 92, 93, 94, 95, 96, 97, 98, 99, or 100% sequence identity to the amino acid sequence of SEQ ID NO: 318.

In some exemplary embodiments, the isolated antibody or antigen-binding antibody fragment, optionally an affinity-matured variant, may be human, humanized, primatized or chimeric.

In some exemplary embodiments, the isolated antibody or antigen-binding antibody fragment, optionally an affinity-matured variant, may be bispecific or multispecific.

In some exemplary embodiments, the isolated antibody or antigen-binding antibody fragment, optionally an affinity-matured variant, may comprise at least one first antigen-binding domain (“ABD”) and at least one second ABD.

Here, the following features (a) and (b) may be met:
(a) the first ABD may comprise the VH CDR1, the VH CDR2, the VH CDR3, the VL CDR1, the VL CDR2, and the VL CDR3 of a first anti-CoV-S antibody selected from any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131; and/or
(b) the second ABD may comprise the VH CDR1, the VH CDR2, the VH CDR3, the VL CDR1, the VL CDR2, and the VL CDR3 of a second anti-CoV-S antibody selected from any one of anti-CoV-S antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131.

Optionally, the first anti-CoV-S antibody may be same as the second anti-CoV-S antibody or may be different from the second anti-CoV-S antibody.

The first anti-CoV-S antibody and the second anti-CoV-S antibody may bind to the same or different coronavirus species. Optionally, the first CoV-S and the second CoV-S may be (i) both of SARS-COV or (ii) both of SARS-COV-2.

Further optionally, the first anti-CoV-S antibody may be same as the second anti-CoV-S antibody or may be different from the second anti-CoV-S antibody. Still further optionally, these antibodies may bind to the same or different epitopes on a CoV-S expressed by said SARS-COV or SARS-COV-2. Alternatively, the first anti-CoV-S antibody and the second anti-CoV-S antibody may bind to different coronaviruses, optionally wherein the first CoV-S and the second CoV-S are (i) SARS-COV and of SARS-COV-2 coronaviruses, respectively, or are (ii) SARS-COV-2 and of SARS-CoV coronaviruses, respectively.

In some embodiments, the bispecific or multispecific isolated antibody or antigen-binding antibody fragment may comprise at least one first ABD and at least one second ABD.

In certain embodiments, (a) the first ABD may comprise the VH CDR1, the VH CDR2, the VH CDR3, the VL CDR1, the VL CDR2, and the VL CDR3 of a first anti-CoV-S antibody selected from any one of anti-CoV-S antibodies selected from the group consisting of ADI-58120, ADI-58121. ADI-58122, ADI-58123, ADI-58124, ADI-58125, ADI-58126, ADI-58127, ADI-58128, ADI-58129, ADI-58130, ADI-58131, or an affinity-matured variant of any of the foregoing; and/or (b) the second ABD binds to an antigen which may not be a CoV-S, optionally wherein the antigen is a cytokine, a cytokine receptor, or an immunomodulatory polypeptide.

In some embodiments, the isolated antibody or antigen-binding antibody fragment may comprise a Fab, Fab′, F(ab′)₂, scFv, sc(Fv)₂, minibody, diabody, sdAb, BITE.

In some embodiments, the isolated antibody or antigen-binding antibody fragment may comprise a constant region or Fc region or at least one domain thereof.

In certain embodiments, the constant region or Fc region may comprise a mutation which impairs or enhances at least one effector function, optionally FcR binding, FcRn binding, complement binding, glycosylation, complement-dependent cytotoxicity (“CDC”), or antibody-dependent cellular cytotoxicity (“ADCC”).

In some embodiments, the constant or Fc region is primate derived, preferably human.

The human constant or Fc region optionally may be selected from a human IgG1, IgG2, IgG3 or IgG4 constant or Fc region which optionally may be modified, optionally such as by domain deletion or by introducing one or more mutations which impair or enhance at least one effector function.

The present disclosure further relates to chimeric antigen receptors (“CARs”) comprising at least one antibody or antigen-binding antibody fragment described herein.

The present disclosure further relates to antibody-drug conjugates (“ADCs”) comprising: (a) at least one antibody or antigen-binding antibody fragment described herein; and (b) a drug.

In some embodiments, the drug may be: (i) an antiviral drug, which is optionally, remdesivir, favipiravir, darunavir, nelfinavir, saquinavir, lopinavir or ritonavir; (ii) an antihelminth drug, which may be optionally ivermectin; (iii) an antiparasite drug, which may be optionally hydroxychloroquine, chloroquine, or atovaquone; (iv) antibacterial vaccine, which may be optionally the tuberculosis vaccine BCG; or (v) an anti-inflammatory drug, which may be optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, which may be optionally bepotastine; (vii) an ACE inhibitor, which may be optionally moexipril; (viii) a drug that inhibits priming of CoV-S, which may be optionally a serine protease inhibitor such as nafamostat; or (ix) a cytotoxic drug, which may be optionally daunorubicin, mitoxantrone, doxorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, or epothilone.

The present disclosure also relates to isolated nucleic acids encoding any of the antibodies or antigen-binding antibody fragments disclosed herein.

In some embodiments, the nucleic acid may comprise:
(1) (a) a nucleic acid sequence with at least 90, 91, 92, 93, 94, 95, 96, 97, 98, 99, or 100% sequence identity to the nucleic acid sequence of SEQ ID NO: 9, and/or (b) a nucleic acid sequence with at least 90, 91, 92, 93, 94, 95, 96, 97, 98, 99, or 100% sequence identity to the nucleic acid sequence of SEQ ID NO: 209;
(2) (a) a nucleic acid sequence with at least 90, 91, 92, 93, 94, 95, 96, 97, 98, 99, or 100% sequence identity to the nucleic acid sequence of SEQ ID NO: 19, and/or (b) a nucleic acid sequence with at least 90, 91, 92, 93, 94, 95, 96, 97, 98, 99, or 100% sequence identity to the nucleic acid sequence of SEQ ID NO: 219;
(3) (a) a nucleic acid sequence with at least 90, 91, 92, 93, 94, 95, 96, 97, 98, 99, or 100% sequence identity to the nucleic acid sequence of SEQ ID NO: 29, and/or (b) a nucleic acid sequence with at least 90, 91, 92, 93, 94, 95, 96, 97, 98, 99, or 100% sequence identity to the nucleic acid sequence of SEQ ID NO: 229;
(4) (a) a nucleic acid sequence with at least 90, 91, 92, 93, 94, 95, 96, 97, 98, 99, or 100% sequence identity to the nucleic acid sequence of SEQ ID NO: 39, and/or (b) a nucleic acid sequence with at least 90, 91, 92, 93, 94, 95, 96, 97, 98, 99, or 100% sequence identity to the nucleic acid sequence of SEQ ID NO: 239;
(5) (a) a nucleic acid sequence with at least 90, 91, 92, 93, 94, 95, 96, 97, 98, 99, or 100% sequence identity to the nucleic acid sequence of SEQ ID NO: 49, and/or (b) a nucleic acid sequence with at least 90, 91, 92, 93, 94, 95, 96, 97, 98, 99, or 100% sequence identity to the nucleic acid sequence of SEQ ID NO: 249;
(6) (a) a nucleic acid sequence with at least 90, 91, 92, 93, 94, 95, 96, 97, 98, 99, or 100% sequence identity to the nucleic acid sequence of SEQ ID NO: 59, and/or (b) a nucleic acid sequence with at least 90, 91, 92, 93, 94, 95, 96, 97, 98, 99, or 100% sequence identity to the nucleic acid sequence of SEQ ID NO: 259;
(7) (a) a nucleic acid sequence with at least 90, 91, 92, 93, 94, 95, 96, 97, 98, 99, or 100% sequence identity to the nucleic acid sequence of SEQ ID NO: 69, and/or (b) a nucleic acid sequence with at least 90, 91, 92, 93, 94, 95, 96, 97, 98, 99, or 100% sequence identity to the nucleic acid sequence of SEQ ID NO: 269;
(8) (a) a nucleic acid sequence with at least 90, 91, 92, 93, 94, 95, 96, 97, 98, 99, or 100% sequence identity to the nucleic acid sequence of SEQ ID NO: 79, and/or (b) a nucleic acid sequence with at least 90, 91, 92, 93, 94, 95, 96, 97, 98, 99, or 100% sequence identity to the nucleic acid sequence of SEQ ID NO: 279;
(9) (a) a nucleic acid sequence with at least 90, 91, 92, 93, 94, 95, 96, 97, 98, 99, or 100% sequence identity to the nucleic acid sequence of SEQ ID NO: 89, and/or (b) a nucleic acid sequence with at least 90, 91, 92, 93, 94, 95, 96, 97, 98, 99, or 100% sequence identity to the nucleic acid sequence of SEQ ID NO: 289;
(10) (a) a nucleic acid sequence with at least 90, 91, 92, 93, 94, 95, 96, 97, 98, 99, or 100% sequence identity to the nucleic acid sequence of SEQ ID NO: 99, and/or (b) a nucleic acid sequence with at least 90, 91, 92, 93, 94, 95, 96, 97, 98, 99, or 100% sequence identity to the nucleic acid sequence of SEQ ID NO: 299;
(11) (a) a nucleic acid sequence with at least 90, 91, 92, 93, 94, 95, 96, 97, 98, 99, or 100% sequence identity to the nucleic acid sequence of SEQ ID NO: 109, and/or (b) a nucleic acid sequence with at least 90, 91, 92, 93, 94, 95, 96, 97, 98, 99, or 100% sequence identity to the nucleic acid sequence of SEQ ID NO: 309; or
(12) (a) a nucleic acid sequence with at least 90, 91, 92, 93, 94, 95, 96, 97, 98, 99, or 100% sequence identity to the nucleic acid sequence of SEQ ID NO: 119, and/or (b) a nucleic acid sequence with at least 90, 91, 92, 93, 94, 95, 96, 97, 98, 99, or 100% sequence identity to the nucleic acid sequence of SEQ ID NO: 319.

The present disclosure also relates to isolated cells which may comprise any of the nucleic acids disclosed herein.

In some embodiments the cell may be a bacterial, yeast, insect, fungal, or mammalian cell, optionally a human cell, further optionally a CHO or HEK cell.

In some embodiments the cell may be a human immune cell, optionally a T, NK, B or dendritic cell.

The present disclosure further relates to methods of expressing the antibody or antigen-binding antibody fragment or the CAR disclosed herein.

In some embodiments, the method may comprise: (a) culturing the cell expressing an antibody or antigen-binding antibody fragment or CAR of the present disclosure under conditions that permit expression; and (b) optionally isolating the antibody or antigen-binding antibody fragment or the CAR from the cell or the culture medium containing the cell.

The present disclosure further relates to methods of identifying an antibody or an antigen-binding antibody fragment which specifically binds to CoV-S.

In some embodiments, the method may comprise: (a) obtaining antisera and/or B cells obtained from a patient infected with SARS-COV or SARS-COV-2, optionally wherein the patient recovered from SARS-COV or SARS-COV-2 infection or the patient is a convalescent patient infected with SARS-COV or SARS-COV-2; (b) contacting the antisera and/or B cells with the CoV-S; and (c) isolating an antibody or antigen-binding fragment thereof which specifically bind to the CoV-S. Optionally, the CoV-S is the spike protein of SARS-COV (“SARS-COV-S”) or of SARS-COV-2 (“SARS-COV-2-S”). Further optionally, the CoV-S may comprise the amino acid sequence of SEQ ID NO: 401 (SARS-COV—S, 1288 amino acids, Accession #PDB: 6VSB_B) or SEQ ID NO: 403 (SARS-CoV-2-S. 1273 amino acids, GenBank: QHD43416.1).

In some embodiments, the method may further detect that the antibody or antigen-binding fragment thereof which specifically binds to CoV-S neutralizes, blocks or inhibits coronavirus infectivity or coronavirus proliferation, optionally wherein the coronavirus is SARS-COV or SARS-CoV-2.

In certain embodiments, the method may further detect whether the antibody or antigen-binding antibody fragment thereof which specifically binds to the CoV-S binds to other coronaviruses, optionally selected from the group consisting of MERS-COV, HCoV-HKU1, HCOV-OC43, HCoV-229E, and HCoV-NL63.

In any of such detection methods, the method may further comprise determining the sequence of the antibody or antigen-binding antibody fragment thereof may be determined.

In some embodiments these sequences may be affinity-matured or mutated to enhance binding affinity and/or potentially increase specificity to a particular CoV-S.

The present disclosure further provides compositions comprising: (a) at least one antibody or antigen-binding antibody fragment of the present disclosure; and (b) a pharmaceutically acceptable carrier or excipient.

The present disclosure further provides methods of determining whether a subject has been infected with SARS-COV or SARS-COV-2 or another coronavirus by detecting whether a biological sample from the subject may comprise SARS-COV-S protein or SARS-COV-S-2 protein or another coronavirus S protein homologous thereto based on its immunoreaction with at least one antibody or antigen-binding antibody fragment disclosed herein. The sample may optionally be blood, plasma, lymph, mucus, urine, and/or feces. Optionally, the SARS-COV S may comprise the amino acid sequence of SEQ ID NO: 401 (SARS-COV—S, 1288 amino acids, Accession #PDB: 6VSB_B),

Alternatively, the SARS-COV-2 may comprise the amino acid sequence of SEQ ID NO: 403 (SARS-COV-2-S, 1273 amino acids, GenBank: QHD43416.1).

Such determination methods optionally may comprise an ELISA or adioimmunoassay.

In such determination methods, the subject optionally may be human, a companion animal (e.g., a dog or cat), a laboratory animal, an agricultural animal, e.g., animals used in meat production,, an animal in police force, military or rescue operations, or may comprise an animal in a zoo, e.g., a tiger or lion.

In such determination methods, the samples optionally may be collected at different times from the subject and the presence or absence or the level of SARS-COV—S or SARS-COV-S-2 or another coronavirus S protein homologous thereto may be detected in order to assess whether the subject has recovered. Here, the SARS-COV S may comprise the amino acid sequence of SEQ ID NO: 401 (SARS-COV-S. 1288 amino acids, Accession #PDB: 6VSB_B), and optionally the SARS-COV-2 may comprise the amino acid sequence of SEQ ID NO: 403 (SARS-COV-2-S. 1273 amino acids, GenBank: QHD43416.1).

The present disclosure further provides methods of inducing an immune response against SARS-COV or SARS-COV-2 or another coronavirus, which may be selected from MERS-COV, HCoV-HKU1, HCoV-OC43, HCoV-229E, and HCoV-NL63, in a subject in need thereof.

In some embodiments, the methods may comprise administering at least one antibody or antigen-binding antibody fragment of the present disclosure.

In some embodiments, the methods may comprise administering a cocktail of different antibodies or antigen-binding antibody fragments of the present disclosure, e.g., which bind to the same or different epitopes on the same or different CoV-Ss.

In certain embodiments, the immune response elicits immunoprotection, optionally prolonged, against at least one coronavirus, optionally SARS-COV or SARS-COV-2, further optionally against another coronavirus.

The present disclosure further provides methods of inhibiting or blocking infection of susceptible cells by SARS-COV or SARS-COV-2 or another coronavirus, such as MERS-COV, HCoV-HKU1, HCoV-OC43, HCoV-229E, and HCoV-NL63, in a subject in need thereof.

In some embodiments, the method may comprise administering at least one antibody or antigen-binding antibody fragment, of the present disclosure, e.g., a cocktail as above-described.

The present disclosure further provides methods of treating infection by SARS-COV or SARS-COV-2 or another coronavirus optionally such as MERS-COV, HCoV-HKU1, HCoV-OC43, HCoV-229E, and HCoV-NL63, or treating a condition, symptom, disease, or disorder associated with said infection in a subject in need thereof.

In some embodiments, the method may comprise administering to the subject a therapeutically effective amount of at least one antibody or antigen-binding antibody fragment, of the present disclosure, e.g., a cocktail as above-described. In some embodiments, the method comprises administering two antibodies, or antigen-binding antibody fragments, of the present disclosure, e.g., ADI-58122 and ADI-58125.

In some embodiments, the condition, symptom, disease, or disorder comprises at least one of bronchitis, pneumonia, respiratory failure, acute respiratory failure, organ failure, multi-organ system failure, pediatric inflammatory multisystem syndrome, acute respiratory distress syndrome, blood clot, a cardiac condition, myocardial injury, myocarditis, heart failure, cardiac arrest, acute myocardial infarction, dysrhythmia, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, a gastrointestinal complication, aspergillosis, increased susceptibility to infection with another virus or bacteria, and/or a pregnancy-related complication.

The present disclosure also provides methods of preventing infection by SARS-COV or SARS-COV-2 or another coronavirus optionally selected from the group consisting of MERS-COV, HCoV-HKU1, HCoV-OC43, HCOV-229E, and HCoV-NL63 in a subject in need thereof.

In some embodiments, the method may comprise administering to the subject a prophylactically effective amount of at least one antibody or antigen-binding antibody fragment, an ADC or a CAR, of the present disclosure, e.g., a cocktail as above-described.

The present disclosure also provides methods of preventing the need for a subject infected with SARS-COV or SARS-COV-2 or another coronavirus optionally selected from the group consisting of MERS-COV, HCoV-HKU1, HCoV-OC43, HCOV-229E, and HCoV-NL63 to be placed on a ventilator, or reducing the time that a subject infected with SARS-COV or SARS-COV-2 or another coronavirus optionally selected from the group consisting of MERS-COV, HCoV-HKU1, HCoV-OC43, HCOV-229E, and HCoV-NL63 is on a ventilator.

In some embodiments, the method may comprise administering to the subject a prophylactically or therapeutically effective amount of at least one antibody or antigen-binding antibody fragment, an ADC or a CAR, of the present disclosure, e.g., a cocktail as above-described.

The present disclosure provides methods of preventing the onset of pneumonia in a subject infected SARS-COV or SARS-COV-2 or another coronavirus optionally selected from the group consisting of MERS-COV. HCoV-HKU1, HCoV-OC43, HCOV-229E, and HCoV-NL63, or treating pneumonia and/or the symptoms of pneumonia in a subject for a subject infected SARS-COV or SARS-COV-2 or another coronavirus optionally selected from the group consisting of MERS-COV. HCoV-HKU1, HCoV-OC43, HCOV-229E, and HCoV-NL63.

In some embodiments, the method may comprise administering to the subject a prophylactically or therapeutically effective amount of at least one antibody or antigen-binding antibody fragment, an ADC or a CAR, of the present disclosure, e.g., a cocktail as above-described.

In any of such methods, the subject optionally may be human or may comprise a companion animal, agricultural animal or animal in a zoo.

Optionally the subject may have at least one risk factor which renders them more prone to a poor clinical outcome. In certain embodiments, wherein the risk factors may comprise one or more of (i) advanced age such as over 55, 60 or 65 years old, (ii) diabetes, (iii) a chronic respiratory condition such as asthma, cystic fibrosis, another fibrotic condition, or COPD, (iv) obesity, (iv) hypertension, (v) a cardiac or cardiovascular condition, such as heart defects or abnormalities, (vi) a chronic inflammatory or autoimmune condition, e.g., lupus or multiple sclerosis, and (vii) an immunocompromised status which optionally may be caused by cancer, chemotherapy, smoking, bone marrow or organ transplantation, immune deficiencies, poorly controlled HIV infection or AIDS, or prolonged use of corticosteroids or other immunosuppressive medications.

In certain embodiments, the subject may further be treated with at least one other drug. In certain embodiments, the method further comprises administering to the subject at least one other drug. Optionally, such one other drug may be: (i) an antiviral drug, optionally, remdesivir, favipiravir, darunavir, nelfinavir, saquinavir, lopinavir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., tocilizumab), or metamizole; (vi) an antihistamine drug, optionally bepotastine; (vii) an ACE inhibitor, optionally moexipril; and/or (viii) a drug that inhibits priming of CoV-S, optionally a serine protease inhibitor, further optionally nafamostat.

In certain embodiments, the subject may further be treated with: (I) an antiviral agent, optionally, remdesivir, favipiravir, darunavir, nelfinavir, saquinavir, lopinavir, or ritonavir; and (II) at least one other drug. In certain embodiments, the method may further comprise administering to the subject (I) an antiviral agent, optionally, remdesivir, favipiravir, darunavir, nelfinavir, saquinavir, lopinavir, or ritonavir; and (II) at least one other drug. Optionally, the at least one other drug may be (i) an antihelminth drug, further optionally ivermectin; (ii) an antiparasitic drug, optionally hydroxychloroquine, chloroquine, or atovaquone; (iii) an antibacterial vaccine, which is optionally the tuberculosis vaccine BCG; or (iv) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (v) an antihistamine drug, optionally bepotastine; (vi) an ACE inhibitor, optionally moexipril; and/or (vii) a drug that inhibits priming of CoV-S, which is optionally a serine protease inhibitor such as nafamostat.

In some embodiments, anti-CoV-S antibodies or antigen-binding antibody fragments of the present disclosure may be characterized by having a certain VH CDR3 sequences or having a VH CDR3 sequences that are similar to a certain VH CDR3.

In certain embodiments, antibody or antigen-binding antibody fragment of the present disclosure may comprise an Fc region. The Fc region may comprise a wild type sequence or a variant sequence and optionally may comprise an amino acid sequence of SEQ ID NOs: 411, 412, 413, 414, 415, 416, or 417.

In certain embodiments, the isolated antibody or antigen-binding antibody fragment may bind to the S1 subunit of SARS-COV—S or of SARS-COV-2-S.

In certain embodiments, the isolated antibody or antigen-binding antibody fragment may bind to the receptor binding domain (RBD) or the N-terminal domain (NTD) of SARS-COV—S or of SARS-CoV-2-S.

In certain embodiments, the isolated antibody or antigen-binding antibody fragment may bind to the ACE2-binding motif of SARS-COV—S or of SARS-COV-2-S and optionally further binds to the epitope of the antibody CR3022.

In further embodiments, the isolated antibody or antigen-binding antibody fragment may compete with ACE2.

In further embodiments, the isolated antibody or antigen-binding antibody fragment may compete with: (i) ACE2 and the antibody CR3022; or (ii) ACE2 but not the antibody CR3022. In certain embodiments, the isolated antibody or antigen-binding antibody fragment (a) may bind to the S protein of SARS-COV and/or of SARS-COV-2; and (b) may not bind to any of the S proteins of HCOV-229E, HCoV-HKU1, HCOV-NL63, and HCoV-OK43.

In certain embodiments, the isolated antibody or antigen-binding antibody fragment may (a) bind to the S protein of SARS-COV and/or of SARS-COV-2; and also (b) bind to the S protein of at least one of HCOV-229E, HCoV-HKU1, HCOV-NL63, and HCoV-OK43.

In further embodiments, the isolated antibody or antigen-binding antibody fragment may neutralize SARS-COV and/or SARS-COV-2.

In further embodiments, the isolated antibody or antigen-binding antibody fragment may neutralize SARS-COV and/or SARS-COV-2 at 100 nM in vitro.

In further embodiments, the isolated antibody or antigen-binding antibody fragment may neutralize SARS-COV and/or SARS-COV-2 at: (i) an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 pM or lower, of about 20 pM or lower, of about 10 pM or lower, of about 5 pM or lower, of about 2 pM or lower, or of about 1 pM or lower; and/or (ii) an IC50 of about 500 ng/ml or lower, of about 200 ng/ml or lower, of about 100 ng/ml or lower, of about 50 ng/ml or lower, of about 20 ng/mL or lower, of about 10 ng/ml or lower, of about 20 ng/ml or lower, of about 10 mg/mL or lower, of about 5 ng/mL or lower, of about 2 ng/mL or lower, or of about 1 ng/mL or lower, in vitro, optionally as measured by any of the neutralization assays described in the Examples herein.

In further embodiments, the isolated antibody or antigen-binding antibody fragment may bind to CoV-S(S protein of any CoV, such as but not limited to SARS-COV—S and/or SARS-COV-2-S) with a KD value of: (i) 100 nM or lower; (ii) 10 nM or lower; (iii) 1 nM or lower; (iv) 100 pM or lower; (v) 10 pM or lower; (vi) 1 pM or lower; or (vii) 0.1 pM or lower.

In some embodiments, the antibody, or antigen-binding fragment thereof, is administered intravenously. In other embodiments, the antibody, or antigen-binding fragment thereof, is administered intramuscularly.

In some embodiments, at least one antibody, or antigen-binding fragment thereof, is administered. In some embodiments, at least two antibodies, or antigen-binding fragment thereof, are administered. In some embodiments, the anti-CoV-S antibody and antigen-binding fragment thereof, e.g., ADI-58125, may be alone or used in combination with a second antibody, or antigen-binding fragment thereof, wherein the second antibody, or antigen-binding fragment thereof, is selected from the group consisting of ADI-58120, ADI-58121. ADI-58122, ADI-58123, ADI-58124, ADI-58126, ADI-58127. ADI-58128, ADI-58129, ADI-58130, ADI-58131, or a combination thereof. In some embodiment, the second antibody, or antigen-binding fragment thereof, is ADI-58122. In one embodiment, the second antibody, or antigen-binding fragment thereof, is ADI-58127. In one embodiment, the second antibody, or antigen-binding fragment thereof, is ADI-58129. In one embodiment, the second antibody, or antigen-binding fragment thereof, is ADI-58131. In some embodiments, the first antibody, or antigen-binding fragment thereof, is, ADI-58125, and the second antibody, or antigen-binding fragment thereof, is ADI-58122.

In some embodiments, the antibody, or antigen-binding fragment thereof, is administered at a dose of about 100 mg to about 5000, about 100 mg to about 4500 mg, about 100 mg to about 2000 mg, about 200 mg to about 1500 mg, about 300 mg to about 600 mg, about 500 mg to about 1200 mg, about 300 mg to about 1200 mg, or about 1200 mg to about 4500 mg. In some embodiments, the antibody, or antigen-binding fragment thereof, is administered at a dose of about 200 mg, about 300 mg, about 400 mg, about 500 mg, about 600 mg, about 700 mg, about 800 mg, about 900 mg, about 1000 mg about 1100 mg, about 1200 mg, about 1300 mg, about 1400 mg, about 1500 mg, about 1600 mg, about 1700 mg, about 1800 mg, about 1900 mg, about 2000 mg, about 2500 mg, about 3000 mg, about 3500 mg, about 4000 mg, about 4500 mg, or about 5000 mg.

In some embodiments, the antibody, or antigen-binding fragment thereof, is administered at a dose of about 300 mg intramuscularly, about 500 mg intravenously, about 600 mg intramuscularly, about 1200 mg intramuscularly, or about 1200 mg intravenously, or about 4500 mg intravenously.

In one embodiment, the antibody, or antigen-binding fragment thereof, is administered once. In one embodiment, the antibody, or antigen-binding fragment thereof, is administered weekly. In another embodiment, the antibody, or antigen-binding fragment thereof, is administered daily, weekly, every two weeks, monthly, or every two months. In one embodiment, the antibody, or antigen-binding fragment thereof, is administered weekly for about four weeks, once weekly for about a month, weekly for about 5 weeks, weekly for about 6 weeks, weekly for about 7 weeks, or weekly for about two months.

In some embodiments, the methods further comprise obtaining a serum sample from the subject.

In some embodiments, the antibody, or antigen-binding fragment thereof, reaches a maximum concentration (Cmax) of about 10 μg/mL to about 1000 μg/mL, about 20 μg/mL to about 500 μg/mL, about 30 μg/mL to about 400 μg/mL, about 40 μg/mL to about 300 μg/mL, about 50 μg/mL to about 200 μg/mL, about 50 μg/mL to about 100 μg/mL, or about 30 μg/mL to about 70 μg/mL in the serum sample of the subject. In some embodiments, the antibody, or antigen-binding fragment thereof, reaches a maximum concentration (Cmax) of about 30 μg/mL, about 40 μg/mL, about 50 μg/mL, about 60 μg/mL, about 70 μg/mL, about 80 μg/mL, about 90 μg/mL, about 100 μg/mL, about 150 μg/mL, or about 200 μg/mL in the serum sample of the subject.

In some embodiments, the median time for the antibody, or antigen-binding fragment thereof, to reach a maximum concentration in the serum sample of the subject is about 5-30 days, about 6-20 days, about 7-18 days, or about 8-15 days, or about 13-15 days after administration. In some embodiments, the median time for the antibody, or antigen-binding fragment thereof, to reach a maximum concentration in the serum sample of the subject is about 6, 7, 8, 9, 10, 11, 12, 13, 14, 15, 16, 17, 18, 19 or 20 days after administration.

In some embodiments, the antibody, or antigen-binding fragment thereof, reaches a maximum concentration (Cmax) of about 30 μg/mL to about 100 μg/mL, about 40 μg/mL to about 80 μg/mL, about 50 μg/mL to about 70 μg/mL, or about 30 μg/mL to about 65 μg/mL in the serum sample of the subject in about 6-20 days, about 7-18 days, about 8-15 or about 13-15 days after administration.

In some embodiments, the area under the serum concentration-time curve from day 0 to day 20 (AUC_{0-20d}) is about 100-2000 day* μg/mL, about 200-1500 day* μg/mL, about 400-1400 day* μg/mL, about 500-1300 day* μg/mL, about 600-1000 day* μg/mL, or about 800-900 day* μg/mL.

In some embodiments, the antibody, or antigen-binding fragment thereof, has a virus neutralizing titer of about 100-2000, about 200-1500, about 300-1500, or about 500-1500 in the serum sample of the subject about 6 months after administration. In some embodiments, the antibody, or antigen-binding fragment thereof, has a virus neutralizing titer of about 100, 200, 300, 400, 500, 600, 700, 800, 900, 1000, 1100, 1200, 1300, 1400, 1500, 1600, 1700, 1800, 1900 or 2000 in the serum sample of the subject about 6 months after administration.

In some embodiments, the 80% virus neutralization titer (MN80) of the antibody, or antigen-binding fragment thereof, at about days 0-14 after administration, is about 200-1500, about 300-1000, about 400-800 or about 500-600 in the serum sample of the subject. In some embodiments, the 80% virus neutralization titer (MN80) of the antibody, or antigen-binding fragment thereof, at about day 13 after administration, is about 400-800 or about 500-600 in the serum sample of the subject.

In some embodiments, the 50% virus neutralization titer (MN50) of the antibody, or antigen-binding fragment thereof, at about days 0-14 after administration, is about 500-2500, about 800-2000, about 1000-1800 or about 1300-1500 in the serum sample of the subject. In some embodiments, the 50% virus neutralization titer (MN50) of the antibody, or antigen-binding fragment thereof, at about day 13 after administration, is about 1000-1800 or about 1300-1500 in the serum sample of the subject.

In some embodiments, the neutralization titer is determined using a plaque reduction neutralization test (PRNT).

In one aspect, the present disclosure also relates to kits comprising: (a) at least one isolated antibody or antigen-binding antibody fragment disclosed herein; and (b) an instruction for using the antibody or antigen-binding antibody fragment.

In some embodiments, the kit may be for use in: (i) determining whether a CoV is present in a subject; (ii) diagnosing whether a subject has CoV infection; (iii) predicting whether a CoV vaccine will elicit a protective immune response; or (iv) predicting whether a CoV vaccine will elicit a neutralizing antibody response.

In one aspect, provided herein are methods of predicting the in vivo efficacy of an anti-CoV-S antibody or antigen-binding antibody fragment in preventing or treating CoV infection.

In some embodiments, the method may comprise: (a) providing at least one first test subject and at least one second subject or a cell sample derived from at least one first test subject and at least one second subject; (b) administering the antibody or antigen-binding antibody fragment to said at least one first test subject and said at least one second subject or contacting a cell sample from said first and second subject with the antibody or antigen-binding antibody fragment; (c) infecting said at least one first test subject and said at least one second subject with CoV or pseudo CoV or a cell sample obtained from said at least one first test subject and said at least one second subject with CoV or pseudo CoV; (d) determining whether administration of the antibody or antigen-binding antibody fragment in (b) results in one or more of the following compared to a suitable control: (I) reduction in a CoV-associated symptom; (II) reduction in the CoV viremia; (III) increase in the survival; (IV) increase in the body weight; or (V) reduced infection of cells or virus proliferation in cells of the tested cell sample compared to a control cell sample not contacted with the antibody or antigen-binding antibody fragment.

In some embodiments, the method may comprise: (a) providing at least one first cell sample and at least one second cell sample; (b) contacting the at least one first cell sample with the antibody or antigen-binding antibody fragment; (c) infecting said at least one first cell sample and at least one second cell sample with CoV or pseudo CoV; (d) determining whether the antibody or antigen-binding antibody fragment results in one or more of the following compared to a suitable control: (I) increase in the cell survival; (II) reduced infection of cells; (III) reduced virus proliferation; (IV) reduced cell stress or death markers; or (V) reduced inflammatory cytokines, in cells of the tested cell sample compared to a control cell sample not contacted with the antibody or antigen-binding antibody fragment.

In some embodiments, the method may comprise: (a) providing at least one first test subject and at least one second subject or a cell sample derived from at least one first test subject and at least one second subject; (b) infecting said at least one first test subject and said at least one second subject with CoV or pseudo CoV or a cell sample derived from at least one first test subject and at least one second subject; (c) administering the antibody or antigen-binding antibody fragment to said at least one second subject or contacting a cell sample derived from at least one first test subject and at least one second subject with the antibody or antigen-binding antibody fragment; (d) determining whether administration of the antibody or antigen-binding antibody fragment in (c) results in one or more of the following: (I) reduction in a CoV-associated symptom; (II) reduction in the CoV viremia; (III) increase in the survival; (IV) increase in the body weight; or (V) reduced infection of cells or virus proliferation in cells in the tested cell sample compared to a control cell sample not contacted with the antibody or antigen-binding antibody fragment.

In some embodiments, the method may comprise: (a) providing at least one first cell sample and at least one second cell sample; (b) infecting said at least one first cell sample and at least one second cell sample with CoV or pseudo CoV; (c) contacting the at least one first cell sample with the antibody or antigen-binding antibody fragment; (d) determining whether the antibody or antigen-binding antibody fragment results in one or more of the following compared to a suitable control: (I) increase in the cell survival; (II) reduced infection of cells; (III) reduced virus proliferation; (IV) reduced cell stress or death markers; or (V) reduced inflammatory cytokines, in cells of the tested cell sample compared to a control cell sample not contacted with the antibody or antigen-binding antibody fragment.

In one aspect, provided herein are methods of screening for an antibody or antigen-binding antibody fragment that binds to a CoV or CoV-S, the method comprising whether an antibody or antigen-binding antibody fragment comprising 1, 2, 3, 4, 5, or 6 CDRs of any of the antibodies disclosed herein may comprise one or more of the following features: (i) binds to the S protein of a CoV; (ii) binds to the S1 subunit of CoV-S; (iii) binds to the RBD of CoV-S; (iv) binds to the NTD of CoV-S; (v) binds to the ACE2-binding motif of CoV-S; (vi) competes with ACE2; (vii) competes with the antibody CR3022; (viii) neutralizes one or more of SARS-COV, SARS-COV-2, MERS-COV, HCOV-229E, HCoV-HKU1, HCoV-NL63, or HCoV-OK43 or variants thereof; (ix) neutralizes a pseudovirus of one or more of SARS-COV, SARS-COV-2, MERS-COV, HCOV-229E, HCoV-HKU1, HCoV-NL63, or HCoV-OK43 or variants thereof; (x) results in reduced infection of cells or virus proliferation in cells in a susceptible tested cell sample compared to a control cell sample not contacted with the antibody or antigen-binding antibody fragment; or (xi) prevents or treats CoV infection in vivo. During the screening, any of the antibodies disclosed herein and/or an antibody comprising one or more of the CDRs of the antibodies disclosed herein may be used as a candidate antibody or a control antibody.

In one aspect, the present disclosure also relates to compositions comprising at least one affinity-matured first anti-CoV-S antibody or antigen-binding antibody fragment and a pharmaceutically acceptable carrier or excipient.

In some embodiments, the at least one first antibody or antigen-binding antibody fragment may comprise: a VH comprising a VH CDR1, a VH CDR2, a VH CDR3; and a VL, comprising a VL CDR1 a VL CDR2, a VL CDR3, and the amino acid sequences of said VH CDR1, said VH CDR2, said VH CDR3, said VL CDR1, said VL CDR2, and said VL CDR3 are identical to the amino acid sequences of the VH CDR1, VH CDR2, VH CDR3, VL CDR1, VL CDR2, and VL CDR3, respectively, of an anti-CoV-S antibody selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131.

In some embodiments, the first antibody or antigen-binding antibody fragment may comprise an Fc region, optionally wherein the Fc region may comprise an amino acid sequence of SEQ ID NOs: 411, 412, 413, 414, 415, 416, or 417.

In one embodiment, the HC and LC of the first antibody or antigen-binding antibody fragment are the HC and LC, respectively, of an anti-CoV-S antibody selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In certain embodiments, the composition may further comprise at least one second antibody or antigen-binding antibody fragment comprising a VH comprising a VH CDR1, a VH CDR2, a VH CDR3 and a VL, comprising a VL CDR1 a VL CDR2, a VL CDR3. In particular embodiments, the amino acid sequences of said VH CDR1, said VH CDR2, said VH CDR3, said VL CDR1, said VL CDR2, and said VL CDR3 may be identical to the amino acid sequences of the VH CDR1, VH CDR2, VH CDR3, VL CDR1, VL CDR2, and VL CDR3, respectively, of an anti-CoV-S antibody selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In some embodiments, the second antibody or antigen-binding antibody fragment may comprise an Fc region, optionally wherein the Fc region may comprise an amino acid sequence of SEQ ID NOs: 411, 412, 413, 414, 415, 416, or 417.

In particular embodiments, the antibody or antigen-binding antibody fragment according to the present disclosure may comprise:
(1) a HC comprising the amino acid sequence of SEQ ID NO: 10 and a LC comprising the amino acid sequence of SEQ ID NO: 210;
(2) a HC comprising the amino acid sequence of SEQ ID NO: 20 and a LC comprising the amino acid sequence of SEQ ID NO: 220;
(3) a HC comprising the amino acid sequence of SEQ ID NO: 30 and a LC comprising the amino acid sequence of SEQ ID NO: 230;
(4) a HC comprising the amino acid sequence of SEQ ID NO: 40 and a LC comprising the amino acid sequence of SEQ ID NO: 240;
(5) a HC comprising the amino acid sequence of SEQ ID NO: 50 and a LC comprising the amino acid sequence of SEQ ID NO: 250;
(6) a HC comprising the amino acid sequence of SEQ ID NO: 60 and a LC comprising the amino acid sequence of SEQ ID NO: 260;
(7) a HC comprising the amino acid sequence of SEQ ID NO: 70 and a LC comprising the amino acid sequence of SEQ ID NO: 270;
(8) a HC comprising the amino acid sequence of SEQ ID NO: 80 and a LC comprising the amino acid sequence of SEQ ID NO: 280;
(9) a HC comprising the amino acid sequence of SEQ ID NO: 90 and a LC comprising the amino acid sequence of SEQ ID NO: 290;
(10) a HC comprising the amino acid sequence of SEQ ID NO: 100 and a LC comprising the amino acid sequence of SEQ ID NO: 300;
(11) a HC comprising the amino acid sequence of SEQ ID NO: 110 and a LC comprising the amino acid sequence of SEQ ID NO: 310; or
(12) a HC comprising the amino acid sequence of SEQ ID NO: 120 and a LC comprising the amino acid sequence of SEQ ID NO: 320.

In further embodiments, the composition according to the present disclosure may comprise: (A) at least one first antibody or antigen-binding antibody fragment selected from the group consisting of the antibodies or antigen-binding antibody fragments comprising the HC and LC combination as described above; and (B) a pharmaceutically acceptable carrier or excipient.

In yet further embodiments, the composition may additionally comprise at least one second antibody or antigen-binding antibody fragment selected from the group consisting of the antibodies or antigen-binding antibody fragments comprising the HC and LC combination as described above.

Additionally, the present disclosure further encompasses isolated antibodies and antigen-binding antibody fragments thereof, which competes for binding with any one or more of the anti-CoV antibodies or antigen-binding antibody fragments thereof as described herein.

The present disclosure also encompasses isolated antibodies or antigen-binding antibody fragments thereof, which bind the same epitope as any one or more of the anti-CoV antibodies or antigen-binding antibody fragments thereof as described herein.

The present disclosure further encompasses affinity matured variants of any one or more of the anti-CoV antibodies or antigen-binding antibody fragments thereof as described herein.

In one aspect, disclosed herein is a method of treating a coronavirus infection by SARS-COV, SARS-COV-2, and/or another coronavirus optionally selected from the group consisting of MERS-CoV. HCoV-HKU1, HCoV-OC43, HCOV-229E, and HCoV-NL63 in a subject in need thereof, the method comprising administering to the subject a therapeutically effective amount of an antibody, or antigen-binding antibody fragment thereof, which binds the same epitope as ADI-58125, and/or which competes for binding with ADI-58125.

In one aspect, disclosed herein is a method of decreasing the risk of mortality, hospitalization, mechanical ventilation, or a combination thereof in a patient infected by SARS-COV, SARS-COV-2, and/or another coronavirus optionally selected from the group consisting of MERS-COV, HCOV-HKU1, HCoV-OC43, HCoV-229E, and HCoV-NL63, the method comprising administering to the subject a therapeutically effective amount of an isolated antibody, or antigen-binding antibody fragment thereof, which binds the same epitope as ADI-58125, and/or which competes for binding with ADI-58125.

In another aspect, disclosed herein is a method of preventing infection of a subject by SARS-CoV. SARS-COV-2, and/or another coronavirus optionally selected from the group consisting of MERS-COV, HCoV-HKU1, HCoV-OC43, HCOV-229E, and HCoV-NL63, the method comprising administering to the subject a therapeutically effective amount of an isolated antibody, or antigen-binding antibody fragment thereof, which binds the same epitope as ADI-58125, and/or which competes for binding with ADI-58125.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the overall formulation development of ADI-58125.

FIG. 2 depicts the visual observations, viscosity and turbidity data from Study 1 at t=0. The formulations are labelled from F1 to F16, the excipient and buffer are listed on the bottom two rows. The measured values for pH, viscosity and turbidity are listed. The shading is in order of decreasing desirability for each of the attributes, i.e., viscosity and turbidity.

FIG. 3 depicts the comparison of SEC data at 40, 25, and 5° C. from Study 1. The formulations highlighted by the oval all have histidine as the buffering agent.

FIG. 4 depicts the comparison of CEC data at 40, 25, and 5° C. from Study 1.

FIG. 5 depicts the comparison of SEC LMW data at 40, 25, and 5° C. from Study 1.

FIG. 6 depicts the visual observations, viscosity and turbidity data from Study 2 at t=0. Formulations are labelled from F1 to F13, and the composition is described in Table 4. For each attribute, the row is color coded based on increasing desirability of the attribute (red, yellow and green). Excipient concentrations are in mM units. Su=sucrose, R=arginine hydrochloride, P=proline, G=glycine, NaCl=sodium chloride, R-E=arginine glutamate.

FIG. 7 depicts the comparison of SEC data at 40, 25, and 5° C. from Study 2.

FIG. 8 depicts the comparison of CEC data at 40, 25, and 5° C. from Study 2.

FIG. 9 depicts the comparison of SEC LMW data at 40, 25, and 5° C. from Study 2.

FIG. 10 depicts the comparison of SEC data between Study 1 and Study 2 at 40, 25 and 5° C. The graphs on the left, middle and right correspond to 40° C. 25° C., and 5° C. SEC data from Study 1 (purple) and Study 2 (orange).

FIG. 11 depicts the visual observations, viscosity and turbidity data from Study 3 after 1 day agitation. Formulations from left to right are water (control), F1 to F5. Formulation compositions (F1 to F5) are described in Table 5. For each attribute, the row is color coded based on increasing desirability of the attribute (red, yellow and green).

FIG. 12 depicts the SEC and iCIEF data at 40, 25, and 5° C. from Study 3.

FIG. 13 depicts the comparison of viscosity and osmolality between formulations with similar target compositions.

FIG. 14 is a schematic depicting the UF/DF process and results with different DF buffers.

FIG. 15 depicts the visual appearance of formulation in the fine-tuning study. The 3 formulations (F6, F7 & F8) are shown along with the opalescence standards (6, 18, 20, & 30 NTU) for comparison.

FIG. 16 depicts the SE-HPLC data for formulations stored at 40° C. for 1w from the fine-tuning study.

FIG. 17 depicts the iCIEF data for formulations stored at 40° C. for 1w from the fine-tuning study.

FIG. 18 depicts the standardized pareto effect of formulation parameters (i.e., protein concentration, pH, sucrose, and arginine) on SEC-HPLC, cIEF, and CE-SDS (non-reduced and reduced) upon storage at 25° C.

FIG. 19 depicts the pH-dependent loss of cIEF main peak of drug product at 25° C.

FIG. 20 depicts the pH-dependent loss of SEC-HPLC main peak of drug product at 25° C.

FIG. 21 depicts the iCIEF data for drug product with 150 mg/mL ADI-58125 in a formulation of 10 mM L-histidine, 75 mM L-arginine hydrochloride, 100 mM sucrose, 0.03% polysorbate 80 (w/v) at a pH of 5.4-5.6 at 5° C.

FIG. 22 depicts the SEC data for drug product with with 150 mg/mL ADI-58125 in a formulation of 10 mM L-histidine, 75 mM L-arginine hydrochloride, 100 mM sucrose, 0.03% polysorbate 80 (w/v) at a pH of 5.4-5.6 at 5° C.

FIG. 23 depicts the CE-SDS (non-reduced and reduced) data for drug product with with 150 mg/mL ADI-58125 in a formulation of 10 mM L-histidine, 75 mM L-arginine hydrochloride, 100 mM sucrose, 0.03% polysorbate 80 (w/v) at a pH of 5.4-5.6 at 5° C.

### DETAILED DESCRIPTION

### A. Definitions

It is to be understood that this disclosure is not limited to the particular methodology, protocols, cell lines, animal species or genera, and reagents described, as such may vary. It is also to be understood that the terminology used herein is for the purpose of describing particular embodiments only and is not intended to limit the scope of the present disclosure, which will be limited only by the appended claims. As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

Spike protein (S protein): As used herein, unless stated otherwise S protein includes any coronavirus form of S protein. The term coronavirus S protein (“CoV-S”) is used to describe the S protein of any coronaviruses. In particular, the “SARS-COV-S” and “SARS-COV-2-S” encompass the S protein of SARS-COV and of SARS-COV-2. SEQ ID NO: 401 is an exemplary polypeptide sequence of SARS-COV—S, comprising 1288 amino acids (Accession #PDB: 6VSB_B). SEQ ID NO: 403 is an exemplary polypeptide sequence of SARS-COV-2-S, comprising 1273 amino acids (GenBank: QHD43416.1). SEQ ID NO: 402 (3864 nucleotides) encodes the SARS-COV-S(SEQ ID NO: 401) and SEQ ID NO: 404 (3822 nucleotides, NC_045512:21563.25384, also see the corresponding region of GenBank: MN908947) encodes SARS-COV-2-S(SEQ ID NO: 403).

In some embodiments, the “SARS-COV-S” and “SARS-COV-2-S” encompass any mutants, splice variants, isoforms, orthologs, homologs, and variants of SEQ ID Nos 401 and 403. In some embodiments, the CoV-S comprises a polypeptide sequence having at least 85%, 90%, 95%, 96%, 97%, 98%, or 99% identity to either SEQ ID NO: 401 or SEQ ID NO: 403.

“Effective treatment or prevention of CoV infection” herein refers to eliminating CoV from the subject or preventing the expansion of CoV in the subject or eliminating or reducing the symptoms such as fever, cough, shortness of breath, runny nose, congestion, conjunctivitis, and/or gastrointestinal symptoms after administration of an effective amount of an anti-CoV-S antibody or antigen-binding fragment thereof. In some instances, effective treatment may eliminate the need for the subject to be placed on a ventilator or reduce the time the subject needs to be on a ventilator. The treatment may be effected as a monotherapy or in association with another active agent such as an antiviral agent or anti-inflammatory agent by way of example.

As used herein. “treatment” is an approach for obtaining beneficial or desired clinical results. For purposes of this disclosure, beneficial or desired clinical results include, but are not limited to, one or more of the following: improvement in any aspect of COV-S-related conditions such as fever or cough. For example, in the context of CoV infection treatment this includes lessening severity, alleviation of fever, cough, shortness of breath, and other associated symptoms, reducing frequency of recurrence, increasing the quality of life of those suffering from the CoV-related symptoms, and decreasing dose of other medications required to treat the CoV-related symptoms. Other associated symptoms include, but are not limited to, diarrhea, conjunctivitis, loss of smell, and loss of taste. Still other symptoms which may be alleviated or prevented include inflammation, cytokine storm and/or sepsis.

“Reducing incidence” or “prophylaxis” or “prevention” means any of reducing severity for a particular disease, condition, symptom, or disorder (the terms disease, condition, and disorder are used interchangeably throughout the application). Reduction in severity includes reducing drugs and/or therapies generally used for the condition by, for example, reducing the need for, amount of, and/or exposure to drugs or therapies. Reduction in severity also includes reducing the duration, and/or frequency of the particular condition, symptom, or disorder (including, for example, delaying or increasing time to next episodic attack in an individual). This further includes eliminating the need for the subject to be placed on a ventilator or reducing the time the subject needs to be on a ventilator.

“Ameliorating” one or more symptoms of CoV infection-related conditions means a lessening or improvement of one or more symptoms of the condition, e.g., fever or cough or shortness of breath as compared to not administering an anti-CoV-S antagonist antibody. “Ameliorating” also includes shortening or reduction in duration of a symptom. Again, this may include eliminating the need for the subject to be placed on a ventilator or reducing the time the subject needs to be on a ventilator.

As used herein, “controlling CoV-related symptom” or “controlling” another CoV-S-related condition refers to maintaining or reducing severity or duration of one or more symptoms of the condition (as compared to the level before treatment). For example, the duration or severity or frequency of symptoms is reduced by at least about any of 10%, 20%, 30%, 40%, 50%, 60%, 70%, 80%, 90%, or 100% in the individual as compared to the level before treatment. The reduction in the duration or severity, or frequency of symptoms can last for any length of time, e.g., 2 weeks, 4 weeks (1 month), 8 weeks (2 months), 16 weeks (3 months), 4 months, 5 months, 6 months, 9 months, 12 months, etc.

As used therein, “delaying” the development of a CoV-S-related condition such as shortness of breath, bronchitis, or pneumonia e.g., interstitial), means to defer, hinder, slow, retard, stabilize, and/or postpone progression of the condition or disease. This delay can be of varying lengths of time, depending on the history of the condition or disease and/or individuals being treated. As is evident to one skilled in the art, a sufficient or significant delay can, in effect, encompass prevention, in that the individual does not develop symptoms. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of a CoV-related condition such as cough or fever means initial manifestations and/or ensuing progression of the disorder. Development of cough or fever can be detectable and assessed using standard clinical techniques as well known in the art. However, development also refers to progression that may be undetectable. For purpose of this disclosure, development, or progression refers to the biological course of the symptoms. “Development” includes occurrence, recurrence, and onset. As used herein “onset” or “occurrence” of a condition includes initial onset and/or recurrence.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological, and/or behavioral symptoms of the disease, its complications and intermediate pathological phenotypes presenting during development of the disease. For therapeutic use, beneficial or desired results include clinical results such as reducing symptom intensity, duration, or frequency, and decreasing one or more symptoms resulting from CoV infection, including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients, eliminating the need for the subject to be placed on a ventilator or reducing the time the subject needs to be on a ventilator.

An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

A “suitable host cell” or “host cell” generally includes any cell wherein the subject anti-CoV-S antibodies and antigen-binding fragments thereof can be produced recombinantly using techniques and materials readily available. For example, the anti-CoV-S antibodies and antigen-binding fragments thereof of the present disclosure can be produced in genetically engineered host cells according to conventional techniques. Suitable host cells are those cell types that can be transformed or transfected with exogenous DNA and grown in culture, and include bacteria, fungal cells (e.g., yeast), and cultured higher eukaryotic cells (including cultured cells of multicellular organisms), particularly cultured mammalian cells, e.g., human or non-human mammalian cells. In an exemplary embodiment these antibodies may be expressed in CHO cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor, N. Y.: Cold Spring Harbor Laboratory Press (1989), and Current Protocols in Molecular Biology, Ausubel et al., editors, New York, NY: Green and Wiley and Sons (1993).

In some exemplary embodiments the antibodies may be expressed in mating competent yeast, e.g., any haploid, diploid or tetraploid yeast that can be grown in culture. Yeast useful in fermentation expression methods may exist in a haploid, diploid, or other polyploid form.

A “selectable marker” herein refers to a gene or gene fragment that confers a growth phenotype (physical growth characteristic) on a cell receiving that gene as, for example through a transformation event. The selectable marker allows that cell to survive and grow in a selective growth medium under conditions in which cells that do not receive that selectable marker gene cannot grow. Selectable marker genes generally fall into several types, including positive selectable marker genes such as a gene that confers on a cell resistance to an antibiotic or other drug, temperature when two temperature sensitive (“ts”) mutants are crossed or a ts mutant is transformed; negative selectable marker genes such as a biosynthetic gene that confers on a cell the ability to grow in a medium without a specific nutrient needed by all cells that do not have that biosynthetic gene, or a mutagenized biosynthetic gene that confers on a cell inability to grow by cells that do not have the wild type gene; and the like.

An “expression vector” herein refers to DNA vectors containing elements that facilitate manipulation for the expression of a foreign protein within the target host cell, e.g., a bacterial, insect, yeast, plant, amphibian, reptile, avian, or mammalian cell, e.g., a CHO or HEK cell. Conveniently, manipulation of sequences and production of DNA for transformation may first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Stearns, T., Methods in yeast genetics: a Cold Spring Harbor Laboratory course manual, Plainview, NY: Cold Spring Harbor Laboratory Press (2000). Expression vectors for use in the methods of the disclosure may include yeast or mammalian specific sequences, including a selectable auxotrophic or drug marker for identifying transformed host strains. A drug marker may further be used to amplify copy number of the vector in a yeast host cell.

The polypeptide coding sequence of interest is operably linked to transcriptional and translational regulatory sequences that provide for expression of the polypeptide in the desired host cells, e.g., yeast or mammalian cells. These vector components may include, but are not limited to, one or more of the following: an enhancer element, a promoter, and a transcription termination sequence. Sequences for the secretion of the polypeptide may also be included, e.g. a signal sequence, and the like. An origin of replication, e.g., a yeast or mammalian origin of replication, is optional, as expression vectors may be integrated into the host cell genome.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or via a PCR/recombination method familiar to those skilled in the art (GATEWAY® Technology (universal method for cloning DNA); Invitrogen, Carlsbad California). If such sites do not exist, the synthetic oligonucleotide adapters or linkers are used in accordance with conventional practice.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The promoter fragment may also serve as the site for homologous recombination and integration of the expression vector into the same site in the host cell, e.g., yeast or mammalian cell, genome; alternatively, a selectable marker may be used as the site for homologous recombination. Suitable promoters for use in different eukaryotic and prokaryotic cells are well known and commercially available.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell, e.g., a mammalian cell, an insect cell, or a yeast cell. Additionally, these signal peptide sequences may be engineered to provide for enhanced secretion in expression systems. Secretion signals of interest also include mammalian and yeast signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et, al., Protein Eng., 11 (2): 75 (1998); and Kobayashi et, al., Therapeutic Apheresis, 2 (4): 257 (1998)).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion, and/or sequenced.

As an alternative to restriction and ligation of fragments, recombination methods based on specific attachment (“att”) sites and recombination enzymes may be used to insert DNA sequences into a vector. Such methods are described, for example, by Landy, Ann. Rev. Biochem., 58:913-949 (1989); and are known to those of skill in the art. Such methods utilize intermolecular DNA recombination that is mediated by a mixture of lambda and E. coli-encoded recombination proteins. Recombination occurs between att sites on the interacting DNA molecules. For a description of att sites see Weisberg and Landy, Site-Specific Recombination in Phage Lambda, in Lambda II, p. 211-250, Cold Spring Harbor, NY: Cold Spring Harbor Press (1983). The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, etc.

The expression host may be further modified by the introduction of sequences encoding one or more enzymes that enhance folding and disulfide bond formation, i.e. foldases, chaperonins, etc. Such sequences may be constitutively or inducibly expressed in the host cell, using vectors, markers, etc, as known in the art. Preferably the sequences, including transcriptional regulatory elements sufficient for the desired pattern of expression, are stably integrated in the yeast genome through a targeted methodology.

For example, the eukaryotic protein disulfide isomerase (“PDI”) is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of immunoglobulin heavy chain binding protein (“BIP”); cyclophilin; and the like.

Cultured mammalian cells are exemplary hosts for production of the disclosed anti-CoV-S antibodies and antigen-binding fragments thereof. As mentioned CHO cells are particularly suitable for expression of antibodies. Many procedures are known in the art for manufacturing monoclonal antibodies in mammalian cells. (Sec, Galfre, G, and Milstein, C., Methods Enzym., 73:3-46, 1981; Basalp et al., Turk. J. Biol., 24:189-196, 2000; Wurm, F. M., Nat. Biotechnol., 22:1393-1398, 2004; and Li et al., mAbs, 2 (5): 466-477, 2010). As mentioned in further detail infra, common host cell lines employed in mammalian monoclonal antibody manufacturing schemes include, but are not limited to, human embryonic retinoblast cell line PER.C6® (Crucell N. V., Leiden, The Netherlands), NS0 murine myeloma cells (Medical Research Council, London, UK), CV1 monkey kidney cell line, 293 human embryonic kidney cell line, BHK baby hamster kidney cell line, VERO African green monkey kidney cell line, human cervical carcinoma cell line HELA, MDCK canine kidney cells, BRL buffalo rat liver cells, W138 human lung cells, HepG2 human liver cells, MMT mouse mammary tumor cells, TRI cells, MRC5 cells, Fs4 cells, myeloma or lymphoma cells, or Chinese Hamster (Cricetulus griseus) Ovary (CHO) cells, and the like. Many different subclones or sub-cell lines of CHO cells known in the art that are useful and optimized for production of recombinant monoclonal antibodies, such as the DP12 (CHO K1 dhfr-) cell line, NS0 cells are a non-Ig secreting, non-light chain-synthesizing subclone of NS-1 cells that are resistant to azaguanine. Other Chinese Hamster and CHO cells are commercially available (from ATCC, etc.), including CHO-DXB11 (CHO-DUKX), CHO-pro3, CHO-DG44, CHO 1-15, CHO DP-12, Lec2, M1WT3, Lec8, pgsA-745, and the like, all of which are genetically altered to optimize the cell line for various parameters. Monoclonal antibodies are commonly manufactured using a batch fed method whereby the monoclonal antibody chains are expressed in a mammalian cell line and secreted into the tissue culture medium in a bioreactor. Medium (or feed) is continuously supplied to the bioreactor to maximize recombinant protein expression. Recombinant monoclonal antibody is then purified from the collected media. In some circumstances, additional steps are needed to reassemble the antibodies through reduction of disulfide bonds, etc. Such production methods can be scaled to be as large as 12,000 L in a single batch or more. It is now routine to obtain as much as 20 pg/cell/day or higher through the use of such cell lines and methodologies, providing titers as high as 10 g/L or more, amounting to 15 to 100 kg from bioreactors of 10 kL to 25 kL. (Li et al., 2010). Various details of this production methodology, including cloning of the polynucleotides encoding the antibodies into expression vectors, transfecting cells with these expression vectors, selecting for transfected cells, and expressing and purifying the recombinant monoclonal antibodies from these cells are provided below.

For recombinant production of an anti-CoV-S antibody or antigen-binding fragment in mammalian cells, nucleic acids encoding the antibody or fragment thereof are generally inserted into a replicable vector for further cloning (amplification of the DNA) or for expression. DNA encoding the antibody is readily isolated or synthesized using conventional procedures (e.g., by using oligonucleotide probes that are capable of binding specifically to DNAs encoding the heavy and light chains of the antibody). The vector components generally include, but are not limited to, one or more of the following: a signal sequence, an origin of replication, one or more marker genes, an enhancer element, a promoter, and a transcription termination sequence. Selection of promoters, terminators, selectable markers, vectors, and other elements is a matter of routine design within the level of ordinary skill in the art. Many such elements are known in the art and are available through commercial suppliers.

The antibodies of this disclosure may be produced recombinantly not only directly, but also as a fusion polypeptide with a heterologous polypeptide, which is preferably a signal sequence or other polypeptide having a specific cleavage site at the N-terminus of the mature protein or polypeptide. The homologous or heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

Such expression vectors and cloning vectors will generally contain a nucleic acid sequence that enables the vector to replicate in one or more selected host cells. Typically, in cloning vectors this sequence is one that enables the vector to replicate independently of the host chromosomal DNA, and includes origins of replication or autonomously replicating sequences. Such sequences are well known for a variety of bacteria, yeast, and viruses, e.g., the origin of replication from the plasmid pBR322 is suitable for most Gram-negative bacteria, the 2mu plasmid origin is suitable for yeast, and various viral origins (Simian Virus 40 (“SV40”), polyoma, adenovirus, vesicular stomatitis virus (“VSV”), or bovine papillomavirus (“BPV”) are useful for cloning vectors in mammalian cells. Generally, the origin of replication component is not needed for mammalian expression vectors (the SV40 origin may typically be used only because it contains the early promoter).

These vectors will also typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” Examples of such dominant selection use the drugs neomycin, mycophenolic acid, and hygromycin. An exemplary selectable marker is a gene encoding resistance to the antibiotic neomycin. Selection is carried out in the presence of a neomycin-type drug, such as G-418 or the like. Those cells that are successfully transformed with a heterologous gene produce a protein conferring drug resistance and thus survive the selection regimen.

Selection systems can also be used to increase the expression level of the gene of interest, a process referred to as “amplification.” Amplification of transfectants typically occurs by culturing the cells in the presence of a low level of the selective agent and then increasing the amount of selective agent to select for cells that produce high levels of the products of the introduced genes. Exemplary suitable selectable markers for mammalian cells are those that enable the identification of cells competent to take up the antibody nucleic acid, such as dihydrofolate reductase (“DHFR”), thymidine kinase, metallothionein-I and -II, preferably primate metallothionein genes, adenosine deaminase, ornithine decarboxylase, etc.

For example, an amplifiable selectable marker for mammalian cells is dihydrofolate reductase, which confers resistance to methotrexate. Other drug resistance genes (e.g. hygromycin resistance, multi-drug resistance, puromycin acetyltransferase) can also be used. Cells transformed with the DHFR selection gene are first identified by culturing all of the transformants in a culture medium that contains methotrexate (“MTX”), a competitive antagonist of DHFR. An appropriate host cell when wild-type DHFR is employed is the Chinese hamster ovary (“CHO”) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (“APH”) can be selected by cell growth in medium containing a selection agent for the selectable marker such as an aminoglycosidic antibiotic, e.g., kanamycin, neomycin, or G-418. See U.S. Pat. No. 4,965,199.

These vectors may comprise an enhancer sequence that facilitates transcription of a DNA encoding the antibody. Many enhancer sequences are known from mammalian genes (for example, globin, elastase, albumin, alpha-fetoprotein, and insulin). A frequently used enhancer is one derived from a eukaryotic cell virus. Examples thereof include the SV40 enhancer on the late side of the replication origin (bp 100-270), the cytomegalovirus early promoter enhancer, the polyoma enhancer on the late side of the replication origin, and adenovirus enhancers (See also Yaniv, Nature, 297:17-18 (1982) on enhancing elements for activation of eukaryotic promoters). The enhancer may be spliced into the vector at a position 5′ or 3′ to the antibody-encoding sequence, but is preferably located at a site 5′ from the promoter.

Expression and cloning vectors will also generally comprise a promoter that is recognized by the host organism and is operably linked to the antibody nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody transcription from vectors in mammalian host cells may be controlled, for example, by promoters obtained from the genomes of viruses such as polyoma virus, fowlpox virus, adenovirus (such as Adenovirus 2), BPV, avian sarcoma virus, cytomegalovirus, a retrovirus, hepatitis-B virus, and most preferably SV40, from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the BPV as a vector is disclosed in U.S. Pat. No. 4,419,446. A modification of this system is described in U.S. Pat. No. 4,601,978. See also Reyes et al., Nature, 297:598-601 (1982) on expression of human beta-interferon cDNA in mouse cells under the control of a thymidine kinase promoter from herpes simplex virus. Alternatively, the Rous sarcoma virus long terminal repeat can be used as the promoter.

Strong transcription promoters can be used, such as promoters from SV40, cytomegalovirus, or myeloproliferative sarcoma virus. See, e.g., U.S. Pat. No. 4,956,288 and U.S. Patent Publication No. 20030103986. Other suitable promoters include those from metallothionein genes (U.S. Pat. Nos. 4,579,821 and 4,601,978) and the adenovirus major late promoter. Expression vectors for use in mammalian cells include pZP-1, pZP-9, and pZMP21, which have been deposited with the American Type Culture Collection, 10801 University Blvd., Manassas, VA. USA under accession numbers 98669, 98668, and PTA-5266, respectively, and derivatives of these vectors.

Expression vectors used in eukaryotic host cells (yeast, fungus, insect, plant, animal, human, or a nucleated cell from other multicellular organism) will also generally contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO 94/11026 and the expression vector disclosed therein.

Suitable host cells for cloning or expressing the subject antibodies include prokaryote, yeast, or higher eukaryote cells described above. However, interest has been greatest in vertebrate cells, and propagation of vertebrate cells in culture has become a routine procedure. Examples of useful mammalian host cell lines are monkey kidney CV1 line transformed by SV40 (COS-1 (ATCC No. CRL 1650); and COS-7, ATCC CRL 1651); human embryonic kidney line (293 or 293 cells subcloned for growth in suspension culture, (ATCC No. CRL 1573; Graham et al., J. Gen. Virol., 36:59-72 (1977)); baby hamster kidney cells (BHK, ATCC CCL 10, ATCC No. CRL 1632; BHK 570, ATCC No. CRL 10314); CHO cells (CHO-K1, ATCC No. CCL 61; CHO-DG44, Urlaub et al., Proc. Natl. Acad. Sci. USA, 77:4216-4220 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). Additional suitable cell lines are known in the art and available from public depositories such as the American Type Culture Collection, Manassas, VA.

Host cells are transformed with the above-described expression or cloning vectors for antibody production and cultured in conventional nutrient media modified as appropriate for inducing promoters, selecting transformants, or amplifying the genes encoding the desired sequences as discussed supra.

The mammalian host cells used to produce the antibody of this disclosure may be cultured in a variety of media. Commercially available media such as Ham's F10 (Sigma-Aldrich Corporation, St. Louis, MO), Minimal Essential Medium ((“MEM” (Sigma-Aldrich Corporation, St. Louis, MO), Roswell Park Memorial Institute-1640 medium (“RPMI-1640”, Sigma-Aldrich Corporation, St. Louis, MO), and Dulbecco's Modified Eagle's Medium ((“DMEM” Sigma-Aldrich Corporation, St. Louis, MO) are suitable for culturing the host cells. In addition, any of the media described in Ham et al., Meth. Enz., 58:44 (1979), Barnes et al., Anal. Biochem., 102:255 (1980), U.S. Pat. Nos. 4,767,704; 4,657,866; 4,927,762; 4,560,655; or 5,122,469; WO 90/03430; WO 87/00195; or U.S. Patent Reexam No. 30,985 can be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. Methods of development and optimization of media and culture conditions are known in the art (See, Gronemeyer et al., Bioengineering, 1 (4): 188-212, 2014).

After culture conditions are optimized and a preferred cell line clone is selected, these cells are cultured (either adherent cells or suspension cultures) most typically in a batch-fed process in a bioreactor (many models are commercially available) that involves continuously feeding the cell culture with medium and feed, optimized for the particular cell line chosen and selected for this purpose. (See, Butler, M., Appl. Microbiol. Biotechnol., 68:283-291, 2005; and Kelley, B., mAb, 1 (5): 443-452, 2009). Perfusion systems are also available in which media and feed are continuously supplied to the culture while the same volume of media is being withdrawn from the bioreactor. (Wurm, 2004). Synthetic media, also commercially available, are available for growing cells in a batch-fed culture, avoiding the possibility of contamination from outside sources, such as with the use of animal components, such as bovine serum albumin, etc. However, animal-component-free hydrolysates are commercially available to help boost cell density, culture viability and productivity. (Li et al., 2010). Many studies have been performed in an effort to optimize cell culture media, including careful attention to head space available in roller bottles, redox potentials during growth and expression phases, presence of reducing agents to maintain disulfide bonds during production, etc. (See, for instance, Hutterer et al., mAbs, 5 (4): 608-613, 2013; and Mullan et al., BMC Proceed., 5 (Suppl 8): P110, 2011). Various methodologies have been developed to address the possibility of harmful oxidation during recombinant monoclonal antibody production. (See, for example, U.S. Pat. No. 8,574,869). Cultured cells may be grown by feeding nutrients continuously or as separately administered amounts. Often various process parameters such as cell concentration, pH, temperature, CO₂, dO₂, osmolality, amount of metabolites such as glucose, lactate, glutamine and glutamate, and the like, are monitored by the use of probes during the cell growth either on-line by direct connection to calibrated analyzers or off-line by intervention of operators. The culturing step also typically involves ensuring that the cells growing in culture maintain the transfected recombinant genes by any means known in the art for cell selection.

Following fermentation, i.e., upon reaching maximum cell growth and recombinant protein expression, the culturing step is typically followed by a harvesting step, whereby the cells are separated from the medium and a harvested cell culture media is thereby obtained. (See, Liu et al., mAbs, 2 (5): 480-499, 2010). Typically, various purification steps, involving column chromatography and the like, follow culturing to separate the recombinant monoclonal antibody from cell components and cell culture media components. The exact purification steps needed for this phase of the production of recombinant monoclonal antibodies depends on the site of expression of the proteins, i.e., in the cytosol of the cells themselves, or the more commonly preferred route of protein excreted into the cell culture medium. Various cell components may be separated using techniques known in the art such as differential centrifugation techniques, gravity-based cell settling, and/or size exclusion chromatograph/filtration techniques that can include tangential flow micro-filtration or depth filtration. (Sec, Pollock et al., Biotechnol. Bioeng., 110:206-219, 2013, and Liu et al., 2010). Centrifugation of cell components may be achieved on a large scale by use of continuous disk stack centrifuges followed by clarification using depth and membrane filters. (See, Kelley, 2009). Most often, after clarification, the recombinant protein is further purified by Protein A chromatography due to the high affinity of Protein A for the Fc domain of antibodies, and typically occurs using a low pH/acidification elution step (typically the acidification step is combined with a precautionary virus inactivation step). Flocculation and/or precipitation steps using acidic or cationic polyelectrolytes may also be employed to separate animal cells in suspension cultures from soluble proteins. (Liu et al., 2010). Lastly, anion- and cation-exchange chromatography, hydrophobic interaction chromatograph (“HIC”), hydrophobic charge induction chromatograph (HCIC), hydroxyapatite chromatography using ceramic hydroxyapatite (Ca₅(PO₄)₃OH)₂, and combinations of these techniques are typically used to polish the solution of recombinant monoclonal antibody. Final formulation and concentration of the desired monoclonal antibody may be achieved by use of ultracentrifugation techniques. Purification yields are typically 70 to 80%. (Kelley, 2009).

The terms “desired protein” or “desired antibody” herein are used interchangeably and refer generally to a parent antibody specific to a target, i.e., CoV—S or a chimeric or humanized antibody or a binding portion thereof derived therefrom as described herein. The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” Examples thereof include chimeric antibodies, human antibodies and other non-human mammalian antibodies, humanized antibodies, single chain antibodies (such as scFvs), camelbodies, nanobodies, IgNAR (single-chain antibodies which may be derived from sharks, for example), small-modular immunopharmaceuticals (“SMIPs”), and antibody fragments such as Fabs, Fab′, F(ab′)₂, and the like (See Streltsov et al., Protein Sci., 14 (11): 2901-9 (2005); Greenberg et al., Nature, 374 (6518): 168-73 (1995); Nuttall et al., Mol. Immunol., 38 (4): 313-26 (2001); Hamers-Casterman et al., Nature, 363 (6428): 446-8 (1993); Gill et al., Curr. Opin. Biotechnol., (6): 653-8 (2006)).

For example, antibodies or antigen-binding fragments thereof may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones that co-express a heavy and light chain (resembling the Fab fragment or antigen-binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (“aa”) substitutions, additions, or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject disclosure are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the VI, and VH regions, obtained from antibody producing cells of one species with the constant light and heavy chain regions from another. Typically, chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the disclosure may be selected from IgG1, IgG2, IgG3, and IgG4 constant regions.

Humanized antibodies are engineered to contain even more human-like immunoglobulin domains, and incorporate only the complementarity determining regions of the animal-derived antibody. This is accomplished by carefully examining the sequence of the hyper-variable loops of the variable regions of the monoclonal antibody and fitting them to the structure of the human antibody chains. Although facially complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment” or minimal immunoglobulins may be designed utilizing recombinant immunoglobulin techniques. For instance, “Fv” immunoglobulins for use in the present disclosure may be produced by synthesizing a fused variable light chain region and a variable heavy chain region. Combinations of antibodies are also of interest, e.g. diabodies, which comprise two distinct Fv specificities. In another embodiment, small molecule immunopharmaceuticals (“SMIPs”), camelbodies, nanobodies, and IgNAR are encompassed by immunoglobulin fragments.

Immunoglobulins and fragments thereof may be modified post-translationally, e.g, to add effector moieties such as chemical linkers, detectable moieties, such as fluorescent dyes, enzymes, toxins, substrates, bioluminescent materials, radioactive materials, chemiluminescent moieties, and the like, or specific binding moieties, such as streptavidin, avidin, or biotin, and the like may be utilized in the methods and compositions of the present disclosure. Examples of additional effector molecules are provided infra.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

A “heterologous” region or domain of a DNA construct is an identifiable segment of DNA within a larger DNA molecule that is not found in association with the larger molecule in nature. Thus, when the heterologous region encodes a mammalian gene, the DNA flanking the gene usually does not flank the mammalian genomic DNA in the genome of the source organism. Another example of a heterologous region is a construct where the coding sequence itself is not found in nature (e.g., a cDNA where the genomic coding sequence contains introns or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of initiating transcription of a downstream (3′ direction) coding sequence, and typically contain additional sites for binding of regulatory molecules, e.g., transcription factors, that affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

The general structure of antibodies in vertebrates now is well understood. See Edelman, G. M., Ann. N.Y. Acad. Sci., 190:5 (1971). Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the F_{ab} region; the stem portion of the “Y” configuration is designated the F_{C} region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (see Kabat, E. A., Structural Concepts in Immunology and Immunochemistry, 2nd Ed., p. 413-436, New York, NY: Holt, Rinehart, Winston (1976)), and other cellular responses (see Andrews et al., Clinical Immunology, pp. 1-18, W. B. Sanders, Philadelphia, PA (1980); Kohl et al., Immunology, 48:187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B-cells.

The expression “variable region” or “VR” refers to the domains within each pair of light and heavy chains in an antibody that are involved directly in binding the antibody to the antigen. Each heavy chain has at one end a variable region (VH) followed by a number of constant domains. Each light chain has a variable region (VL) at one end and a constant domain at its other end; the constant domain of the light chain is aligned with the first constant domain of the heavy chain, and the light chain variable domain is aligned with the variable domain of the heavy chain.

The expressions “complementarity-determining region.” “hypervariable region,” or “CDR” refer to one or more of the hyper-variable or complementarity-determining regions (“CDRs”) found in the variable regions of light or heavy chains of an antibody (See Kabat et al., Sequences of Proteins of Immunological Interest, 4th ed., Bethesda, MD: U.S. Dept. of Health and Human Services, Public Health Service, National Institutes of Health (1987)). These expressions include the hypervariable regions as defined by Kabat et al., (Sequences of Proteins of Immunological Interest, NIH Publication No. 91-3242, Bethesda, MD: U.S. Dept. of Health and Human Services, National Institutes of Health (1983)) or the hypervariable loops in 3-dimensional structures of antibodies (Chothia and Lesk, J. Mol. Biol., 196:901-917 (1987)). The CDRs in each chain are held in close proximity by framework regions (“FRs”) and, with the CDRs from the other chain, contribute to the formation of the antigen binding site. Within the CDRs there are select amino acids that have been described as the selectivity determining regions (“SDRs”) that represent the critical contact residues used by the CDR in the antibody-antigen interaction (see Kashmiri et al., Methods, 36 (1): 25-34 (2005)).

An “epitope” or “binding site” is an area or region on an antigen to which an antigen-binding peptide (such as an antibody) specifically binds. A protein epitope may comprise amino acid residues directly involved in the binding (also called immunodominant component of the epitope) and other amino acid residues, which are not directly involved in the binding, such as amino acid residues that are effectively blocked by the specifically antigen binding peptide (in other words, the amino acid residue is within the “footprint” of the specifically antigen binding peptide). The term epitope herein includes both types of amino acid binding sites in any particular region of CoV-S, e.g., SARS-COV—S or SARS-COV-2-S, that specifically binds to an anti-CoV-S antibody. CoV-S may comprise a number of different epitopes, which may include, without limitation, (1) linear peptide antigenic determinants, (2) conformational antigenic determinants that consist of one or more non-contiguous amino acids located near each other in a mature CoV-S conformation; and (3) post-translational antigenic determinants that consist, either in whole or part, of molecular structures covalently attached to a CoV-S protein such as carbohydrate groups. In particular, the term “epitope” includes the specific residues in a protein or peptide, e.g., CoV-S, which are involved in the binding of an antibody to such protein or peptide as determined by known and accepted methods such as alanine scanning techniques or the use of various S protein portions with varying lengths.

The phrase that an antibody (e.g., first antibody) binds “substantially” or “at least partially” the same epitope as another antibody (e.g., second antibody) means that the epitope binding site for the first antibody comprises at least 10%, 20%, 30%, 40%, 50%, 60%, 70%, 80%, 90%, or more of the amino acid residues on the antigen that constitutes the epitope binding site of the second antibody. Also, that a first antibody binds substantially or partially the same or overlapping epitope as a second antibody means that the first and second antibodies compete in binding to the antigen, as described above. Thus, the term “binds to substantially the same epitope or determinant as” a monoclonal antibody means that an antibody “competes” with the antibody.

The phrase “binds to the same or overlapping epitope or determinant as” an antibody of interest means that an antibody “competes” with said antibody of interest for at least one, (e.g., at least 2, at least 3, at least 4, at least 5) or all residues on CoV-S to which said antibody of interest specifically binds. The identification of one or more antibodies that bind(s) to substantially or essentially the same epitope as the monoclonal antibodies described herein can be readily determined using alanine scanning. Additionally, any one of variety of immunological screening assays in which antibody competition can be assessed. A number of such assays are routinely practiced and well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same or overlapping epitope as the monoclonal antibody described herein.

For example, where the test antibodies to be examined are obtained from different source animals, or are even of a different Ig isotype, a simple competition assay may be employed in which the control antibody is mixed with the test antibody and then applied to a sample containing CoV-S. Protocols based upon ELISAs, radioimmunoassays, Western blotting, and the use of BIACORE® (GE Healthcare Life Sciences, Marlborough, MA) analysis are suitable for use in such simple competition studies.

In certain embodiments, the control anti-CoV-S antibody is pre-mixed with varying amounts of the test antibody (e.g., in ratios of about 1:1, 1:2, 1:10, or about 1:100) for a period of time prior to applying to the CoV-S (e.g., SARS-COV—S or SARS-COV-2-S) antigen sample. In other embodiments, the control and varying amounts of test antibody can simply be added separately and admixed during exposure to the SARS-COV—S or SARS-COV-2-S antigen sample. As long as bound antibodies can be distinguished from free antibodies (e.g., by using separation or washing techniques to eliminate unbound antibodies) and control antibody from the test antibody (e.g., by using species specific or isotype specific secondary antibodies or by specifically labeling the control antibody with a detectable label) it can be determined if the test antibody reduces the binding of the control antibody to the SARS-COV—S or SARS-COV-2-S antigens, indicating that the test antibody recognizes substantially the same epitope as the control anti-CoV-S antibody. The binding of the (labeled) control antibody in the presence of a completely irrelevant antibody (that does not bind CoV-S) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of the control antibody to SARS-COV—S or SARS-COV-2-S by at least about 50%, such as at least about 60%, or more preferably at least about 70% (e.g., about 65-100%), at any ratio of test antibody between about 1:1 or 1:10 and about 1:100 is considered to be an antibody that binds to substantially the same or overlapping epitope or determinant as the control antibody.

Preferably, such test antibody will reduce the binding of the control antibody to SARS-COV—S or SARS-COV-2-S (or another CoV-S) antigen preferably at least about 50%, at least about 60%, at least about 80%, or at least about 90% (e.g., about 95%) of the binding of the control antibody observed in the absence of the test antibody.

A simple competition assay in which a test antibody is applied at saturating concentration to a surface onto which SARS-COV—S or SARS-COV-2-S (or another CoV-S) is immobilized also may be advantageously employed. The surface in the simple competition assay is preferably a BIACORE® (GE Healthcare Life Sciences, Marlborough, MA) chip (or other media suitable for surface plasmon resonance (“SPR”) analysis). The binding of a control antibody that binds SARS-COV—S or SARS-CoV-2-S to the COV-S-coated surface is measured. This binding to the SARS-COV-S- or SARS-CoV-2-S-containing surface of the control antibody alone is compared with the binding of the control antibody in the presence of a test antibody. A significant reduction in binding to the SARS-COV-S- or SARS-COV-2-S-containing surface by the control antibody in the presence of a test antibody indicates that the test antibody recognizes substantially the same epitope as the control antibody such that the test antibody “competes” with the control antibody. Any test antibody that reduces the binding of control antibody by at least about 20% or more, at least about 40%, at least about 50%, at least about 70%, or more, can be considered to be an antibody that binds to substantially the same epitope or determinant as the control antibody. Preferably, such test antibody will reduce the binding of the control antibody to SARS-COV—S or SARS-COV-2-S by at least about 50% (e.g., at least about 60%, at least about 70%, or more). It will be appreciated that the order of control and test antibodies can be reversed; i.e, the control antibody can be first bound to the surface and then the test antibody is brought into contact with the surface thereafter in a competition assay. Preferably, the “sandwich-style” binding assay infra is used. Alternatively, the antibody having greater affinity for SARS-COV—S or SARS-COV-2-S antigen is bound to the SARS-COV-S- or SARS-COV-2-S-containing surface first, as it will be expected that the decrease in binding seen for the second antibody (assuming the antibodies are competing) will be of greater magnitude. Further examples of such assays are provided in e.g., Saunal and Regenmortel, J. Immunol. Methods, 183:33-41 (1995), the disclosure of which is incorporated herein by reference.

In addition, whether an antibody binds the same or overlapping epitope(s) on COV-S as another antibody or the epitope bound by a test antibody may in particular be determined using a Western-blot based assay. In this assay a library of peptides corresponding to the antigen bound by the antibody, the CoV-S protein, is made, that comprise overlapping portions of the protein, typically 10-25, 10-20, or 10-15 amino acids long. These different overlapping amino acid peptides encompassing the CoV-S sequence are synthesized and covalently bound to a PEPSPOTS™ nitrocellulose membrane (JPT Peptide Technologies, Berlin, Germany). Blots are then prepared and probed according to the manufacturer's recommendations.

Essentially, the immunoblot assay then detects by fluorometric means what peptides in the library bind to the test antibody and thereby can identify what residues on the antigen, i.e., COV-S, interact with the test antibody. (See U.S. Pat. No. 7,935,340, incorporated by reference herein).

Various epitope mapping techniques are known in the art. By way of example, X-ray co-crystallography of the antigen and antibody; NMR; SPR (e.g., at 25° or 37° C.); array-based oligo-peptide scanning (or “pepscan analysis”); site-directed mutagenesis (e.g., alanine scanning); mutagenesis mapping; hydrogen-deuterium exchange; phage display; and limited proteolysis are all epitope mapping techniques that are well known in the art (See, e.g., Epitope Mapping Protocols: Second Edition, Methods in Molecular Biology,, editors Mike Schutkowski and Ulrich Reineke, 2nd Ed., New York, NY: Humana Press (2009), and Epitope Mapping Protocols, Methods in Molecular Biology, editor Glenn Morris, 1st Ed., New York, NY: Humana Press (1996), both of which are herein incorporated by referenced in their entirety).

The identification of one or more antibodies that bind(s) to substantially or essentially the same epitope as the monoclonal antibodies described herein, e.g., any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, can be readily determined using any one of variety of immunological screening assays in which antibody competition can be assessed. A number of such assays are routinely practiced and well known in the art (see, e.g., U.S. Pat. No. 5,660,827, issued Aug. 26, 1997, which is incorporated herein by reference). It will be understood that determining the epitope to which an antibody described herein binds is not in any way required to identify an antibody that binds to the same or substantially the same epitope as the monoclonal antibody described herein.

For example, where the test antibodies to be examined are obtained from different source animals, or are even of a different Ig isotype, a simple competition assay may be employed in which the control antibody (one of antibodies selected from the group consisting of ADI-58120, ADI-58121. ADI-58122, ADI-58123, ADI-58124, ADI-58125, ADI-58126, ADI-58127, ADI-58128, ADI-58129, ADI-58130, ADI-58131, for example) is mixed with the test antibody and then applied to a sample containing either or both SARS-COV—S or SARS-COV-2-S, each of which is known to be bound by antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131. Protocols based upon ELISAs, radioimmunoassays, Western blotting, and BIACORE® (GE Healthcare Life Sciences, Marlborough, MA) analysis (as described in the Examples section herein) are suitable for use in such simple competition studies.

In certain embodiments, the method comprises pre-mixing the control antibody with varying amounts of the test antibody (e.g., in ratios of about 1:1, 1:2, 1:10, or about 1:100) for a period of time prior to applying to the CoV-S antigen sample. In other embodiments, the control and varying amounts of test antibody can be added separately and admixed during exposure to the CoV-S antigen sample. As long as bound antibodies can be distinguished from free antibodies (e.g., by using separation or washing techniques to eliminate unbound antibodies) and control antibody from the test antibody (e.g., by using species specific or isotype specific secondary antibodies or by specifically labelling the control antibody with a detectable label), the method can be used to determine that the test antibody reduces the binding of the control antibody to the COV-S antigen, indicating that the test antibody recognizes substantially the same epitope as the control antibody (e.g., antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131). The binding of the (labeled) control antibody in the presence of a completely irrelevant antibody (that does not bind CoV-S) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, to both of SARS-COV—S or SARS-COV-2-S antigens by at least about 50%, such as at least about 60%, or more preferably at least about 70% (e.g., about 65-100%), at any ratio of control antibody selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, test antibody between about 1:1 or 1:10 and about 1:100 is considered to be an antibody that binds to substantially the same epitope or determinant as any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, respectively. Preferably, such test antibody will reduce the binding of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, to at least one, preferably each, of the SARS-COV—S or SARS-COV-2-S antigens preferably at least about 50%, at least about 60%, at least about 80% or at least about 90% (e.g., about 95%) of the binding of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131, observed in the absence of the test antibody. These methods can be adapted to identify and/or evaluate antibodies that compete with other control antibodies.

A simple competition assay in which a test antibody is applied at saturating concentration to a surface onto which either SARS-COV—S or SARS-COV-2-S, or both, are immobilized also may be advantageously employed. The surface in the simple competition assay is preferably of a media suitable for OCTET® and/or PROTEON®. The binding of a control antibody (e.g., any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131) to the CoV-S-coated surface is measured. This binding to the CoV-S-containing surface of the control antibody alone is compared with the binding of the control antibody in the presence of a test antibody. A significant reduction in binding to the CoV-S-containing surface by the control antibody in the presence of a test antibody indicates that the test antibody recognizes substantially the same epitope as the control antibody such that the test antibody “competes” with the control antibody. Any test antibody that reduces the binding of control antibody (such as anyone of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131) to both of SARS-COV—S and SARS-COV-2-S antigens by at least about 20% or more, at least about 40%, at least about 50%, at least about 70%, or more, can be considered to be an antibody that binds to substantially the same epitope or determinant as the control antibody (e.g., any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131). Preferably, such test antibody will reduce the binding of the control antibody (e.g., any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, ADI-58131) to the CoV-S antigen by at least about 50% (e.g., at least about 60%, at least about 70%, or more). It will be appreciated that the order of control and test antibodies can be reversed; i.e, the control antibody can be first bound to the surface and then the test antibody is brought into contact with the surface thereafter in a competition assay. Preferably, the antibody having higher affinity for SARS-COV—S and SARS-COV-2-S is bound to the CoV-S-containing surface first, as it will be expected that the decrease in binding seen for the second antibody (assuming the antibodies are competing) will be of greater magnitude. Further examples of such assays are provided in, e.g., Saunal and Regenmortel, J. Immunol. Methods, 183:33-41 (1989), the disclosure of which is incorporated herein by reference.

Determination of whether an antibody, antigen-binding fragment thereof, or antibody derivative, e.g., an affinity-matured antibody or antigen binding fragment of any of the anti-CoV-S antibodies exemplified herein, binds within one of the epitope regions defined above can be carried out in ways known to the person skilled in the art. In another example of such mapping/characterization methods, an epitope region for an anti-CoV-S antibody may be determined by epitope “footprinting” using chemical modification of the exposed amines/carboxyls in the SARS—CoV—S and SARS-COV-2-S protein. One specific example of such a foot-printing technique is the use of hydrogen-deuterium exchange detected by mass spectrometry (“HXMS”), wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry (See, e.g., Ehring H., Analytical Biochemistry, 267 (2): 252-259 (1999) and Engen, J. R. & Smith, D. L., Anal. Chem., 73: 256A-265A (2001)). Another example of a suitable epitope identification technique is nuclear magnetic resonance epitope mapping (“NMR”), where typically the position of the signals in two-dimensional NMR spectras of the free antigen and the antigen complexed with the antigen binding peptide, such as an antibody, are compared. The antigen typically is selectively isotopically labeled with ¹⁵N so that only signals corresponding to the antigen and no signals from the antigen binding peptide are seen in the NMR-spectrum. Antigen signals originating from amino acids involved in the interaction with the antigen binding peptide typically will shift position in the spectras of the complex compared to the spectras of the free antigen, and the amino acids involved in the binding can be identified that way. See, e.g., Ernst Schering Res. Found. Workshop, (44): 149-67 (2004); Huang et al., J. Mol. Biol., 281 (1): 61-67 (1998); and Saito and Patterson, Methods, 9 (3): 516-24 (1996). Epitope mapping/characterization also can be performed using mass spectrometry (“MS”) methods (See, e.g., Downard, J. Mass Spectrom., 35 (4): 493-503 (2000) and Kiselar and Downard, Anal. Chem., 71 (9): 1792-801 (1999)).

Protease digestion techniques also can be useful in the context of epitope mapping and identification. Antigenic determinant-relevant regions/sequences can be determined by protease digestion, e.g. by using trypsin in a ratio of about 1:50 to SARS-COV—S or SARS-COV-2-S overnight (“o/n”) digestion at 37° C., and pH 7-8, followed by mass spectrometry (“MS”) analysis for peptide identification. The peptides protected from trypsin cleavage by the anti-CoV-S antibody can subsequently be identified by comparison of samples subjected to trypsin digestion and samples incubated with antibody and then subjected to digestion by e.g, trypsin (thereby revealing a footprint for the antibody). Other enzymes like chymotrypsin or pepsin can be used in similar epitope characterization methods. Moreover, enzymatic digestion can provide a quick method for analyzing whether a potential antigenic determinant sequence is within a region of CoV-S in the context of a CoV-S-binding polypeptide. If the polypeptide is not surface exposed, it is most likely not relevant in terms of immunogenicity/antigenicity (See, e.g., Manca, Ann. 1st. Super. Sanità., 27 (1): 15-9 (1991) for a discussion of similar techniques).

Site-directed mutagenesis is another technique useful for characterization of a binding epitope. For example, in “alanine-scanning” site-directed mutagenesis (also known as alanine scanning, alanine scanning mutagenesis, alanine scanning mutations, combinatorial alanine scanning, or creation of alanine point mutations, for example), each residue within a protein segment is replaced with an alanine residue (or another residue such as valine where alanine is present in the wild-type sequence) through such methodologies as direct peptide or protein synthesis, site-directed mutagenesis, the GENEART™ Mutagenesis Service (Thermo Fisher Scientific, Waltham, MA U.S.A.) or shotgun mutagenesis, for example. A series of single point mutants of the molecule is thereby generated using this technique; the number of mutants generated is equivalent to the number of residues in the molecule, each residue being replaced, one at a time, by a single alanine residue. Alanine is generally used to replace native (wild-type) residues because of its non-bulky, chemically inert, methyl functional group that can mimic the secondary structure preferences that many other amino acids may possess. Subsequently, the effects replacing a native residue with an alanine has on binding affinity of an alanine scanning mutant and its binding partner can be measured using such methods as, but not limited to, SPR binding experiments. If a mutation leads to a significant reduction in binding affinity, it is most likely that the mutated residue is involved in binding. Monoclonal antibodies specific for structural epitopes (i.e., antibodies that do not bind the unfolded protein) can be used as a positive control for binding affinity experiments to verify that the alanine-replacement does not influence the overall tertiary structure of the protein (as changes to the overall fold of the protein may indirectly affect binding and thereby produce a false positive result). See, e.g., Clackson and Wells, Science, 267:383-386 (1995); Weiss et al., Proc. Natl. Acad. Sci. USA, 97 (16): 8950-8954 (2000); and Wells, Proc. Natl. Acad. Sci. USA, 93:1-6 (1996). Example 5 identifies the specific epitope or residues of CoV-S which specifically interact with the anti-CoV-S antibodies disclosed herein.

Electron microscopy can also be used for epitope “footprinting”. For example, Wang et al., Nature, 355:275-278 (1992) used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include SPR (sold commercially as the BIACORE® system, GE Healthcare Life Sciences, Marlborough, MA) and reflectometric interference spectroscopy (“RifS”) (See, e.g., Fagerstam et al., Journal of Molecular Recognition, 3:208-14 (1990); Nice et al., J. Chromatogr., 646:159-168 (1993); Leipert et al., Angew. Chem. Int. Ed., 37:3308-3311 (1998); Kroger et al., Biosensors and Bioelectronics, 17:937-944 (2002)).

The expressions “framework region” or “FR” refer to one or more of the framework regions within the variable regions of the light and heavy chains of an antibody (See Kabat et al., Sequences of Proteins of Immunological Interest, 4th edition, Bethesda, MD: U.S. Dept. of Health and Human Services, Public Health Service, National Institutes of Health (1987)). These expressions include those amino acid sequence regions interposed between the CDRs within the variable regions of the light and heavy chains of an antibody.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th edition, Bethesda, MD: U.S. Dept. of Health and Human Services, Public Health Service, National Institutes of Health (1991). The Fc region of an immunoglobulin generally comprises two constant domains, CH2 and CH3.

The terms “Fc receptor” and “FcR” describe a receptor that binds to the Fc region of an antibody. The preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92 (1991); Capel et al., Immunomethods, 4:25-34 (1994); and de Haas et al., J. Lab. Clin. Med., 126:330-41 (1995). “FcR” also includes the neonatal receptor, FcRn, which is responsible for the transfer of maternal IgGs to the fetus (Guyer et al., J. Immunol., 117:587 (1976); and Kim et al., J. Immunol., 24:249 (1994)), and which primarily functions to modulate and/or extend the half-life of antibodies in circulation. To the extent that the disclosed anti-CoV-S antibodies are aglycosylated, as a result of the expression system and/or sequence, the subject antibodies are expected to bind FcRn receptors, but not to bind (or to minimally bind) Fcγ receptors.

A “functional Fc region” possesses at least one effector function of a native sequence Fc region. Exemplary “effector functions” include Clq binding; complement dependent cytotoxicity (“CDC”); Fc receptor binding; antibody-dependent cell-mediated cytotoxicity (“ADCC”); phagocytosis; down-regulation of cell surface receptors (e.g. B cell receptor (“BCR”)), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence that differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, or at least about 99% sequence identity therewith.

In some embodiments, the Fc region of an antibody or antigen-binding antibody fragment of the present disclosure may bind to an Fc receptor (FcR). The FcR may be, but is not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn). The Fc may be an IgM, IgD, IgG, IgE, or IgA isotype. An IgG isotype may be an IgG1, IgG2, IgG3, or IgG4.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X, et al., Protein Cell. 2018 January; 9 (1): 63-73; Dall'Acqua W. F, et al., J Biol Chem. 2006 Aug. 18; 281 (33): 23514-24. Epub 2006 Jun. 21; Monnet C, et al, Front Immunol. 2015 Feb. 4; 6:39, doi: 10.3389/fimmu.2015.00039, eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289 (6): 3571-3590).

Any of the antibody variable region sequences disclosed herein may be used in combination with a wild-type (WT) Fc or a variant Fc. In particular embodiments, an Fc selected from the Fc sequences described in Table 38 may be used. Any of the variable region sequences disclosed herein may be used in combination with any appropriate Fc including any of the Fc variants provided in Table 38 to form an antibody or an antigen-binding antibody fragment of the present disclosure. The lysine (K) at the C-terminus of each Fc may be present or absent.

An IgG1-type Fc optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, 1332E, S298A, E333A, K334A, L234Y, L235Q, G236W. S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to the EU index as in Kabat) (Dall'Acqua W. F, et al., J Biol Chem. 2006 Aug. 18; 281 (33): 23514-24. Epub 2006 Jun. 21; Wang X, et al., Protein Cell. 2018 January; 9 (1): 63-73), or for example, N434A, Q438R. S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fc include, but not limited to: M252Y, S254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat). In particular embodiments, an IgG1-type Fc variant may comprise the amino acid sequence of SEQ ID NOS: 411, 412, 413, 414, 415, 416, or 417. In one embodiment, the Fc variant is an LA variant and comprises the amino acid sequence of SEQ ID NO: 413.

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, S254T. T256E, and/or any combination thereof (the residue numbering is according to the EU index as in Kabat). The Fc region optionally may further comprise one or more additional amino acid substitutions. Such substitutions may include but are not limited to M252Y, S254T. T256E, and/or any combination thereof (the residue numbering is according to the EU index as in Kabat).

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P. F234V, L235A, G237A, E318A, S228P, L236E, S241P. L248E, T394D, M252Y, S254T. T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T, et al., Proc Natl Acad Sci USA. 2017 Mar. 28; 114 (13): 3485-3490, doi: 10.1073/pnas. 1702173114. Epub 2017 Mar. 13).

An “isolated” antibody, as used herein, refers to an antibody that is substantially free of other antibodies having different antigenic specificities. In some embodiments, an isolated antibody is substantially free of other unintended cellular material and/or chemicals.

As used herein, “specific binding” or “specifically binds” means that the interaction of the antibody, or antigen-binding portion thereof, with an antigen is dependent upon the presence of a particular structure (e.g., antigenic determinant or epitope). For example, the antibody, or antigen-binding portion thereof, binds to a specific protein, rather than proteins generally. In some embodiments, an antibody, or antigen-binding portion thereof, specifically binds a target, e.g., SARS—CoV—S and/or SARS-COV-S-2. In some embodiments, an antibody, or antigen-binding portion thereof, specifically binds to more than one coronavirus spike protein, e.g., the spike protein of SARS-COV—S and the spike protein of SARS-COV-2-S, for example. In some embodiments, the antibody, or antigen-binding portion thereof, specifically binds to two different, but related, antigens, e.g., the spike protein of SARS-COV1-S and the spike protein of SARS-COV2-S, e.g., via a conserved epitope.

### B. Pharmaceutical Formulations

The present disclosure provides pharmaceutical formulations, in particular, a high concentration formulation, comprising a therapeutic polypeptide. As discussed herein, one challenge associated with high-concentration formulations is increased electrostatic interaction between proteins and excipients and is a result of increased protein-charge density at high-protein concentrations. Such interactions can create an offset between excipient levels in final products and diafiltration buffers during the ultrafiltration process. The inventors of the present disclosure, however, have surprisingly and unexpectedly overcome this challenge and achieved a final formulation having a high concentration of therapeutic polypeptide, e.g., greater than 100 mg/mL, e.g., 150 mg/mL, or 200 mg/mL; a viscosity of under 20 cPoise; a physiological isotonicity, i.e., between 270-400 mOsm/kg; an opalescence less than 60 NTU opalescence; and a high level of stability at about 2-8° C., e.g., a shelf-life of at least 2 years, and at about 25° C., e.g., a shelf-life of at least 2 weeks. The high concentration formulation was desirable to enable intramuscular (IM) route of administration. In addition, the high concentration formulation of the present disclosure is also stable upon freezing and thawing, thereby enabling the drug substance (i.e., stored frozen) to have the same formulation and product concentration as the drug product.

Accordingly to certain embodiments of the present disclosure, the therapeutic polypeptide is an isolated antibody, or antigen-binding fragment thereof, which specifically binds to the spike protein of a coronavirus (“CoV-S”). More specifically, the present disclosure provides pharmaceutical formulations that comprise: (i) an isolated antibody, or antigen-binding fragment thereof, which specifically binds to the spike protein of a coronavirus (“CoV-S”); (ii) one or more buffers selected from the group consisting of an acetate buffer, a succinate buffer, a citrate buffer, a histidine buffer, and a phosphate buffer having a pH of about 4.5-7.0; (iii) one or more pharmaceutically acceptable excipients selected from the group consisting of sucrose, mannitol, glycine, proline, sodium chloride, arginine hydrochloride, arginine-glutamate, and sorbitol; and/or (iv) a surfactant. Specific exemplary components and formulations included within the present disclosure are described in detail below.

As used herein the term “pharmaceutical formulation” refers to preparations which are in such form as to permit the active ingredients to be effective, and which contains no additional components which are toxic to the subjects to which the formulation would be administered.

a. Anti-CoV-S Antibodies and Binding Fragments Thereof Having Binding Activity for CoV-S

The pharmaceutical formulations of the present disclosure may comprise an anti-CoV-S antibody or binding fragment thereof having binding activity for CoV-S.

As used herein, “CoV-S” refers to the S protein of a coronavirus which is expressed on the surface of virions as a structural protein. As mentioned previously, the S protein plays an essential role for coronaviruses in binding to receptors on the host cell and determines host tropism (Zhu Z, et al., Infect Genet Evol. 2018 July; 61:183-184). SARS-COV and SARS-COV-2 bind to angiotensin-converting enzyme 2 (ACE2) of the host cell via the S protein's receptor-binding domains (RBDs) and uses ACE2 as a receptor to enter the host cells (Ge X. Y, et al., Nature. 2013 Nov. 28; 503 (7477): 535-8, doi: 10.1038/nature12711. Epub 2013 Oct. 30.; Hoffmann M, et al., Cell. 2020 Mar. 4, pii: S0092-8674 (20) 30229-4). SARS-COV can also use CD209L (also known as L-SIGN) as an alternative receptor (Jeffers S. A, et al., Proc Natl Acad Sci USA. 2004 Nov. 2; 101 (44): 15748-53. Epub 2004 Oct. 20). MERS-COV binds dipeptidyl peptidase 4 (“DPP4”, also known as CD26) of the host cells via a different RBD of the S protein. Cell entry of coronaviruses depends on not only binding of the S protein to a host cell receptor but often also priming of the S protein by host cell proteases, and recently SARS-COV-2 was found to use the serine protease TMPRSS2 for S protein priming and then ACE2 for entry (Wu A, et al., Cell Host Microbe. 2020 Mar. 11; 27 (3): 325-328; Hoffmann M, et al., Cell. 2020 Mar. 4, pii: S0092-8674 (20) 30229-4).

The S protein of SARS-COV is referred to as SARS-COV—S and may for example comprise the amino acid sequence of SEQ ID NO: 401 (1288 amino acids). The S protein of SARS-COV-2 is referred to as SARS-COV-2-S and may for example comprise the amino acid sequence of SEQ ID NO: 403 (1273 amino acids).

The present disclosure provides exemplary antibodies and antigen-binding antibody fragments that specifically bind to CoV for use in the pharmaceutical formulations of the present disclosure, wherein at least some of these antibodies and antigen-binding antibody fragments specifically bind to SARS-COV-2-S and/or SARS-COV-2-S. Due to the sequence similarity among different CoV species, such antibodies or antigen-binding antibody fragments of the present disclosure may also cross react with the S protein of other CoV species.

The exemplary S proteins of CoV that the antibodies or antigen-binding antibody fragments in pharmaceutical formulations of the present disclosure may specifically bind include by way of example, Bat SARS CoV (GenBank Accession No. FJ211859), SARS CoV (GenBank Accession No. FJ211860), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rfl (GenBank Accession No. DQ412042), BtCoV.273.2005 (GenBank Accession No. DQ648856), BtSARS.Rp3 (GenBank Accession No. DQ071615), SARS COV.A022 (GenBank Accession No. AY686863), SARSCOV.CUHK-W1 (GenBank Accession No. AY278554), SARSCOV.GDO1 (GenBank Accession No. AY278489), SARSCOV.HC.SZ.61.03 (GenBank Accession No. AY515512), SARSCOV.SZ16 (GenBank Accession No. AY304488), SARSCOV. Urbani (GenBank Accession No. AY278741), SARSCOV.civet010 (GenBank Accession No. AY572035), or SARSCOV.MA.15 (GenBank Accession No. DQ497008), Rs SHC014 (GenBank® Accession No. KC881005), Rs3367 (GenBank® Accession No. KC881006), WiV1 S (GenBank® Accession No. KC881007).

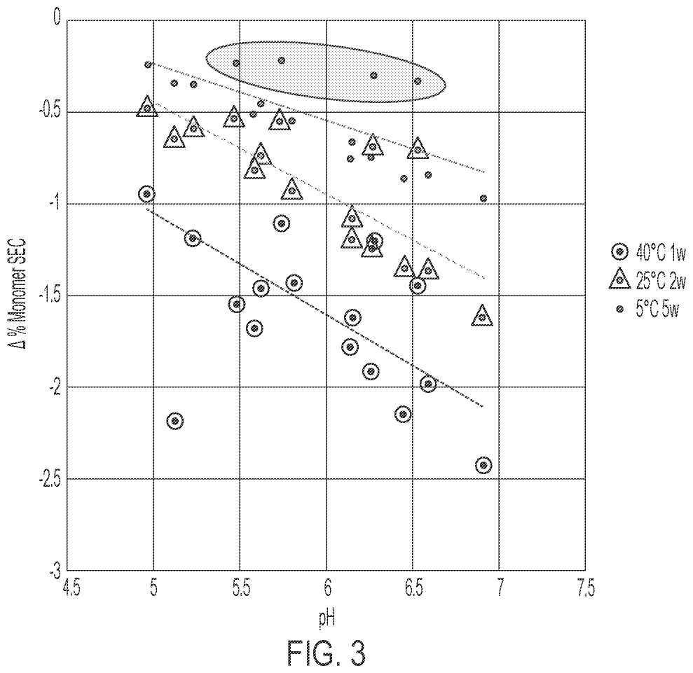

In some embodiments, the antibodies and antigen-binding antibody fragments provided herein may also bind to and neutralize existing bat CoV or pre-emergent bat CoVs. Antibodies and antigen-binding antibody fragments with such binding and/or neutralization abilities would be particularly useful in a future pandemic that may be caused by a spillover from an animal reservoir, like a bat. In fact, ADI-55688, ADI-55689, ADI-55993, ADI-5600, ADI-56046, ADI-55690, ADI-56010, and ADI-55951 were shown to neutralize authentic bat coronavirus, WIV1 (see FIG. 3E of Wec A, et al., Science. 2020 Jun. 15; eabc7424, doi: 10.1126/science.abc7424).

Alternatively, the S proteins of CoV to which the antibodies or antigen-binding antibody fragments in the pharmaceutical formulations of the present disclosure may specifically bind to and neutralize pre-emergent coronaviruses from other species, e.g., bats.

Still alternatively, the S proteins of CoV to which the antibodies or antigen-binding antibody fragments in the pharmaceutical formulations of the present disclosure may specifically bind to may include, for example, Middle East respiratory syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_17_2013 (GenBank Accession No. KF600647.1), Middle East respiratory syndrome coronavirus isolate Al-Hasa_15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-COV_SA-N1 (GenBank Accession No. KC667074), following isolates of Middle East Respiratory Syndrome Coronavirus (GenBank Accession No: KF600656.1. GenBank Accession No: KF600655.1. GenBank Accession No: KF600654.1. GenBank Accession No: KF600649.1, GenBank Accession No: KF600648.1, GenBank Accession No: KF600646.1. GenBank Accession No: KF600643.1, GenBank Accession No: KF600642.1, GenBank Accession No: KF600640.1, GenBank Accession No: KF600639.1. GenBank Accession No: KF600638.1, GenBank Accession No: KF600637.1. GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1. GenBank Accession No: KF600619.1, GenBank Accession No: KF600618.1, GenBank Accession No: KF600616.1, GenBank Accession No: KF600615.1, GenBank Accession No: KF600614.1. GenBank Accession No: KF600641.1. GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA_003/Bisha/Saudi Arabia/2013 GenBank Accession No: KF493888.1. Bat coronavirus Pikuh/CII_KSA_001/Riyadh/Saudi Arabia/2013 GenBank Accession No: KF493887.1. Bat coronavirus Rhhar/CII_KSA_002/Bisha/Saudi Arabia/2013 GenBank Accession No: KF493886.1. Bat Coronavirus Rhhar/CII_KSA_004/Bisha/Saudi Arabia/2013 GenBank Accession No: KF493884.1. BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV 133.2005 (GenBank Accession No. NC 008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1. GenBank Accession No: KC522085.1. GenBank Accession No: KC522084.1. GenBank Accession No: KC522083.1, GenBank Accession No: KC522082.1, GenBank Accession No: KC522081.1. GenBank Accession No: KC522080.1, GenBank Accession No: KC522079.1. GenBank Accession No: KC522078.1. GenBank Accession No: KC522077.1. GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1. GenBank Accession No: KC522100.1. GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1. GenBank Accession No: KC522092.1. GenBank Accession No: KC522091.1, GenBank Accession No: KC522090.1. GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1. GenBank Accession No: KC522107.1. GenBank Accession No: KC522106.1. GenBank Accession No: KC522105.1) Pipistrellus bat coronavirus HKU4 isolates (GenBank Accession No: KC522048.1, GenBank Accession No: KC522047.1, GenBank Accession No: KC522046.1, GenBank Accession No: KC522045.1, GenBank Accession No: KC522044.1, GenBank Accession No: KC522043.1. GenBank Accession No: KC522042.1, GenBank Accession No: KC522041.1, GenBank Accession No: KC522040.1 GenBank Accession No: KC522039.1. GenBank Accession No: KC522038.1. GenBank Accession No: KC522037.1. GenBank Accession No: KC522036.1. GenBank Accession No: KC522048.1 GenBank Accession No: KC522047.1 GenBank Accession No: KC522046.1 GenBank Accession No: KC522045.1 GenBank Accession No: KC522044.1 GenBank Accession No: KC522043.1 GenBank Accession No: KC522042.1 GenBank Accession No: KC522041.1 GenBank Accession No: KC522040.1. GenBank Accession No: KC522039.1 GenBank Accession No: KC522038.1 GenBank Accession No: KC522037.1 GenBank Accession No: KC522036.1. GenBank Accession No: KC522061.1 GenBank Accession No: KC522060.1 GenBank Accession No: KC522059.1 GenBank Accession No: KC522058.1 GenBank Accession No: KC522057.1 GenBank Accession No: KC522056.1 GenBank Accession No: KC522055.1 GenBank Accession No: KC522054.1 GenBank Accession No: KC522053.1 GenBank Accession No: KC522052.1 GenBank Accession No: KC522051.1 GenBank Accession No: KC522050.1 GenBank Accession No: KC522049.1 GenBank Accession No: KC522074.1, GenBank Accession No: KC522073.1 GenBank Accession No: KC522072.1 GenBank Accession No: KC522071.1 GenBank Accession No: KC522070.1 GenBank Accession No: KC522069.1 GenBank Accession No: KC522068.1 GenBank Accession No: KC522067.1, GenBank Accession No: KC522066.1 GenBank Accession No: KC522065.1 GenBank Accession No: KC522064.1. GenBank Accession No: KC522063.1, or GenBank Accession No: KC522062.1.

Alternatively, the S proteins of CoV to which the antibodies or antigen-binding antibody fragments in the pharmaceutical formulations of the present disclosure may specifically bind may include for example, FCov.FIPV.79.1146. VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), or porcine reproductive and respiratory syndrome virus (PRRSV) (GenBank Accession No. NC_001961.1; GenBank Accession No. DQ811787).

Alternatively, the S proteins of CoV to which the antibodies or antigen-binding antibody fragments in the pharmaceutical formulations of the present disclosure may specifically bind may include, for example, BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1. GenBank Accession No. N601061.1. GenBank Accession No. JN601060.1. GenBank Accession No. JN601059.1. GenBank Accession No. JN601058.1, GenBank Accession No. JN601057.1. GenBank Accession No. JN601056.1. GenBank Accession No. JN601055.1. GenBank Accession No. JN601054.1. GenBank Accession No. JN601053.1. GenBank Accession No. JN601052.1. GenBank Accession No. JN400902.1. GenBank Accession No. JN547395.1, GenBank Accession No. FJ687473.1. GenBank Accession No. FJ687472.1, GenBank Accession No. FJ687471.1, GenBank Accession No. FJ687470.1. GenBank Accession No. FJ687469.1. GenBank Accession No. FJ687468.1, GenBank Accession No. FJ687467.1. GenBank Accession No. FJ687466.1. GenBank Accession No. FJ687465.1. GenBank Accession No. FJ687464.1. GenBank Accession No. FJ687463.1, GenBank Accession No. FJ687462.1, GenBank Accession No. FJ687461.1. GenBank Accession No. FJ687460.1, GenBank Accession No. FJ687459.1. GenBank Accession No. FJ687458.1. GenBank Accession No. FJ687457.1. GenBank Accession No. FJ687456.1. GenBank Accession No. FJ687455.1, GenBank Accession No. FJ687454.1, GenBank Accession No. FJ687453 GenBank Accession No. FJ687452.1. GenBank Accession No. FJ687451.1. GenBank Accession No. FJ687450.1, GenBank Accession No. FJ687449.1, GenBank Accession No. AF500215.1. GenBank Accession No. KF476061.1. GenBank Accession No. KF476060.1, GenBank Accession No. KF476059.1. GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1. GenBank Accession No. KF177258.1, GenBank Accession No. KF177257.1, GenBank Accession No. KF177256.1. GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), or BtCoV.HKU2.GD.430.2006 (GenBank Accession No. EF203064).

Alternatively, the S proteins of CoV to which the antibodies or antigen-binding antibody fragments in the pharmaceutical formulations of the present disclosure may specifically bind may include, for example, HCoV.HKU1.C.N5 (GenBank Accession No. DQ339101), MHV.A59 (GenBank Accession No. NC 001846), PHEV.VW572 (GenBank Accession No. NC 007732), HCoV.OC43.ATCC. VR.759 (GenBank Accession No. NC_005147), or bovine enteric coronavirus (BCoV.ENT) (GenBank Accession No. NC_003045).

Alternatively, the S proteins of CoV to which the antibodies or antigen-binding antibody fragments in the pharmaceutical formulations of the present disclosure may specifically bind may include, for example, BtCoV.HKU9.2 (GenBank Accession No. EF065514), BtCoV.HKU9.1 (GenBank Accession No. NC_009021), BtCoV.HKU9.3 (GenBank Accession No. EF065515), or BtCoV.HKU9.4 (GenBank Accession No. EF065516).

In some instances, an anti-CoV-S antibody or antigen-binding fragment thereof in the pharmaceutical formulations of the present disclosure binds to CoV-S (e.g., SARS-COV—S and/or SARS-COV-2-S, and/or any of the CoV S proteins listed above) with a dissociation constant (KD) of (i) 100 nM or lower; (ii) about 10 nM or lower; (iii) about 1 nM or lower; (iv) about 100 pM or lower; (v) about 10 pM or lower; (vi) about 1 pM or lower; or (vii) about 0.1 pM or lower.

The present disclosure provides exemplary antibodies or antigen-binding fragments thereof that bind CoV-S, including human CoV-S, which optionally may be affinity-matured. Other antibodies or antigen-binding fragments thereof that bind CoV-S, including those having different CDRs, and epitopic specificity may be obtained using the disclosure of the present specification, and using methods that are generally known in the art. Such antibodies and antigen-binding fragments thereof antagonize the biological effects of CoV-S in vivo and therefore are useful in treating or preventing COV-S-related conditions including, particularly coronavirus infection. In preferred embodiments, the antibody or antigen-binding fragment thereof according to the disclosure comprises one or more CDRs, a V_{L} chain and/or V_{H} chain of the anti-CoV-S antibodies and antigen-binding fragments thereof described herein.

In some embodiments, an anti-CoV-S antibody or antigen-binding fragment thereof in the pharmaceutical formulations according to the disclosure will interfere with, block, reduce, or modulate the interaction between COV-S and its receptor(s) (e.g., ACE2, CD209L, L-SIGN, DPP4, or CD26) on host cells or a S protein-priming protein on host cells (e.g., TMPRSS2). If binding of the S protein to its receptor is blocked or reduced, CoV virions may be prohibited from entering the cells, i.e., infection to further cells is prevented. Also, if the S protein is prevented from binding to a S protein-priming protein, the S protein would not be activated and therefore the host cell entry via the receptor may be reduced, i.e., infection to further cells is prevented.

In some instance, an anti-CoV-S antibody or antigen-binding fragment thereof in the pharmaceutical formulations according to the disclosure is “neutralizing”, e.g., it substantially or totally prevents the specific interaction of CoV-S with the host receptors or priming protein. As a result, CoV virions may be substantially or totally cleared by immune cells of the host, such as phagocytes via, for example, Fc receptor mediated phagocytosis or mere phagocytosis due to increased time of virions outside the cells. In some embodiments, the antibody or antigen-binding fragment thereof in the pharmaceutical formulations of the present disclosure neutralizes CoV-S, e.g., by remaining bound to CoV-S in a location and/or manner that prevents CoV-S from specifically binding to its receptor or priming protein on host cells. As a result, CoV virions may be substantially or totally prevented from entering the cells, i.e. infection to further cells is prevented. In certain embodiments, an anti-CoV-S antibody or antigen-binding fragment thereof in the pharmaceutical formulations according to the disclosure neutralizes CoV (e.g., SARS-COV and/or SARS-COV-2) at an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 PM or lower, of about 20 pM or lower, of about 10 pM or lower, of about 5 pM or lower, of about 2 pM or lower, or of about 1 pM or lower, or at an IC50 of about 500 ng/ml or lower, of about 200 ng/ml or lower, of about 100 ng/ml or lower, of about 50 ng/ml or lower, at about 20 ng/ml or lower, at about 10 ng/mL or lower, at about 20 ng/mL or lower, at about 10 mg/mL or lower, at about 5 ng/mL or lower, at about 2 ng/ml or lower, or at about 1 ng/ml or lower, in vitro, as measured by any of the neutralization assays known in the art.

In some instances, an anti-CoV-S antibody or antigen-binding fragment thereof in the pharmaceutical formulations according to the disclosure or cocktail thereof, when administered to a coronavirus infected host or one susceptible to coronavirus infection such as a health care worker may promote a neutralization response in the host against the coronavirus which is sufficient to permit the host to be able to mount an effective cell-mediated immune response against the virus, e.g., T cell-mediated or cytokine-mediated immune response against the coronavirus and/or to be more responsive to other treatment methods such as drugs, antivirals or other biologics.

As mentioned, the anti-CoV-S antibodies or antigen-binding fragments thereof in the pharmaceutical formulations according to the disclosure have a variety of uses. For example, the subject antibodies and fragments can be useful in prophylactic or therapeutic applications, as well as diagnostically in binding assays. The subject anti-CoV-S antibodies or antigen-binding fragments thereof are useful for affinity purification of CoV-S, in particular human CoV-S or its ligands and in screening assays to identify other antagonists of CoV-S activity. Some of the antibodies or antigen-binding fragments thereof are useful for inhibiting binding of CoV-S to its receptor(s) (e.g., ACE2, CD209L, L-SIGN, DPP4, or CD26) on host cells or a S protein-priming protein on host cells (e.g., TMPRSS2) or inhibiting COV-S-mediated activities and/or biological effects.

As used herein, the term “one or more biological effects associated with COV-S refers to any biological effect mediated, induced, or otherwise attributable to COV-S, e.g., binding properties, functional properties, and other properties of biological significance. Non-limiting exemplary biological effects of COV-S include COV-S binding to its receptor(s) (e.g., ACE2, CD209L, L-SIGN, DPP4, or CD26) on host cells or a S protein-priming protein on host cells (e.g., TMPRSS2), activation of host cells for allowing virus entry, activation of immune cells as a result of the entry of CoV into the cell, e.g., via presentation of CoV antigen(s) on the host cells' MHC molecule, and resulting inflammation. The subject anti-CoV-S antibodies are capable of inhibiting one, a combination of, or all of these exemplary CoV-S biological activities. For example, the anti-CoV-S antibodies and antigen-binding fragments thereof provided herein may neutralize CoV virions or reduce the infectivity of CoV virions.

The pharmaceutical formulations according to the disclosure can be used in a variety of therapeutic applications. For example, in some embodiments the pharmaceutical formulations are useful for treating conditions associated with CoV-S, such as, but not limited to, symptoms associated with CoV infection. The CoV may be any CoV, including SARS-COV. SARS-COV-2, MERS-COV. HCoV-HKU1, HCoV-OC43, HCOV-229E, and HCoV-NL63, and also may be any of the CoV species listed above herein.

Specific examples of CoV infection-associated symptoms are fever, cough, dry cough, shortness of breath or difficulty of breath, fatigue, aches, runny nose, congestion, sore throat, conjunctivitis, chest pain, headache, muscle ache, chills, loss of smell, and loss of taste, and gastrointestinal symptoms including diarrhea. Complications and/or diseases/disorders associated with coronavirus infection may include, for example, bronchitis, pneumonia, respiratory failure, acute respiratory failure, organ failure, multi-organ system failure, pediatric inflammatory multisystem syndrome, acute respiratory distress syndrome (a severe lung condition that causes low oxygen in the blood and organs), blood clots, cardiac conditions, myocardial injury, myocarditis, heart failure, cardiac arrest, acute myocardial infarction, dysrhythmias, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, gastrointestinal complications, aspergillosis, increased susceptibility to infection with another virus or bacteria, and/or pregnancy-related complications. Certain diseases and conditions, such as high blood pressure, type 1 diabetes, liver disease, overweight, chronic lung diseases including cystic fibrosis, pulmonary fibrosis, and asthma, compromised immune system due to transplant, use of an immunosuppressant, or HIV infection, and brain and nervous system condition, may increase the risk of CoV infection-associated complications and diseases.

The pharmaceutical formulations may be used alone or in association with other active agents or drugs, including other biologics, to treat any subject in which blocking, inhibiting, or neutralizing the in vivo effect of CoV-S or blocking or inhibiting the interaction of CoV-S and its receptor(s) (e.g., ACE2, CD209L, L-SIGN, DPP4, or CD26) on host cells or a S protein-priming protein on host cells (e.g., TMPRSS2), is therapeutically desirable. In some embodiment, the pharmaceutical formulations comprising an anti-CoV-S antibody and antigen-binding fragment thereof, e.g., ADI-58125, may be used in combination with a second pharmaceutical formulation comprising a second antibody, or antigen-binding fragment thereof, wherein the second antibody, or antigen-binding fragment thereof, is selected from the group consisting of ADI-58120, ADI-58121, ADI-58122, ADI-58123, ADI-58124, ADI-58126, ADI-58127, ADI-58128, ADI-58129, ADI-58130, ADI-58131, or a combination thereof. In some embodiment, the second antibody, or antigen-binding fragment thereof, is ADI-58122. In one embodiment, the second antibody, or antigen-binding fragment thereof, is ADI-58127. In one embodiment, the second antibody, or antigen-binding fragment thereof, is ADI-58129. In one embodiment, the second antibody, or antigen-binding fragment thereof, is ADI-58131.

Exemplary anti-CoV antibodies and antigen-binding fragments thereof according to the disclosure, and the specific CDRs thereof are identified in this section.

The anti-CoV-S antibodies and antigen-binding fragments thereof in the pharmaceutical formulations according to the disclosure have binding affinity for CoV-S, such as SARS-COV—S or SARS-COV-S2. Some antibodies of the present disclosure bind to SARS-COV—S or SARS-COV-S2 with a similar K_{D} (M), while some antibodies of the present disclosure bind to SARS-COV—S with a lower K_{D} (M) (i.e., higher affinity) than to SARS-COV-S2, and some antibodies of the present disclosure bind to SARS-COV-S-2 with a lower K_{D} (M) (i.e., higher affinity) than to SARS-COV-S.

In some embodiments, the pharmaceutical formulations of the present disclosure comprise anti-CoV-S antibodies, and antigen-binding fragments thereof, specifically provided herein including: antibodies ADI-58120, ADI-58121, ADI-58122, ADI-58123, ADI-58124, ADI-58125, ADI-58126, ADI-58127, ADI-58128, ADI-58129, ADI-58130, ADI-58131, and antigen-binding fragments thereof. Any Fc variant including but not limited to those specifically disclosed in Table 38 may be used in combination with any of the variable sequences disclosed herein. In some embodiments, the Fc variant is an LA variant and comprises the amino acid sequence of SEQ ID NO: 413. In one embodiment, the antibody ADI-58125 comprises an Fc variant of SEQ ID NO:413.

Tables 30-37 show the SEQ ID NOs assigned to individual amino acid sequences of the HC, VH, VH FR1, VH CDR1, VH FR2, VH CDR2, VH FR3, VH CDR3, VH FR4, LC, VL, VL FR1, VL CDR1. VL FR2, VL CDR2, VL FR3, VL CDR3, and VL FR4 for individual antibodies, and the SEQ ID NOs assigned to the nucleic acid sequences of the VH and VL of individual antibodies.

In some embodiments, the pharmaceutical formulations of the present disclosure comprise anti-CoV-S antibodies or antigen-binding antibody fragments comprising (i) a VH CDR that is same as the VH CDR3 of, (ii) a VH CDR3 and VL CDR3, both of which as same as both of the VH CDR3 and the VL CDR3 of, (iii) at least 1, 2, 3, 4, 5, or 6 CDRs that are same as the corresponding CDR(s) of, or (iv) 6 CDRs that are all the same as the 6 CDRs of any one of the disclosed antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In further embodiments, the pharmaceutical formulations of the present disclosure may comprise anti-CoV-S antibodies or antigen-binding antibody fragments, which optionally may be affinity-matured, comprising one of the CDR requirements (i)-(iv) of the immediately above paragraph, further wherein (a) the VH comprises an amino acid sequence with at least 80, 85, 90, 91, 92, 93, 94, 95, 96, 97, 98, 99, or 100% sequence identity to the amino acid sequence of the VH of, and (b) the VL comprises an amino acid sequence with at least 80, 85, 90, 91, 92, 93, 94, 95, 96, 97, 98, 99, or 100% sequence identity to the amino acid sequence of the VL of any one of the disclosed antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In further embodiments, the pharmaceutical formulations of the present disclosure comprise anti-CoV-S antibodies or antigen-binding antibody fragments, which optionally may be affinity-matured, comprising one of the VH and VL requirements (i)-(iv) of the immediately above paragraph, further wherein (a) the heavy chain comprises an amino acid sequence with at least 80, 85, 90, 91, 92, 93, 94 95, 96, 97, 98, 99, or 100% sequence identity to the amino acid sequence of the heavy chain of, and (b) the light chain comprises an amino acid sequence with at least 80, 85, 90, 91, 92, 93, 94, 95, 96, 97, 98, 99, or 100% sequence identity to the amino acid sequence of the light chain of any one of the disclosed antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In further embodiments, the pharmaceutical formulations of the present disclosure comprise anti-CoV-S antibodies or antigen-binding antibody fragments, which optionally may be affinity-matured, comprising one of the CDR requirements (i)-(iv) of the immediately above paragraph, further wherein (a) the VH is identical to the VH of, and (b) the VL is identical to the VL of any one of the disclosed antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In other embodiments, the pharmaceutical formulations of the present disclosure comprise antibodies and antigen-binding fragments, which optionally may be affinity-matured, having binding specificity to CoV-S that bind the same epitope as one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131.

In other embodiments, the pharmaceutical formulations of the present disclosure comprise antibodies and antigen-binding fragments having binding specificity to CoV-S, which optionally may be affinity-matured, that bind the same epitope as any one of antibodies selected from the group consisting of ADI-58120, ADI-58121, ADI-58122, ADI-58123, ADI-58124, ADI-58125, ADI-58126.

In other embodiments, the pharmaceutical formulations of the present disclosure comprise anti-CoV-S antibodies and antigen-binding fragments which optionally may be affinity-matured, comprise, or alternatively consist of, combinations of one or more of the FRs, CDRs, the VH and VL sequences, and the heavy chain and light chain sequences set forth above, including all of them, or sequences that are at least 90%, 91%, 92%, 93%, 94%, 95%, 96%, 97%, 98%, or 99% identical thereto.

In a further embodiment, the pharmaceutical formulations of the present disclosure comprise antigen-binding fragments comprising, or alternatively consisting of, Fab fragments having binding specificity for CoV-S. The Fab fragment preferably includes the VH and the VL sequence of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, or sequences that are at least 90%, 91%, 92%, 93%, 94%, 95%, 96%, 97%, 98%, or 99% identical thereto. This embodiment further includes Fabs containing additions, deletions, and variants of such VH and VL sequence while retaining binding specificity for CoV-S.

In some embodiments, Fab fragments may be produced by enzymatic digestion (e.g., papain) of the parent full antibody. In another embodiment, anti-CoV-S antibodies, such as anyone of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, and Fab fragments thereof may be produced via expression in mammalian cells, such as CHO, NS0, or HEK 293 cells, fungal, insect, or microbial systems, such as yeast cells.

In additional embodiments, disclosed herein are polynucleotides encoding antibody polypeptides having binding specificity to CoV-S, including the VH and VL of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, as well as fragments, variants, optionally affinity-matured variants, and combinations of one or more of the FRs, CDRs, the VH and VL sequences, and the heavy chain and light chain sequences set forth above, including all of them, or sequences that are at least 90%, 91%, 92%, 93%, 94%, 95%, 96%, 97%, 98%, or 99% identical thereto.

In other embodiments, the disclosure contemplates isolated anti-CoV-S antibodies and antigen binding fragments comprising (i) a VH which is same as the VH of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131; and (ii) a VL which is same as the VL of another antibody selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, or a variant thereof, wherein optionally one or more of the framework region residues (“FR residues”) and/or CDR residues in said V_{H} or V_{L} polypeptide has been substituted with another amino acid residue resulting in an anti-CoV-S antibody that specifically binds COV-S.

The disclosure also includes humanized, primatized and other chimeric forms of these antibodies. The chimeric and humanized antibodies may include an Fc derived from IgG1, IgG2, IgG3, or IgG4 constant regions.

In some embodiments, the chimeric or humanized antibodies or fragments or VH or VL polypeptides originate or are derived from one or more human antibodies, e.g., a human antibody identified from a clonal human B cell population.

In some aspects, the disclosure provides vectors comprising a nucleic acid molecule encoding an anti-CoV-S antibody or fragment thereof as disclosed herein. In some embodiments, the disclosure provides host cells comprising a nucleic acid molecule encoding an anti-CoV-S antibody or fragment thereof as disclosed herein.

In some aspects, the disclosure provides isolated antibodies or antigen binding fragments thereof that competes for binding to CoV-S with an antibody or antigen binding fragment thereof disclosed herein.

In some aspects, the disclosure provides a nucleic acid molecule encoding any of the antibodies or antigen binding fragments disclosed herein.

In some aspects, the disclosure provides a pharmaceutical or diagnostic composition comprising at least one antibody or antigen binding fragment thereof as disclosed herein.

In some aspects, the disclosure provides a method for treating or preventing a condition associated with elevated CoV-S levels in a subject, comprising administering to a subject in need thereof an effective amount of the pharmaceutical formulations as disclosed herein.

In some aspects, the disclosure provides a method of inhibiting binding of CoV-S to its receptor (e.g., ACE2, L-SIGN, CD209L, DPP4, CD26) or an S protein-priming protein (e.g., TMPRSS2) in a subject comprising administering an effective amount of the pharmaceutical formulations as disclosed herein. For example, administering a pharmaceutical formulation of the present disclosure comprising one or more of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131 may inhibit binding of CoV-S to its receptor, e.g., ACE2.

In some aspects, the disclosure provides pharmaceutical formulations comprising an antibody or antigen binding fragment thereof that selectively binds to CoV-S, wherein the antibody or antigen binding fragment thereof binds to CoV-S with a Kp of less than or equal to 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, or 10⁻¹³ M; preferably, with a Kp of less than or equal to 5×10⁻¹⁰ M, 10⁻¹⁰ M, 5×10⁻¹¹ M, 10⁻¹¹ M, 5×10⁻¹² M, or 10⁻¹² M; more preferably, with a Kp that is less than about 100 pM, less than about 50 pM, less than about 40 pM, less than about 25 pM, less than about 1 pM, between about 10 pM and about 100 pM, between about 1 pM and about 100 pM, or between about 1 pM and about 10 pM. Preferably, the anti-CoV-S antibody or antigen binding fragment has cross-reactivity to the S protein of CoV other than SARS-COV—S or SARS-COV-2-S.

The antibodies and antigen binding fragments thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Antibodies and antigen binding fragments thereof may also be chemically modified to provide additional advantages such as increased solubility, stability and circulating time (in vivo half-life) of the polypeptide, or decreased immunogenicity (See U.S. Pat. No. 4,179,337). The chemical moieties for derivatization may be selected from water soluble polymers such as polyethylene glycol, ethylene glycol/propylene glycol copolymers, carboxymethylcellulose, dextran, polyvinyl alcohol, and the like. The antibodies and fragments thereof may be modified at random positions within the molecule, or at predetermined positions within the molecule and may include one, two, three, or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the case in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. Branched polyethylene glycols are described, for example, in U.S. Pat. No. 5,643,575; Morpurgo et al., Appl. Biochem. Biotechnol., 56:59-72 (1996); Vorobjev et al., Nucleosides and Nucleotides, 18:2745-2750 (1999); and Caliceti et al., Bioconjug. Chem., 10:638-646 (1999), the disclosures of each of which are incorporated herein by reference.

There are a number of attachment methods available to those skilled in the art (See e.g., EP 0 401 384, herein incorporated by reference, disclosing a method of coupling PEG to G-CSF; and Malik et al., Exp. Hematol., 20:1028-1035 (1992) (reporting pegylation of GM-CSF using tresyl chloride)). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As described above, polyethylene glycol may be attached to proteins via linkage to any of a number of amino acid residues. For example, polyethylene glycol can be linked to polypeptides via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) or to more than one type of amino acid residue (e.g., lysine, histidine, aspartic acid, glutamic acid, cysteine and combinations thereof).

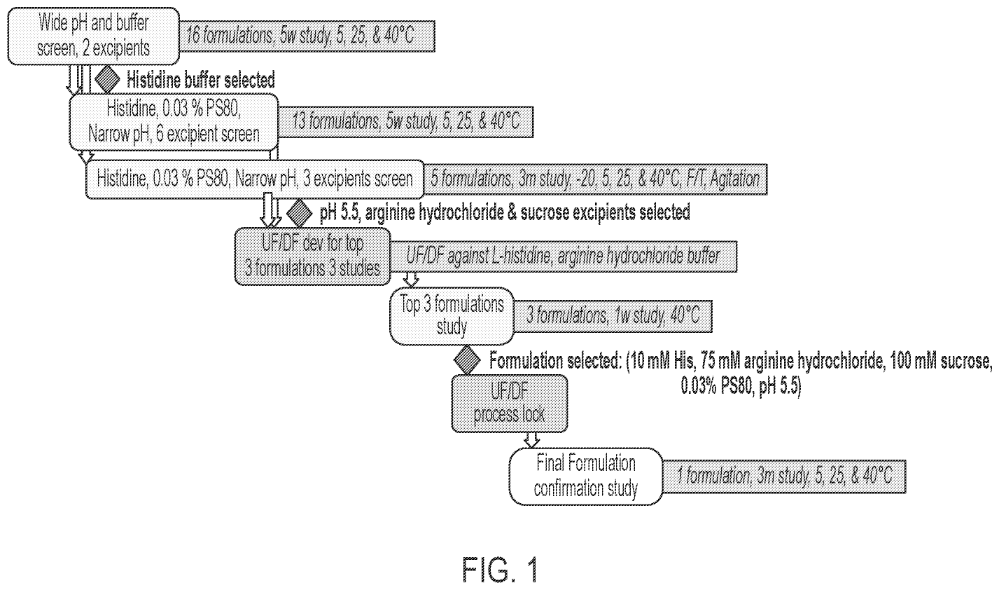

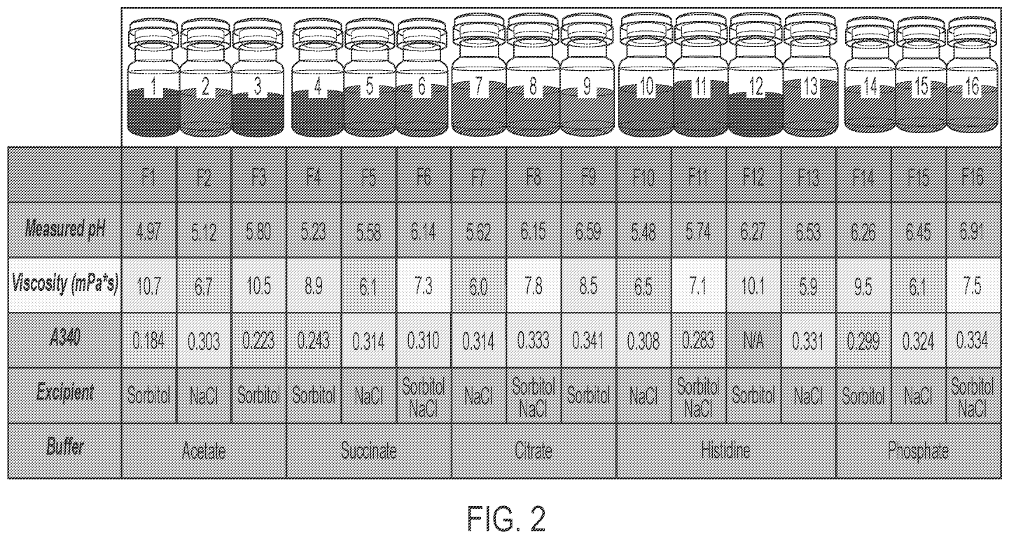

Alternatively, antibodies or antigen binding fragments thereof having increased in vivo half-lives may be produced via fusion with albumin (including but not limited to recombinant human serum albumin or fragments or variants thereof (See, e.g., U.S. Pat. No. 5,876,969, EP 0 413 622, and U.S. Pat. No. 5,766,883, herein incorporated by reference in their entirety)), or other circulating blood proteins such as transferrin or ferritin. In a preferred embodiment, polypeptides and/or antibodies of the present disclosure (including fragments or variants thereof) are fused with the mature form of human serum albumin (i.e., amino acids 1-585 of human serum albumin as shown in FIGS. 1 and 2 of EP 0 322 094) which is herein incorporated by reference in its entirety. Polynucleotides encoding fusion proteins of the disclosure are also encompassed by the disclosure.

Regarding detectable moieties, further exemplary enzymes include, but are not limited to, horseradish peroxidase, acetylcholinesterase, alkaline phosphatase, beta-galactosidase, and luciferase. Further exemplary fluorescent materials include, but are not limited to, rhodamine, fluorescein, fluorescein isothiocyanate, umbelliferone, dichlorotriazinylamine, phycoerythrin, and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine 125 (¹²⁵I), Carbon 14 (¹⁴C), Sulfur 35 (³⁵S), Tritium (³H) and Phosphorus 32 (³²P).

Methods are known in the art for conjugating an antibody or antigen binding fragment thereof to a detectable moiety and the like, such as for example those methods described by Hunter et al., Nature, 144:945 (1962); David et al., Biochemistry, 13:1014 (1974); Pain et al., J. Immunol. Meth., 40:219 (1981); and Nygren, J., Histochem, and Cytochem., 30:407 (1982).

Embodiments described herein further include variants and equivalents that are substantially homologous to the antibodies, antibody fragments, diabodies, SMIPs, camelbodies, nanobodies, IgNAR, polypeptides, variable regions, and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. The intent of a conservative amino acid substitution is well known in the art.

In other embodiments, the disclosure contemplates polypeptide sequences having at least 90% or greater sequence homology to any one or more of the polypeptide sequences of antigen binding fragments, variable regions and CDRs set forth herein. More preferably, the disclosure contemplates polypeptide sequences having at least 95% or greater sequence homology, even more preferably at least 98% or greater sequence homology, and still more preferably at least 99% or greater sequence homology to any one or more of the polypeptide sequences of antigen binding fragments, variable regions, and CDRs set forth herein.

Methods for determining homology between nucleic acid and amino acid sequences are well known to those of ordinary skill in the art.

In other embodiments, the disclosure further contemplates the above-recited polypeptide homologs of the antigen binding fragments, variable regions and CDRs set forth herein further having anti-CoV-S activity. Non-limiting examples of anti-CoV-S activity are set forth herein, e.g., ability to inhibit CoV-S binding to its receptor such as ACE2 or L-SIGN or an S protein-priming protein, thereby resulting in the reduced entry of CoV into cells.

In other embodiments, the disclosure further contemplates the generation and use of antibodies that bind any of the foregoing sequences, including, but not limited to, anti-idiotypic antibodies. In an exemplary embodiment, such an anti-idiotypic antibody could be administered to a subject who has received an anti-CoV-S antibody to modulate, reduce, or neutralize, the effect of the anti-CoV-S antibody. Such antibodies could also be useful for treatment of an autoimmune disease characterized by the presence of anti-CoV-S antibodies. A further exemplary use of such antibodies, e.g., anti-idiotypic antibodies, is for detection of the anti-CoV-S antibodies of the present disclosure, for example to monitor the levels of the anti-CoV-S antibodies present in a subject's blood or other bodily fluids. For example, in one embodiment, the disclosure provides a method of using the anti-idiotypic antibody to monitor the in vivo levels of said anti-CoV-S antibody or antigen binding fragment thereof in a subject or to neutralize said anti-CoV-S antibody in a subject being administered said anti-CoV-S antibody or antigen binding fragment thereof.

The present disclosure also contemplates anti-CoV-S antibodies comprising any of the polypeptide or polynucleotide sequences described herein substituted for any of the other polynucleotide sequences described herein. For example, without limitation thereto, the present disclosure contemplates antibodies comprising the combination of any of the VL and VH sequences described herein, and further contemplates antibodies resulting from substitution of any of the CDR sequences described herein for any of the other CDR sequences described herein.

Another embodiment of the disclosure contemplates these polynucleotides incorporated into an expression vector for expression in mammalian cells such as CHO, NS0, or HEK-293 cells, or in fungal, insect, or microbial systems such as yeast cells. In one embodiment of the disclosure described herein, Fab fragments can be produced by enzymatic digestion (e.g., papain) of any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, following expression of the full-length polynucleotides in a suitable host. In another embodiment, anti-CoV-S antibodies, such as anyone of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, or Fab fragments thereof, can be produced via expression of the polynucleotides encoding any one of antibodies selected from the group consisting of ADI-58120, ADI-58121, ADI-58122, ADI-58123, ADI-58124, ADI-58125, ADI-58126, ADI-58127, ADI-58128, ADI-58129, ADI-58130, and ADI-58131, in mammalian cells such as CHO, NS0, or HEK 293 cells, fungal, insect, or microbial systems such as yeast cells.

Host cells and vectors comprising said polynucleotides are also contemplated.

The disclosure further contemplates vectors comprising the polynucleotide sequences encoding the variable heavy and light chain polypeptide sequences, as well as the individual CDRs (hypervariable regions), as set forth herein, as well as host cells comprising said vector sequences. In one embodiment, the host cells are mammalian cells, such as CHO cells. In one embodiment, the host cells are yeast cells.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used.

In some embodiments, the pharmaceutical formulations of the present disclosure comprise about 10-500 mg/mL, about 50-400 mg/mL, about 100-300 mg/mL, about 100-200 mg/mL, about 130-170 mg/mL, or about 125-175 mg/mL antibody, or antigen-binding fragment thereof. In other embodiments, the pharmaceutical formulations of the present disclosure comprise about 10 mg/mL, about 50 mg/mL, about 100 mg/mL, about 125 mg/mL, about 130 mg/mL about 150 mg/mL, about 170 mg/mL, about 175 mg/mL, about 200 mg/mL, about 250 mg/mL, about 300 mg/mL, about 400 mg/mL, or about 500 mg/mL antibody, or antigen-binding fragment thereof.

i. B-cell Screening and Isolation

In one embodiment, the present disclosure contemplates the preparation and isolation of a clonal population of antigen-specific B-cells that may be used for isolating at least one CoV-S antigen-specific cell, which can be used to produce a monoclonal antibody against CoV-S, which is specific to a desired CoV-S antigen, or a nucleic acid sequence corresponding to such an antibody, for use in the pharmaceutical formulations of the present disclosure. Methods of preparing and isolating said clonal population of antigen-specific B-cells are taught, for example, in U.S. Patent Publication No. US2007/0269868 to Carvalho-Jensen et al., the disclosure of which is herein incorporated by reference in its entirety. Methods of preparing and isolating said clonal population of antigen-specific B-cells are also taught herein in the examples. Methods of “enriching” a cell population by size or density are known in the art. See, e.g., U.S. Pat. No. 5,627,052. These steps can be used in addition to enriching the cell population by antigen-specificity.

ii. Methods of Producing Antibodies and Fragments Thereof

In another embodiment, the present disclosure contemplates methods for producing anti-CoV-S antibodies and fragments thereof for use in the pharmaceutical formulations of the present disclosure. Methods of producing antibodies are well known to those of ordinary skill in the art. For example, methods of producing chimeric antibodies are now well known in the art (See, for example, U.S. Pat. No. 4,816,567 to Cabilly et al.; Morrison et al., Proc. Natl. Acad. Sci. U.S.A., 81:8651-55 (1984); Neuberger et al., Nature, 314:268-270 (1985); Boulianne, G. L, et al., Nature, 312:643-46 (1984), the disclosures of each of which are herein incorporated by reference in their entireties).

As mentioned above, methods of producing humanized antibodies are now well known in the art (See, for example, U.S. Pat. Nos. 5,530,101, 5,585,089, 5,693,762, and 6,180,370 to Queen et al; U.S. Pat. Nos. 5,225,539 and 6,548,640 to Winter; U.S. Pat. Nos. 6,054,297, 6,407,213 and 6,639,055 to Carter et al; U.S. Pat. No. 6,632,927 to Adair; Jones, P. T, et al., Nature, 321:522-525 (1986); Reichmann, L, et al., Nature, 332:323-327 (1988); Verhoeyen, M, et al., Science, 239:1534-36 (1988), the disclosures of each of which are herein incorporated by reference in their entireties).

Antibody polypeptides of the disclosure having CoV-S binding specificity may also be produced by constructing, using conventional techniques well known to those of ordinary skill in the art, an expression vector containing a promoter (optionally as a component of a eukaryotic or prokaryotic operon) and a DNA sequence encoding an antibody heavy chain in which the DNA sequence encoding the CDRs required for antibody specificity is derived from a non-human cell source, e.g., a rabbit or rodent B-cell source, while the DNA sequence encoding the remaining parts of the antibody chain is derived from a human cell source.

A second expression vector is produced using the same conventional means well known to those of ordinary skill in the art, said expression vector containing a promoter (optionally as a component of a eukaryotic or prokaryotic operon) and a DNA sequence encoding an antibody light chain in which the DNA sequence encoding the CDRs required for antibody specificity is derived from a non-human cell source, e.g., a rabbit or rodent B-cell source, while the DNA sequence encoding the remaining parts of the antibody chain is derived from a human cell source.

The expression vectors are transfected into a host cell by convention techniques well known to those of ordinary skill in the art to produce a transfected host cell, said transfected host cell cultured by conventional techniques well known to those of ordinary skill in the art to produce said antibody polypeptides.

The host cell may be co-transfected with the two expression vectors described above, the first expression vector containing DNA encoding a promoter (optionally as a component of a eukaryotic or prokaryotic operon) and a light chain-derived polypeptide and the second vector containing DNA encoding a promoter (optionally as a component of a eukaryotic or prokaryotic operon) and a heavy chain-derived polypeptide. The two vectors contain different selectable markers, but preferably achieve substantially equal expression of the heavy and light chain polypeptides. Alternatively, a single vector may be used, the vector including DNA encoding both the heavy and light chain polypeptides. The coding sequences for the heavy and light chains may comprise cDNA, genomic DNA, or both.

The host cells used to express the antibody polypeptides may be either a bacterial cell such as E. coli, or a eukaryotic cell such as P, pastoris. In one embodiment, a mammalian cell of a well-defined type for this purpose, such as a myeloma cell, a CHO cell line, a NS0 cell line, or a HEK293 cell line may be used.

The general methods by which the vectors may be constructed, transfection methods required to produce the host cell and culturing methods required to produce the antibody polypeptides from said host cells all include conventional techniques. Although preferably the cell line used to produce the antibody is a mammalian cell line, any other suitable cell line, such as a bacterial cell line such as an E. coli-derived bacterial strain, or a yeast cell line, may alternatively be used.

Similarly, once produced the antibody polypeptides may be purified according to standard procedures in the art, such as for example cross-flow filtration, ammonium sulphate precipitation, affinity column chromatography, hydrophobic interaction chromatography (“HIC”), and the like.

The antibody polypeptides described herein may also be used for the design and synthesis of either peptide or non-peptide mimetics that would be useful for the same therapeutic applications as the antibody polypeptides of the disclosure (See, for example, Saragobi et al., Science, 253:792-795 (1991), the contents of which are herein incorporated by reference in its entirety).

In another embodiment, the present disclosure contemplates methods for humanizing antibody heavy and light chains which bind to CoV-S. Exemplary methods for humanizing antibody heavy and light chains that may be applied to anti-CoV-S antibodies are identified herein and are conventional in the art.

iii. Screening Assays

The screening assays described here may be used to identify high affinity anti-CoV-S Abs for use in the pharmaceutical formulations of the present disclosure which may be useful in the treatment of diseases and disorders associated with CoV-S in subjects exhibiting symptoms of a CoV-S associated disease or disorder.

In some embodiments, the pharmaceutical formulations of the present disclosure are used as a diagnostic tool. The pharmaceutical formulation comprising the anti-CoV-S antibody can be used to assay the amount of CoV-S present in a sample and/or subject. As will be appreciated by one of skill in the art, such antibodies need not be neutralizing antibodies. In some embodiments, the diagnostic antibody is not a neutralizing antibody. In some embodiments, the diagnostic antibody binds to a different epitope than the neutralizing antibody binds to. In some embodiments, the two antibodies do not compete with one another.

In some embodiments, the pharmaceutical formulations disclosed herein are used or provided in an assay kit and/or method for the detection of CoV-S in mammalian tissues or cells in order to screen/diagnose for a disease or disorder associated with changes in levels of CoV-S. The kit comprises an antibody that binds CoV-S and means for indicating the binding of the antibody with CoV-S, if present, and optionally CoV-S protein levels. Various means for indicating the presence of an antibody can be used. For example, fluorophores, other molecular probes, or enzymes can be linked to the antibody and the presence of the antibody can be observed in a variety of ways. The method for screening for such disorders can involve the use of the kit, or simply the use of one of the disclosed antibodies and the determination of whether the antibody binds to CoV-S in a sample. As will be appreciated by one of skill in the art, high or elevated levels of CoV-S will result in larger amounts of the antibody binding to CoV-S in the sample. Thus, degree of antibody binding can be used to determine how much CoV-S is in a sample. Subjects or samples with an amount of CoV-S that is greater than a predetermined amount (e.g., an amount or range that a person without a CoV-S-related disorder would have) can be characterized as having a CoV-S-mediated disorder.

The present disclosure further provides for a kit for detecting binding of an anti-CoV-S antibody of the disclosure to CoV-S. In particular, the kit may be used to detect the presence of CoV-S specifically reactive with an anti-CoV-S antibody or an immunoreactive fragment thereof. The kit may also include an antibody bound to a substrate, a secondary antibody reactive with the antigen and a reagent for detecting a reaction of the secondary antibody with the antigen. Such a kit may be an ELISA kit and can comprise the substrate, primary and secondary antibodies when appropriate, and any other necessary reagents such as detectable moieties, enzyme substrates, and color reagents, for example as described herein. The diagnostic kit may also be in the form of an immunoblot kit. The diagnostic kit may also be in the form of a chemiluminescent kit (Meso Scale Discovery, Gaithersburg. MD). The diagnostic kit may also be a lanthanide-based detection kit (PerkinElmer, San Jose, CA).

A skilled clinician would understand that a biological sample includes, but is not limited to, sera, plasma, urine, fecal sample, saliva, mucous, pleural fluid, synovial fluid, and spinal fluid.

b. Excipients

The pharmaceutical formulation of the present disclosure comprises one or more excipients. The term “excipient”, as used herein, refers to any non-therapeutic agent added to the formulation to provide a desired consistency, viscosity, osmolarity or stabilizing effect.

The pharmaceutical formulations of the present disclosure may comprise a buffer or buffer system, which serves to maintain a stable pH and to help stabilize the antibody. In some embodiments, a buffer is selected to stabilize the antibody by maintaining the antibody in its native conformation. As used herein, the term “native” or “native conformation” refers to an antibody that is not aggregated or degraded. This is generally determined by an assay that measures the relative size of the antibody entity, such as by size exclusion chromatography. The non-aggregated and non-degraded antibody elutes at a fraction that equates to the native antibody, and is generally the main elution fraction. Aggregated antibody elutes at a fraction that indicates a size greater than the native antibody. Degraded antibody elutes at a fraction that indicates a size less than the native antibody.

In some embodiments, a buffer is selected to stabilize the antibody by minimizing the formation of high molecular species (e.g., dimer and aggregates as measured by size exclusion chromatography) of the antibody.

In some embodiments, a buffer is selected to stabilize the antibody by maintaining the antibody in its main charge form. As used herein, the term “main charge” or “main charge form”, refers to the fraction of antibody that elutes from an ion exchange resin in the main peak, which is generally flanked by more “basic” peaks on one side and more “acidic” peaks on the other side. The charged species is generally determined by an ion exchange chromatography, e.g., cation exchange chromatography, anion exchange chromatography, or by imaged capillary isoelectric focusing (iCIEF).

In some embodiments, a buffer is selected to stabilize the antibody by minimizing the formation of charge variants (e.g., acidic and basic peaks from cation exchange chromatography or imaged capillary isoelectric focusing (iCIEF)) of the antibody.

The pharmaceutical formulations of the present disclosure may have a pH of about 5.0-7.0, about 5.0-6.0, about 5.2-6.0, about 5.2-5.8, about 5.4-5.7, or about 5.4-5.6. For example, the formulations of the present disclosure may have a pH of about 4.0, about 4.5, about 5.0, about 5.1; about 5.2; about 5.3; about 5.4; about 5.5; about 5.6; about 5.7; about 5.8; about 5.9; about 6.0; about 6.1; about 6.2; about 6.3; about 6.4; about 6.5; or about 7.0. In some embodiments, the pH is about 5.0-6.0. In some embodiments, the pH is about 5.1. In some embodiments, the pH is about 5.2. In some embodiments, the pH is about 5.3. In some embodiments, the pH is about 5.4. In some embodiments, the pH is about 5.5. In some embodiments, the pH is about 5.6. In some embodiments, the pH is about 5.7. In some embodiments, the pH is about 5.8. In some embodiments, the pH is about 5.9. In some embodiments, the pH is about 6.0.

In some embodiments, the pharmaceutical formulation of the present disclosure comprises one or more buffers selected from the group consisting of an acetate buffer, a succinate buffer, a citrate buffer, a histidine buffer, and a phosphate buffer.

The concentration of the buffer within the pharmaceutical formulations of the present disclosure may vary depending on the specific properties desired of the formulations, as well as the particular circumstances and purposes for which the formulations are intended to be used. In certain embodiments, the formulations comprise a buffer concentration of about 0-50 mM, about 0-30 mM, about 0-20 mM, about 0-10 mM, or about 5-15 mM. In some embodiments, the formulations comprise a buffer concentration of about 0 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM or about 50 mM.

In some embodiments, the buffer comprises a histidine buffer. In some embodiments, the histidine is present at a concentration of about 0-20 mM or about 5-15 mM. In other embodiments, the histidine is present at a concentration of about 10 mM. In certain embodiments, the buffer system comprises acetate at about 10 mM and a pH of about 5.0-6.0, about 5.2-6.0, or about 5.2-5.8, or about 5.4-5.6. In certain embodiments, the buffer system comprises histidine at about 10 mM and a pH of about 5.4. In certain embodiments, the buffer system comprises histidine at about 10 mM and a pH of about 5.5. In certain embodiments, the buffer system comprises histidine at about 10 mM and a pH of about 5.6.

In some embodiments, the buffer comprises an acetate buffer. In some embodiments, the acetate is present at a concentration of about 0-20 mM, or about 5-15 mM. In other embodiments, the acetate is present at a concentration of about 10 mM. In certain embodiments, the buffer system comprises acetate at about 10 mM and a pH of about 5.0-6.0, about 5.2-6.0, or about 5.2-5.8, or about 5.4-5.6. In certain embodiments, the buffer system comprises histidine at about 10 mM and a pH of about 5.4. In certain embodiments, the buffer system comprises acetate at about 10 mM and a pH of about 5.5. In certain embodiments, the buffer system comprises acetate at about 10 mM and a pH of about 5.6.

In some embodiments, the pharmaceutical formulation of the present disclosure comprises one or more stabilizers and/or viscosity modulating excipients selected from the group consisting of sucrose, mannitol, glycine, proline, sodium chloride, arginine hydrochloride, arginine-glutamate, and sorbitol.

In some embodiments, a stabilizer is selected to stabilize the antibody by maintaining the antibody in its native conformation, e.g., the non-aggregated or the non-degraded form, which is generally determined by an assay that measures the relative size of the antibody entity, such as by size exclusion chromatography. In some embodiments, a stabilizer is selected to stabilize the antibody by minimizing formation of the high molecular species (e.g., dimer and aggregates as measured by size exclusion chromatography) of the antibody.

In some embodiments, a viscosity modulating excipient is selected to modulate, e.g., increase or decrease, the viscosity of the formulation, and/or to maintain the viscosity of the formulation at a certain level, e.g., less than 20.

In some embodiments, the stabilizer and/or the viscosity modulating excipient is selected in order to maintain a physiologically isotonic liquid formulation, i.e., between 270-400 mOsm/kg, or between 280-370 mOsm/kg, or between 280-380 mOsm/kg.

In some embodiments, the stabilizer and/or the viscosity modulating excipient is selected in order to modulate, e.g., increase or decrease, the turbidity or opalescence of the formulation, and/or to maintain the turbidity or opalescence of the formulation. In some embodiments, the opalescence of the formulation is between the 6NTU and 60NTU opalescence, e.g., about 6 NTU, about 7 NTU, about 8 NTU, about 9 NTU, about 10 NTU, about 11 NTU, about 12 NTU, about 13 NTU, about 14 NTU, about 15 NTU, about 16 NTU, about 17 NTU, about 18 NTU, about 19 NTU, about 20 NTU, about 21 NTU, about 22 NTU, about 23 NTU, about 24 NTU, about 25 NTU, about 26 NTU, about 27 NTU, about 28 NTU, about 29 NTU, about 30 NTU, about 35 NTU, about 40 NTU, about 45 NTU, about 50 NTU, or about 60 NTU.

The type of the stabilizers and/or viscosity modulating excipients and the amount of which contained within the formulation can vary depending on the specific circumstances and intended purposes for which the formulation is used.

In some embodiments, the stabilizer and/or the viscosity modulating excipient is sucrose. In some embodiments, the sucrose concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the sucrose concentration is about 0 mM, about 50 mM, about 60 mM, about 75 mM, about 80 mM, about 90 mM, about 100 mM, about 110 mM, about 125 mM, about 145 mM, about 150 mM, about 200 mM, about 235 mM, about 250 mM, or about 300 mM.

In some embodiments, the stabilizer and/or the viscosity modulating excipient is mannitol. In some embodiments, the mannitol concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the mannitol concentration is about 0 mM, about 50 mM, about 60 mM, about 75 mM, about 80 mM, about 90 mM, about 100 mM, about 110 mM, about 125 mM, about 145 mM, about 150 mM, about 200 mM, about 235 mM, about 250 mM, or about 300 mM.

In some embodiments, the stabilizer and/or the viscosity modulating excipient is arginine hydrochloride. In some embodiments, the arginine hydrochloride concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the arginine hydrochloride concentration is about 0 mM, about 50 mM, about 75 mM, about 80 mM, about 90 mM, about 100 mM, about 110 mM, about 125 mM, about 150 mM, about 200 mM, about 250 mM, about 270 mM, or about 300 mM.

In some embodiments, the stabilizer and/or the viscosity modulating excipient is glycine. In some embodiments, the glycine concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the glycine concentration is about 0 mM, about 50 mM, about 75 mM, about 80 mM, about 90 mM, about 100 mM, about 110 mM, about 125 mM, about 150 mM, about 200 mM, about 250 mM, about 270 mM, or about 300 mM.

In some embodiments, the stabilizer and/or the viscosity modulating excipient is proline. In some embodiments, the proline concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the proline concentration is about 0 mM, about 50 mM, about 75 mM, about 80 mM, about 90 mM, about 100 mM, about 110 mM, about 125 mM, about 150 mM, about 200 mM, about 250 mM, about 270 mM, or about 300 mM.

In some embodiments, the stabilizer and/or the viscosity modulating excipient is sodium chloride. In some embodiments, the sodium chloride concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the sodium chloride concentration is about 0 mM, about 50 mM, about 75 mM, about 80 mM, about 90 mM, about 100 mM, about 110 mM, about 125 mM, about 140 mM, about 150 mM, about 200 mM, about 250 mM, about 270 mM, or about 300 mM.

In some embodiments, the stabilizer and/or the viscosity modulating excipient is arginine glutamate. In some embodiments, the arginine glutamate concentration is about 0-300 mM, about 0-250 mM, about 0-150 mM, about 50-150 mM, about 50-100 mM, about 75-125 mM, about 50-200 mM, about 100-300 mM, or about 150-300 mM. In some embodiments, the arginine glutamate concentration is about 0 mM, about 50 mM, about 75 mM, about 80 mM, about 90 mM, about 100 mM, about 110 mM, about 125 mM, about 140 mM, about 150 mM, about 200 mM, about 250 mM, about 270 mM, or about 300 mM.

In some embodiments, the stabilizer and/or the viscosity modulating excipient is sorbitol. In some embodiments, the sorbitol concentration is about 0-300 mM, about 0-260 mM, about 0-150 mM, about 50-150 mM, about 50-100 mM, about 75-125 mM, about 50-200 mM, about 100-300 mM, or about 150-300 mM. In some embodiments, the sorbitol concentration is about 0 mM, about 50 mM, about 75 mM, about 80 mM, about 90 mM, about 100 mM, about 110 mM, about 125 mM, about 140 mM, about 150 mM, about 180 mM, about 200 mM, about 250 mM, about 260 mM, about 270 mM, or about 300 mM.

In some embodiments, the pharmaceutical formulation of the present disclosure comprises a surfactant. Exemplary surfactants that can be included in the formulations of the present disclosure include, but are not limited to, polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycol (PEG).

The amount of surfactant within the pharmaceutical formulations of the present disclosure may vary depending on the specific properties desired of the formulations, as well as the particular circumstances and purposes for which the formulations are intended to be used. In certain embodiments, the formulations comprise about 0.01% to 1% w/v, about 0.01-0.06% w/v, about 0.01-0.05% w/v, about 0.015-0.045% w/v, or about 0.02-0.04% w/v surfactant. For example, the formulations of the present disclosure may comprise about 0.01%; about 0.015%; about 0.02%; about 0.025%; about 0.03%; about 0.035%; about 0.04%; about 0.045%; about 0.05%; about 0.055%; about 0.06%; about 0.065%; about 0.07%; about 0.75%; about 0.08%; about 0.085%; about 0.09%; about 0.095%; about 0.1%; about 0.11%; about 0.12%; about 0.13%; about 0.14%; about 0.15%; about 0.16%; about 0.17%; about 0.18%; about 0.19%; about 0.20%; about 0.3%; about 0.4%; about 0.5%; about 0.6%; about 0.7%; about 0.8%; about 0.9%; about 1.0% w/v surfactant. In some embodiments, the formulation comprises about 0.03% w/v surfactant, e.g., polysorbate 80 (PS80).

Accordingly to one aspect of the present disclosure, the pharmaceutical formulation of the present disclosure is a high concentration liquid formulation having a viscosity of under 20 cPoise, a physiological isotonicity, i.e., between 270-400 mOsm/kg, or between 280-380 mOsm/kg, and an opalescence less than 60 NTU opalescence.

In some embodiments, the pharmaceutical formulation comprises (i) an isolated antibody, or antigen-binding fragment thereof, which specifically binds to the spike protein of a coronavirus (“CoV-S”); (ii) one or more buffers selected from the group consisting of an acetate buffer, a succinate buffer, a citrate buffer, a histidine buffer, and a phosphate buffer having a pH of about 4.5-7.0; (iii) one or more pharmaceutically acceptable excipients selected from the group consisting of sucrose, mannitol, glycine, proline, sodium chloride, arginine hydrochloride, arginine-glutamate, and sorbitol; and/or (iv) a surfactant.

In some embodiments, the pharmaceutical formulation comprises (i) an isolated antibody, or antigen-binding fragment thereof, which specifically binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) a histidine or acetate buffer having a concentration of about 0-20 mM and a pH of about 5.0-6.0; (iii) about 50-150 mM sucrose or mannitol; (iv) about 50-100 mM arginine hydrochloride; and (v) about 0.01-0.05% w/v polysorbate 80.

In some embodiments, the pharmaceutical formulation comprises (i) an isolated antibody, or antigen-binding fragment thereof, which specifically binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) a histidine or acetate buffer having a concentration of about 0-20 mM and a pH of about 5.2-5.8; (iii) about 50-150 mM sucrose or mannitol; (iv) about 50-100 mM arginine hydrochloride; and (v) about 0.01-0.05% w/v polysorbate 80.

In some embodiments, the pharmaceutical formulation comprises (i) an isolated antibody, or antigen-binding fragment thereof, which specifically binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) a histidine or acetate buffer having a concentration of about 0-20 mM and a pH of about 5.4-5.6; (iii) about 50-150 mM sucrose or mannitol; (iv) about 50-100 mM arginine hydrochloride; and (v) about 0.01-0.05% w/v polysorbate 80.

In some embodiments, the pharmaceutical formulation comprises (i) an isolated antibody, or antigen-binding fragment thereof, which specifically binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) a histidine or acetate buffer having a concentration of about 0-20 mM and a pH of about 5.4; (iii) about 50-150 mM sucrose or mannitol; (iv) about 50-100 mM arginine hydrochloride; and (v) about 0.01-0.05% w/v polysorbate 80. In one embodiment, the pharmaceutical formulation comprises (i) an isolated antibody, or antigen-binding fragment thereof, which specifically binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) a histidine or acetate buffer having a concentration of about 10 mM and a pH of about 5.5; (iii) about 100 mM sucrose or mannitol; (iv) about 75 mM arginine hydrochloride; and (v) about 0.03% w/v polysorbate 80.

In one embodiment, the pharmaceutical formulation comprises (i) an isolated antibody, or antigen-binding fragment thereof, which specifically binds to the spike protein of a coronavirus (“CoV-S”), wherein the antibody, or antigen-binding fragment thereof, comprises a heavy chain variable region (VH) comprising a VH CDR1 comprising SEQ ID NO:52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256; (ii) a histidine or acetate buffer having a concentration of about 10 mM and a pH of about 5.6; (iii) about 100 mM sucrose or mannitol; (iv) about 75 mM arginine hydrochloride; and (v) about 0.03% w/v polysorbate 80.

In some embodiments, the concentration of the antibody, or antigen-binding fragment thereof, is about 10-500 mg/mL, about 50-400 mg/mL, about 100-300 mg/mL, about 100-200 mg/mL, or about 125-175 mg/mL, or about 130-170 mg/mL. In some embodiments, the concentration of the antibody, or antigen-binding fragment thereof, is about 10 mg/mL, about 50 mg/mL, about 100 mg/mL, about 125 mg/mL, about 130 mg/mL, about 150 mg/mL, about 170 mg/mL, about 175 mg/mL, about 200 mg/mL, about 250 mg/mL, about 300 mg/mL, about 400 mg/mL, or about 500 mg/mL. In one embodiment, the concentration of the antibody, or antigen-binding fragment thereof, is about 150 mg/mL. In one embodiment, the concentration of the antibody, or antigen-binding fragment thereof, is about 200 mg/mL. In one embodiment, the concentration of the antibody, or antigen-binding fragment thereof, is about 250 mg/mL.

The pharmaceutical formulations of the present disclosure exhibit high levels of stability. The term “stable”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable”.

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion high performance liquid chromatography [SE-HPLC]), such that native means non-aggregated and non-degraded. An “acceptable degree of stability”, as used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 7 days, at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C., to about 45° C., e.g., storage at about −80° C., about −50° C., about −40° C., about −30° C., about −20° C., about 0° C., about 2°−8° C., about 5° C., about 25° C., about 35° C., about 37° C., about 40° C., or about 45° C. In some embodiments, the pharmaceutical formulation of the present disclosure is stable after 12 months of storage at about 2-8° C. In some embodiments, the pharmaceutical formulation of the present disclosure is stable after 24 months of storage at about 2-8° C. In some embodiments, the pharmaceutical formulation of the present disclosure is stable after 36 months of storage at about 2-8° C. In some embodiments, the pharmaceutical formulation of the present disclosure is stable after 1 week of storage at about 25° C. In some embodiments, the pharmaceutical formulation is stable at about ≤−30° C. for at least 1, 2, 3, 4 or 5 years.

In some embodiments, the pharmaceutical formulation of the present disclosure is stable after 2 weeks of storage at about 25° C. In some embodiments, the pharmaceutical formulation of the present disclosure is stable after 4 weeks of storage at about 25° C. In some embodiments, the pharmaceutical formulation of the present disclosure is stable after 1 week of storage at about 40° C.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC]). An “acceptable degree of stability”, as used herein, means that at most 10% of the antibody is in an aggregated form detected in the formulation after storage for a defined amount of time at a given temperature. In certain embodiments an acceptable degree of stability means that at most about 10%, 9%, 8%, 7%, 6%, 5%. 4%, 3%, 2%, 1%, 0.5%, or 0.1% of the antibody can be detected in an aggregate in the formulation after storage for a defined amount of time at a given temperature. The defined amount of time after which stability is measured can be at least 1 week, at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months or more. The temperature at which the pharmaceutical formulation may be stored when assessing stability can be any temperature from about −80° C., to about 45° C., e.g., storage at about −80° C., about −30° C., about −20° C., about 0° C., about 2°−8° C., about 5° C., about 25° C., about 35° C., about 37° C., about 40° C., or about 45° C.

Stability can be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., PNAS, Apr. 16, 2002, 99 (8): 5283-5288). The percentage of “acidified” antibody can be determined by ion exchange chromatography (e.g., cation exchange high performance liquid chromatography [CEX-HPLC]). An “acceptable degree of stability”, as used herein, means that at most 40% of the antibody is in a more acidic form detected in the formulation after storage for a defined amount of time at a defined temperature. In certain embodiments an acceptable degree of stability means that at most about 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 1 week, at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C., to about 45° C., e.g., storage at about −80° C., about −30° C., about −20° C., about 0° C., about 2°−8° C., about 5° C., about 25° C., about 30° C., about 37° C., about 40° C., or about 45° C.

Measuring the binding affinity of the antibody to its target may also be used to assess stability. For example, a pharmaceutical formulation of the present disclosure may be regarded as stable if, after storage at e.g., −80° C., −40° C., −30° C., −20° C., 0° C., 2−8° C., 5° C., 25° C. 37° C., 40° C., 45° C., etc. for a defined amount of time (e.g., 14 days, 1 month, 2 months, 6 months, or 12 months), the anti-CoV-S antibody contained within the formulation binds to the spike protein of a coronavirus (“CoV-S”) with an affinity that is at least 80%, 85%, 90%, 95%, or more of the binding affinity of the antibody prior to said storage. Binding affinity may be determined by any method, such as e.g., ELISA or plasmon resonance. Biological activity may be determined by an activity assay, such as by contacting a cell that expresses CoV-S with the formulation comprising the anti-CoV-S antibody. The binding of the antibody to such a cell may be measured directly, such as via FACS analysis. Alternatively, the neutralization activity of the antibody in a host against a coronavirus or a pseudovirus may be measured.

In some embodiments, stability of the pharmaceutical formulation, e.g., the percentage of native, aggregated or degraded antibody, the percentage of antibody in its major charge form or charge variant forms, or the binding affinity of the antibody, can also be measured by subjecting the pharmaceutical formulation to conditions such as multiple cycles, e.g., 1, 2, 3, 4, or 5 cycles, of freeze/thaw, agitation and/or thermal stresses, as described in Example 1.

C. Methods of Ameliorating or Reducing Symptoms of, or Treating, or Preventing, Diseases and Disorders Associated with CoV

In another embodiment, the pharmaceutical formulations of the present disclosure are useful for ameliorating or reducing the symptoms of, or treating, or preventing, diseases and disorders associated with CoV-S. The pharmaceutical formulations of the present disclosure comprise anti-CoV-S antibodies described herein, or antigen-binding fragments thereof, as well as combinations, can also be administered in a therapeutically effective amount to patients in need of treatment of diseases and disorders associated with CoV-S in the form of a pharmaceutical composition as described in greater detail below.

Symptoms of CoV infection may include fever, cough, runny nose, congestion, sore throat, bronchitis, pneumonia, shortness of breath, chest pain, headache, muscle ache, chills, fatigue, conjunctivitis, diarrhea, loss of smell, and loss of taste. Complications and/or diseases/disorders associated with coronavirus infection may include, for example, bronchitis, pneumonia, respiratory failure, acute respiratory failure, organ failure, multi-organ system failure, pediatric inflammatory multisystem syndrome, acute respiratory distress syndrome (a severe lung condition that causes low oxygen in the blood and organs), blood clots, cardiac conditions, myocardial injury, myocarditis, heart failure, cardiac arrest, acute myocardial infarction, dysrhythmias, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, gastrointestinal complications, aspergillosis, increased susceptibility to infection with another virus or bacteria, and/or pregnancy-related complications. Certain diseases and conditions, such as high blood pressure, type 1 diabetes, liver disease, overweight, chronic lung diseases including cystic fibrosis, pulmonary fibrosis, and asthma, compromised immune system due to transplant, use of an immunosuppressant, or HIV infection, and brain and nervous system condition, may increase the risk of CoV infection-associated complications and diseases.

Also, the pharmaceutical formulations of the present disclosure comprising the anti-CoV-S antibodies and antigen-binding fragments may be used alone or in conjunction with other active agents, e.g., opioids and non-opioid analgesics such as NSAIDs to elicit analgesia. In some embodiments, aspirin and/or acetaminophen may be taken in conjunction with the subject anti-CoV-S antibody or antigen-binding fragment. Aspirin is another type of non-steroidal anti-inflammatory compound.

The pharmaceutical formulations of the present disclosure comprising the antibodies potentially optionally may be combined with one or more of the following: (i) an antiviral drug, optionally, remdesivir, favipiravir, darunavir, nelfinavir, saquinavir, lopinavir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, which is optionally moexipril; or (viii) a drug that inhibits priming of CoV-S, optionally a serine protease inhibitor, further optionally nafamostat. in order to increase or enhance pain management. This may allow for such analgesic compounds to be administered for longer duration or at reduced dosages thereby potentially alleviating adverse side effects associated therewith.

The subject to which the pharmaceutical formulation is administered can be, e.g., any human or non-human animal needing such treatment, prevention and/or amelioration, or who would otherwise benefit from the inhibition or attenuation of CoV-S-mediated activity. For example, the subject can be an individual that is diagnosed with, or who is deemed to be at risk of being afflicted by any of the aforementioned diseases or disorders. In some instances, the subject may be in an advanced state of CoV infection, e.g., a subject who is on a ventilator. In some instances, the subject can be one having one or more risk factors (such as advanced age, obesity, diabetes, etc, and others previously identified) which correlate to a poor CoV treatment or recovery prognosis. The present disclosure further includes the use of any of the pharmaceutical formulations disclosed herein in the manufacture of a medicament for the treatment, prevention and/or amelioration of any disease or disorder associated with CoV or CoV-S activity (including any of the above-mentioned exemplary diseases, disorders and conditions).

### D. Administration

In one embodiment, the pharmaceutical formulations comprising the anti-CoV-S antibodies, or antigen-binding fragments thereof, described herein, as well as combinations of said antibodies or antigen-binding fragments thereof, are administered to a subject at a concentration of between 0.1 mg/ml and about any one of 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190, 200, or 250 mg/ml, +/−10% error.

In another embodiment, the pharmaceutical formulations of the present disclosure comprising the anti-CoV-S antibodies and fragments thereof described herein are administered to a subject at a dose of between about 0.01 and 100.0 or 200.0 mg/kg of body weight of the recipient subject. In certain embodiments, depending on the type and severity of the CoV-S-related disease, about 1 μg/kg to 50 mg/kg (e.g., 0.1−20 mg/kg) of antibody is an initial candidate dosage for administration to the patient, whether, for example, by one or more separate administrations, or by continuous infusion. In another embodiment, about 1 μg/kg to 15 mg/kg (e.g., 0.1 mg/kg-10 mg/kg) of antibody is an initial candidate dosage for administration to the patient. A typical daily dosage might range from about 1 μg/kg to 100 mg/kg or more, depending on several factors, e.g., the particular mammal being treated, the clinical condition of the individual patient, the cause of the disorder, the site of delivery of the agent, the method of administration, the scheduling of administration, and other factors known to medical practitioners. However, other dosage regimens may be useful.

For example, in addition to the relative dosages (mg/kg) discussed herein, the pharmaceutical formulations of the present disclosure comprising the anti-CoV-S antibodies and antigen-binding fragments thereof can be administered to a subject at an absolute dose (mg). Accordingly, in one embodiment, the pharmaceutical formulations of the present disclosure comprising the anti-CoV-S antibodies and antigen-binding fragments thereof described herein are administered to a subject at a dose of between about 1 microgram and about 2000 milligrams regardless of the route of administration.

In some embodiments, the pharmaceutical formulations are administered at a dose of about 100 mg to about 2000 mg, about 100 mg to about 500 mg, about 200 mg to about 400 mg, about 250 mg to about 350 mg, about 200 mg to about 1500 mg, about 300 mg to about 600 mg, about 500 mg to about 1200 mg, or about 300 mg to about 1200 mg. In some embodiments, the pharmaceutical formulations are administered at a dose of about 200 mg, about 250 mg, about 300 mg, about 350 mg, about 400 mg, about 500 mg, about 600 mg, about 700 mg, about 800 mg, about 900 mg, about 1000 mg about 1100 mg, about 1200 mg, about 1300 mg, about 1400 mg, about 1500 mg, about 1600 mg, about 1700 mg, about 1800 mg, about 1900 mg, or about 2000 mg.

In some embodiments, the pharmaceutical formulations are administered intravenously. In other embodiments, the pharmaceutical formulations are administered intramuscularly.

In some embodiments, the pharmaceutical formulation is administered at a dose of about 300 mg intramuscularly, about 300 mg intravenously, about 500 mg intravenously, about 600 mg intramuscularly, or about 1200 mg intravenously.

In some embodiments, a pharmaceutical formulation comprising at least one antibody, or antigen-binding fragment thereof, is administered. In some embodiments, a pharmaceutical formulations comprising at least two antibodies, or antigen-binding fragments thereof, is administered. In some embodiments, the pharmaceutical formulation comprising the anti-CoV-S antibody and antigen-binding fragment thereof, e.g., ADI-58125, may be used in combination with a second pharmaceutical formulation comprising a second antibody, or antigen-binding fragment thereof, wherein the second antibody, or antigen-binding fragment thereof, is selected from the group consisting of ADI-58120, ADI-58121, ADI-58122, ADI-58123, ADI-58124, ADI-58126, ADI-58127, ADI-58128, ADI-58129, ADI-58130, ADI-58131, or a combination thereof. In some embodiment, the second antibody, or antigen-binding fragment thereof, is ADI-58122. In one embodiment, the second antibody, or antigen-binding fragment thereof, is ADI-58127. In one embodiment, the second antibody, or antigen-binding fragment thereof, is ADI-58129. In one embodiment, the second antibody, or antigen-binding fragment thereof, is ADI-58131. In some embodiments, the first antibody, or antigen-binding fragment thereof, is ADI-58125, and the second antibody, or antigen-binding fragment thereof, is ADI-58122.

In one embodiment, the pharmaceutical formulation is administered once. In one embodiment, the pharmaceutical formulation is administered twice. In one embodiment, the pharmaceutical formulation is administered weekly. In another embodiment, the pharmaceutical formulation is administered daily, weekly, every two weeks, monthly, or every two months. In one embodiment, the pharmaceutical formulation is administered weekly for about four weeks, once weekly for about a month, weekly for about 5 weeks, weekly for about 6 weeks, weekly for about 7 weeks, or weekly for about two months.

In one embodiment, the pharmaceutical formulations comprising anti-CoV-S antibodies described herein, or anti-CoV-S antigen-binding fragments thereof, as well as combinations of said antibodies or antigen-binding fragments thereof, are administered to a recipient subject with a frequency of once every twenty-six weeks or less, such as once every sixteen weeks or less, once every eight weeks or less, once every four weeks or less, once every two weeks or less, once every week or less, or once daily or less.

According to preferred embodiments, the antibody containing medicament or pharmaceutical formulation is peripherally administered to a subject via a route selected from one or more of: orally, sublingually, buccally, topically, rectally, via inhalation, transdermally, subcutaneously, intravenously, intra-arterially, or intramuscularly, via intracardiac administration, intraosseously, intradermally, intraperitoneally, transmucosally, vaginally, intravitreally, epicutaneously, intra-articularly, peri-articularly, or locally.

The pharmaceutical formulation may be administered every year or less, every 6 months or less, every three months or less, every one month or less, every two weeks or less, every week or less, once daily or less, multiple times per day, and/or every few hours. In one embodiment, the administration is given every month, every 2 months, every 3 months, every 4 months, every 5 months, every 6 months, every 7 months, every eight months, every 9 months, every 10 months, every 11 months, or once a year.

In some embodiments, the pharmaceutical formulation may be administered at a dose of about 100 mg to about 5000 mg, about 100 mg to about 4500 mg, about 100 mg to about 2000 mg, about 200 mg to about 1500 mg, about 300 mg to about 600 mg, about 500 mg to about 1200 mg, or about 300 mg to about 1200 mg, or about 1200 mg to about 4500 mg. In some embodiments, the pharmaceutical formulation may be administered at a dose of about 200 mg, about 300 mg, about 400 mg, about 500 mg, about 600 mg, about 700 mg, about 800 mg, about 900 mg, about 1000 mg about 1100 mg, about 1200 mg, about 1300 mg, about 1400 mg, about 1500 mg, about 1600 mg, about 1700 mg, about 1800 mg, about 1900 mg, about 2000 mg, about 2500 mg, about 3000 mg, about 3500 mg, about 4000 mg, about 4500 mg, or about 5000 mg.

In some embodiments, the pharmaceutical formulation may be is administered at a dose of about 300 mg intramuscularly, about 500 mg intravenously, about 600 mg intramuscularly, about 1200 mg intramuscularly, about 1200 mg intravenously, or about 4500 mg intravenously.

It is to be understood that the concentration of the antibody administered to a given patient may be greater or lower than the exemplary administration concentrations set forth above.

A person of skill in the art would be able to determine an effective dosage and frequency of administration through routine experimentation, for example guided by the disclosure herein and the teachings in, Goodman & Gilman's The Pharmacological Basis of Therapeutics, Brunton, L. L, et al. editors, 11th edition, New York, New York: McGraw-Hill (2006); Howland, R. D, et al., Pharmacology, Volume 864, Lippincott's illustrated reviews., Philadelphia, PA: Lippincott Williams & Wilkins (2006); and Golan, D. E., Principles of pharmacology: the pathophysiologie basis of drug therapy, Philadelphia, PA: Lippincott Williams & Wilkins (2007).

In another embodiment, the pharmaceutical formulation comprising anti-CoV-S antibodies described herein, or CoV-S binding fragments thereof, as well as combinations of said antibodies or antigen-binding fragments thereof, are administered to a subject. In a preferred embodiment, the subject is a human.

A “pharmaceutical composition” or “medicament” refers to a chemical or biological composition suitable for administration to a subject, preferably a mammal, more preferably a human. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can occur by means of injection, powder, liquid, gel, drops, or other means of administration.

In one embodiment, the pharmaceutical formulation comprising the anti-CoV-S antibodies or antigen-binding fragments thereof, as well as combinations of said antibodies or antigen-binding fragments thereof, may be optionally administered in combination with one or more active agents. Such active agents include (i) an antiviral drug, optionally, remdesivir, favipiravir, darunavir, nelfinavir, saquinavir, lopinavir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, optionally moexipril; or (viii) a drug that inhibits priming of CoV-S, optionally a serine protease inhibitor, further optionally nafamostat.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratadine, methscopolamine, mizolastine, norastemizole, phenindamine, promethazine, pyrilamine, terfenadine, and tranilast.

In CoV infection, respiratory symptoms are often exacerbated by additional bacterial infection. Therefore, such active agents may also be antibiotics, which include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, methicillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilamide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclomethasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

A “pharmaceutical excipient” or a “pharmaceutically acceptable excipient” is a carrier, usually a liquid, in which an active therapeutic agent is formulated. In one embodiment, the active therapeutic agent is a humanized antibody described herein, or one or more fragments thereof. The excipient generally does not provide any pharmacological activity to the formulation, though it may provide chemical and/or biological stability, and release characteristics. Exemplary formulations can be found, for example, in Remington's Pharmaceutical Sciences, Gennaro, A, editor, 19th edition, Philadelphia, PA: Williams and Wilkins (1995), which is incorporated by reference.

As used herein “pharmaceutically acceptable carrier” or “excipient” includes any and all solvents, dispersion media, coatings, antibacterial and antifungal agents, isotonic, and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the disclosure is contemplated. Supplementary active compounds can also be incorporated into the compositions.

Pharmaceutical formulations typically must be sterile and stable under the conditions of manufacture and storage. The disclosure contemplates that the pharmaceutical formulations is present in liquid form. The formulation can be formulated as a solution, microemulsion, liposome, a lyophilized form, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The disclosure further contemplates the inclusion of a stabilizer in the pharmaceutical composition. The proper fluidity can be maintained, for example, by the maintenance of the required particle size in the case of dispersion and by the use of surfactants.

In some embodiments, the pharmaceutical formulations are stable at about 2−8° C. for at least 1 week. 2 weeks, 3 weeks, or 1, 2, 3, 4, 5, 6, 7, 8, 9, 10, 11 or 12 months. In some embodiments, the pharmaceutical formulations are stable at about 2−8° C. for at least 1 or 2 or 3 years. In some embodiments, the pharmaceutical formulations are stable at about ≤−30° C. for at least 1, 2, 3, 4, or 5 years. In some embodiments, the pharmaceutical formulations are stable at about 25° C. for at least 1, 2, 3, 4, 5, 6, 7, 8, 9, 10, 11 or 12 weeks. In some embodiments, the pharmaceutical formulations are stable at about 40° C. for at least 1, 2, 3, 4, 5, 6, 7, 8, 9, 10, 11 or 12 weeks.

In many cases, it will be preferable to include isotonic agents, for example, sugars, polyalcohols such as mannitol, and sorbitol, or sodium chloride in the composition. Absorption of the injectable compositions can be prolonged by including an agent that delays absorption, for example, monostearate salts and gelatin. Moreover, the alkaline polypeptide can be formulated in a time-release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, polylactic, and polyglycolic copolymers (“PLG”). Many methods for the preparation of such formulations are known to those skilled in the art.

For each of the recited embodiments, the compounds can be administered by a variety of dosage forms. Any biologically acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

The above description of various illustrated embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings provided herein of the disclosure can be applied to other purposes, other than the examples described herein.

Certain anti-CoV-S antibody polynucleotides and polypeptides are disclosed in the sequence listing accompanying this patent application filing, and the disclosure of said sequence listing is herein incorporated by reference in its entirety.

The entire disclosure of each document cited (including patents, patent applications, journal articles, abstracts, manuals, books, or other disclosures) in the Background, Detailed Description, and Examples is herein incorporated by reference in their entireties.

The following examples are put forth so as to provide those of ordinary skill in the art with a complete disclosure and description of how to make and use the subject disclosure and are not intended to limit the scope of what is regarded as the invention. Efforts have been made to ensure accuracy with respect to the numbers used (e.g. amounts, temperature, concentrations, etc.), but some experimental errors and deviations should be allowed for. Unless otherwise indicated, parts are parts by weight, molecular weight is average molecular weight, temperature is in degrees centigrade; and pressure is at or near atmospheric.

### Examples

### Example 1: Development of a Stable Liquid Formulation for Anti-Coronavirus Antibodies

This Example provides the ADI-58125 commercial liquid drug product formulation development. The objective of this work was to develop a high concentration formulation (e.g., >100 mg/mL) with a favorable stability profile for long term storage of drug product (i.e., a projected shelf-life of at least 2 years at 2−8° C.) and for drug substance (i.e., about 3 to 5 years at ≤−30° C.). The high concentration was desirable to enable intramuscular (IM) route of administration. A formulation with acceptable stability upon freeze/thaw was another desirable attribute, to enable drug substance (stored frozen) to have the same formulation and product concentration as drug product.

Prior to development of the commercial formulation, two drug products for clinical use were developed and the details are listed in Table 1.

TABLE 1
ADI-58125 clinical drug product description

| | | Fill volume/ | Route of | Long-term |
|---|---|---|---|---|
| ADI-58125 | Formulation | Container | Administration, | storage |
| concentration | Composition | closure | Dose | temperature |
| 30 mg/mL | 20 mM Histidine, | 4 mL, 10R | IV, IM | ≤−60° C. |
| | 8% sucrose | Type I glass | (300 mg) | |
| | 0.01% PS80 | vial, 20 mM | | |
| | pH 6.5 | stopper | | |
| 100 mg/mL | 20 mM Histidine, | 3 mL, 8R | IV (1500 mg+) | ≤−60° C. |
| | 150 mM NaCl, | Type I glass | IM 2 inj (600 | |
| | 0.01% PS80 | vial, 20 mm | mg) | |
| | pH 6.5 | stopper | | |

The overall goal of the commercial formulation development was to meet the requirements listed in the Quality Target Product Profile (QTPP) listed in Table 2.

TABLE 2
Quality Target Product Profile

| Attribute | Criteria | |
|---|---|---|
| Dosage form | Single-use, liquid | |
| Route of Administration | IM and IV | |
| Label claim | 300 mg ADI-58125 | |
| Protein concentration | 150 | mg/mL |
| Container Closure | Type I glass vial, 13 mm stopper | |
| Osmolality | 270-400 | mOsm/kg |
| Viscosity | <20 | cP |
| Storage stability requirements | | |
| Long-term, 2-8° C. | ≥2 | y |
| Short-term, 25° C. | ≥2 | w |
| Long-term, ≤−20° C. | ≥2 | y |
| (drug substance target only) | | |
| Long-term, ≤−30° C. | 3-5 | y |
| (drug substance target only) | | |

FIG. 1 shows the overview of the development studies at 150 mg/mL ADI-58125. In brief, the first screening study identified histidine as the buffer, the second and third studies identified the optimal pH as 5.5 and the optimal excipients as sucrose and arginine hydrochloride. Surfactants are typically added to antibody liquid formulations to protect against interfacial stresses during freeze thaw and transportation. Based on prior knowledge, PS80 at a concentration of 0.03% w/v was selected for this formulation.

The next step in the formulation development was to select the concentrations of arginine and sucrose to provide optimal stability, viscosity, and osmolality for the formulation. One challenge associated with high-concentration formulations is increased electrostatic interactions between proteins and excipients and the volume exclusion effect. Such interactions can create an offset between excipient levels in final products and diafiltration buffers in ultrafiltration processes. Several small scale UF/DF experiments were conducted to identify the composition of the diafiltration buffers and process that would result in the target formulations. This process was used to produce three formulations with different ratios of arginine hydrochloride and sucrose, which were then put on stability to identify the optimal formulation. From this study, a ratio of 75 mM arginine and 100 mM sucrose was selected. Based on the data from all of the above studies, the optimal formulation to meet the QTPP was selected as 150 mg/mL ADI-58125, in 10 mM histidine, 75 mM arginine hydrochloride, 100 mM sucrose, with 0.03% PS80. A confirmation study was then initiated by testing stability of the selected formulation produced by a process that was representative of the manufacturing process. This stability was monitored for at least 12 months.

### A. Formulation Screening Studies

### Formulation Study 1

The first screening study was performed at 150 mg/mL ADI-58125 and Table 3 lists composition of the 16 formulations that were tested. Buffer choices were limited to those found in marketed products, and those having suitable buffering capacities in the pH region of interest for formulating a mAb (here pH 4.5−7.0). Concentrations of the buffering species were fixed at 20 mM to ensure adequate buffering capacity in the presence of 150 mg/mL protein. The stability impacts of a polyol (sorbitol) or electrolyte (NaCl) added to any given formulation were also simultaneously evaluated. Sorbitol was selected for this study since it is chemically stable over a range of pH while sucrose is not. The storage stability of the formulations listed in Table 3 were examined after 1 week at 40° C. 2 weeks at 25° C., and 5 weeks at 2−8° C. using size exclusion chromatography (SEC) and cation exchange chromatography (CEX). In addition to these measurements, osmolality, pH, protein content, viscosity, turbidity (A340), and visual appearance were also assessed.

The visual analysis, pH, viscosity, and turbidity measurements were shown in FIG. 2. This data shows that the viscosity was increased when sorbitol was used as an excipient and was decreased when sodium chloride was used. The opposite trend was seen for the turbidity/opalescence of the solutions.

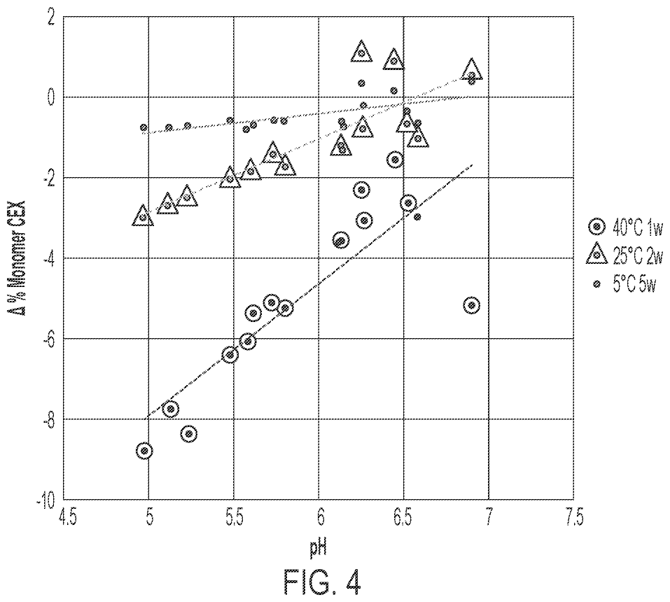

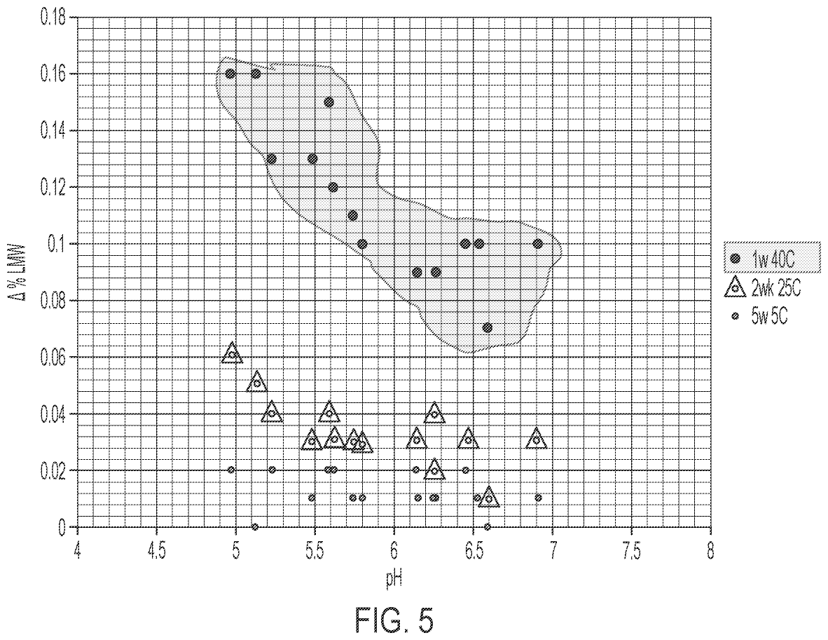

The SEC data and CEX data were analyzed as a percent loss of monomer at 1w at 40° C., 2 w at 25° C., and 5w at 5° C. from t=0. This data was graphed as a function of pH and are shown in FIG. 3 and FIG. 4. The SEC main peak decreased with increasing pH and temperature. There was one outlier at pH 5.12 at 40° C. which showed a large decrease in monomer peak. The slopes of the trend lines at each temperature were similar indicating that the 40° C., and 25° C., accelerated stability trends with respect to pH are similar to that at 2−8° C. The decrease in the monomer was primarily due to increase in the higher molecular weight species including dimer and aggregate species and less due to the increase in fragments. The percentage increase in fragment peak from time zero (attributed to the Fab arm) was shown in FIG. 5. At 40° C. fragmentation increased at lower pH values (<6.0), but at the lower temperatures, this trend flattened out with minimal pH dependent differences in the rates at 2−8° C. Overall, the retention of monomer was maximized at the lower pH values and among the buffer species, histidine provided the best stabilization. In total, the best performing formulations at 5° C. lost ˜ 0.2% purity to HMW 2 over the course of 5 weeks.

The CEX data on the other hand showed an opposite trend with the main peak being retained at the higher pH values. The decrease in the main peak was mainly due to increase in the basic peaks. When comparing the data across the three temperatures, although a trend of lower main peak with decreasing pH was observed the slopes decrease with temperature, indicating that the higher temperature degradation may not be indicative of degradation at the lower temperatures.

The summary for this study is that the physical stability is best in the histidine buffers at the lower pH values. The aggregation trends at 40° C., are good indicator of stability at 5° C. Chemical stability as monitored by the charge variants had an opposing trend to that of aggregation. The pH dependence of the charge variants was temperature-dependent, and at the lower temperatures, the differences in the rates were lower than at 40° C. Addition of sorbitol or sodium chloride resulted in opposing trends for viscosity and turbidity. Sorbitol formulations had higher viscosity and lower turbidity than the sodium chloride formulations. Viscosity for all formulations was below 10 cP.

TABLE 3
Composition of formulations screened in Study 1

| | | Protein | Acetate | Succinate | Citrate | Histidine | Phosphate | NaCl | Sorbitol |
|---|---|---|---|---|---|---|---|---|---|
| Form | pH | (mg/mL) | (mM) | (mM) | (mM) | (mM) | (mM) | (mM) | (mM) |
| F01 | 4.5 | 150 | 20 | 0 | 0 | 0 | 0 | 0 | 260 |
| F02 | 5 | 150 | 20 | 0 | 0 | 0 | 0 | 130 | 0 |
| F03 | 5.5 | 150 | 20 | 0 | 0 | 0 | 0 | 0 | 260 |
| F04 | 5 | 150 | 0 | 20 | 0 | 0 | 0 | 0 | 260 |
| F05 | 5.5 | 150 | 0 | 20 | 0 | 0 | 0 | 130 | 0 |
| F06 | 6 | 150 | 0 | 20 | 0 | 0 | 0 | 50 | 180 |
| F07 | 5.5 | 150 | 0 | 0 | 20 | 0 | 0 | 130 | 0 |
| F08 | 6 | 150 | 0 | 0 | 20 | 0 | 0 | 50 | 180 |
| F09 | 6.5 | 150 | 0 | 0 | 20 | 0 | 0 | 0 | 260 |
| F10 | 5 | 150 | 0 | 0 | 0 | 20 | 0 | 130 | 0 |
| F11 | 5.5 | 150 | 0 | 0 | 0 | 20 | 0 | 50 | 180 |
| F12 | 6 | 150 | 0 | 0 | 0 | 20 | 0 | 0 | 260 |
| F13 | 6.5 | 150 | 0 | 0 | 0 | 20 | 0 | 130 | 0 |
| F14 | 6 | 150 | 0 | 0 | 0 | 0 | 20 | 0 | 260 |
| F15 | 6.5 | 150 | 0 | 0 | 0 | 0 | 20 | 130 | 0 |
| F16 | 7 | 150 | 0 | 0 | 0 | 0 | 20 | 50 | 180 |

### Formulation Study 2

The second study investigated the stability of ADI-58125 in the presence of added stabilizers and viscosity reducing excipients. Table 4 lists the thirteen formulation compositions examined in this study. Histidine was utilized as the buffer of choice for all formulations, as it was found to be the most stabilizing buffer from Study 1. The pH of the formulations in Table 4 were varied over the effective buffering range of histidine, and histidine concentration was varied from 10 to 30 mM in an attempt to further elucidate the effect of buffer concentration on stability. The stabilizers and viscosity modulating excipients examined in this study included sucrose, glycine, proline, NaCl, arginine-HCl, and arginine-glutamate. Polysorbate 80 (PS80) was included at a platform level in all formulations (0.03% w/v), as it will necessarily be included as an interfacial stabilizer in any final formulation of ADI-58125.

Although the target concentration of ADI-58125 was 150 mg/mL, three formulations were at 180 mg/mL to further elucidate the viscosity and stability behavior of ADI-58125 as a function of increasing concentration. The storage stability of the formulations listed in Table 4 were examined after 1 week at 40° C., 2 weeks at 25° C., and 4 weeks at 2−8° C.

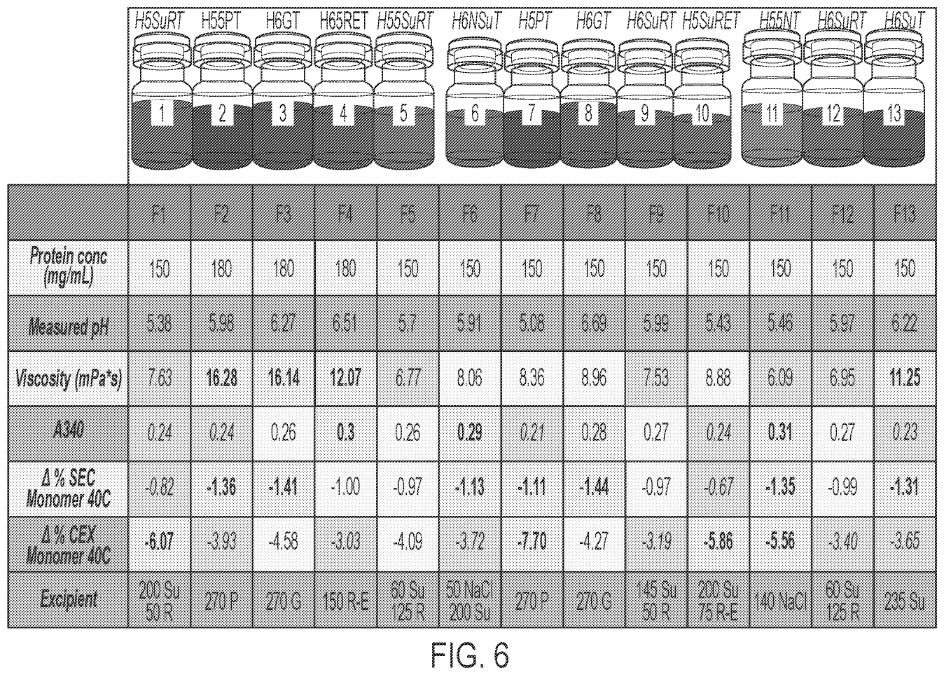

The visual analysis, measured pH, viscosity and turbidity were shown in FIG. 6. Inspection of viscosity of the 150 mg/mL formulations indicates that inclusion of NaCl or arginine-HCl into a formulation significantly reduced viscosity. This is especially noteworthy for the arginine-HCl formulations, as they were all co-formulations with sucrose, a known viscosity enhancer. Similar to Study 1, opalescence was more pronounced in some formulations versus others, and was affected by both the excipient type and concentration. In general, formulations containing NaCl, arginine-HCl, arginine-glutamate, and glycine had more visual opalescence than those with proline or sucrose. Furthermore, increasing the relative concentration of NaCl, arginine-HCl, or arginine-glutamate in a given formulation appeared to result in increased opalescence (visually) as well.

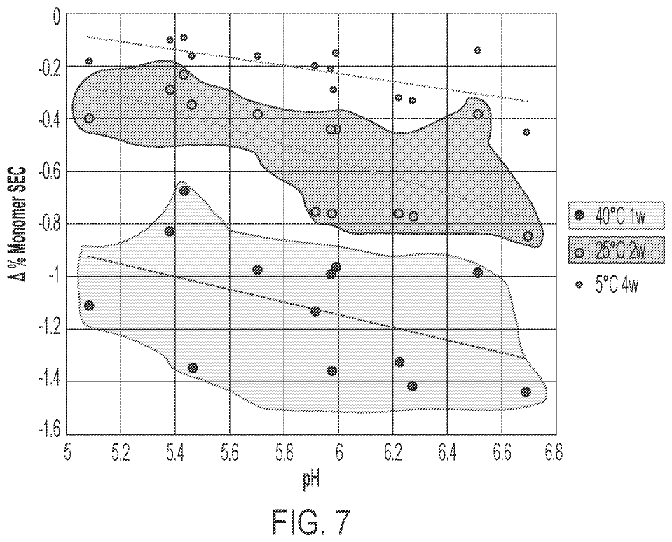

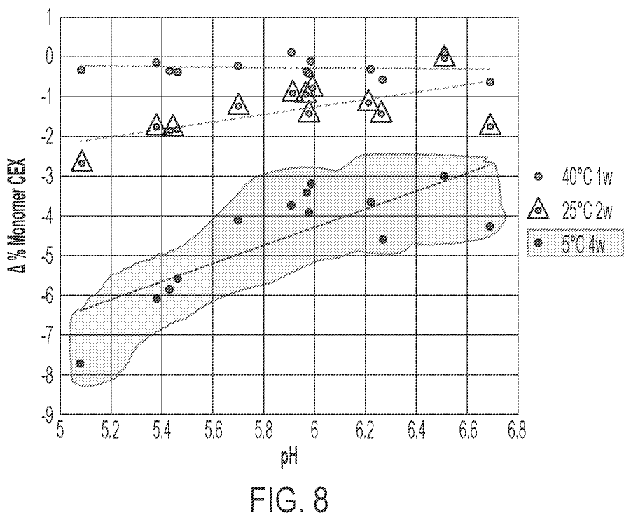

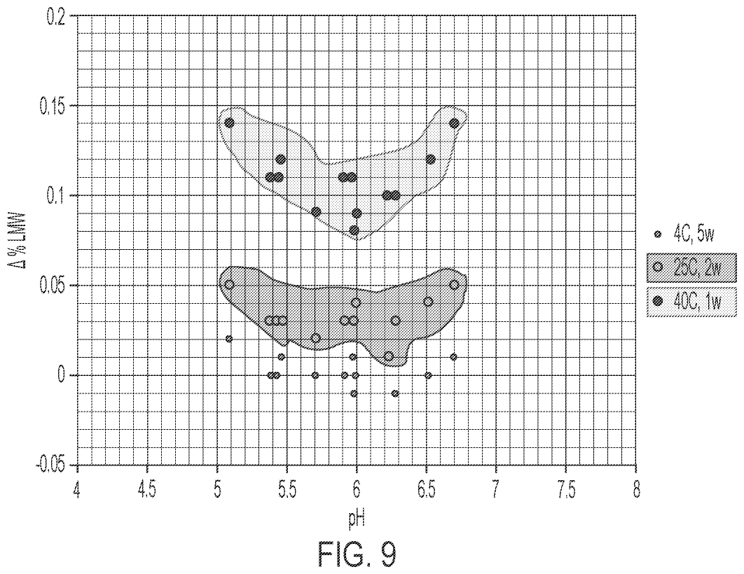

FIG. 7 and FIG. 8 showed the SEC and CEX data analysis. The difference in main peak from t=1w at 40° C., t=2w at 25° C., and t=5w at 5° C., to the initial timepoint was plotted as a function of pH. The results were similar to that seen from Study 1, i.e., the SEC monomer was better retained at the low pH formulations and the trends were similar between the three temperatures whereas the CEX monomer showed both a pH and temperature dependence. Similar to Study L1, the aggregation contributed more to the loss of main peak in the SEC when compared to the fragmentation. The fragmentation rates were shown in FIG. 9 and showed a minimum from pH 5.8 to 6.2 at 40° C., however this trand flattened out at the lower temperatures extending the acceptable pH range from 5.3 to 6.5.

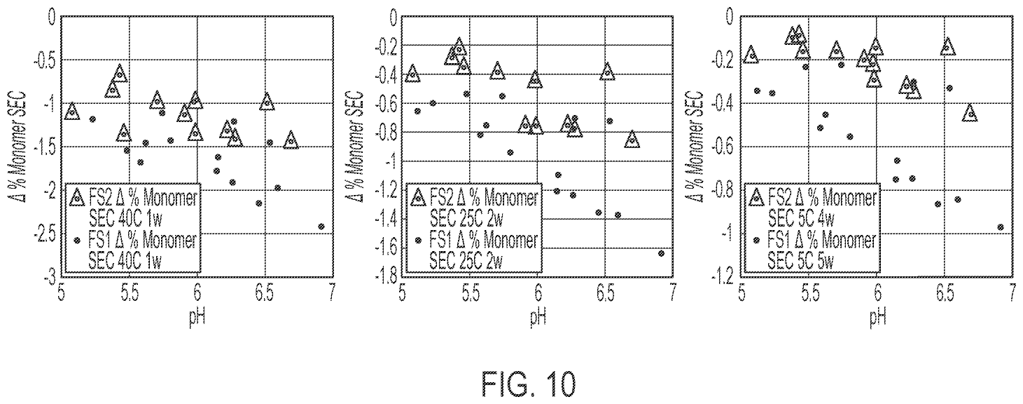

FIG. 10 showed a comparison of the SEC data for Studies 1 and 2 at 40, 25 and 5° C. These graphs showed that almost all the formulations in Study 2 performed better in retaining the monomer peak when compared to those in Study 1. On further examination, the formulations containing both sucrose and arginine hydrochloride or arginine glutamate performed the best in retaining monomer. The data from Study 1 and Study 2 can be summarized in the following points:
(1) Histidine buffer was the optimal buffer, but could also use acetate instead of histidine.
(2) A mixture of sucrose and arginine provided the best stability, viscosity and opalescence.
(3) pH dependence of the rate decreased with temperature for CEX degradation, therefore rates at 40° C. should not be relied on as a selection criterion since they do not reflect the rates at 2−8° C.
(4) Among the selected formulations (histidine, sucrose, arginine) at 5° C., SEC monomer may show a larger pH dependence than CEX monomer
(5) High molecular species (dimer and aggregates from SEC) and charge variants (acidic and basic peaks from CEX) were both considered as presumptive CQAs (Critical Quality. Attributes) and at the optimal formulation pH, formation of these should be minimized. An optional pH depends on the end goal and can be selected differently.

For example, if reduction of dimer or aggregate formation is a key, then a lower pH would be preferred. On the other hand, if minizing changes in charge variant profile is the target, then a higher pH could be optimal.

TABLE 4
Formulation design for Study 2

| | | | | | | Arg- | | | | |
|---|---|---|---|---|---|---|---|---|---|---|
| | Protein | | His | NaCl | Sucrose | HCl | Pro | Gly | Arg-Glu | PS 80 |
| Form | (mg/mL) | pH | (mM) | (mM) | (mM) | (mM) | (mM) | (mM) | (mM) | (%, w/v) |
| F1 | 150 | 5.0 | 20 | 0 | 200 | 50 | 0 | 0 | 0 | 0.03 |
| F2 | 180 | 5.5 | 10 | 0 | 0 | 0 | 270 | 0 | 0 | 0.03 |
| F3 | 180 | 6.0 | 10 | 0 | 0 | 0 | 0 | 270 | 0 | 0.03 |
| F4 | 180 | 6.5 | 30 | 0 | 0 | 0 | 0 | 0 | 150 | 0.03 |
| F5 | 150 | 5.5 | 10 | 0 | 60 | 125 | 0 | 0 | 0 | 0.03 |
| F6 | 150 | 6.0 | 20 | 50 | 200 | 0 | 0 | 0 | 0 | 0.03 |
| F7 | 150 | 5.0 | 30 | 0 | 0 | 0 | 270 | 0 | 0 | 0.03 |
| F8 | 150 | 6.5 | 20 | 0 | 0 | 0 | 0 | 270 | 0 | 0.03 |
| F9 | 150 | 6.0 | 10 | 0 | 145 | 75 | 0 | 0 | 0 | 0.03 |
| F10 | 150 | 5.5 | 20 | 0 | 200 | 0 | 0 | 0 | 75 | 0.03 |
| F11 | 150 | 5.5 | 30 | 140 | 0 | 0 | 0 | 0 | 0 | 0.03 |
| F12 | 150 | 6.0 | 10 | 0 | 60 | 125 | 0 | 0 | 0 | 0.03 |
| F13 | 150 | 6.0 | 10 | 0 | 235 | 0 | 0 | 0 | 0 | 0.03 |

### Formulation Study 3

This study was conducted in parallel with Study 2. This study had 5 formulations at 150 mg/mL ADI-58125 in 10 mM Histidine buffer over a pH range of 5.5 to 6.0. The excipients were either a mixture of sucrose and arginine hydrochloride or sucrose and sodium chloride. PS80 at a level of 0.03% was added to all the formulations. The compositions were shown in Table 5. The conditions tested in this study were more comprehensive than the previous two studies, thermal stability was tested at −40,−20, 5, 25 and 40° C., and freeze/thaw (5 cycles) and agitation stresses (300 rpm, 3d) were included. A larger panel of analytical methods were used and included, pH, protein concentration, viscosity, visual appearance, SEC, iCIEF, CE-SDS, DLS, MF1 and ELISA.

TABLE 5
Study Design for Study 3

| | | | | | Arginine | |
|---|---|---|---|---|---|---|
| | | Histidine | Sucrose | NaCl | hydrochloride | PS80 |
| Formulation | pH | (mM) | (mM) | (mM) | (mM) | (% w/v) |
| F1 | 6.5 | 10 | 146 | 50 | 0 | 0.03 |
| F2 | 6.0 | 10 | 146 | 50 | 0 | 0.03 |
| F3 | 6.0 | 10 | 58 | 0 | 125 | 0.03 |
| F4 | 5.5 | 10 | 58 | 0 | 125 | 0.03 |
| F5 | 5.5 | 10 | 146 | 50 | 0 | 0.03 |

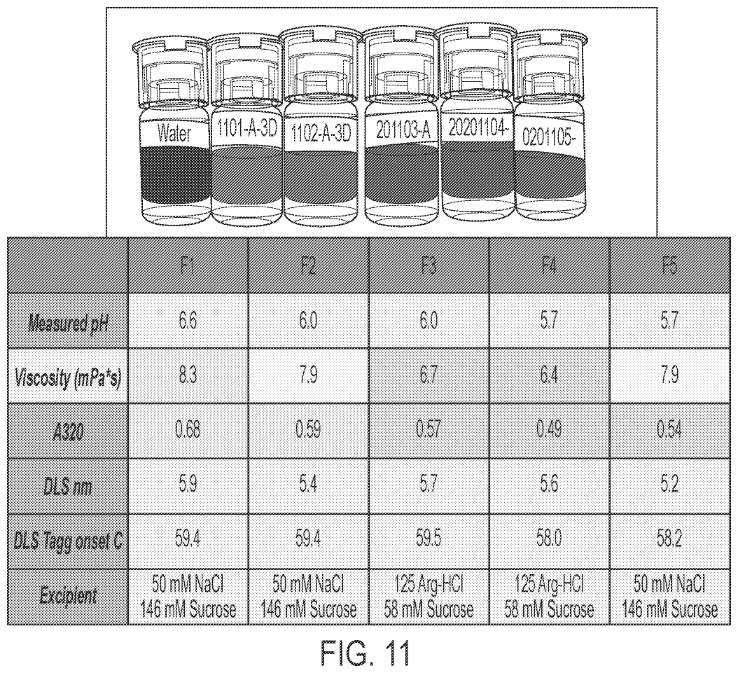

The data obtained at t0 (pH, viscosity, turbidity, hydrodynamic radius and Tonset were shown in Table 6 and FIG. 11. The formulations (F3 & F4) with the arginine hydrochloride and sucrose had lower viscosities and turbidity than the formulations (F1, F2, F5) with sodium chloride and sucrose.

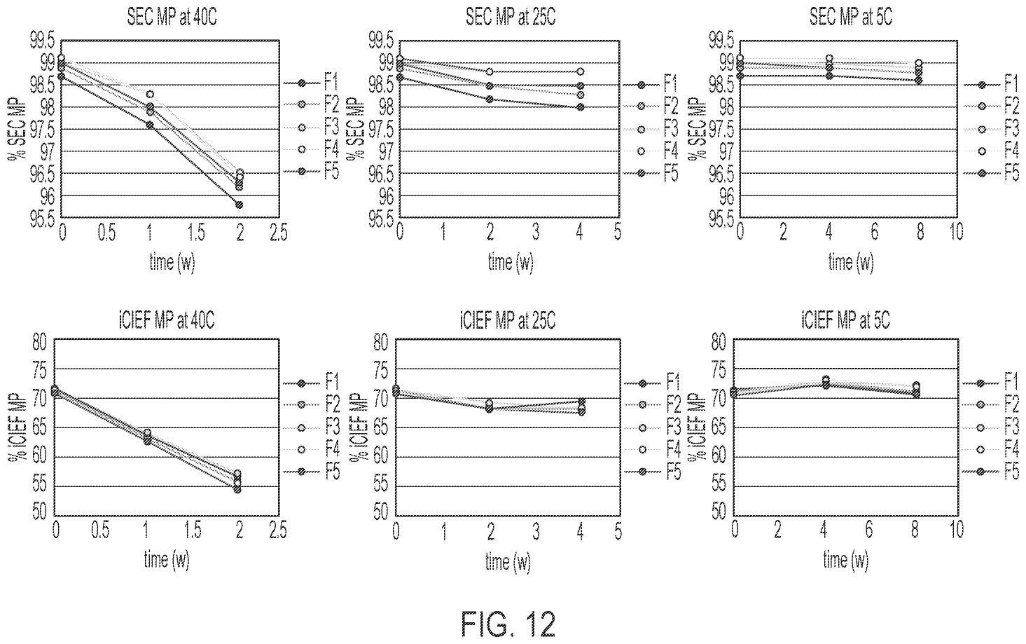

The SEC and iCEIF data as function of time and temperature was shown in FIG. 12. The decrease in main peak for SEC and CEX showed a temperature dependence. For the SEC data, the F3 and F4 formulations performed slightly better than the other three formulations. The F1 formulation at pH 6.6 showed the largest decrease in the monomer. For the iCIEF data, the trends and rates of degradations for all the formulations were very similar. As observed in the previous 2 studies, differences were only seen at 40° C. F1 at the highest pH showed the smallest change in main peak. No changes were seen at either −20° C., or −40° C. conditions. In addition, no differences between the formulations were seen using the CE-SDS, ELISA or MFI techniques.

The 5× freeze/thaw and the agitation stress study did not show any differences between the formulations.

The summary from this study showed that formulations with a mixture of sucrose and arginine hydrochloride provided the best stability and lowest viscosity.

The three screening studies narrowed the formulation to 10 mM histidine, pH 5.5,0.03% PS80 with a combination of arginine and sucrose as excipients. At higher protein concentrations, due to the Donnan effect and the volume exclusion effects, the formulation preparation process needs to be taken into account to achieve the target pH and the excipient concentrations. To finalize the concentrations of arginine and sucrose UF/DF process studies were performed and are addressed in the next section.

TABLE 6
Study 3: Physical properties of formulations.

| | F1 | F2 | F3 | F4 | F5 |
|---|---|---|---|---|---|
| Measured | 6.6 | 6.0 | 6.0 | 5.7 | 5.7 |
| pH | | | | | |
| Viscosity | 8.3 | 7.9 | 6.7 | 6.4 | 7.9 |
| (mPa*s) | | | | | |
| A320 | 0.68 | 0.59 | 0.57 | 0.49 | 0.54 |
| DLS nm | 5.9 | 5.4 | 5.7 | 5.6 | 5.2 |
| DLS Tagg | 59.4 | 59.4 | 59.5 | 58.0 | 58.2 |
| onset C | | | | | |
| Excipient | 50 mM NaCl | 50 mM NaCl | 125 mM | 125 mM | 50 mM NaCl |
| | 146 mM | 146 mM | Arg-HCl | Arg-HCl | 146 mM |
| | Sucrose | Sucrose | 58 mM | 58 mM | Sucrose |
| | | | Sucrose | Sucrose | |

### B. Ultrafiltration/diafiltration (UF/DF) Studies

Ultrafiltration/diafiltration (UF/DF) is a typical step in protein drug manufacturing process to concentrate and exchange the protein solution into a desired formulation. One challenge associated with high-concentration formulations is increased electrostatic interaction between proteins and excipients and is a result of increased protein-charge density at high-protein concentrations. Such interactions can create an offset between excipient levels in final products and diafiltration buffers during the ultrafiltration process. The effect of such electrostatic interactions in a membrane process is known as the Donnan effect. To compensate for the pH offset caused by the Donnan effect, diafiltration buffers with pH and excipient values offset from the ultrafiltrate pool specifications can be used. It is important to understand this effect and develop a UF/DF process that achieves the target pH and excipient concentrations in the DP.

These effects were observed in the screening studies as evidenced by the pH offset between the DF buffers and the osmolality differences based on the formulation preparation process. The processes used to prepare the formulations at Study 1, Study 2 and Study 3 were shown in Table 7.

TABLE 7
Formulation preparation process for screening
studies (Study 1, Study 2 and Study 3)

| Studies 1 and 2 Procedure | Study 3 Procedure |
|---|---|
| Initial UF/DF pool initially | The starting protein concentration |
| diluted 2X with water to reduce | was around 50 mg/mL. Buffer was then |
| osmolality, then concentrated | exchanged by dialysis 3 times (30 mL |
| using UF to 210 mg/mL | protein in 5 L buffer). 3 buffers: |
| | 10 mM His buffer with pH 5.5, 6.0 |
| | and 6.5 |
| Concentrated protein was | After the buffer exchange, the |
| dialyzed against formulation | protein was concentrated to ~180 |
| buffers (including excipients) | mg/mL |
| for 24 h, 4 exchanges | |
| Protein concentration was | Stock excipient solutions were |
| measured and then adjusted | spiked in to the 180 mg/mL solution |
| concentration to 150 mg/mL | so that resulting solutions have |
| | 150 mg/mL protein with the target |
| | excipient concentrations |

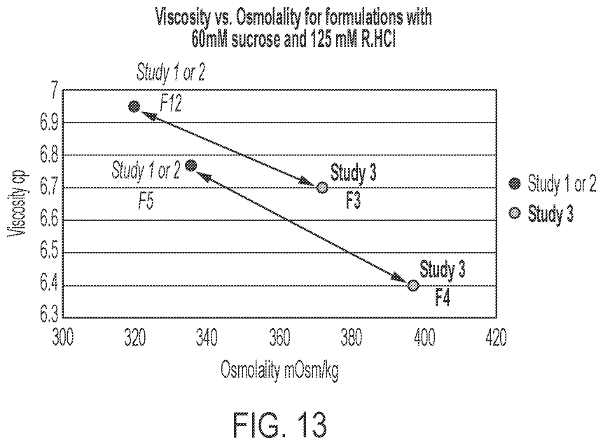

Seven formulations from the three screening studies had sucrose and arginine hydrochloride and arginine glutamate as excipients. The composition of these formulations along with the measured pH, osmolality and viscosity were shown in Table 8 and FIG. 13.

TABLE 8
Comparison of pH offsets, osmolality and viscosity in formulations with
sucrose arginine-hydrochloride and &arginine-glutamate as excipients

| | | | | | Measured | Measured | | | | Arg- | |
|---|---|---|---|---|---|---|---|---|---|---|---|
| | Form | protein | | Measured | Osmolality | viscosity | His | Sucrose | Arg | Glu | PS 80 |
| Study | No | (mg/mL) | pH | pH | (mOsm/kg) | (cP) | (mM) | (mM) | HCl (mM) | (mM) | (%) |
| 1 and 2 | F1 | 150 | 5 | 5.38 | 379 | 7.63 | 20 | 200 | 50 | 0 | 0.03 |
| 1 and 2 | F5 | 150 | 5.5 | 5.7 | 336 | 6.77 | 10 | 60 | 125 | 0 | 0.03 |
| 1 and 2 | F9 | 150 | 6 | 5.99 | 349 | 7.53 | 10 | 145 | 75 | 0 | 0.03 |
| 1 and 2 | F10 | 150 | 5.5 | 5.43 | 457 | 8.88 | 20 | 200 | 0 | 75 | 0.03 |
| 1 and 2 | F12 | 150 | 6 | 5.97 | 320 | 6.95 | 10 | 60 | 125 | 0 | 0.03 |
| 3 | F3 | 150 | 6 | 6 | 372 | 6.7 | 10 | 58 | 125 | 0 | 0.03 |
| 3 | F4 | 150 | 5.5 | 5.7 | 397 | 6.4 | 10 | 58 | 125 | 0 | 0.03 |

Comparing the formulations with similar composition and target pH values (Studies 1 and 2 F5 with Study 3 F4 and Studies 1 and 2 F12 with Study 3 F3) clearly demonstrates that formulation preparation method impacts the outcome.

In a typical manufacturing process, after the viral filtration step, the protein is concentrated to an intermediate concentration, buffer exchanged and the further concentrated by UF/DF to a concentration 20−30% of the target concentration to allow for spiking in of the excipients. The actual over concentration target will need to be determined based on the final formulation and the ability to reach the target by UF/DF.

Several UF/DF studies were conducted for ADI-58125 to determine the highest concentration that could be achieved and were summarized in Table 9. The data showed that a minimum concentration of 50 mM arginine hydrochloride was required to achieve a protein concentration of ˜ 180 mg/mL.

TABLE 9
UF/DF experiments to concentrate ADI-58125

| | | Final | Start | Final |
|---|---|---|---|---|
| | | [ADI- | feed | feed |
| | | 58125] | flux | flux |
| Experiment | DF buffer | mg/mL | (LMH) | (LMH) |
| 1 | 20 mM Histidine, pH 5.5 | 168 | 200 | 34 |
| 2 | 20 mM Histidine, 50 mM | 183 | | |
| | arginine hydrochloride pH 5.5 | | | |
| 3 | 20 mM Histidine, 110 mM | 180 | | |
| | arginine hydrochloride pH 5.5 | | | |

### C. Fine Tuning and Formulation Selection

Osmolality data for formulations from the screening studies was >350 mOsm/kg. The goal for the UF/DF studies was to identify a concentrations of arginine hydrochloride and sucrose such that the target formulation would meet the following criteria.
1. Osmolality was within 300−350 mOsm/kg
2. Viscosity at 25° C. <10 cP
3. Opalescence <18 NTU

To achieve this, 3 formulations were proposed for the UF/DF studies as shown in Table 10.

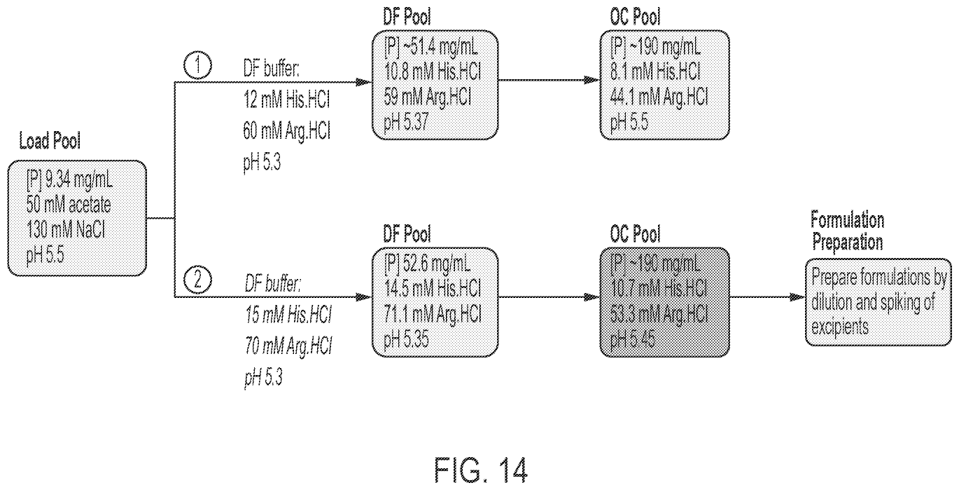

Based on these target formulations, a second set of UF/DF studies were performed to obtain an over concentrated protein pool at a concentration of 190 mg/mL in 10 mM histidine at pH 5.5. These were illustrated in FIG. 14. In brief, the load pool at ˜10 mg/mL ADI-58125 was concentrated to 50 mg/mL and diafiltered against one of two buffers at pH 5.3 with slightly different concentrations of histidine and arginine hydrochloride and then further concentrated to 190 mg/mL. The results showed that the target pH and concentration of protein and excipients were reached using a DF buffer of 15 mM histidine, 70 mM arginine hydrochloride at pH 5.3.

TABLE 10
Composition of formulations for fine-tuning study

| | [ADI- | | | | [arginine | |
|---|---|---|---|---|---|---|
| | 58125] | Target | [histidine] | [sucrose] | hydrochloride] | [PS80] % |
| Formulation | mg/mL | pH | mM | mM | mM | w/v |
| F6 | 150 | 5.5 | 10 | 50 | 110 | 0.03 |
| F7 | 150 | 5.5 | 10 | 100 | 75 | 0.03 |
| F8 | 150 | 5.5 | 10 | 150 | 50 | 0.03 |

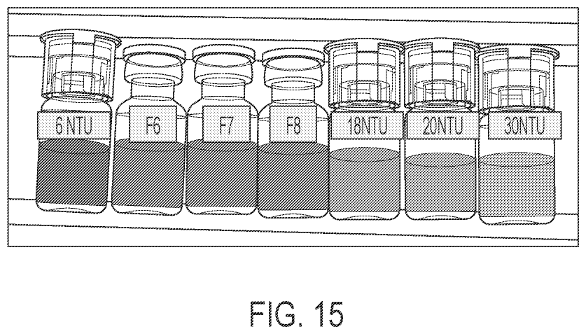

This over concentrated pool was used to prepare the 3 formulations in Table 10 by spiking in the excipients and diluting to 150 mg/mL. The results after dilution were shown in Table 11 and the appearance was shown is FIG. 15.

TABLE 11
Fine-tuning study: Physical properties of formulations

| | [ADI- | | | | | |
|---|---|---|---|---|---|---|
| | 58125] | | Osmolality | Viscosity | | |
| Formulation | mg/mL | pH | (mOsm/kg) | (cP) | A340 | Tg′ (C.) |
| F6 | 145.3 | 5.4 | 342 | 7.3 | 0.29 | −22.9 |
| F7 | 157.7 | 5.4 | 344 | 8.3 | 0.3 | −22.9 |
| F8 | 148.7 | 5.4 | 369 | 8.9 | 0.32 | −23.7 |

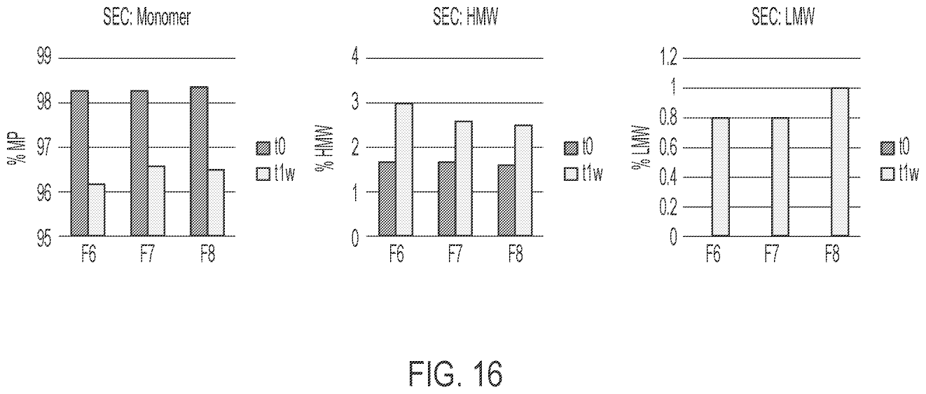

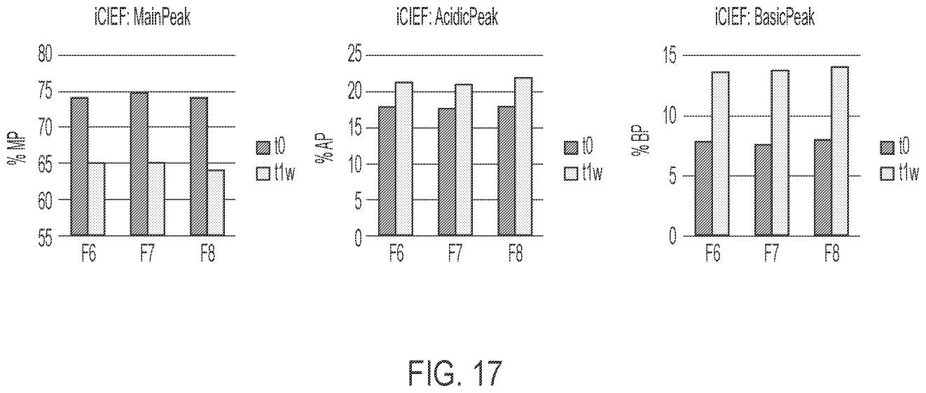

The osmolality for F6 and F7 formulations was <350 mOsm/kg. Viscosity increased with increasing sucrose and decreasing arginine hydrochloride concentrations. However, the viscosity was <10 cP for all three formulations. The turbidity at A320 also showed a small increase in going from F6 to F7 to F8 whereas visually they were indistinguishable and opalescence was between the 6 NTU and 18 NTU opalescence standards. These formulations were stored at 40° C. for one week and analyzed by SEC and iCIEF. The data was graphed in FIG. 16 and FIG. 17.

The SEC data showed that the monomer in F7 was slightly more stable than F6 and F8, and the iCIEF data showed that the degradation was similar for all three formulations. Based on the data obtained, F7 was selected as the optimal formulation that met the criteria for long-term storage of ADI-58125 at 150 mg/mL at 2−8° C., and the composition was 10 mM L-histidine, 75 mM L-arginine hydrochloride, 100 mM sucrose, 0.03% PS80 at pH 5.5. The formulation selection was incorporated into the batch records for drug substance and drug product lots.

### D. Formulation Confirmation Study

The formulation selection was confirmed by preparing ADI-58125 at 150 mg/mL using the process that was representative of the manufacturing process and evaluating stability when subjected to freeze/thaw, agitation and thermal stresses. The stability was evaluated by a panel of methods that includes appearance, pH, protein concentration, SE-HPLC, iCIEF, CE-SDS (R & NR) and PS80 concentration. Select timepoints were analyzed by the potency assay. At t0, osmolality and concentrations of histidine, sucrose and arginine were measured. The study is a 12 m study and data at two-month time point (t=2 m) is discussed here.

ADI-58125 was filled (2.3 mL) into 2R vials (Schott-1557174) and stoppered with a 13 mm nest stopper (Daikyo, Cap-13GL-2-A-100H/C). The study plan was shown in Table 12.

TABLE 12
Formulation confirmation study plan

| Container | Storage | Time Points (months) | | | | | | | |
|---|---|---|---|---|---|---|---|---|---|
| Closure | Conditions | 0 | 0.5 | 1 | 2 | 3 | 6 | 9 | 12 |
| 2R glass vial | 5 ± 3° C. | X, Y, Z | X | XY | X | XY | XY | X | XY |
| with 13 mm | inverted | | | | | | | | |
| stopper | 5 ± 3° C. | | NT | NT | NT | NT | XY | NT | XY |
| | upright | | | | | | | | |
| | 25 ± 3° C. | | X | X | X | X | NT | NT | NT |
| | upright | | | | | | | | |
| | 25 ± 3° C. | | XY | XY | XY | XY | NT | NT | NT |
| | inverted | | | | | | | | |
| | 40° C. | | XY | XY | NT | NT | NT | NT | NT |
| | inverted | | | | | | | | |

X = appearance, subvisible particulate matter, pH, concentration, reduced and non-reduced CE-SDS, SEC, cIEF
Y = binding ELISA
Z = osmolality, concentration of histidine, sucrose, arginine
NT: not tested

The selected formulation was subjected to stress conditions, including freeze/thaw and agitation at 25° C. (higher temperature than intended drug product storage). All studies were performed using a 2R Schott glass vial with a 13 mm Diakyo stopper.

For the agitation stress study, agitation was performed at 100 rpm at 25° C. Samples were tested after 3 and 7 days and the data is shown in Table 13. There were no significant differences observed between the control and these samples demonstrating the formulation can withstand agitation stress.

For the freeze/thaw study, five cycles of freeze/thaw (−40° C., to room temp) were performed with analysis after the fifth cycle, and the data was shown in Table 14. There were no significant differences observed between the control and these samples demonstrating the formulation can withstand freeze/thaw with no changes to the product quality, supporting freezing of the formulation at the intended drug substance storage condition of ≤−30° C., and the drug substance thaw modeling the initial step in the drug product manufacturing process.

TABLE 13
Formulation confirmation study, Agitation data
10 mM L-histidine, 75 mM L-arginine hydrochloride, 100 mM sucrose, 0.03% PS80, pH 5.5

| Product/strength: ADI-58125 | | Container/Closure: 2R Schott | | |
|---|---|---|---|---|
| for injection, 150 mg/mL | | glass vial, 13 mm Daikyo stopper | | |
| | Acceptance | Time Points (days) | | |
| Test | Criteria^{a} | Initial | 3 | 7 |
| Appearance | Color: Report Result | Slight yellow | Slight yellow | Slight yellow |
| | Clarity: ≤60 NTU ^{b} | Slight | Slight | Slight |
| | | opalescence | opalescence | opalescence |
| | Visible Particles: | Essentially | Essentially | Essentially |
| | Essentially free of | free of visible | free of visible | free of visible |
| | visible particles | particles | particles | particles |
| Subvisible | ≥10 μm: ≤6000 | 283 | 659 | 973 |
| Particulate | particles/container | | | |
| Matter | ≥25 μm: ≤600 | 21 | 16 | 21 |
| | particles/container | | | |
| Osmolality | 280-380 mOsm/kg | 345 | NT | NT |
| pH | 5.0-6.0 | 5.4 | 5.4 | 5.4 |
| Protein | 130-170 mg/mL | 150 | 150 | 150 |
| Concentration | | | | |
| Non-reduced | Main peak ≥90.0% | 96.2 | 96.5 | 96.2 |
| CE-SDS | | | | |
| Reduced | Purity (LC + HC) | 98.4 | 98.3 | 98.2 |
| CE-SDS | ≥90.0% | | | |
| SEC-HPLC | Monomer ≥90.0% | 98.7 | 98.8 | 98.7 |
| | Dimer %^{c} | 1.0 | 1.0 | 1.1 |
| | Total HMW % ≤10.0%^{c} | 1.3 | 1.1 | 1.2 |
| | LMW %: ≤10.0% | 0.0 | 0.0 | 0.1 |
| cIEF | Main peak ≥40.0% | 71.7 | 69.9 | 69.5 |
| | Acidic peaks ≤40.0% | 17.1 | 18.4 | 18.3 |
| | Basic peaks ≤30.0% | 11.2 | 11.7 | 12.2 |
| Binding | 60%-140% of | 114 | NT | 111 |
| ELISA | reference standard | | | |
| | activity | | | |
| PS80 | 0.015%-0.045% (w/v) | 0.024 | 0.022 | 0.024 |

cIEF: capillary isoelectric focusing;
HMW: high molecular weight;
LMW: low molecular weight;
NR: non-reduced;
R: reduced;
SEC-HPLC: size exclusion chromatography by HPLC;
PS80: polysorbate80;
NT: not tested
^{a}PPQ drug product specification acceptance criteria are provided as a reference
^{b} Visual assessment was performed (different method from release method)
^{c}Total HMW includes dimer along with other HMW species

TABLE 14
Formulation confirmation study, freeze/thaw data
10 mM L-histidine, 75 mM L-arginine hydrochloride,
100 mM sucrose, 0.03% PS80, pH 5.5

| | | Container/Closure: 2R Schott | |
|---|---|---|---|
| Product/strength: ADI-58125 | | glass vial, 13 mm Daikyo stopper | |
| for injection, 150 mg/mL | | Results | |
| | Acceptance | | 5 Freeze/Thaw |
| Test | Criteria^{a} | Initial | Cycles |
| Appearance | Color: Report Result | Slight yellow | Slight yellow |
| | Clarity: ≤60 NTU ^{b} | Slight | Slight |
| | | opalescence | opalescence |
| | Visible Particles: | Essentially | Essentially |
| | Essentially free | free of visible | free of visible |
| | of visible particles | particles | particles |
| Subvisible | ≥10 μm: ≤6000 | 283 | 161 |
| Particulate | particles/container | | |
| Matter | ≥25 μm: ≤600 | 21 | 5 |
| | particles/container | | |
| Osmolality | 280-380 mOsm/kg | 345 | NT |
| pH | 5.0-6.0 | 5.4 | 5.4 |
| Protein | 130-170 mg/mL | 150 | 150 |
| Concentration | | | |
| NR CE-SDS | Main peak ≥90.0% | 96.2 | 96.1 |
| R CE-SDS | Purity (LC + HC) | 98.4 | 98.3 |
| | ≥90.0% | | |
| SEC-HPLC | Monomer ≥90.0% | 98.7 | 98.6 |
| | Dimer %^{c} | 1.0 | 1.1 |
| | Total HMW % ≤10.0%^{c} | 1.3 | 1.3 |
| | LMW %: ≤10.0% | 0.0 | 0.1 |
| cIEF | Main peak ≥40.0% | 71.7 | 72.7 |
| | Acidic peaks ≤40.0% | 17.1 | 16.6 |
| | Basic peaks ≤30.0% | 11.2 | 10.7 |
| Binding | 60%-140% of | 114 | 107 |
| ELISA | reference standard | | |
| | activity | | |
| PS80 | 0.015%-0.045% (w/v) | 0.024 | 0.024 |

cIEF: capillary isoelectric focusing;
HMW: high molecular weight;
LMW: low molecular weight;
NR: non-reduced;
R: reduced;
SEC-HPLC: size exclusion chromatography by HPLC;
PS80: polysorbate80;
NT: not tested
^{a}PPQ drug product specification acceptance criteria are provided as a reference
^{b} Visual assessment was performed (different method from release method)
^{c}Total HMW includes dimer along with other HMW species

Study results through the 2-month time point at 2°−8° C. were shown in Table 15. SEC monomer peak decreased by 0.2% over 2 months and the increase can be attributed to formation of the dimer species. No HMW (high molecular weight) species were observed. There were no changes in the charge variants as monitored by cIEF and purity as measured by CE-SDS (non-reduced and reduced). All other attributes were unchanged over the 2-month period. The data obtained to date support the long term recommended storage temperature of 2°−8° C. The table included the acceptance criteria for GMP drug product lot 20210303 as a reference; all results to date met the current acceptance criteria. The study will continue according to the plan through additional time points, and results will be monitored.

Study results through the 2-month time point at the accelerated temperature of 25° C. (upright and inverted) were shown in Table 16 and Table 17 respectively, and study results through the 1-month time point at 40° C. (last time point for this temperature) were shown in Table 18. SEC-HPLC monomer peak decreased with increase in temperature and this can be attributed to the increase in dimer at 25° C., and dimer and high molecular weight (HMW) species at 40° C., and an increase in the low molecular weight (LMW) at both temperatures. There was a significant temperature dependent decrease in the cIEF main peak along with an increase in the acidic peak and basic peaks. Purity as measured by CE-SDS (reduced and non-reduced) decreased by less than 1% at 25° C. over 2 months and less than 3% at 40° C. over 1 month. No significant difference in product quality was observed for the 25° C. condition in the upright versus inverted configuration. Overall, the results at 25° C. support storage and processing at this temperature through 0.5 months (2 weeks).

TABLE 15
Formulation confirmation study, 5° C. stability data
Formulation: 150 mg/mL ADI-58125 in 10 mM L-histidine, 75 mM
L-arginine hydrochloride, 100 mM sucrose, 0.03% PS80, pH 5.5
Container/Closure: 2R Schott glass vial, 13 mm Daikyo stopper

| | Acceptance | | Time Points (Months) | | | |
|---|---|---|---|---|---|---|
| Test | Criteria ^{a} | | Initial | 0.5 | 1 | 2 |
| Appearance | Color: Report | | Slight | Slight | Slight | Slight |
| | Result | | yellow | yellow | yellow | yellow |
| | Clarity: ≤60 | | Slight | Slight | Slight | Slight |
| | NTU ^{b} | | opalescence | opalescence | opalescence | opalescence |
| | Visible Particles: | | Essentially | Essentially | Essentially | Essentially |
| | Essentially free of | | free of visible | free of visible | free of | free of |
| | visible particles | | particles | particles | visible | visible |
| | | | | | particles | particles |
| Subvisible | ≥10 μm: ≤6000 | | 283 | 74 | 115 | 76 |
| Particulate | particles/container | | | | | |
| Matter | ≥25 μm: ≤600 | | 21 | 9 | 7 | 5 |
| | particles/container | | | | | |
| Osmolality | Report Result | | 345 | NT | NT | NT |
| | (mOsm/kg) | | | | | |
| pH | 5.0-6.0 | | 5.4 | 5.4 | 5.4 | 5.4 |
| Protein | 150 ± 20 mg/mL | | 150.0 | 150.6 | 152.7 | 150.8 |
| Concentration | | | | | | |
| Non-reduced | Main peak % | | 96.2 | 96.3 | 96.1 | 96.1 |
| CE-SDS | ≥90.0% | | | | | |
| Reduced CE- | Purity %(LC + HC) | | 98.4 | 98.2 | 98.3 | 98.2 |
| SDS | ≥90.0% | | | | | |
| SEC-HPLC | Monomer ≥90.0% | | 98.7 | 98.7 | 98.6 | 98.5 |
| | HMW % | Dimer % ^{c} | 1.0 | 1.1 | 1.2 | 1.2 |
| | ≤10.0% ^{d} | Other | | | | |
| | | HMW % ^{c} | 0.2 | 0.2 | 0.2 | 0.2 |

| | | | | | |
|---|---|---|---|---|---|
| | LMW %: ≤10.0% | 0.0 | 0.0 | 0.1 | 0.1 |
| cIEF | Main peak %: | 71.7 | 70.8 | 72.5 | 71.5 |
| | ≥40.0% | | | | |
| | Acidic peaks %: | 17.1 | 17.8 | 16.3 | 17.0 |
| | ≤40.0% | | | | |
| | Basic peaks %: | 11.2 | 11.4 | 11.2 | 11.6 |
| | ≤30.0% | | | | |
| Binding | 60%-140% of | 114 | NT | 101 | NT |
| ELISA | reference standard | | | | |
| | activity | | | | |
| PS80 | 0.015%-0.045% | 0.024 | 0.025 | 0.024 | 0.023 |

^{a} Current drug product lot 20210303 acceptance criteria are provided as a reference.
^{b} Visual assessment was performed (different method from release method)
^{c} Dimer and other HMW reporting is not on the current specification; it was integrated separately
from other high molecular weight species for this study.
^{d} The HMW % reporting in this table is equivalent to the sum of % dimer and % other HMW (i.e. total HMW %)
cIEF: capillary isoelectric focusing;
HMW: high molecular weight;
LMW: low molecular weight;
NR: non-reduced;
R: reduced;
SEC-HPLC: size exclusion chromatography by HPLC;
PS80: polysorbate 80;
NT: not tested

TABLE 16
Formulation confirmation study 25° C. stability data (upright)
10 mM L-histidine, 75 mM L-arginine hydrochloride, 100 mM sucrose, 0.03% PS80, pH 5.5
Container/Closure: 2R Schott glass vial, 13 mm Daikyo stopper

| | | Time Points (Month) | | | |
|---|---|---|---|---|---|
| Test | | Initial | 0.5 | 1 | 2 |
| Appearance | Color | Slight yellow | Slight yellow | Slight yellow | Slight yellow |
| | Clarity | Slight | Slight | Slight | Slight |
| | | opalescence | opalescence | opalescence | opalescence |
| | Visible Particles: | Essentially | Essentially | Essentially | Essentially |
| | | free of visible | free of visible | free of visible | free of visible |
| | | particles | particles | particles | particles |
| Subvisible | ≥10 μm: | 283 | 44 | 87 | 60 |
| Particulate | particles/container | | | | |
| Matter | ≥25 μm: | 21 | 2 | 7 | 2 |
| | particles/container | | | | |
| Osmolality | mOsm/kg | 345 | NT | NT | NT |
| pH | | 5.4 | 5.4 | 5.4 | 5.4 |
| Protein | mg/mL | 150.0 | 149.3 | 152.1 | 150.0 |
| Concentration | | | | | |
| Non-reduced | Main peak % | 96.2 | 95.9 | 95.8 | 95.6 |
| CE-SDS | | | | | |
| Reduced CE- | Purity % | 98.4 | 98.1 | 98.2 | 97.9 |
| SDS | (LC + HC) | | | | |
| SEC-HPLC | Monomer % | 98.7 | 98.4 | 98.2 | 97.4 |
| | Dimer % | 1.0 | 1.3 | 1.5 | 1.5 |
| | Total HMW % ^{a} | 1.3 | 1.5 | 1.7 | 1.8 |
| | LMW % | 0.0 | 0.1 | 0.1 | 0.9 |
| cIEF | Main peak % | 71.7 | 68.6 | 67.3 | 58.3 |
| | Acidic peaks % | 17.1 | 18.0 | 18.3 | 23.4 |
| | Basic peaks % | 11.2 | 13.4 | 14.4 | 18.3 |
| Binding | Reference | 114 | NT | NT | NT |
| ELISA | Standard Activity | | | | |
| | % | | | | |
| PS80 | % w/v | 0.024 | 0.024 | 0.019 | 0.018 |

cIEF: capillary isoelectric focusing;
HMW: high molecular weight;
LMW: low molecular weight;
NR: non-reduced;
R: reduced;
SEC-HPLC: size exclusion chromatography by HPLC;
PS80: polysorbate80;
NT: not tested
^{a} Total HMW includes dimer and other HMW species

TABLE 17
Formulation confirmation study 25° C. stability data (inverted)
10 mM L-histidine, 75 mM L-arginine hydrochloride, 100 mM sucrose, 0.03% PS80, pH 5.5
Container/Closure: 2R Schott glass vial, 13 mm Daikyo stopper

| | | Time Points (Months) | | | |
|---|---|---|---|---|---|
| Test | | Initial | 0.5 | 1 | 2 |
| Appearance | Color | Slight | Slight | Slight | Slight |
| | | yellow | yellow | yellow | yellow |
| | Clarity | Slight | Slight | Slight | Slight |
| | | opalescence | opalescence | opalescence | opalescence |
| | Visible Particles: | Essentially | Essentially | Essentially | Essentially |
| | | free of visible | free of visible | free of visible | free of visible |
| | | particles | particles | particles | particles |
| Subvisible | ≥10 μm: ≤6000 | 283 | 60 | 90 | 69 |
| Particulate | particles/container | | | | |
| Matter | ≥25 μm: ≤600 | 21 | 2 | 7 | 2 |
| | particles/container | | | | |
| Osmolality | (mOsm/kg) | 345 | NT | NT | NT |
| pH | 5.2-5.8 | 5.4 | 5.4 | 5.4 | 5.4 |
| Protein | mg/mL | 150.0 | 149.4 | 152.2 | 150.2 |
| Concentration | | | | | |
| Non-reduced | Main peak % | 96.2 | 96.0 | 95.9 | 95.5 |
| CE-SDS | | | | | |
| Reduced CE- | Purity % (LC + HC) | 98.4 | 98.1 | 98.2 | 97.9 |
| SDS | | | | | |
| SEC-HPLC | Monomer % | 98.7 | 98.4 | 98.2 | 97.4 |
| | Dimer % | 1.0 | 1.3 | 1.5 | 1.5 |
| | Total HMW % ^{a} | 1.3 | 1.7 | 1.7 | 1.7 |
| | LMW % | 0.0 | 0.1 | 0.1 | 0.9 |
| cIEF | Main peak % | 71.7 | 68.7 | 67.2 | 58.4 |
| | Acidic peaks % | 17.1 | 18.3 | 18.3 | 23.3 |
| | Basic peaks % | 11.2 | 13.0 | 14.6 | 18.2 |
| Binding | Reference Standard | 114 | 109 | 102 | 111 |
| ELISA | activity % | | | | |
| PS80 | % w/v | 0.024 | 0.024 | 0.021 | 0.019 |

cIEF: capillary isoelectric focusing;
HMW: high molecular weight;
LMW: low molecular weight;
NR: non-reduced;
R: reduced;
SEC-HPLC: size exclusion chromatography by HPLC;
PS80: polysorbate80;
NT: not tested
^{a} Total HMW includes dimer and other HMW species

TABLE 18
Formulation confirmation study 40° C. stability data (inverted)
10 mM L-histidine, 75 mM L-arginine hydrochloride,
100 mM sucrose, 0.03% PS80, pH 5.5
Container/Closure: 2R Schott glass vial, 13 mm Daikyo stopper

| | | Time Points (Month) | | |
|---|---|---|---|---|
| Test | | Initial | 0.5 | 1 |
| Appearance | Color | Slight yellow | Slight yellow | Slight yellow |
| | Clarity | Slight | Slight | Slight |
| | | opalescence | opalescence | opalescence |
| | Visible Particles: | Essentially | Essentially | Essentially |
| | | free of visible | free of visible | free of visible |
| | | particles | particles | particles |
| Subvisible | ≥10 μm: | 283 | 37 | 108 |
| Particulate | particles/container | | | |
| Matter | ≥25 μm: | 21 | 2 | 7 |
| | particles/container | | | |
| Osmolality | mOsm/kg | 345 | NT | NT |
| pH | | 5.4 | 5.4 | 5.4 |
| Protein | mg/mL | 150.0 | 149.2 | 152.4 |
| Concentration | | | | |
| Non-reduced | Main peak % | 96.2 | 95.0 | 93.8 |
| CE-SDS | | | | |
| Reduced CE- | Purity % | 98.4 | 96.8 | 96.2 |
| SDS | (LC + HC) | | | |
| SEC-HPLC | Monomer % | 98.7 | 95.7 | 93.1 |
| | Dimer % | 1.0 | 1.8 | 2.3 |
| | Total HMW % ^{a} | 1.3 | 2.8 | 4.5 |
| | LMW % | 0.0 | 1.5 | 2.4 |
| cIEF | Main peak % | 71.7 | 50.4 | 44.1 |
| | Acidic peaks % | 17.1 | 27.2 | 36.7 |
| | Basic peaks % | 11.2 | 22.4 | 19.2 |
| Binding | Reference | 114 | 99 | 94 |
| ELISA | Standard Activity | | | |
| | % | | | |
| PS80 | % w/v | 0.024 | 0.020 | 0.016 |

cIEF: capillary isoelectric focusing;
HMW: high molecular weight;
LMW: low molecular weight;
NR: non-reduced;
R: reduced;
SEC-HPLC: size exclusion chromatography by HPLC;
PS80: polysorbate80;
NT: not tested
^{a} Total HMW includes dimer and other HMW species

### CONCLUSION

A high concentration formulation (150 mg/mL) was successfully developed for ADI-58125, and other anti-CoVS antibodies described herein, that meets the Quality Target Product Profile for a liquid, 2−8° C., drug product presentation. The composition of this formulation was 10 mM L-histidine, 75 mM L-arginine hydrochloride, 100 mM sucrose, 0.03% PS80.

To achieve this, three screening studies with a total of 34 formulations over a pH range of 4.5. to 7.0 and four excipient classes (polyol, sugars, salts, and amino acids) and PS80 as a surfactant were conducted. Once the pH and excipients were selected, UF/DF process studies were performed to determine the composition of the DF buffers required to achieve the target excipient levels. The UF/DF process was used to create three formulations to optimize the excipient levels for stability, osmolality and viscosity. From this, the optimal formulation was selected.

### Example 2. Further Development of Commercial Formulation

Development of the commercial formulation was conducted in parallel to the initial formulation development studies supporting selection of clinical formulations. A primary goal was to stabilize ADI-58125 against pathways of degradation (aggregation, charge-based isoforms, and fragmentation) such that the drug product would be stable at 2 to 8° C. for long term storage. Since the formulation was intended for both drug substance (stored frozen) and drug product (stored at 2 to 8° C.), stability through at least 2 freeze/thaw cycles was required.

The final formulation was selected after initial screening of multiple excipient and pH combinations at high protein concentrations. As demonstrated in Exampel 1, lead formulations were evaluated to ensure acceptable performance of the drug substance UF/DF process step. The selected formulation was evaluated under stress conditions such as freeze/thaw and agitation, to confirm stability under conditions that modeled anticipated processing conditions.

In this Example, a long term non-GMP formulation confirmation stability study was performed to confirming stability at 2 to 8° C. for up to 12 months. A formulation robustness study was also performed to evaluate the final formulation excipient concentration and pH ranges. Results from this study were used to make a minor change in the target pH from 5.5 to 5.6.

The studies described herein support the selection of the final formulation and storage conditions: 150 mg/mL ADI-58125 in 10 mM L-histidine, 75 mM L-arginine hydrochloride, 100 mM sucrose, 0.03% polysorbate 80 (w/v), pH 5.6 for long-term storage at ≤−30° C. for drug substance and at 2 to 8° C. for drug product.

### Formulation Confirmation Stability Study

A non-GMP formulation confirmation stability study was performed in a container closure system and formulation representative of the commercial drug product. The drug substance used was a non-GMP sample manufactured by a representative drug substance process (lot 2312SD210208K01Z01D01).

Study results through the 12-month time point at 2 to 8° C., inverted and upright, are shown in Table 19 and Table 20, respectively. The tables include PPQ drug product release specification acceptance criteria as a reference; all results meet the acceptance criteria. The following observations were made:
a. A decrease of 0.4% in the SEC-HPLC monomer peak was observed over 12 months and the increase can be attributed to formation of the dimer, which is a biologically active product-related substance.
b. All other product attributes are unchanged over the 12-month period.

Study results at the accelerated temperature of 25° C. include data up to 6 months for upright vials (Table 21) and 3 months, inverted (Table 22), and up to 1 month at 40° C., inverted (Table 23). The results are consistent with earlier formulation development studies and observations include:
a. Minimal changes in quality attributes were observed after 2 weeks (0.5 month) storage at 25° C.
b. SEC-HPLC monomer peak decreased with increasing temperature and this could be attributed to an increase in dimer, high molecular weight (HMW) species larger than dimer, and low molecular weight (LMW) species at 25° C., and 40° C.,
c. There was a significant temperature-dependent decrease in the cIEF main peak along with an increase in the acidic and basic peaks, both of which are biologically active product-related substances.
d. Purity as measured by CE-SDS (reduced and non-reduced) decreased by less than 2% at 25° C. over 6 months and less than 3% at 40° C., after 1 month.
e. The polysorbate 80 concentration decreased over time at both the 25° C., and 40° C. condition in a temperature dependent fashion.

In addition to confirming long-term storage conditions (2 to 8° C., data), the results from the 25° C. study further established a time out of refrigeration (TOR) for drug product manufacturing of approximately 0.5 months (2 weeks).

TABLE 19
Formulation Confirmation Stability Study Results At 2 to 8° C., Inverted

| | | | Time Points (Months) | | | | | | | |
|---|---|---|---|---|---|---|---|---|---|---|
| Test | Acceptance Criteria^{a} | | Initial | 0.5 | 1 | 2 | 3 | 6 | 9 | 12 |
| Appearance | Color: Report Result | | Slight | Slight | Slight | Slight | Slight | Slight | Slight | Slight |
| | | | yellow | yellow | yellow | yellow | yellow | yellow | yellow | yellow |
| | Clarity: ≤60 NTU ^{b} | | Slight | Slight | Slight | Slight | Slight | Slight | Slight | Slight |
| | | | opal- | opal- | opal- | opal- | opal- | opal- | opal- | opal- |
| | | | escence | escence | escence | escence | escence | escence | escence | escence |
| | Visible Particles: | | Essentially | Essentially | Essentially | Essentially | Essentially | Essentially | Essentially | Essentially |
| | Essentially free of visible | | free of | free of | free of | free of | free of | free of | free of | free of |
| | particles | | visible | visible | visible | visible | visible | visible | visible | visible |
| | | | particles | particles | particles | particles | particles | particles | particles | particles |
| Subvisible | ≥10 μm: ≤6000 | | 283 | 74 | 115 | 76 | 83 | 18 | 37 | 39 |
| Particulate | particles/container | | | | | | | | | |
| Matter | ≥25 μm: ≤600 | | 21 | 5 | 7 | 5 | 2 | 2 | 5 | 5 |
| | particles/container | | | | | | | | | |
| Osmolality | 280-380 mOsm/kg | | 345 | NT | NT | NT | NT | NT | NT | NT |
| pH | 5.0-6.0 | | 5.4 | 5.4 | 5.4 | 5.4 | 5.4 | 5.4 | 5.4 | 5.4 |
| Protein | 130-170 mg/mL | | 150 | 151 | 153 | 151 | 155 | 152 | 157 | 152 |
| Conc. | | | | | | | | | | |
| Non- | Main peak ≥90.0% | | 96.2 | 96.3 | 96.1 | 96.1 | 95.9 | 96.1 | 95.9 | 96.4 |
| reduced | | | | | | | | | | |
| CE-SDS | | | | | | | | | | |
| Reduced | Purity (LC + HC) ≥90.0% | | 98.4 | 98.2 | 98.3 | 98.2 | 98.3 | 98.2 | 98.2 | 98.4 |
| CE-SDS | | | | | | | | | | |
| SEC-HPLC | Monomer ≥90.0% | | 98.7 | 98.7 | 98.6 | 98.5 | 98.6 | 98.5 | 98.4 | 98.3 |
| | | Dimer % ^{c} | 1.0 | 1.1 | 1.2 | 1.2 | 1.1 | 1.2 | 1.3 | 1.4 |
| | HMW | Other | 0.3 | 0.2 | 0.1 | 0.2 | 0.3 | 0.2 | 0.2 | 0.3 |
| | ≤10.0% ^{c} | HMW % ^{c} | | | | | | | | |

| | | | | | | | | | |
|---|---|---|---|---|---|---|---|---|---|
| | LMW ≤10.0% | 0.0 | 0.0 | 0.1 | 0.1 | 0.1 | 0.1 | 0.1 | 0.1 |
| cIEF | Main peak ≥40.0% | 71.7 | 70.8 | 72.5 | 71.5 | 71.1 | 71.2 | 70.3 | 69.8 |
| | Acidic peaks ≤40.0% | 17.1 | 17.8 | 16.3 | 17.0 | 18.1 | 16.7 | 17.3 | 17.3 |
| | Basic peaks ≤30.0% | 11.2 | 11.4 | 11.2 | 11.6 | 10.8 | 12.1 | 12.4 | 12.9 |
| Binding | 60%-140% of reference | 114 | NT | 101 | NT | 111 | 108 | NT | 93 |
| ELISA | standard activity | | | | | | | | |
| Polysorbate 80 | 0.015%-0.045% (w/v) | 0.024 | 0.025 | 0.024 | 0.023 | 0.026 | 0.024 | 0.022 | 0.025 |

cIEF: capillary isoelectric focusing;
HMW: high molecular weight;
LMW: low molecular weight;
NR: non-reduced;
R: reduced;
SEC-HPLC: size exclusion chromatography by HPLC;
NT: not tested per protocol
^{a}PPQ drug product specification acceptance criteria are provided as a reference
^{b} Visual assessment was performed (different method from release method)
^{c} Separate dimer and other HMW species reporting are not on the GMP drug product specification; however, these species were integrated separately for this study.

TABLE 20
Formulation Confirmation Stability Study Results At 2 to 8° C., Upright

| | Acceptance | | Time Points (Months) | | |
|---|---|---|---|---|---|
| Test | Criteria^{a} | | Initial | 6 | 12 |
| Appearance | Color: Report Result | | Slight yellow | Slight yellow | Slight yellow |
| | Clarity: ≤60 NTU ^{b} | | Slight | Slight | Slight |
| | | | opalescence | opalescence | opalescence |
| | Visible Particles: | | Essentially | Essentially | Essentially |
| | Essentially free of | | free of visible | free of visible | free of visible |
| | visible particles | | particles | particles | particles |
| Subvisible | ≥10 μm: ≤6000 | | 283 | 44 | 28 |
| Particulate | particles/container | | | | |
| Matter | ≥25 μm: ≤600 | | 21 | 2 | 2 |
| | particles/container | | | | |
| Osmolality | 280-380 mOsm/kg | | 345 | NT | NT |
| pH | 5.0-6.0 | | 5.4 | 5.4 | 5.4 |
| Protein | 130-170 mg/mL | | 150 | 153 | 152 |
| Concentration | | | | | |
| Non-reduced | Main peak ≥90.0% | | 96.2 | 96.1 | 96.3 |
| CE-SDS | | | | | |
| Reduced | Purity (LC + HC) | | 98.4 | 98.3 | 98.5 |
| CE-SDS | ≥90.0% | | | | |
| SEC-HPLC | Monomer ≥90.0% | | 98.7 | 98.5 | 98.3 |
| | HMW ≤10.0% | Dimer % | 1.0 | 1.2 | 1.4 |
| | | Other | 0.3 | 0.2 | 0.3 |
| | | HMW % ^{c} | | | |

| | | | | |
|---|---|---|---|---|
| | LMW ≤10.0% | 0.0 | 0.1 | 0.1 |
| cIEF | Main peak ≥40.0% | 71.7 | 71.1 | 69.7 |
| | Acidic peaks ≤40.0% | 17.1 | 16.9 | 17.6 |
| | Basic peaks ≤30.0% | 11.2 | 12.0 | 12.7 |
| Binding ELISA | 60%-140% of | 114 | NT | NT |
| | reference standard | | | |
| | activity | | | |
| Polysorbate 80 | 0.015%-0.045% (w/v) | 0.024 | 0.024 | 0.026 |

cIEF: capillary isoelectric focusing;
HMW: high molecular weight;
LMW: low molecular weight;
NR: non-reduced;
R: reduced;
SEC-HPLC: size exclusion chromatography by HPLC;
NT: not tested perprotocol
^{a}PPQ drug product specification acceptance criteria are provided as a reference
^{b} Visual assessment was performed (different method from release method)
^{c} Separate dimer and other HMW species reporting are not on the GMP specification; however, these species were integrated separately for this study.

TABLE 21
Formulation Confirmation Stability Study Results At 25° C., Upright

| | Acceptance | Time Points (Month) | | | | | |
|---|---|---|---|---|---|---|---|
| Test | Criteria^{a} | Initial | 0.5 | 1 | 2 | 3 | 6 |
| Appearance | Color: Report Result | Slight yellow | Slight yellow | Slight yellow | Slight yellow | Slight yellow | Slight yellow |
| | Clarity: ≤60 NTU ^{b} | Slight | Slight | Slight | Slight | Slight | Slight |
| | | opalescence | opalescence | opalescence | opalescence | opalescence | opalescence |
| | Visible Particles: | Essentially free | Essentially free | Essentially free | Essentially free | Essentially free | Essentially free |
| | Essentially free of | of visible | of visible | of visible | of visible | of visible | of visible |
| | visible particles | particles | particles | particles | particles | particles | particles |
| Subvisible | ≥10 μm: ≤6000 | 283 | 44 | 87 | 60 | 62 | 2047 |
| Particulate | particles/container | | | | | | |
| Matter | ≥25 μm: ≤600 | 21 | 2 | 7 | 2 | 2 | 129 |
| | particles/container | | | | | | |
| Osmolality | 280-380 mOsm/kg | 345 | NT | NT | NT | NT | NT |
| pH | 5.0-6.0 | 5.4 | 5.4 | 5.4 | 5.4 | 5.3 | 5.4 |
| Protein | 130-170 mg/mL | 150 | 149 | 152 | 150 | 154 | 154 |
| Concentration | | | | | | | |
| Non-reduced CE- | Main peak ≥90.0% | 96.2 | 95.9 | 95.8 | 95.6 | 95.1 | 94.3 |
| SDS | | | | | | | |
| Reduced CE-SDS | Purity (LC + HC) | 98.4 | 98.1 | 98.2 | 97.9 | 97.8 | 96.9 |
| | ≥90.0% | | | | | | |
| SEC-HPLC | Monomer ≥90.0% | 98.7 | 98.4 | 98.2 | 97.4 | 97.0 | 95.8 |
| | Dimer % ^{c} | 1.0 | 1.3 | 1.5 | 1.5 | 1.7 | 2.3 |
| | Total HMW % | 1.3 | 1.5 | 1.7 | 1.8 | 1.9 | 2.4 |
| | ≤10.0% ^{c} | | | | | | |
| | LMW %: ≤10.0% | 0.0 | 0.1 | 0.1 | 0.9 | 1.1 | 1.8 |
| cIEF | Main peak ≥40.0% | 71.7 | 68.6 | 67.3 | 58.3 | 54.5 | 48.3 |
| | Acidic peaks | 17.1 | 18.0 | 18.3 | 23.4 | 26.8 | 32.5 |
| | ≤40.0% | | | | | | |
| | Basic peaks ≤30.0% | 11.2 | 13.4 | 14.4 | 18.3 | 18.7 | 19.3 |
| Binding ELISA | 60%-140% of | 114 | NT | NT | NT | NT | 91 |
| | reference standard | | | | | | |
| | activity | | | | | | |
| Polysorbate 80 | 0.015%-0.045% | 0.024 | 0.024 | 0.019 | 0.018 | 0.013 | 0.010 |
| | (w/v) | | | | | | |

cIEF: capillary isoelectric focusing;
HMW: high molecular weight;
LMW: low molecular weight;
NR: non-reduced;
R: reduced;
SEC-HPLC: size exclusion chromatography by HPLC;
NT: not tested per protocol
^{a}PPQ drug product specification acceptance criteria are provided as a reference
^{b} Visual assessment was performed (different method from release method)
^{c} Total HMW includes dimer and other HMW species

TABLE 22
Formulation Confirmation Stability Study Results At 25° C., Inverted

| | | Time Points (Months) | | | | |
|---|---|---|---|---|---|---|
| Test | Acceptance Criteria^{a} | Initial | 0.5 | 1 | 2 | 3 |
| Appearance | Color: Report Result | Slight yellow | Slight yellow | Slight yellow | Slight yellow | Slight yellow |
| | Clarity: ≤60 NTU ^{b} | Slight | Slight | Slight | Slight | Slight |
| | | opalescence | opalescence | opalescence | opalescence | opalescence |
| | Visible Particles: | Essentially | Essentially | Essentially | Essentially | Essentially |
| | Essentially free | free of | free of | free of | free of | free of |
| | of visible | visible | visible | visible | visible | visible |
| | particles | particles | particles | particles | particles | particles |
| Subvisible | ≥10 μm: ≤6000 | 283 | 58 | 90 | 69 | 41 |
| Particulate | particles/container | | | | | |
| Matter | ≥25 μm: ≤600 | 21 | 2 | 2 | 2 | 2 |
| | particles/container | | | | | |
| Osmolality | 280-380 mOsm/kg | 345 | NT | NT | NT | NT |
| pH | 5.0-6.0 | 5.4 | 5.4 | 5.4 | 5.4 | 5.3 |
| Protein | 130-170 mg/mL | 150 | 149 | 152 | 150 | 154 |
| Concentration | | | | | | |
| Non-reduced | Main peak ≥90.0% | 96.2 | 96.0 | 95.9 | 95.5 | 94.9 |
| CE-SDS | | | | | | |
| Reduced CE- | Purity (LC + HC) ≥90.0% | 98.4 | 98.1 | 98.2 | 97.9 | 97.8 |
| SDS | | | | | | |
| SEC-HPLC | Monomer ≥90.0% | 98.7 | 98.4 | 98.2 | 97.4 | 97.0 |
| | Dimer % ^{c} | 1.0 | 1.3 | 1.5 | 1.5 | 1.7 |
| | Total HMW % ≤10.0% ^{c} | 1.3 | 1.5 | 1.7 | 1.7 | 1.9 |
| | LMW %: ≤10.0% | 0.0 | 0.1 | 0.1 | 0.9 | 1.1 |
| cIEF | Main peak ≥40.0% | 71.7 | 68.7 | 67.2 | 58.4 | 55.7 |
| | Acidic peaks ≤40.0% | 17.1 | 18.0 | 18.3 | 23.3 | 25.8 |
| | Basic peaks ≤30.0% | 11.2 | 13.0 | 14.6 | 18.2 | 18.6 |
| Binding ELISA | 60%-140% of reference | 114 | 109 | 102 | 111 | 94 |
| | standard activity | | | | | |
| Polysorbate 80 | 0.015%-0.045% (w/v) | 0.024 | 0.024 | 0.021 | 0.019 | 0.012 |

cIEF: capillary isoelectric focusing;
HMW: high molecular weight;
LMW: low molecular weight;
NR: non-reduced;
R: reduced;
SEC-HPLC: size exclusion chromatography by HPLC;
NT: not tested per protocol
^{a}PPQ drug product specification acceptance criteria are provided as a reference
^{b} Visual assessment was performed (different method from release method)
^{c} Total HMW includes dimer and other HMW species

TABLE 23
Formulation Confirmation Stability Study Results At 40° C., Inverted

| | Acceptance | Time Points (Month) | | |
|---|---|---|---|---|
| Test | Criteria^{a} | Initial | 0.5 | 1 |
| Appearance | Color: Report Result | Slight yellow | Slight yellow | Slight yellow |
| | Clarity: ≤60 NTU ^{b} | Slight | Slight | Slight |
| | | opalescence | opalescence | opalescence |
| | Visible Particles: | Essentially | Essentially | Essentially |
| | Essentially free of | free of visible | free of visible | free of visible |
| | visible particles | particles | particles | particles |
| Subvisible | ≥10 μm: ≤6000 | 283 | 37 | 108 |
| Particulate | particles/container | | | |
| Matter | ≥25 μm: ≤600 | 21 | 2 | 7 |
| | particles/container | | | |
| Osmolality | 280-380 mOsm/kg | 345 | NT | NT |
| pH | 5.0-6.0 | 5.0-6.0 | 5.5 | 5.4 |
| Protein | 130-170 mg/mL | 150 | 149 | 152 |
| Concentration | | | | |
| Non-reduced | Main peak ≥90.0% | 96.2 | 95.0 | 93.8 |
| CE-SDS | | | | |
| Reduced | Purity (LC + HC) | 98.4 | 96.8 | 96.2 |
| CE-SDS | ≥90.0% | | | |
| SEC-HPLC | Monomer ≥90.0% | 98.7 | 95.7 | 93.1 |
| | Dimer %^{c} | 1.0 | 1.8 | 2.3 |
| | Total HMW % ≤10.0%^{c} | 1.3 | 2.8 | 4.5 |
| | LMW %: ≤10.0% | 0.0 | 1.5 | 2.4 |
| cIEF | Main peak ≥40.0% | 71.7 | 50.4 | 44.1 |
| | Acidic peaks ≤40.0% | 17.1 | 27.2 | 36.7 |
| | Basic peaks ≤30.0% | 11.2 | 22.4 | 19.2 |
| Binding | 60%-140% of | 114 | 99 | 94 |
| ELISA | reference standard | | | |
| | activity | | | |
| Polysorbate 80 | 0.015%-0.045% (w/v) | 0.024 | 0.020 | 0.016 |

cIEF: capillary isoelectric focusing;
HMW: high molecular weight;
LMW: low molecular weight;
NR: non-reduced;
R: reduced;
SEC-HPLC: size exclusion chromatography by HPLC;
NT: not tested per protocol
^{a}PPQ drug product specification acceptance criteria are provided as a reference
^{b} Visual assessment was performed (different method from release method)
^{c}Total HMW includes dimer and other HMW species

### Formulation Robustness Study

Robustness of the chosen formulation was assessed by evaluating excipient and pH ranges of the final selected formulation stability at both 2 to 8° C., and 25° C. Presumptive critical formulation parameters and ranges were selected as: pH (5.2−5.8), protein concentration (130−170 mg/mL), arginine HCl (60−90 mM), sucrose (90−110 mM) and PS80 (0.015−0.045%).

A partial factorial (4-factor) design of experiment (DOE) was created to evaluate impact of protein, arginine and sucrose concentrations, as well as pH, yielding 8 formulation variants to be tested. PS80 was evaluated as an independent parameter at the target concentration of the other excipients. In total, 12 formulation compositions were evaluated (8 from the DOE, 2 for PS80 evaluation and 2 at the target composition) as shown in Table 24. The formulations were filled (2.3 mL) into 2R glass vials, stoppered, and sealed, representative of GMP drug product.

TABLE 24
Formulation Composition for Robustness Study

| | | Protein | | | | |
|---|---|---|---|---|---|---|
| Formulation | | Concentration | Arginine- | Sucrose | Histidine | PS80 |
| No. | pH | (mg/mL) | HCl (mM) | (mM) | (mM) | (%, w/v) |
| F1 | 5.2 | 170 | 90 | 90 | 10 | 0.03 |
| F2 | 5.8 | 170 | 90 | 110 | 10 | 0.03 |
| F3 | 5.8 | 130 | 60 | 110 | 10 | 0.03 |
| F4 | 5.2 | 170 | 60 | 110 | 10 | 0.03 |
| F5 | 5.2 | 130 | 60 | 90 | 10 | 0.03 |
| F6 | 5.8 | 170 | 60 | 90 | 10 | 0.03 |
| F7 | 5.2 | 130 | 90 | 110 | 10 | 0.03 |
| F8 | 5.8 | 130 | 90 | 90 | 10 | 0.03 |
| F9 | 5.5 | 150 | 75 | 100 | 10 | 0.03 |
| F10 | 5.5 | 150 | 75 | 100 | 10 | 0.03 |
| F11 | 5.5 | 150 | 75 | 100 | 10 | 0.045 |
| F12 | 5.5 | 150 | 75 | 100 | 10 | 0.015 |

Formulation robustness samples stored at 2 to 8° C. were analyzed at TO (initial), 1, 3, 6, and 12 months. The tests applied were color, clarity, appearance, particulate matter, pH, protein concentration, non-reduced and reduced CE-SDS, SEC-HPLC, cIEF, protein binding by ELISA and PS80 concentration. Analysis of the data through 12 months demonstrates:
a. The SEC monomer peak decreased between 0.6−0.8% over 12 months for all formulation variations; the decrease was due to an increase in dimer (by 0.1 to 0.3%) as well as formation of LMW species.
b. There were no changes within assay variability in the charge variants as monitored by cIEF for higher pH formulations (at pH 5.4 and 5.8), while some change was observed at low pH formulations (pH 5.2).
c. There were no significant changes in purity as measured by CE-SDS (non-reduced and reduced) and all other attributes were unchanged over the 12-month period.

A summary of SEC-HPLC data is provided in Table 25 and a summary of cIEF data is provided in Table 26 over the 12-month period. The data support the robustness of the formulation relative to the factors studied at long term storage temperature of 2 to 8° C.

TABLE 25
Formulation Robustness Stability Study Results At 2 to 8° C.; Comparison
of SEC-HPLC Data at T = 0 and T = 12 m

| | | Formulation No./pH | | | | | | | | | | | |
|---|---|---|---|---|---|---|---|---|---|---|---|---|---|
| | Acceptance | F1 | F2 | F3 | F4 | F5 | F6 | F7 | F8 | F9 | F10 | F11 | F12 |
| Test | Criteria^{a} | 5.2 | 5.8 | 5.8 | 5.2 | 5.2 | 5.8 | 5.2 | 5.8 | 5.5 | 5.5 | 5.5 | 5.5 |

T = 0 (initial)

| | | | | | | | | | | | | | |
|---|---|---|---|---|---|---|---|---|---|---|---|---|---|
| SEC-HPLC | Monomer ≥90.0% | 98.8 | 98.8 | 98.8 | 98.8 | 98.9 | 98.7 | 98.9 | 98.9 | 98.9 | 98.8 | 98.8 | 98.9 |
| | Dimer % | 1.0 | 1.1 | 1.1 | 1.0 | 0.9 | 1.2 | 0.9 | 1.0 | 1.0 | 1.0 | 1.0 | 1.0 |
| | Total HMW % | 1.1 | 1.2 | 1.2 | 1.1 | 1.0 | 1.3 | 1.0 | 1.1 | 1.1 | 1.2 | 1.2 | 1.1 |
| | ≤10.0% ^{b} | | | | | | | | | | | | |
| | LMW %: ≤10.0% | 0.0 | 0.0 | 0.0 | 0.0 | 0.0 | 0.0 | 0.0 | 0.0 | 0.0 | 0.0 | 0.0 | 0.0 |

T = 12 m

| | | | | | | | | | | | | | |
|---|---|---|---|---|---|---|---|---|---|---|---|---|---|
| SEC-HPLC | Monomer ≥90.0% | 98.1 | 98.0 | 98.2 | 98.0 | 98.2 | 98.0 | 98.2 | 98.2 | 98.1 | 98.1 | 98.1 | 98.1 |
| | Dimer % | 1.3 | 1.3 | 1.2 | 1.3 | 1.2 | 1.4 | 1.2 | 1.2 | 1.3 | 1.3 | 1.3 | 1.3 |
| | Total HMW % | 1.4 | 1.5 | 1.4 | 1.5 | 1.3 | 1.6 | 1.3 | 1.4 | 1.4 | 1.5 | 1.5 | 1.4 |
| | ≤10.0% ^{b} | | | | | | | | | | | | |
| | LMW %: ≤10.0% | 0.5 | 0.5 | 0.5 | 0.5 | 0.5 | 0.5 | 0.5 | 0.5 | 0.5 | 0.5 | 0.5 | 0.5 |

HMW: high molecular weight;
LMW: low molecular weight;
SEC-HPLC: size exclusion chromatography by HPLC
^{a}PPQ drug product specification acceptance criteria are provided as a reference
^{b} Total HMW includes dimer and other HMW species

TABLE 26
Formulation Robustness Stability Study Results At 2 to 8° C.;
Comparison of cIEF Data at T = 0 and T = 12 m

| | | Formulation No./pH | | | | | | | | | | | |
|---|---|---|---|---|---|---|---|---|---|---|---|---|---|
| | Acceptance | F1 | F2 | F3 | F4 | F5 | F6 | F7 | F8 | F9 | F10 | F11 | F12 |
| Test | Criteria^{a} | 5.2 | 5.8 | 5.8 | 5.2 | 5.2 | 5.8 | 5.2 | 5.8 | 5.5 | 5.5 | 5.5 | 5.5 |

T = 0 (initial)

| | | | | | | | | | | | | | |
|---|---|---|---|---|---|---|---|---|---|---|---|---|---|
| cIEF | Main peak %: | 73.6 | 73.4 | 74.2 | 74.1 | 74.7 | 73.3 | 73.8 | 74.1 | 73.4 | 74.5 | 74.2 | 74.0 |
| | ≥40.0% | | | | | | | | | | | | |
| | Acidic peaks %: | 17.3 | 17.5 | 17.2 | 16.9 | 16.5 | 17.7 | 17.2 | 17.2 | 17.7 | 16.4 | 16.9 | 17.3 |
| | ≤40.0% | | | | | | | | | | | | |
| | Basic peaks %: | 9.1 | 9.1 | 8.6 | 9.0 | 8.8 | 8.9 | 8.9 | 8.7 | 8.9 | 9.1 | 8.9 | 8.7 |
| | ≤30.0% | | | | | | | | | | | | |

T = 12 m

| | | | | | | | | | | | | | |
|---|---|---|---|---|---|---|---|---|---|---|---|---|---|
| cIEF | Main peak %: | 71.0 | 74.2 | 74.2 | 72.1 | 72.2 | 74.0 | 71.8 | 74.3 | 72.7 | 72.6 | 73.2 | 72.5 |
| | ≥40.0% | | | | | | | | | | | | |
| | Acidic peaks %: | 17.8 | 17.2 | 17.3 | 16.8 | 17.0 | 17.6 | 17.1 | 17.2 | 17.1 | 17.1 | 16.7 | 17.3 |
| | ≤40.0% | | | | | | | | | | | | |
| | Basic peaks %: | 11.2 | 8.6 | 8.5 | 11.1 | 10.8 | 8.4 | 11.1 | 8.4 | 10.2 | 10.3 | 10.1 | 10.2 |
| | ≤30.0% | | | | | | | | | | | | |

cIEF: capillary isoelectric focusing
^{a}PPQ drug product specification acceptance criteria are provided as a reference

Formulation robustness samples stored at 25° C. were analyzed at TO (initial), 1, 2, 3 and 6 months. The tests applied were the same as that for the samples stored at 2 to 8° C., through 3 months with a subset (visual tests, cIEF and SEC-HPLC) at 6 months. In summary, the data demonstrated:
a. The protein purity (SEC-HPLC, reduced & non-reduced CE-SDS) showed a decreasing trend with increasing incubation time.
b. The SEC-HPLC main peak decreased by 2.2% to 2.8% after 6 months at 25° C.
c. THE CE-SDS-Reduced and CE-SDS non-reduced main peak decreased by <0.5% and 1%, respectively, within 3 months (no 6-month data available).
d. The cIEF trend analysis results showed that the main peak decreased by 19.1% to 26.8%, after 6 months, concurrent with increases in both acidic and basic peaks.
e. All other attributes were unchanged over the 3-month period at 25° C.

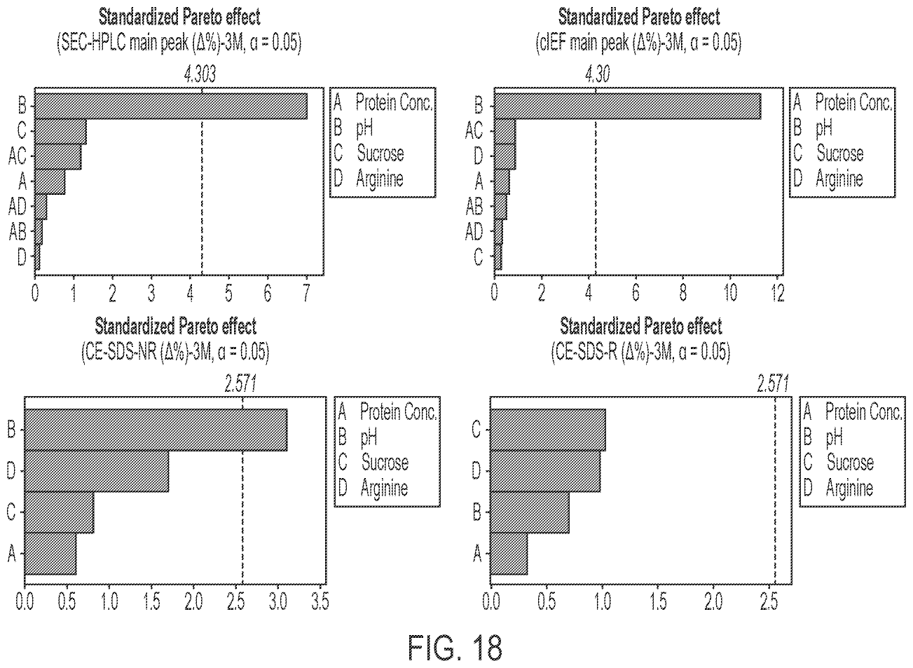

Statistical analysis of the 25° C. formulation robustness data using data from F1-F10 showed that, of the parameters evaluated (including protein concentration, pH, sucrose content, and arginine content), only pH had a significant effect on the SEC-HPLC, cIEF main peak and CE-SDS-NR purity after 3 months. There was no effect from the interaction of these characterized factors, as shown in FIG. 18. All the statistical differences showed pH-dependent changes, with better stability at higher pH.

In addition to storage at long-term and accelerated conditions, the formulation robustness samples were subjected to agitation stress (300 rpm at 25° C.). Analysis was performed using the same set of tests as the 2 to 8° C. stability samples. There were no significant differences observed between the control and these samples, demonstrating all 12 formulations can withstand agitation stress.

In addition, three cycles of freeze (−40° C.) and thaw (room temperature) were performed on the 12 formulations then analyzed after the third cycle. No differences were observed between the samples and control, which supports the frozen storage of drug substance at the intended condition of ≤−30° C.

In summary, the robustness study demonstrated the product formulation was stable within the characterized ranges of formulation parameters under the following conditions: agitation at 300 rpm at 25° C. for 3 days, 3 freeze/thaw cycles (−40° C., to room temperature) and storage at 2 to 8° C. for 12 months. Data at 25° C. showed significant change across all formulations and also revealed that the pH has a statistically significant impact on ADI-58125 stability; the higher pH formulations generally had better stability as indicated by a higher percentage main peak for SEC-HPLC, cIEF and CE-SDS-Non-reduced.

### Robustness Study at pH 5.6 and pH 6.0

Two additional formulations were prepared with components at target (150 mg/mL ADI-58125 in 10 mM L-histidine, 75 mM L-arginine hydrochloride, 100 mM sucrose, 0.03% polysorbate 80 (w/v)) but with a pH of 5.6 and 6.0. The measured concentrations for each component are shown in Table 27. The formulations were filled (2.3 mL) into 2R glass vials, stoppered, and sealed, representative of GMP drug product.

TABLE 27
Composition for pH 5.6 and 6.0 Formulations

| | | Protein | Arginine- | | | | | |
|---|---|---|---|---|---|---|---|---|
| Formulation | | Concentration | HCl | Sucrose | Histidine | PS80 | | Viscosity |
| No. | pH | (mg/mL) | (mM) | (mM) | (mM) | (%, w/v) | Osmolality | (cP) |
| F13 | 5.6 | 148 | 81 | 108 | 11.4 | 0.034 | 337 | 7.9 |
| F14 | 6.0 | 152 | 75 | 99 | 10.5 | 0.035 | 342 | 7.3 |

The two formulations were subject to agitation and free/thaw studies as described in Example 1. The agitation results were summarized in Table 28. All the samples were slightly yellow, slightly opalescent, and free of visible particles after agitation for 3 days (300 rpm horizontal speed at 25° C.). No changes in protein concentration and pH were observed post-agitation. The protein purity (SEC-HPLC, reduced & non-reduced CE-SDS) remained unchanged for all samples, and no obvious changes in the cIEF results were observed.

TABLE 28
Formulation confirmation study, agitation data

| | | F13 (pH 5.6) | | F14 (pH 6.0) | |
|---|---|---|---|---|---|
| | | Time Points (days) | | Time Points (days) | |
| Test | | Initial | 3 | Initial | 3 |
| Appearance | Color: | Slight yellow | Slight yellow | Slight yellow | Slight yellow |
| | Clarity: | Slight | Slight | Slight | Slight |
| | | opalescence | opalescence | opalescence | opalescence |
| | Visible | Essentially | Essentially | Essentially | Essentially |
| | Particles: | free of visible | free of visible | free of visible | free of visible |
| | | particles | particles | particles | particles |
| pH | | 5.6 | 5.6 | 6.0 | 6.0 |
| Non-reduced | Main peak | 96.6 | 96.5 | 96.8 | 96.8 |
| CE-SDS | | | | | |
| Reduced | Purity | 98.2 | 98.3 | 98.5 | 98.5 |
| CE-SDS | (LC + HC) | | | | |
| SEC-HPLC | Main peak | 98.8 | 98.8 | 98.5 | 98.5 |
| | HMW % | 1.2 | 1.1 | 1.5 | 1.5 |
| | LMW % | 0.0 | 0.0 | 0.0 | 0.0 |
| cIEF | Main peak | 73.7 | 73.7 | 75.5 | 75.5 |
| | Acidic peaks | 16.5 | 15.7 | 15.9 | 16.3 |
| | Basic peaks | 9.8 | 10.6 | 8.6 | 8.2 |

The results for samples after three freeze/thaw cycles were summarized in Table 29. All the samples were slightly yellow, slightly opalescent, and free of visible particles after freezing and thawing for 3 cycles. No changes in protein concentration and pH were observed. The protein purity (SEC-HPLC, reduced & non-reduced CE-SDS) remained unchanged for all samples, and no obvious changes in the cIEF results were observed. The sub-visible particles showed no significant increase trend after freeze/thaw for 3 cycles when compared with TO.

TABLE 29
Formulation confirmation study, freeze/thaw data

| Test | | F13 (pH 5.6) | F14 (pH 6.0) |
|---|---|---|---|
| Appearance | Color: | Slight yellow | Slight yellow |
| | Clarity: | Slight | Slight |
| | | opalescence | opalescence |
| | Visible | Essentially | Essentially |
| | Particles: | free of visible | free of visible |
| | | particles | particles |
| pH | | 5.6 | 6.0 |
| Non-reduced | Main peak | 96.5 | 96.9 |
| CE-SDS | | | |
| Reduced | Purity | 98.1 | 98.4 |
| CE-SDS | (LC + HC) | | |
| SEC-HPLC | Main peak | 98.9 | 98.5 |
| | HMW % | 1.1 | 1.4 |
| | LMW % | 0.0 | 0.0 |
| cIEF | Main peak | 74.2 | 75.5 |
| | Acidic peaks | 16.3 | 16.5 |
| | Basic peaks | 9.5 | 8.1 |

Samples stored at 2 to 8° C. were analyzed at TO (initial), 1, 3, 6, 9 months, and would be analyzed subsequently at 12, 18 and 24 months. The tests applied were color, clarity, visible particles, particulate matter, pH, protein concentration, non-reduced and reduced CE-SDS, SEC-HPLC, cIEF, protein binding by ELISA and PS80 concentration. Analysis of the data through 9 months demonstrated:
a. Both pH 5.6 and pH 6.0 formulation were stable after storage at 2 to 8° C. for 9 months.
b. A slight decrease in cIEF for pH 5.6 formulation was observed, while no change was observed for pH 6.0 formulation after storage at 2 to 8° C. for 9 months.
c. No changes or trend were observed in all other assays for both formulations.

Samples stored at 25° C. were analyzed at TO (initial), 1, 2, 3, 6 months. The tests applied were the same as that for the samples stored at 2 to 8° C. In summary, the data demonstrated:
a. No changes or trend were observed in color, clarity, visible particles, particulate matter, pH, protein concentration, and ELISA for both formulations after stored at 25° C. for 6 months.
b. Slight decreases were observed in purity by SEC-HPLC, non-reduced and reduced CE-SDS for both formulations, while a significant decrease in purity by cIEF was observed with less purity loss in pH 6.0 formulation.

These studies demonstrate that the stability of these additional pH 5.6 and pH 6.0 formulations follow the similar trend as formulation robustness study (pH 5.2−5.8), suggesting that the pH of the formulation could expand the range from 5.2 to 6.0. Based on the formulation robustness study results and this data, it is suggested that ADI-58125 formulation is robust within the characterized ranges of critical parameters, and higher pH within characterized ranges achieves better stability under accelerated thermal condition.

### Optimization of Target pH

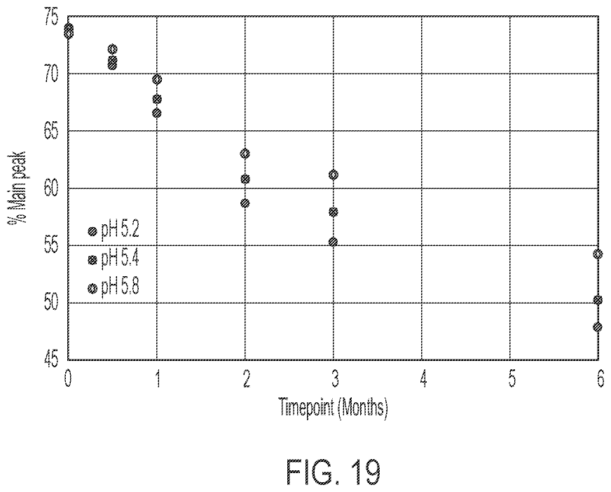

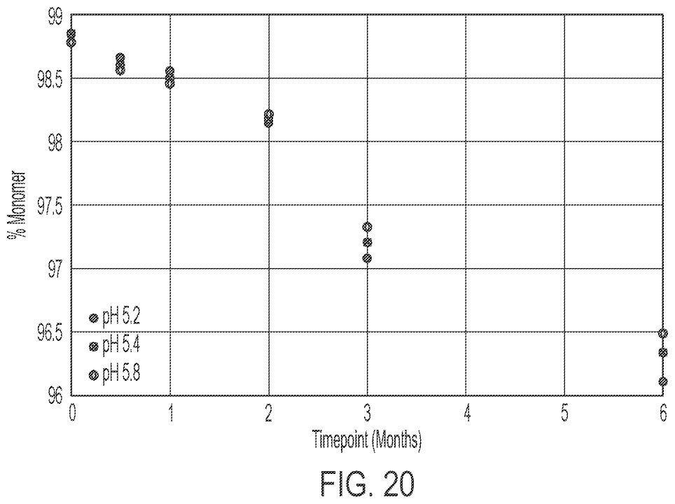

The formulation robustness data demonstrated that, of the factors investigated, pH was the only one that had a statistically significant effect on the stability of the formulation. This effect was further evaluated by assessing SEC-HPLC and cIEF data from the formulation robustness study for lots F1 to F12 (Table 24) as a function of pH. In this case, the data from each condition was aggregated according to the pH (5.2, 5.4 and 5.8) of the test solution. FIG. 19 shows cIEF % main peak for the average of 4 formulations at each pH stored at 25° C. over 6 months. The decrease in main peak was pH-dependent, with higher-pH formulations (at pH 5.8) being the slowest. FIG. 20 shows SEC-HPLC monomer (% main peak) for the average of 4 formulations at each pH stored at 25° C. over 6 months. A slight pH-dependent decrease in monomer was observed, with the slowest decrease occurring at higher pH. Note that a similar analysis of the results for the samples stored at 2 to 8° C., indicates no pH effect for SEC and similar but very minimal pH effect for cIEF over the 12 month study period.

As demonstrated by FIGS. 19 and 20, there was an improvement in drug product stability at higher pH over the pH range of 5.2 to 5.8. Based on these data, a slight adjustment to the target formulation pH from 5.5 to 5.6 was implemented prior to the DS PPQ lots for the process. The value of 5.6 was selected to benefit from the pH effect while remaining within the upper value of 6.0 (supported by robustness study at pH 6.0).

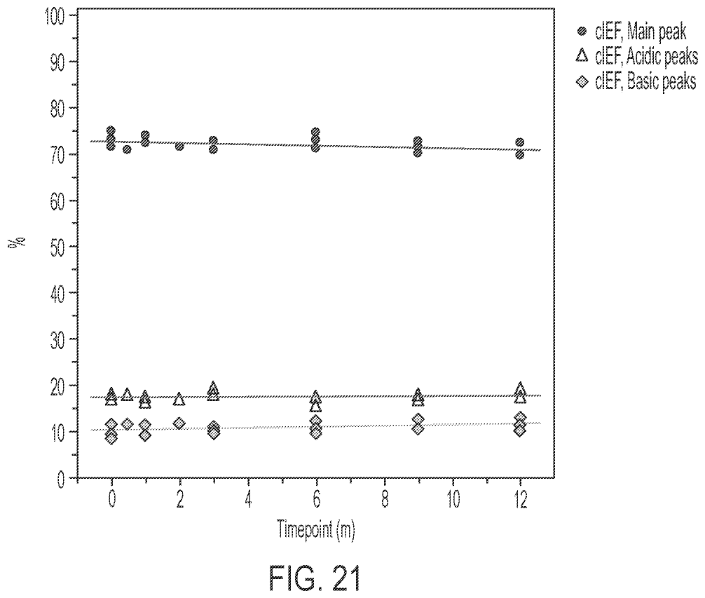

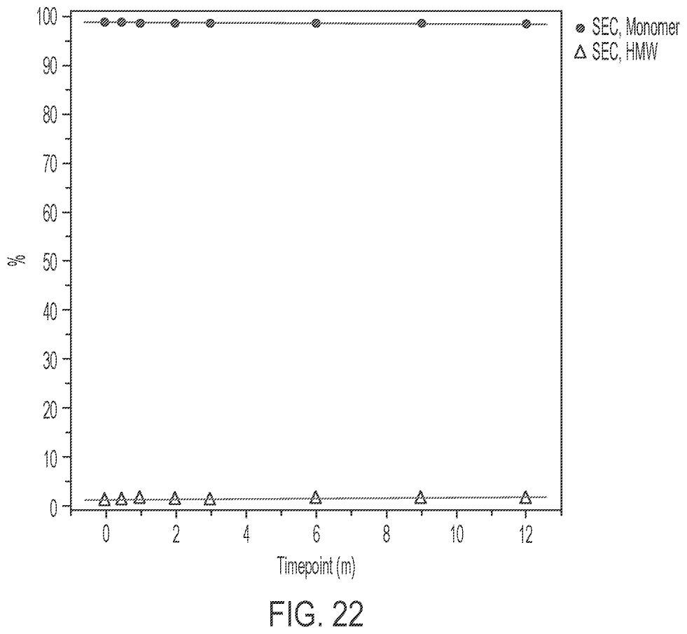

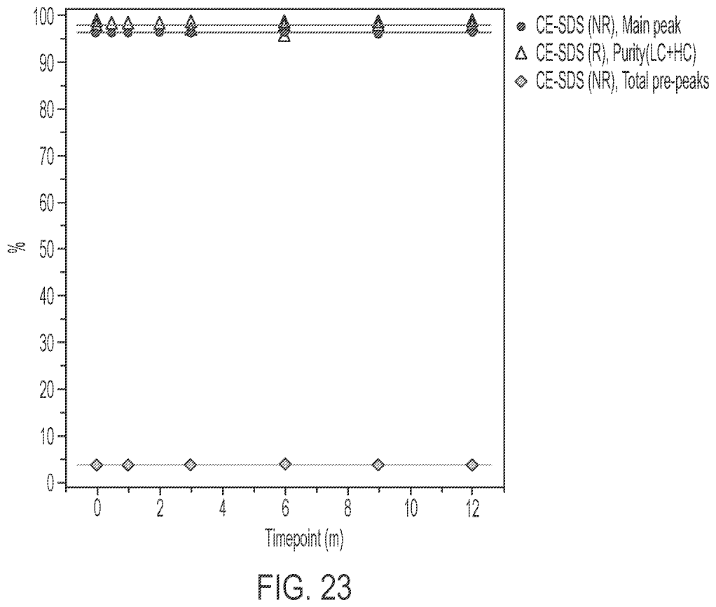

The cIEF, SEC, and CE-SDS (non-reduced and reduced) data for the drug product of 150 mg/mL ADI-58125 in a formulation of 10 mM L-histidine, 75 mM L-arginine hydrochloride, 100 mM sucrose, 0.03% polysorbate 80 (w/v) with a pH of 5.4−5.6 over a time period of 12 months at 5° C. were collected and shown in FIGS. 21-23. A shown in FIGS. 21-23, the drug product remained very stable at 5° C. for at least 12 months.

In conclusion, the studies described herein support the selection of the final formulation and storage conditions: 150 mg/mL ADI-58125 in 10 mM L-histidine, 75 mM L-arginine hydrochloride, 100 mM sucrose, 0.03% polysorbate 80 (w/v), a pH of about 5.4−5.6, e.g., about 5.4, about 5.5 or about 5.6, for long-term storage at ≤−30° C. for drug substance and at 2 to 8° C. for drug product.

Having fully described and enabled the invention, the invention is further described by the claims that follow. In general, in the following claims, the terms used should not be construed to limit the disclosure to the specific embodiments disclosed in the specification and the claims. Accordingly, the invention is not limited by the disclosure, but instead the scope of the invention is to be determined entirely by the following claims.

The disclosure may be practiced in ways other than those particularly described in the foregoing description and examples. Numerous modifications and variations of the disclosure are possible in light of the above teachings and, therefore, are within the scope of the appended claims.

TABLE 30
INFORMAL SEQUENCE LISTING
Antibody sequences

| | | SEQ | | SEQ | | SEQ | | SEQ | | SEQ | | SEQ | | SEQ |
|---|---|---|---|---|---|---|---|---|---|---|---|---|---|---|
| ADI | | ID | VH | ID | | ID | | ID | | ID | | ID | | ID |
| ID | VH FR1 | NO | CDR1 | NO | VH FR2 | NO | VH CDR2 | NO | VH FR3 | NO | VH CDR3 | NO | VH FR4 | NO |
| ADI- | EVQLVESG | 1 | FTFSR | 2 | WVRQA | 3 | AINLNGDS | 4 | RFTISRDNS | 5 | VKDGGY | 6 | WGQGT | 7 |
| 5812 | GGLVQPG | | FAMH | | PGKGLE | | KYYTDSVR | | KNTLYLQM | | YDSSGPG | | LVTVSS | |
| 0 | GSLRLSCS | | | | YVS | | G | | SSVRPDDT | | H | | | |
| | ASG | | | | | | | | AFYYC | | | | | |
| ADI- | EVQLVESG | 11 | FTFSR | 12 | WVRQA | 13 | AINLNGDS | 14 | RFTISRDNS | 15 | VKDGGY | 16 | WGQGT | 17 |
| 5812 | GGLVQPG | | FAMH | | PGKGLE | | KYYTDSVR | | KNTLYLQM | | YDSSGPG | | LVTVSS | |
| 1 | GSLRLSCS | | | | YVS | | G | | SSVRPDDT | | H | | | |
| | ASG | | | | | | | | AFYYC | | | | | |
| ADI- | EVQLVESG | 21 | FTFSR | 22 | WVRQA | 23 | AINLNGDS | 24 | RFTISRDNS | 25 | VKDGGY | 26 | WGQGT | 27 |
| 5812 | GGLVQPG | | FAMH | | PGKGLE | | KYYTDSVR | | KNTLYLQM | | YDSSGPG | | LVTVSS | |
| 2 | GSLRLSCS | | | | YVS | | G | | SSVRPDDT | | H | | | |
| | ASG | | | | | | | | AFYYC | | | | | |
| ADI- | EVQLVESG | 31 | FTFSR | 32 | WVRQA | 33 | AINLNGDS | 34 | RFTISRDNS | 35 | VKDGGY | 36 | WGQGT | 37 |
| 5812 | GGLVQPG | | FAMH | | PGKGLE | | KYYTDSVR | | KNTLYLQM | | YDSSGPG | | LVTVSS | |
| 3 | GSLRLSCS | | | | YVS | | G | | SSVRPDDT | | H | | | |
| | ASG | | | | | | | | AFYYC | | | | | |
| ADI- | EVQLVESG | 41 | FTFSS | 42 | WVRQA | 43 | SISEDGYST | 44 | RFTISRDSA | 45 | ARDFSGH | 46 | WGQGT | 47 |
| 5812 | GGLVKPG | | YYM | | PGKGLE | | YYPDSLKG | | KNSLYLQM | | TAWAGT | | LVTVSS | |
| 4 | GSLRLSCA | | N | | WVS | | | | NSLRADDT | | GFEY | | | |
| | ASG | | | | | | | | AVYYC | | | | | |
| ADI- | EVQLVESG | 51 | FTFSS | 52 | WVRQA | 53 | SISEDGYST | 54 | RFTISRDSA | 55 | ARDFSGH | 56 | WGQGT | 57 |
| 5812 | GGLVKPG | | YYM | | PGKGLE | | YYPDSLKG | | KNSLYLQM | | TAWAGT | | LVTVSS | |
| 5 | GSLRLSCA | | N | | WVS | | | | NSLRADDT | | GFEY | | | |
| | ASG | | | | | | | | AVYYC | | | | | |
| ADI- | EVQLVESG | 61 | FPFK | 62 | WVRQA | 63 | IIYSGGDTY | 64 | RFTISRDNS | 65 | ARDREM | 66 | WGQGT | 67 |
| 5812 | GGLIQPGG | | GTYM | | PGKGLE | | YADSVKG | | KNTLFLQM | | AIITERTY | | TVTVSS | |
| 6 | SLRLSCAA | | T | | WVS | | | | NSLRVEDT | | GLDV | | | |
| | SG | | | | | | | | AMYYC | | | | | |
| ADI- | EVQLVESG | 71 | FPFK | 72 | WVRQA | 73 | IIYSGGDTY | 74 | RFTISRDNS | 75 | ARDREM | 76 | WGQGT | 77 |
| 5812 | GGLIQPGG | | GTYM | | PGKGLE | | YADSVKG | | KNTLFLQM | | AIITERTY | | TVTVSS | |
| 7 | SLRLSCAA | | T | | WVS | | | | NSLRVEDT | | GLDV | | | |
| | SG | | | | | | | | AMYYC | | | | | |
| ADI- | QVQLVQS | 81 | GTFS | 82 | WVRQA | 83 | GIIPIFVTAN | 84 | RVTITADKS | 85 | ARGRMA | 86 | WGQGT | 87 |
| 5812 | GAEVKKP | | SYAIS | | PGQGLE | | YAQKFQG | | TTTVYMEL | | TIRGGQN | | TVTVSS | |
| 8 | GSSVKVSC | | | | WMG | | | | SSLRSEDTA | | YYYYYG | | | |
| | KASG | | | | | | | | VYYC | | MDV | | | |
| ADI- | QVQLVQS | 91 | GTFS | 92 | WVRQA | 93 | GIIPIFVTAN | 94 | RVTITADKS | 95 | ARGRMA | 96 | WGQGT | 97 |
| 5812 | GAEVKKP | | SYAIS | | PGQGLE | | YAQKFQG | | TTTVYMEL | | TIRGGQN | | TVTVSS | |
| 9 | GSSVKVSC | | | | WMG | | | | SSLRSEDTA | | YYYYYG | | | |
| | KASG | | | | | | | | VYYC | | MDV | | | |
| ADI- | EVQLVESG | 101 | FTFD | 102 | WVRQA | 103 | GISWNSGSI | 104 | RFTISRDNA | 105 | ASDSNYR | 106 | WGQGT | 107 |
| 5813 | GGLVQPGR | | DYA | | PGKGLE | | NYADSVKG | | KNSLYLQM | | DYYYHY | | TVTVSS | |
| 0 | SLRLSCAA | | MH | | WVS | | | | NSLRAEDT | | GMDV | | | |
| | SG | | | | | | | | ALYYC | | | | | |
| ADI- | EVQLVESG | 111 | FTFD | 112 | WVRQA | 113 | GISWNSGSI | 114 | RFTISRDNA | 115 | ASDSNYR | 116 | WGQGT | 117 |
| 5813 | GGLVQPGR | | DYA | | PGKGLE | | NYADSVKG | | KNSLYLQM | | DYYYHY | | TVTVSS | |
| 1 | SLRLSCAA | | MH | | WVS | | | | NSLRAEDT | | GMDV | | | |
| | SG | | | | | | | | ALYYC | | | | | |

TABLE 31
Antibody sequences

| | | SEQ |
|---|---|---|
| ADI ID | VH Sequence | ID NO |
| ADI- | EVQLVESGGGLVQPGGSLRLSCSASGFTFSRFAMHWVRQAPGKGLEYVSAINLNGDSKYYTDSVRGRFTISRDNSKNTLYLQM | 8 |
| 58120 | SSVRPDDTAFYYCVKDGGYYDSSGPGHWGQGTLVTVSS | |
| ADI- | EVQLVESGGGLVQPGGSLRLSCSASGFTFSRFAMHWVRQAPGKGLEYVSAINLNGDSKYYTDSVRGRFTISRDNSKNTLYLQM | 18 |
| 58121 | SSVRPDDTAFYYCVKDGGYYDSSGPGHWGQGTLVTVSS | |
| ADI- | EVQLVESGGGLVQPGGSLRLSCSASGFTFSRFAMHWVRQAPGKGLEYVSAINLNGDSKYYTDSVRGRFTISRDNSKNTLYLQM | 28 |
| 58122 | SSVRPDDTAFYYCVKDGGYYDSSGPGHWGQGTLVTVSS | |
| ADI- | EVQLVESGGGLVQPGGSLRLSCSASGFTFSRFAMHWVRQAPGKGLEYVSAINLNGDSKYYTDSVRGRFTISRDNSKNTLYLQM | 38 |
| 58123 | SSVRPDDTAFYYCVKDGGYYDSSGPGHWGQGTLVTVSS | |
| ADI- | EVQLVESGGGLVKPGGSLRLSCAASGFTFSSYYMNWVRQAPGKGLEWVSSISEDGYSTYYPDSLKGRFTISRDSAKNSLYLQM | 48 |
| 58124 | NSLRADDTAVYYCARDFSGHTAWAGTGFEYWGQGTLVTVSS | |
| ADI- | EVQLVESGGGLVKPGGSLRLSCAASGFTFSSYYMNWVRQAPGKGLEWVSSISEDGYSTYYPDSLKGRFTISRDSAKNSLYLQM | 58 |
| 58125 | NSLRADDTAVYYCARDFSGHTAWAGTGFEYWGQGTLVTVSS | |
| ADI- | EVQLVESGGGLIQPGGSLRLSCAASGFPFKGTYMTWVRQAPGKGLEWVSIIYSGGDTYYADSVKGRFTISRDNSKNTLFLQMN | 68 |
| 58126 | SLRVEDTAMYYCARDREMAIITERTYGLDVWGQGTTVTVSS | |
| ADI- | EVQLVESGGGLIQPGGSLRLSCAASGFPFKGTYMTWVRQAPGKGLEWVSIIYSGGDTYYADSVKGRFTISRDNSKNTLFLQMN | 78 |
| 58127 | SLRVEDTAMYYCARDREMAIITERTYGLDVWGQGTTVTVSS | |
| ADI- | QVQLVQSGAEVKKPGSSVKVSCKASGGTFSSYAISWVRQAPGQGLEWMGGIIPIFVTANYAQKFQGRVTITADKSTTTVYMEL | 88 |
| 58128 | SSLRSEDTAVYYCARGRMATIRGGQNYYYYYGMDVWGQGTTVTVSS | |
| ADI- | QVQLVQSGAEVKKPGSSVKVSCKASGGTFSSYAISWVRQAPGQGLEWMGGIIPIFVTANYAQKFQGRVTITADKSTTTVYMEL | 98 |
| 58129 | SSLRSEDTAVYYCARGRMATIRGGQNYYYYYGMDVWGQGTTVTVSS | |
| ADI- | EVQLVESGGGLVQPGRSLRLSCAASGFTFDDYAMHWVRQAPGKGLEWVSGISWNSGSINYADSVKGRFTISRDNAKNSLYLQ | 108 |
| 58130 | MNSLRAEDTALYYCASDSNYRDYYYHYGMDVWGQGTTVTVSS | |
| ADI- | EVQLVESGGGLVQPGRSLRLSCAASGFTFDDYAMHWVRQAPGKGLEWVSGISWNSGSINYADSVKGRFTISRDNAKNSLYLQ | 118 |
| 58131 | MNSLRAEDTALYYCASDSNYRDYYYHYGMDVWGQGTTVTVSS | |

TABLE 32
Antibody sequences

| | | SEQ |
|---|---|---|
| ADI | | ID |
| ID | VH DNA | NO |
| ADI- | GAGGTGCAGCTGGTGGAGTCTGGGGGAGGCTTGGTCCAGCCTGGGGGGTCCCTGAGActtTCCTGTTCAGCCTCTGGATTCA | 9 |
| 58120 | CCTTCAGTaggtttGCTATGCACTGGGTCaggCAGGCTCCAGGGAAGGGACTGGAATATGTTTCAGCTATTaatctgAATGG | |
| | GgacAGCaaaTACTACaccGACTCCGTGaggGGCAGATTCACCATCTCCAGAGACAATTCCAAGAACACGCTGTATCTTCAA | |
| | ATGAGCAGTgtgAGAcccgacGACACGGCTttcTATTACTGTgtaaaagacgggggtTACTACgattcctccGGTcctgggc | |
| | acTGGGGGCAAGGGACCttgGTCACCGTCTCCTCA | |
| ADI- | GAGGTGCAGCTGGTGGAGTCTGGGGGAGGCTTGGTCCAGCCTGGGGGGTCCCTGAGAttgTCCTGTTCAGCCTCTGGATTCA | 19 |
| 58121 | CCTTCAGTaggtttGCTATGCACTGGGTCaggCAGGCTCCAGGGAAGGGACTGGAATATGTTTCAGCTATTaatctgAATGG | |
| | GgatAGCaagTACTACacaGACTCCGTGagaGGCAGATTCACCATCTCCAGAGACAATTCCAAGAACACGCTGTATCTTCAA | |
| | ATGAGCAGTgtaAGAccagacGACACGGCTttcTATTACTGTgttaaggatggcggcTACTACgatagttccGGTcctggtc | |
| | acTGGGGGCAAGGGACCcttGTCACCGTCTCCTCA | |
| ADI- | GAGGTGCAGCTGGTGGAGTCTGGGGGAGGCTTGGTCCAGCCTGGGGGGTCCCTGAGActtTCCTGTTCAGCCTCTGGATTCA | 29 |
| 58122 | CCTTCAGTagattcGCTATGCACTGGGTCaggCAGGCTCCAGGGAAGGGACTGGAATATGTTTCAGCTATTaatcttAATGG | |
| | GgacAGCaagTACTACaccGACTCCGTGaggGGCAGATTCACCATCTCCAGAGACAATTCCAAGAACACGCTGTATCTTCAA | |
| | ATGAGCAGTgttAGAcctgacGACACGGCTttcTATTACTGTgtaaaagacggaggaTACTACgacagttccGGTcccggac | |
| | acTGGGGGCAAGGGACCcttGTCACCGTCTCCTCA | |
| ADI- | GAGGTGCAGCTGGTGGAGTCTGGGGGAGGCTTGGTCCAGCCTGGGGGGTCCCTGAGAttgTCCTGTTCAGCCTCTGGATTCA | 39 |
| 58123 | CCTTCAGTagatttGCTATGCACTGGGTCaggCAGGCTCCAGGGAAGGGACTGGAATATGTTTCAGCTATTaacttgAATGG | |
| | GgatAGCaaaTACTACacgGACTCCGTGagaGGCAGATTCACCATCTCCAGAGACAATTCCAAGAACACGCTGTATCTTCAA | |
| | ATGAGCAGTgttAGAccggacGACACGGCTttcTATTACTGTgtcaaagacgggggaTACTACgatagtagtGGTcctggcc | |
| | acTGGGGGCAAGGGACCcttGTCACCGTCTCCTCA | |
| ADI- | GAGGTGCAGCTGGTGGAGTCTGGGGGAGGCCTGGTCAAGCCTGGGGGGTCCCTGAGActtTCCTGTGCAGCCTCTGGATTCA | 49 |
| 58124 | CCTTCAGTAGCTATtatATGAACTGGGTCagaCAGGCTCCAGGGAAGGGGCTGGAGTGGGTCTCATCCATTAGTgaggatgg | |
| | atacagtacaTACTACcctGACTCActtAAGGGCagaTTCACCATCTCCAGAGACagtGCCAAGAACTCACTGTATCTGCAA | |
| | ATGAACAGCCTGAGAGCCgacGACACGGCTGTGTATTACTGTgccagagatttcagtggtcacaccgcttgggccggtacgg | |
| | gaTTTgaaTACTGGGGCCAAGGAACCCTGGTCACCGTCTCCTCA | |
| ADI- | GAGGTGCAGCTGGTGGAGTCTGGGGGAGGCCTGGTCAAGCCTGGGGGGTCCCTGAGActgTCCTGTGCAGCCTCTGGATTCA | 59 |
| 58125 | CCTTCAGTAGCTATtatATGAACTGGGTCaggCAGGCTCCAGGGAAGGGGCTGGAGTGGGTCTCATCCATTAGTgaggacgg | |
| | atacagcaccTACTACcccGACTCActgAAGGGCagaTTCACCATCTCCAGAGACagcGCCAAGAACTCACTGTATCTGCAA | |
| | ATGAACAGCCTGAGAGCCgatGACACGGCTGTGTATTACTGTgccagagatttttcaggccacacggcgtgggcaggaacgg | |
| | gaTTTgagTACTGGGGCCAAGGAACCCTGGTCACCGTCTCCTCA | |
| ADI- | GAGGTGCAGCTGGTGGAGTCTGGAGGAGGCTTGATCCAGCCTGGGGGGTCCCTGAGActgTCCTGTGCAGCCTCTGGGTTCc | 69 |
| 58126 | cgtttaagggcaccTACATGaccTGGGTCagaCAGGCTCCAGGGAAGGGGCTGGAGTGGGTCTCAatcATTTATAGCGGTGG | |
| | TgatACATACTACGCAGACTCCGTGAAGGGCagaTTCACCATCTCCAGAGACAATTCCAAGAACACGCTGttcCTTCAAATG | |
| | AACAGCCTGAGAgtgGAGGACACGGCCatgTATTACTGTgccagggatagagagatggcgataatcacggaaaggactTACG | |
| | GTttgGACGTCTGGGGGCAAGGGACCACGGTCACCGTCTCCTCA | |
| ADI- | GAGGTGCAGCTGGTGGAGTCTGGAGGAGGCTTGATCCAGCCTGGGGGGTCCCTGAGActgTCCTGTGCAGCCTCTGGGTTCc | 79 |
| 58127 | ccttcaagggaactTACATGacaTGGGTCaggCAGGCTCCAGGGAAGGGGCTGGAGTGGGTCTCAatcATTTATAGCGGTGG | |
| | TgatACATACTACGCAGACTCCGTGAAGGGCagaTTCACCATCTCCAGAGACAATTCCAAGAACACGCTGttcCTTCAAATG | |
| | AACAGCCTGAGAgtcGAGGACACGGCCatgTATTACTGTgccagagatagggagatggcgatcattaccgagaggacgTACG | |
| | GTcttGACGTCTGGGGGCAAGGGACCACGGTCACCGTCTCCTCA | |
| ADI- | CAGGTGCAGCTGGTGCAGTCTGGGGCTGAGGTGAAGAAGCCTGGGTCCagcGTGAAGGTCTCCTGCAAGGCTTCTGGAGGCA | 89 |
| 58128 | CCTTCAGCAGCTATGCTATCAGCTGGGTGaggCAGGCCCCTGGACAAGGGCTTGAGTGGATGGGAGGGATCATCCCTATCTT | |
| | TgttACAGCAAACTACGCACAGAAGTTCCAGGGCAGAGTCACGATTACCGCGGACAAATCCACGacaACAgtaTACATGGAG | |
| | CTGAGCAGCCTGAGATCTGAGGACACGGCCGTGTATTACTGTgctagaggaaggatggccacaataagagggggcagaacTA | |
| | CTACTACTACTACGGTATGGACGTCTGGGGGCAAGGGACCACGGTCACCGTCTCCTCA | |
| ADI- | CAGGTGCAGCTGGTGCAGTCTGGGGCTGAGGTGAAGAAGCCTGGGTCCtcaGTGAAGGTCTCCTGCAAGGCTTCTGGAGGCA | 99 |
| 58130 | CCTTCAGCAGCTATGCTATCAGCTGGGTGagaCAGGCCCCTGGACAAGGGCTTGAGTGGATGGGAGGGATCATCCCTATCTT | |
| | TgtaACAGCAAACTACGCACAGAAGTTCCAGGGCAGAGTCACGATTACCGCGGACAAATCCACGacaACAgttTACATGGAG | |
| | CTGAGCAGCCTGAGATCTGAGGACACGGCCGTGTATTACTGTgccagaggtaggatggccacgattaggggtggacagaatT | |
| | ACTACTACTACTACGGTATGGACGTCTGGGGGCAAGGGACCACGGTCACCGTCTCCTCA | |
| ADI- | GAAGTGCAGCTGGTGGAGTCTGGGGGAGGCTTGGTACAGCCTGGCAGGTCCCTGAGActgTCCTGTGCAGCCTCTGGATTCA | 109 |
| 58129 | CCTTTGATGATTATGCCATGCACTGGGTCagaCAAGCTCCAGGGAAGGGCCTGGAGTGGGTCTCAGGTATTAGTTGGAATAG | |
| | TGGTAGCATAaatTATGCGGACTCTGTGAAGGGCaggTTCACCATCTCCAGAGACAACGCCAAGAACTCCCTGTATCTGCAA | |
| | ATGAACAGTCTGAGAGCTGAGGACACGGCCTTGTATTACTGTgcgtccgatagtaactatagggacTACTACTACcatTACG | |
| | GTATGGACGTCTGGGGGCAAGGGACCACGGTCACCGTCTCCTCA | |
| ADI- | GAAGTGCAGCTGGTGGAGTCTGGGGGAGGCTTGGTACAGCCTGGCAGGTCCCTGAGAttgTCCTGTGCAGCCTCTGGATTCA | 119 |
| 58131 | CCTTTGATGATTATGCCATGCACTGGGTCaggCAAGCTCCAGGGAAGGGCCTGGAGTGGGTCTCAGGTATTAGTTGGAATAG | |
| | TGGTAGCATAaacTATGCGGACTCTGTGAAGGGCagaTTCACCATCTCCAGAGACAACGCCAAGAACTCCCTGTATCTGCAA | |
| | ATGAACAGTCTGAGAGCTGAGGACACGGCCTTGTATTACTGTgcttctgatagtaattatagggacTACTACTACcatTACG | |
| | GTATGGACGTCTGGGGGCAAGGGACCACGGTCACCGTCTCCTCA | |

TABLE 33
Antibody sequences

| | | SEQ | | SEQ | | SEQ | | SEQ | | SEQ | | SEQ | | SEQ |
|---|---|---|---|---|---|---|---|---|---|---|---|---|---|---|
| ADI | | ID | VL | ID | | ID | VL | ID | | ID | VL | ID | VL | ID |
| ID | VL FR1 | NO | CDR1 | NO | VL FR2 | NO | CDR2 | NO | VL FR3 | NO | CDR3 | NO | FR4 | NO |
| ADI- | EIVLTQSPAT | 201 | RASEN | 202 | WYQQK | 203 | DASR | 204 | GIPGRFSGSGAGT | 205 | QQRA | 206 | FGGG | 207 |
| 5812 | LSLSPGERAT | | ILHYL | | PGQAPR | | RAT | | DFTLTINSLEPEDF | | NWPQ | | TKVE | |
| 0 | LSC | | A | | LVIY | | | | AVYYC | | N | | IK | |
| ADI- | EIVLTQSPAT | 211 | RASEN | 212 | WYQQK | 213 | DASR | 214 | GIPGRFSGSGAGT | 215 | QQRA | 216 | FGGG | 217 |
| 5812 | LSLSPGERAT | | ILHYL | | PGQAPR | | RAT | | DFTLTINSLEPEDF | | NWPQ | | TKVE | |
| 1 | LSC | | A | | LVIY | | | | AVYYC | | N | | IK | |
| ADI- | EIVLTQSPAT | 221 | RASEN | 222 | WYQQK | 223 | DASR | 224 | GIPGRFSGSGAGT | 225 | QQRA | 226 | FGGG | 227 |
| 5812 | LSLSPGERAT | | ILHYL | | PGQAPR | | RAT | | DFTLTINSLEPEDF | | NWPQ | | TKVE | |
| 2 | LSC | | A | | LVIY | | | | AVYYC | | N | | IK | |
| ADI- | EIVLTQSPAT | 231 | RASEN | 232 | WYQQK | 233 | DASR | 234 | GIPGRFSGSGAGT | 235 | QQRA | 236 | FGGG | 237 |
| 5812 | LSLSPGERAT | | ILHYL | | PGQAPR | | RAT | | DFTLTINSLEPEDF | | NWPQ | | TKVE | |
| 3 | LSC | | A | | LVIY | | | | AVYYC | | N | | IK | |
| ADI- | QSVLTQPPS | 241 | TGSSS | 242 | WYQQLP | 243 | GSSS | 244 | GVPDRFSGSKSGT | 245 | QSYDS | 246 | FGTG | 247 |
| 5812 | VSGAPGQRI | | NIGAG | | GTAPKL | | RNS | | SASLAITGLQAED | | SLSVL | | TKVT | |
| 4 | TISC | | YDVH | | LIY | | | | EADYYC | | YT | | VL | |
| ADI- | QSVLTQPPS | 251 | TGSSS | 252 | WYQQLP | 253 | GSSS | 254 | GVPDRFSGSKSGT | 255 | QSYDS | 256 | FGTG | 257 |
| 5812 | VSGAPGQRI | | NIGAG | | GTAPKL | | RNS | | SASLAITGLQAED | | SLSVL | | TKVT | |
| 5 | TISC | | YDVH | | LIY | | | | EADYYC | | YT | | VL | |
| ADI- | QSVLTQPPS | 261 | SGGSS | 262 | WYQQLP | 263 | SNSQ | 264 | GVPDRFSGSKSGT | 265 | AAWD | 266 | FGTG | 267 |
| 5812 | ASGTPGQRV | | NIGSN | | GTAPKL | | RPS | | SASLAISGLQSEDE | | DSLNT | | TKVT | |
| 6 | TISC | | SVN | | LIY | | | | ADYYC | | FRYV | | VL | |
| ADI- | QSVLTQPPS | 271 | SGGSS | 272 | WYQQLP | 273 | SNSQ | 274 | GVPDRFSGSKSGT | 275 | AAWD | 276 | FGTG | 277 |
| 5812 | ASGTPGQRV | | NIGSN | | GTAPKL | | RPS | | SASLAISGLQSEDE | | DSLNT | | TKVT | |
| 7 | TISC | | SVN | | LIY | | | | ADYYC | | FRYV | | VL | |
| ADI- | DIQMTQSPSS | 281 | QASQ | 282 | WYQQK | 283 | DASN | 284 | GVPSRFSGSGSGT | 285 | QQYD | 286 | FGPG | 287 |
| 5812 | LSASVGDRV | | DISNY | | PGKAPK | | LET | | DFTFTISSLQPEDI | | | | TKVD | |
| 8 | TITC | | LN | | LLIY | | | | ATYYC | | NLPLT | | IK | |
| ADI- | DIQMTQSPSS | 291 | QASQ | 292 | WYQQK | 293 | DASN | 294 | GVPSRFSGSGSGT | 295 | QQYD | 296 | FGPG | 297 |
| 5812 | LSASVGDRV | | DISNY | | PGKAPK | | LET | | DFTFTISSLQPEDI | | NLPLT | | TKVD | |
| 9 | TITC | | LN | | LLIY | | | | ATYYC | | | | IK | |
| ADI- | DIVMTQSPL | 301 | RSSQS | 302 | WYLQKP | 303 | LGSN | 304 | GVPDRFSGSGSGT | 305 | MQAL | 306 | FGQG | 307 |
| 5813 | SLPVTPGEPA | | LLHSN | | GQSPQL | | RAS | | DFTLKISRVEAED | | QTPRT | | TKVE | |
| 0 | SISC | | GYNY | | LIY | | | | VGVYYC | | | | IK | |
| | | | LD | | | | | | | | | | | |
| ADI- | DIVMTQSPL | 311 | RSSQS | 312 | WYLQKP | 313 | LGSN | 314 | GVPDRFSGSGSGT | 315 | MQAL | 316 | FGQG | 317 |
| 5813 | SLPVTPGEPA | | LLHSN | | GQSPQL | | RAS | | DFTLKISRVEAED | | QTPRT | | TKVE | |
| 1 | SISC | | GYNY | | LIY | | | | VGVYYC | | | | IK | |
| | | | LD | | | | | | | | | | | |

TABLE 34
Antibody VL sequences

| | | SEQ |
|---|---|---|
| | | ID |
| ADI ID | VL Protein | NO |
| ADI- | EIVLTQSPATLSLSPGERATLSCRASENILHYLAWYQQKPGQAPRLVIYDASRRATGIPGRESGSGAGTDFTLTINSLEPEDFA | 208 |
| 58120 | VYYCQQRANWPQNFGGGTKVEIK | |
| ADI- | EIVLTQSPATLSLSPGERATLSCRASENILHYLAWYQQKPGQAPRLVIYDASRRATGIPGRFSGSGAGTDFTLTINSLEPEDFA | 218 |
| 58121 | VYYCQQRANWPQNFGGGTKVEIK | |
| ADI- | EIVLTQSPATLSLSPGERATLSCRASENILHYLAWYQQKPGQAPRLVIYDASRRATGIPGRESGSGAGTDFTLTINSLEPEDFA | 228 |
| 58122 | VYYCQQRANWPQNFGGGTKVEIK | |
| ADI- | EIVLTQSPATLSLSPGERATLSCRASENILHYLAWYQQKPGQAPRLVIYDASRRATGIPGRFSGSGAGTDFTLTINSLEPEDFA | 238 |
| 58123 | VYYCQQRANWPQNFGGGTKVEIK | |
| ADI- | QSVLTQPPSVSGAPGQRITISCTGSSSNIGAGYDVHWYQQLPGTAPKLLIYGSSSRNSGVPDRFSGSKSGTSASLAITGLQAED | 248 |
| 58124 | EADYYCQSYDSSLSVLYTFGTGTKVTVL | |
| ADI- | QSVLTQPPSVSGAPGQRITISCTGSSSNIGAGYDVHWYQQLPGTAPKLLIYGSSSRNSGVPDRESGSKSGTSASLAITGLQAED | 258 |
| 58125 | EADYYCQSYDSSLSVLYTFGTGTKVTVL | |
| ADI- | QSVLTQPPSASGTPGQRVTISCSGGSSNIGSNSVNWYQQLPGTAPKLLIYSNSQRPSGVPDRFSGSKSGTSASLAISGLQSEDE | 268 |
| 58126 | ADYYCAAWDDSLNTFRYVFGTGTKVTVL | |
| ADI- | QSVLTQPPSASGTPGQRVTISCSGGSSNIGSNSVNWYQQLPGTAPKLLIYSNSQRPSGVPDRFSGSKSGTSASLAISGLQSEDE | 278 |
| 58127 | ADYYCAAWDDSLNTFRYVFGTGTKVTVL | |
| ADI- | DIQMTQSPSSLSASVGDRVTITCQASQDISNYLNWYQQKPGKAPKLLIYDASNLETGVPSRFSGSGSGTDFTFTISSLQPEDIA | 288 |
| 58128 | TYYCQQYDNLPLTFGPGTKVDIK | |
| ADI- | DIQMTQSPSSLSASVGDRVTITCQASQDISNYLNWYQQKPGKAPKLLIYDASNLETGVPSRFSGSGSGTDFTFTISSLQPEDIA | 298 |
| 58129 | TYYCQQYDNLPLTFGPGTKVDIK | |
| ADI- | DIVMTQSPLSLPVTPGEPASISCRSSQSLLHSNGYNYLDWYLQKPGQSPQLLIYLGSNRASGVPDRFSGSGSGTDFTLKISRVE | 308 |
| 58130 | AEDVGVYYCMQALQTPRTFGQGTKVEIK | |
| ADI- | DIVMTQSPLSLPVTPGEPASISCRSSQSLLHSNGYNYLDWYLQKPGQSPQLLIYLGSNRASGVPDRFSGSGSGTDFTLKISRVE | 318 |
| 58131 | AEDVGVYYCMQALQTPRTFGQGTKVEIK | |

TABLE 35
Antibody VL sequences

| | | SEQ |
|---|---|---|
| ADI | | ID |
| ID | VL DNA | NO |
| ADI- | GAAATTGTGTTGACACAGTCTCCAGCCACCCTGTCTTTGTCTCCAGGGGAAAGAGCCACCctgTCCTGCAGGGCCAGTgaaaat | 209 |
| 58120 | atattgcacTACttgGCCTGGTACCAACAGAAACCTGGCCAGGCTCCCAGGcttgtcATCTATGATGCATCCaggAGGGCCACTGGCAT | |
| | CCCAggaAGGTTCAGTGGCAGTGGGgcgGGGACAGACTTCACTctgACCATCaatAGCttgGAGCCTGAAGATTTTGCAGTTTATT | |
| | ACTGTcaacaaagagccaattggccccaaaacTTCGGCGGAGGGACCAAGGTGGAGATCAAA | |
| ADI- | GAAATTGTGTTGACACAGTCTCCAGCCACCCTGTCTTTGTCTCCAGGGGAAAGAGCCACCcttTCCTGCAGGGCCAGTgagaac | 219 |
| 58121 | atcctgcacTACttgGCCTGGTACCAACAGAAACCTGGCCAGGCTCCCAGGttggtaATCTATGATGCATCCaggAGGGCCACTGGCAT | |
| | CCCAggtAGGTTCAGTGGCAGTGGGgcgGGGACAGACTTCACTctgACCATCaacAGCctgGAGCCTGAAGATTTTGCAGTTTATT | |
| | ACTGTcaacaaagagccaattggccacagaatTTCGGCGGAGGGACCAAGGTGGAGATCAAA | |
| ADI- | GAAATTGTGTTGACACAGTCTCCAGCCACCCTGTCTTTGTCTCCAGGGGAAAGAGCCACCcttTCCTGCAGGGCCAGTgagaata | 229 |
| 58122 | ttttgcacTACttgGCCTGGTACCAACAGAAACCTGGCCAGGCTCCCAGGctggttATCTATGATGCATCCagaAGGGCCACTGGCATC | |
| | CCAgggAGGTTCAGTGGCAGTGGGgccGGGACAGACTTCACTctgACCATCaatAGCctgGAGCCTGAAGATTTTGCAGTTTATTA | |
| | CTGTcaacagagggcgaactggcctcaaaacTTCGGCGGAGGGACCAAGGTGGAGATCAAA | |
| ADI- | GAAATTGTGTTGACACAGTCTCCAGCCACCCTGTCTTTGTCTCCAGGGGAAAGAGCCACCcttTCCTGCAGGGCCAGTgaaaata | 239 |
| 58123 | tcttgcatTACttgGCCTGGTACCAACAGAAACCTGGCCAGGCTCCCAGGctggtgATCTATGATGCATCCaggAGGGCCACTGGCATC | |
| | CCAggcAGGTTCAGTGGCAGTGGGgcaGGGACAGACTTCACTttgACCATCaacAGCcttGAGCCTGAAGATTTTGCAGTTTATTA | |
| | CTGTcaacagagagcaaattggcctcaaaatTTCGGCGGAGGGACCAAGGTGGAGATCAAA | |
| ADI- | CAGTCTGTGCTGACGCAGCCGCCCTCAGTGTCTGGGGCCCCAGGGCAGAGGattACCATCTCCTGCACTGGGAGCAGCTCCA | 249 |
| 58124 | ACATCGGGGCAGGTTATGATGTACACTGGTACCAGCAGCTTCCAGGAACAGCCCCCAAActgttgATCTATGGTagtAGCtccagg | |
| | aatTCAGGGGTCCCTGACagaTTCTCTGGCTCCAAGTCTGGCACCTCAGCCTCCCTGGCCATCACTGGGttgCAGGCTGAGGATG | |
| | AGGCTGATTATTACTGCcaatcatacgacagctcactgtccgtgcttTATacgTTCGGAACTGGGACCAAGGTCACCGTCctg | |
| ADI- | CAGTCTGTGCTGACGCAGCCGCCCTCAGTGTCTGGGGCCCCAGGGCAGAGGatcACCATCTCCTGCACTGGGAGCAGCTCC | 259 |
| 58125 | AACATCGGGGCAGGTTATGATGTACACTGGTACCAGCAGCTTCCAGGAACAGCCCCCAAActtctgATCTATGGTtctAGCtccag | |
| | gaacTCAGGGGTCCCTGACaggTTCTCTGGCTCCAAGTCTGGCACCTCAGCCTCCCTGGCCATCACTGGGcttCAGGCTGAGGAT | |
| | GAGGCTGATTATTACTGCcaaagttatgactctagtctgtccgtcttgTATacgTTCGGAACTGGGACCAAGGTCACCGTCctg | |
| ADI- | CAGTCTGTGCTGACTCAGCCACCCTCAGCGTCTGGGACCCCCGGGCAGAGGGTCACCATCTCTTGTTCTGGAggtAGCTCCA | 269 |
| 58126 | ACATCGGAAGTAATtcaGTAAACTGGTACCAGCAGttgCCAGGAACGGCCCCCAAActtcttATCTATAGTAATtctCAGagaCCCTC | |
| | AGGGGTCCCTGACaggTTCTCTGGCTCCAAGTCTGGCACCTCAGCCTCCCTGGCCATCAGTGGGttgCAGTCTGAGGATGAGG | |
| | CTGATTATTACTGTgcagcatgggacgattccttgaatactttcagaTATGTCTTCGGAACTGGGACCAAGGTCACCGTCctg | |
| ADI- | CAGTCTGTGCTGACTCAGCCACCCTCAGCGTCTGGGACCCCCGGGCAGAGGGTCACCATCTCTTGTTCTGGAggcAGCTCCA | 279 |
| 58127 | ACATCGGAAGTAATagcGTAAACTGGTACCAGCAGttgCCAGGAACGGCCCCCAAAttgttgATCTATAGTAATagtCAGaggCCCT | |
| | CAGGGGTCCCTGACagaTTCTCTGGCTCCAAGTCTGGCACCTCAGCCTCCCTGGCCATCAGTGGGcttCAGTCTGAGGATGAG | |
| | GCTGATTATTACTGTgctgcatgggacgattctttgaacactttcaggTATGTCTTCGGAACTGGGACCAAGGTCACCGTCett | |
| ADI- | GACATCCAGATGACCCAGTCTCCATCCTCCCTGTCTGCATCTGTAGGAGACAGAGTCACCATCACTTGCCAGGCGAGTCAG | 289 |
| 58128 | GACATTAGCAACTATcttAATTGGTATCAGCAGAAACCAGGGAAAGCCCCTAAGttgCTGATCTACGATGCATCCAATTTGGA | |
| | AACAGGGGTCCCATCAAGGTTCAGTGGAAGTGGATCTGGGACAGATTTTACTTTCACCATCAGCAGCCTGCAGCCTGAAG | |
| | ATATTGCAACATATTACTGTcaacaatacgataatctgcccttgACTTTCGGCCCTGGGACCAAAGTGGATATCAAA | |
| ADI- | GACATCCAGATGACCCAGTCTCCATCCTCCCTGTCTGCATCTGTAGGAGACAGAGTCACCATCACTTGCCAGGCGAGTCAG | 299 |
| 58129 | GACATTAGCAACTATttgAATTGGTATCAGCAGAAACCAGGGAAAGCCCCTAAGctgCTGATCTACGATGCATCCAATTTGGA | |
| | AACAGGGGTCCCATCAAGGTTCAGTGGAAGTGGATCTGGGACAGATTTTACTTTCACCATCAGCAGCCTGCAGCCTGAAG | |
| | ATATTGCAACATATTACTGTcaacagtatgataacctgccgettACTTTCGGCCCTGGGACCAAAGTGGATATCAAA | |
| ADI- | GATATTGTGATGACTCAGTCTCCActtTCCCTGCCCGTCACCCCTGGAGAGCCGGCCTCCATCTCCTGCAGGTCTAGTCAGAG | 309 |
| 58130 | CctgCTGCATAGTAATGGATACAACTATTTGGATTGGTACCTGCAGAAGCCAGGGCAGTCTCCACAGcttCTGATCTATTTGG | |
| | GTTCTAATaggGCCTCCGGGGTCCCTGACAGGTTCAGTGGCAGTGGATCAGGCACAGATTTTACACTGAAAATCAGCAGAG | |
| | TGGAGGCTGAGGATGTTGGGGTTTATTACTGCatgcaagccctgcagactccaaggACGTTCGGCCAAGGGACCAAGGTGGAAATCAA | |
| | A | |
| ADI- | GATATTGTGATGACTCAGTCTCCActgTCCCTGCCCGTCACCCCTGGAGAGCCGGCCTCCATCTCCTGCAGGTCTAGTCAGA | 319 |
| 58131 | GCctgCTGCATAGTAATGGATACAACTATTTGGATTGGTACCTGCAGAAGCCAGGGCAGTCTCCACAGcttCTGATCTATTTG | |
| | GGTTCTAATagaGCCTCCGGGGTCCCTGACAGGTTCAGTGGCAGTGGATCAGGCACAGATTTTACACTGAAAATCAGCAGA | |
| | GTGGAGGCTGAGGATGTTGGGGTTTATTACTGCatgcaggctttgcagacacctagaACGTTCGGCCAAGGGACCAAGGTGGAAATCAA | |
| | A | |

TABLE 36
Antibody HC Sequences

| | | SEQ |
|---|---|---|
| ADI | | ID |
| ID | HC Sequence | NO |
| ADI- | EVQLVESGGGLVQPGGSLRLSCSASGFTFSRFAMHWVRQAPGKGLEYVSAINLNGDSKYYTDSVRGRFTISRDNSKNTLYLQMSS | 10 |
| 58120 | VRPDDTAFYYCVKDGGYYDSSGPGHWGQGTLVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKRVEPKSCDKTHTCPPCPAPELLGGPSVFLFPPKPKDTLM | |
| | ISRTPEVTCVVVDVSHEDPEVKFNWYVDGVEVHNAKTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNGQPENNYKTTPPVLDSDGSFFLYSKLTVDKSRWQQGN | |
| | VFSCSVMHEALHNHYTQKSLSLSPGK | |
| ADI- | EVQLVESGGGLVQPGGSLRLSCSASGFTFSRFAMHWVRQAPGKGLEYVSAINLNGDSKYYTDSVRGRFTISRDNSKNTLYLQMSS | 20 |
| 58121 | VRPDDTAFYYCVKDGGYYDSSGPGHWGQGTLVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKRVEPKSCDKTHTCPPCPAPELLGGPSVFLFPPKPKDTLY | |
| | ITREPEVTCVVVDVSHEDPEVKFNWYVDGVEVHNAKTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNGQPENNYKTTPPVLDSDGSFFLYSKLTVDKSRWQQGN | |
| | VFSCSVMHEALHNHYTQKSLSLSPGK | |
| ADI- | EVQLVESGGGLVQPGGSLRLSCSASGFTFSRFAMHWVRQAPGKGLEYVSAINLNGDSKYYTDSVRGRFTISRDNSKNTLYLQMSS | 30 |
| 58122 | VRPDDTAFYYCVKDGGYYDSSGPGHWGQGTLVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKRVEPKSCDKTHTCPPCPAPELLGGPSVFLFPPKPKDTLM | |
| | ISRTPEVTCVVVDVSHEDPEVKFNWYVDGVEVHNAKTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNGQPENNYKTTPPVLDSDGSFFLYSKLTVDKSRWQQGN | |
| | VFSCSVLHEALHAHYTQKSLSLSPGK | |
| ADI- | EVQLVESGGGLVQPGGSLRLSCSASGFTFSRFAMHWVRQAPGKGLEYVSAINLNGDSKYYTDSVRGRFTISRDNSKNTLYLQMSS | 40 |
| 58123 | VRPDDTAFYYCVKDGGYYDSSGPGHWGQGTLVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKRVEPKSCDKTHTCPPCPAPELLGGPSVFLFPPKPKDTLM | |
| | ISRTPEVTCVVVDVSHEDPEVKFNWYVDGVEVHNAKTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNGQPENNYKTTPPVLDSDGSFFLYSKLTVDKSRWQQGN | |
| | VFSCSVLHEALHSHYTQKSLSLSPGK | |
| ADI- | EVQLVESGGGLVKPGGSLRLSCAASGFTFSSYYMNWVRQAPGKGLEWVSSISEDGYSTYYPDSLKGRFTISRDSAKNSLYLQMNS | 50 |
| 58124 | LRADDTAVYYCARDFSGHTAWAGTGFEYWGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKRVEPKSCDKTHTCPPCPAPELLGGPSVFLFPPKPKD | |
| | TLMISRTPEVTCVVVDVSHEDPEVKFNWYVDGVEVHNAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVEWESNGQPENNYKTTPPVLDSDGSFFLYSKLTVDKSRW | |
| | QQGNVFSCSVMHEALHNHYTQKSLSLSPGK | |
| ADI- | EVQLVESGGGLVKPGGSLRLSCAASGFTFSSYYMNWVRQAPGKGLEWVSSISEDGYSTYYPDSLKGRFTISRDSAKNSLYLQMNS | 60 |
| 58125 | LRADDTAVYYCARDFSGHTAWAGTGFEYWGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKRVEPKSCDKTHTCPPCPAPELLGGPSVFLFPPKPKD | |
| | TLMISRTPEVTCVVVDVSHEDPEVKFNWYVDGVEVHNAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVEWESNGQPENNYKTTPPVLDSDGSFFLYSKLTVDKSRW | |
| | QQGNVFSCSVLHEALHAHYTQKSLSLSPGK | |
| ADI- | EVQLVESGGGLIQPGGSLRLSCAASGFPFKGTYMTWVRQAPGKGLEWVSIIYSGGDTYYADSVKGRFTISRDNSKNTLFLQMNSL | 70 |
| 58126 | RVEDTAMYYCARDREMAIITERTYGLDVWGQGTTVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKRVEPKSCDKTHTCPPCPAPELLGGPSVFLFPPKPKD | |
| | TLMISRTPEVTCVVVDVSHEDPEVKFNWYVDGVEVHNAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVEWESNGQPENNYKTTPPVLDSDGSFFLYSKLTVDKSRW | |
| | QQGNVFSCSVMHEALHNHYTQKSLSLSPGK | |
| ADI- | EVQLVESGGGLIQPGGSLRLSCAASGFPFKGTYMTWVRQAPGKGLEWVSIIYSGGDTYYADSVKGRFTISRDNSKNTLFLQMNSL | 80 |
| 58127 | RVEDTAMYYCARDREMAIITERTYGLDVWGQGTTVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKRVEPKSCDKTHTCPPCPAPELLGGPSVFLFPPKPKD | |
| | TLMISRTPEVTCVVVDVSHEDPEVKFNWYVDGVEVHNAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVEWESNGQPENNYKTTPPVLDSDGSFFLYSKLTVDKSRW | |
| | QQGNVFSCSVLHEALHAHYTQKSLSLSPGK | |
| ADI- | QVQLVQSGAEVKKPGSSVKVSCKASGGTFSSYAISWVRQAPGQGLEWMGGIIPIFVTANYAQKFQGRVTITADKSTTTVYMELSS | 90 |
| 58128 | LRSEDTAVYYCARGRMATIRGGQNYYYYYGMDVWGQGTTVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNTKVDKRVEPKSCDKTHTCPPCPAPELLGGPSVFLFP | |
| | PKPKDTLMISRTPEVTCVVVDVSHEDPEVKFNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIAVEWESNGQPENNYKTTPPVLDSDGSFFLYSKLTVD | |
| | KSRWQQGNVFSCSVMHEALHNHYTQKSLSLSPGK | |
| ADI- | QVQLVQSGAEVKKPGSSVKVSCKASGGTFSSYAISWVRQAPGQGLEWMGGIIPIFVTANYAQKFQGRVTITADKSTTTVYMELSS | 100 |
| 58129 | LRSEDTAVYYCARGRMATIRGGQNYYYYYGMDVWGQGTTVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNTKVDKRVEPKSCDKTHTCPPCPAPELLGGPSVFLFP | |
| | PKPKDTLMISRTPEVTCVVVDVSHEDPEVKFNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIAVEWESNGQPENNYKTTPPVLDSDGSFFLYSKLTVD | |
| | KSRWQQGNVFSCSVLHEALHAHYTQKSLSLSPGK | |
| ADI- | EVQLVESGGGLVQPGRSLRLSCAASGFTFDDYAMHWVRQAPGKGLEWVSGISWNSGSINYADSVKGRFTISRDNAKNSLYLQM | 110 |
| 58130 | NSLRAEDTALYYCASDSNYRDYYYHYGMDVWGQGTTVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVDKRVEPKSCDKTHTCPPCPAPELLGGPSVFLFPPKP | |
| | KDTLMISRTPEVTCVVVDVSHEDPEVKFNWYVDGVEVHNAKTKPREEQYNSTYRVVSVLTVLHQDWLNGKEYKCKVSNKALP | |
| | APIEKTISKAKGQPREPQVYTLPPSREEMTKNQVSLTCLVKGFYPSDIAVEWESNGQPENNYKTTPPVLDSDGSFFLYSKLTVDKS | |
| | RWQQGNVFSCSVMHEALHNHYTQKSLSLSPGK | |
| ADI- | EVQLVESGGGLVQPGRSLRLSCAASGFTFDDYAMHWVRQAPGKGLEWVSGISWNSGSINYADSVKGRFTISRDNAKNSLYLQM | 120 |
| 58131 | NSLRAEDTALYYCASDSNYRDYYYHYGMDVWGQGTTVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVDKRVEPKSCDKTHTCPPCPAPELLGGPSVFLFPPKP | |
| | KDTLMISRTPEVTCVVVDVSHEDPEVKFNWYVDGVEVHNAKTKPREEQYNSTYRVVSVLTVLHQDWLNGKEYKCKVSNKALP | |
| | APIEKTISKAKGQPREPQVYTLPPSREEMTKNQVSLTCLVKGFYPSDIAVEWESNGQPENNYKTTPPVLDSDGSFFLYSKLTVDKS | |
| | RWQQGNVFSCSVLHEALHAHYTQKSLSLSPGK | |

TABLE 37
Antibody LC Sequences

| | | SEQ |
|---|---|---|
| ADI | | ID |
| ID | LC Sequence | NO |
| ADI- | EIVLTQSPATLSLSPGERATLSCRASENILHYLAWYQQKPGQAPRLVIYDASRRATGIPGRFSGSGAGTDFTLTINSLEPEDFAVYYC | 210 |
| 58120 | QQRANWPQNFGGGTKVEIKRTVAAPSVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGEC | |
| ADI- | EIVLTQSPATLSLSPGERATLSCRASENILHYLAWYQQKPGQAPRLVIYDASRRATGIPGRFSGSGAGTDFTLTINSLEPEDFAVYYC | 220 |
| 58121 | QQRANWPQNFGGGTKVEIKRTVAAPSVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGEC | |
| ADI- | EIVLTQSPATLSLSPGERATLSCRASENILHYLAWYQQKPGQAPRLVIYDASRRATGIPGRESGSGAGTDFTLTINSLEPEDFAVYYC | 230 |
| 58122 | QQRANWPQNFGGGTKVEIKRTVAAPSVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGEC | |
| ADI- | EIVLTQSPATLSLSPGERATLSCRASENILHYLAWYQQKPGQAPRLVIYDASRRATGIPGRESGSGAGTDFTLTINSLEPEDFAVYYC | 240 |
| 58123 | QQRANWPQNFGGGTKVEIKRTVAAPSVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGEC | |
| ADI- | QSVLTQPPSVSGAPGQRITISCTGSSSNIGAGYDVHWYQQLPGTAPKLLIYGSSSRNSGVPDRFSGSKSGTSASLAITGLQAEDEAD | 250 |
| 58124 | YYCQSYDSSLSVLYTFGTGTKVTVLGQPKAAPSVTLFPPSSEELQANKATLVCLISDFYPGAVTVAWKADSSPVKAGVETTTPSK | |
| | QSNNKYAASSYLSLTPEQWKSHRSYSCQVTHEGSTVEKTVAPTECS | |
| ADI- | QSVLTQPPSVSGAPGQRITISCTGSSSNIGAGYDVHWYQQLPGTAPKLLIYGSSSRNSGVPDRFSGSKSGTSASLAITGLQAEDEAD | 260 |
| 58125 | YYCQSYDSSLSVLYTFGTGTKVTVLGQPKAAPSVTLFPPSSEELQANKATLVCLISDFYPGAVTVAWKADSSPVKAGVETTTPSK | |
| | QSNNKYAASSYLSLTPEQWKSHRSYSCQVTHEGSTVEKTVAPTECS | |
| ADI- | QSVLTQPPSASGTPGQRVTISCSGGSSNIGSNSVNWYQQLPGTAPKLLIYSNSQRPSGVPDRFSGSKSGTSASLAISGLQSEDEADYY | 270 |
| 58126 | CAAWDDSLNTFRYVFGTGTKVTVLGQPKAAPSVTLFPPSSEELQANKATLVCLISDFYPGAVTVAWKADSSPVKAGVETTTPSKQ | |
| | SNNKYAASSYLSLTPEQWKSHRSYSCQVTHEGSTVEKTVAPTECS | |
| ADI- | QSVLTQPPSASGTPGQRVTISCSGGSSNIGSNSVNWYQQLPGTAPKLLIYSNSQRPSGVPDRFSGSKSGTSASLAISGLQSEDEADYY | 280 |
| 58127 | CAAWDDSLNTFRYVFGTGTKVTVLGQPKAAPSVTLFPPSSEELQANKATLVCLISDFYPGAVTVAWKADSSPVKAGVETTTPSKQ | |
| | SNNKYAASSYLSLTPEQWKSHRSYSCQVTHEGSTVEKTVAPTECS | |
| ADI- | DIQMTQSPSSLSASVGDRVTITCQASQDISNYLNWYQQKPGKAPKLLIYDASNLETGVPSRFSGSGSGTDFTFTISSLQPEDIATYYC | 290 |
| 58128 | QQYDNLPLTFGPGTKVDIKRTVAAPSVFIFPPSDEQLKSGTASVVCLLNNFYPREAKVQWKVDNALQSGNSQESVTEQDSKDSTY | |
| | SLSSTLTLSKADYEKHKVYACEVTHQGLSSPVTKSFNRGEC | |
| ADI- | DIQMTQSPSSLSASVGDRVTITCQASQDISNYLNWYQQKPGKAPKLLIYDASNLETGVPSRFSGSGSGTDFTFTISSLQPEDIATYYC | 300 |
| 58129 | QQYDNLPLTFGPGTKVDIKRTVAAPSVFIFPPSDEQLKSGTASVVCLLNNFYPREAKVQWKVDNALQSGNSQESVTEQDSKDSTY | |
| | SLSSTLTLSKADYEKHKVYACEVTHQGLSSPVTKSFNRGEC | |
| ADI- | DIVMTQSPLSLPVTPGEPASISCRSSQSLLHSNGYNYLDWYLQKPGQSPQLLIYLGSNRASGVPDRFSGSGSGTDFTLKISRVEAED | 310 |
| 58130 | VGVYYCMQALQTPRTFGQGTKVEIKRTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| ADI- | DIVMTQSPLSLPVTPGEPASISCRSSQSLLHSNGYNYLDWYLQKPGQSPQLLIYLGSNRASGVPDRFSGSGSGTDFTLKISRVEAED | 320 |
| 58131 | VGVYYCMQALQTPRTFGQGTKVEIKRTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |

TABLE 38
Exemplary Fc Variants

| Fc | SEQ | | Diff- |
|---|---|---|---|
| Variant | ID | | erences |
| Name | NO: | Fc | from WT |
| WT | 411 | PPCPAPELLGGPSVFLFPPKPKDTLMISRTPEVTCVVVDVSHEDPEVKFNWYVDGVEVHNAKTKPREEQYNSTYRVVS | 0 |
| | | VLTVLHQDWLNGKEYKCKVSNKALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHYTQKSLSLSPG | |
| YTE | 412 | PPCPAPELLGGPSVFLFPPKPKDTLYITREPEVTCVVVDVSHEDPEVKFNWYVDGVEVHNAKTKPREEQYNSTYRVVS | 3 |
| | | VLTVLHQDWLNGKEYKCKVSNKALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHYTQKSLSLSPG | |
| LA | 413 | PPCPAPELLGGPSVFLFPPKPKDTLMISRTPEVTCVVVDVSHEDPEVKFNWYVDGVEVHNAKTKPREEQYNSTYRVVS | 2 |
| | | VLTVLHQDWLNGKEYKCKVSNKALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVLHEALHAHYTQKSLSLSPG | |
| LS | 414 | PPCPAPELLGGPSVFLFPPKPKDTLMISRTPEVTCVVVDVSHEDPEVKFNWYVDGVEVHNAKTKPREEQYNSTYRVVS | 2 |
| | | VLTVLHQDWLNGKEYKCKVSNKALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVLHEALHSHYTQKSLSLSPG | |
| LA-RE | 415 | PPCPAPELLGGPSVFLFPPKPKDTLMISRTPEVTCVVVDVSHEDPEVKFNWYVDGVEVHNAKTKPREEQYNSTYRVVS | 4 |
| | | VLTVLHQDWLNGKEYKCKVSNKALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVLHEALHAHYTRKELSLSPG | |
| DEL | 416 | PPCPAPELLGGPSVFLFPPKPKDTLMISRTPEVTCVVVDVSHEDPEVKFNWYVDGVEVHNAKTKPREEQYNSTYRVVS | 2 |
| | | VLTVLHQDWLNGKEYKCKVSNKALPAPIEKTISKAKGQPREPQVYTLPPSRDELTKNQVSLTCLVKGFYPSDIAVEWE | |
| | | SNGQPENNYKTTPPVLDSDGSFFLYSKLTVDKSRWQQGNVFSCSVMHEALHNHYTQKSLSLSPG | |
| LALA- | 417 | PPCPAPEAAGGPSVFLFPPKPKDTLMISRTPEVTCVVVDVSHEDPEVKFNWYVDGVEVHNAKTKPREEQYNSTYRVVS | 4 |
| DEL | | VLTVLHQDWLNGKEYKCKVSNKALPAPIEKTISKAKGQPREPQVYTLPPSRDELTKNQVSLTCLVKGFYPSDIAVEWE | |
| | | SNGQPENNYKTTPPVLDSDGSFFLYSKLTVDKSRWQQGNVFSCSVMHEALHNHYTQKSLSLSPG | |

| SEQ | |
|---|---|
| ID NO: | |
| 401 | MFVFLVLLPLVSSQCVNLTTRTQLPPAYTNSFTRGVYYPDKVFRSSVLHSTQDLFLPFFSNVTWFHAIHVSGTNGTKRFDNPV |
| SARS- | LPFNDGVYFASTEKSNIIRGWIFGTTLDSKTQSLLIVNNATNVVIKVCEFQFCNDPFLGVYYHKNNKSWMESEFRVYSSANNC |
| COV-S: | TFEYVSQPFLMDLEGKQGNFKNLREFVFKNIDGYFKIYSKHTPINLVRDLPQGFSALEPLVDLPIGINITRFQTLLALHRSYL |
| | TPGDSSSGWTAGAAAYYVGYLQPRTFLLKYNENGTITDAVDCALDPLSETKCTLKSFTVEKGIYQTSNFRVQPTESIVRFPNI |
| | TNLCPFGEVENATRFASVYAWNRKRISNCVADYSVLYNSASFSTFKCYGVSPTKLNDLCFTNVYADSFVIRGDEVRQIAPGQT |
| | GKIADYNYKLPDDFTGCVIAWNSNNLDSKVGGNYNYLYRLFRKSNLKPFERDISTEIYQAGSTPCNGVEGENCYFPLQSYGFQ |
| | PTNGVGYQPYRVVVLSFELLHAPATVCGPKKSTNLVKNKCVNFNFNGLTGTGVLTESNKKFLPFQQFGRDIADTTDAVRDPQT |
| | LEILDITPCSFGGVSVITPGTNTSNQVAVLYQDVNCTEVPVAIHADQLTPTWRVYSTGSNVFQTRAGCLIGAEHVNNSYECDI |
| | PIGAGICASYQTQTNSPGSASSVASQSIIAYTMSLGAENSVAYSNNSIAIPTNFTISVTTEILPVSMTKTSVDCTMYICGDST |
| | ECSNLLLQYGSFCTQLNRALTGIAVEQDKNTQEVFAQVKQIYKTPPIKDFGGFNFSQILPDPSKPSKRSFIEDLLFNKVTLAD |
| | AGFIKQYGDCLGDIAARDLICAQKFNGLTVLPPLLTDEMIAQYTSALLAGTITSGWTFGAGAALQIPFAMQMAYRFNGIGVTQ |
| | NVLYENQKLIANQFNSAIGKIQDSLSSTASALGKLQDVVNQNAQALNTLVKQLSSNFGAISSVLNDILSRLDPPEAEVQIDRL |
| | ITGRLQSLQTYVTQQLIRAAEIRASANLAATKMSECVLGQSKRVDFCGKGYHLMSFPQSAPHGVVFLHVTYVPAQEKNFTTAP |
| | AICHDGKAHFPREGVFVSNGTHWFVTQRNFYEPQIITTDNTFVSGNCDVVIGIVNNTVYDPLQPELDSFKEELDKYFKNHTSP |
| | DVDLGDISGINASVVNIQKEIDRLNEVAKNLNESLIDLQELGKYEQGSGYIPEAPRDGQAYVRKDGEWVLLSTFLGRSLEVLF |
| | QGPGHHHHHHHHSAWSHPQFEKGGGSGGGGSGGSAWSHPQFEK |
| 402 | atgttcgtgttcctggtgctcctgcctctggtgagcagccagtgcgtgaacctgaccacccgaacccagctcccaccagccta |
| | caccaacagctttacacggggcgtgtactaccctgacaaggtgttcagatctagcgtcctgcacagcactcaggacctcttcc |
| | tgccgttcttcagcaacgtgacatggttccacgccatccacgtgagcggcacaaacggaaccaagcggtttgataaccccgtc |
| | ctgccattcaatgatggagtttacttcgccagtaccgagaagagtaacatcatccggggctggatcttcggcaccaccctgga |
| | tagcaaaacacagagcctcctgatcgtgaacaatgccacgaacgtcgtgatcaaggtgtgcgagttccagttttgcaatgatc |
| | ctttcctgggtgtgtactaccacaagaacaacaagagctggatggaaagcgagttcagagtctacagcagcgccaacaactgc |
| | acattcgagtacgtctctcagccttttctgatggaccttgaggggaaacaaggcaacttcaagaacctgagagaattcgtgtt |
| | caagaacatcgacggctacttcaaaatctactccaagcacacacccatcaacctggtccgggacctccctcagggcttcagcg |
| | ccctggaacccctggtcgacctgcccataggcatcaacataacgcggttccaaaccctgctggccctgcatagatcctacctg |
| | actcctggcgacagcagcagcggatggaccgccggagctgcagcctactatgtgggctacctgcaacctagaaccttcctgct |
| | gaagtacaacgagaacggcacaatcacagacgccgtcgactgcgccctggaccctctctctgagacaaagtgcaccctgaagt |
| | ccttcaccgtggaaaagggcatctaccagaccagcaacttccgggtgcagcctacagagagcatcgtgcgatttccaaacatt |
| | accaacctctgccccttcggcgaggtgtttaacgccacaagatttgcctccgtttacgcctggaatagaaagagaatcagcaa |
| | ttgtgtggccgactactccgtgctgtataacagcgcctctttcagcaccttcaagtgctacggcgtttccccaacaaagctga |
| | atgacctgtgcttcaccaacgtgtacgccgactccttcgtaattagaggcgatgaggtgcggcagatcgcaccaggccagacc |
| | ggtaagatcgctgactacaactataagctgcctgatgattttacaggctgcgtgatcgcctggaactctaacaacctggatag |
| | caaggtgggggcaactacaactacctgtaccggctgtttcgcaagtctaacctgaaacctttcgagagagacatctccacaga |
| | gatctaccaggccggttctacaccttgtaacggggtggaaggcttcaactgttacttccctctgcaaagctacggcttccagc |
| | ctaccaatggagtcggctaccagccataccgggtggtcgtgctgtccttcgagttactccacgcccccgccaccgtctgcggt |
| | cctaagaagtccaccaatctggttaagaacaaatgcgtgaacttcaacttcaacggcctgaccgggaccggcgtgctgaccga |
| | aagcaacaaaaagttcctccccttccagcagttcggccgtgatatcgctgacaccacagatgccgtcagagatccacagaccc |
| | tggaaatcctggatattacaccctgctccttcggaggagtttctgtgatcacccccgggaccaataccagcaaccaggtggct |
| | gtgctgtaccaagatgttaactgcaccgaggttcctgtggccatccacgccgatcagctgacacctacttggagagtgtactc |
| | cactggctccaatgtgttccagaccagggccggatgtctgatcggcgccgagcacgtgaataacagttacgagtgcgacatcc |
| | ctatcggcgccggcatctgtgccagctaccagacccagacaaacagccctgggtctgcttcctctgtagctagccagagcatc |
| | atcgcctacaccatgagcctgggcgcagagaacagcgtggcctattccaacaactctatcgccattcccaccaactttacaat |
| | tagcgtcacaacagagatcctgcccgtgagcatgaccaagaccagcgtggactgtacaatgtacatctgtggcgacagcactg |
| | aatgcagcaacctgctgctgcaatacggctccttttgcacccaactgaaccgggcgctgaccggaatcgccgtggaacaggac |
| | aaaaatacccaggaggtgttcgcccaagtgaagcagatctacaagaccccacctatcaaggacttcggcggctttaactttag |
| | ccagattctccctgatccttctaagcctagcaagcggagctttatcgaggatctgctgttcaacaaggtcaccctggccgatg |
| | ccggctttatcaaacagtatggcgattgcctgggcgacatagccgccagagatctgatctgcgcccagaaattcaacggcctg |
| | acagttctcccacctctgctgaccgacgagatgatcgctcagtacacctctgccctgctggctggcaccatcacatctgggtg |
| | gacatttggcgccggcgccgccctgcagatcccctttgccatgcagatggcctatagattcaacggaatcggcgtgacccaga |
| | acgtgctgtatgaaaaccagaagctgatcgctaaccagttcaattctgccatcggcaagatccaggactccctctcctctacc |
| | gccagcgccctgggcaaactgcaggacgtggtgaatcagaacgcccaagccctgaacaccctggtgaagcagctcagcagcaa |
| | ttttggcgccatcagctctgtgctgaacgatatcctgtctagactggaccctccagaagccgaagtccagatcgatagactga |
| | tcacaggcagactgcagtccctgcaaacctacgtgacccaacagctgatcagggccgctgaaataagagccagcgccaatctc |
| | gccgctaccaagatgtccgagtgtgtgctgggacagtctaaacgcgttgacttctgcggcaaaggctatcacctgatgagctt |
| | cccccagagcgcgccgcacggcgtggtgttcctgcatgtgacatacgtgcctgcccaagagaagaatttcacaaccgcccctg |
| | ccatctgccacgacggcaaggcccacttcccaagagagggcgttttcgtttccaatggcacacactggttcgtgacacaaaga |
| | aacttctacgaaccccagattatcaccaccgacaacaccttcgtgagtggcaattgtgacgtggtcatcggaatcgtgaacaa |
| | cacagtgtacgaccctctgcaacctgagctggactcttttaaggaagagctggacaagtactttaaaaaccacaccagccccg |
| | atgtggacctgggcgacatcagtggcattaacgccagcgtggtgaacatccaaaaggaaatcgacagactgaacgaggtggcc |
| | aagaacctgaacgagtccctgatcgacctgcaggagctcggcaaatacgagcagggatccggatacatccccgaggcccccag |
| | agatggccaggcctacgtgcggaaggacggcgagtgggtactgctgagcacattcctgggcagatccctggaggtgctgttcc |
| | agggcccaggccatcaccaccatcaccaccatcatagcgcctggtcccacccccagttcgagaagggcggcggtagtggaggg |
| | ggcggatctggcggctcagcttggagccacccccagttcgaaaag |
| 403 | MFVFLVLLPLVSSQCVNLTTRTQLPPAYTNSFTRGVYYPDKVFRSSVLHSTQDLFLPFFSNVTWFHAIHVSGTNGTKRFDNPV |
| SARS- | LPFNDGVYFASTEKSNIIRGWIFGTTLDSKTQSLLIVNNATNVVIKVCEFQFCNDPFLGVYYHKNNKSWMESEFRVYSSANNC |
| COV-2- | TFEYVSQPFLMDLEGKQGNFKNLREFVFKNIDGYFKIYSKHTPINLVRDLPQGFSALEPLVDLPIGINITRFQTLLALHRSYL |
| S | TPGDSSSGWTAGAAAYYVGYLQPRTFLLKYNENGTITDAVDCALDPLSETKCTLKSFTVEKGIYQTSNFRVQPTESIVRFPNI |
| | TNLCPFGEVENATRFASVYAWNRKRISNCVADYSVLYNSASFSTFKCYGVSPTKLNDLCFTNVYADSFVIRGDEVRQIAPGQT |
| | GKIADYNYKLPDDFTGCVIAWNSNNLDSKVGGNYNYLYRLFRKSNLKPFERDISTEIYQAGSTPCNGVEGENCYFPLQSYGFQ |
| | PTNGVGYQPYRVVVLSFELLHAPATVCGPKKSTNLVKNKCVNFNFNGLTGTGVLTESNKKFLPFQQFGRDIADTTDAVRDPQT |
| | LEILDITPCSFGGVSVITPGTNTSNQVAVLYQDVNCTEVPVAIHADQLTPTWRVYSTGSNVFQTRAGCLIGAEHVNNSYECDI |
| | PIGAGICASYQTQTNSPRRARSVASQSIIAYTMSLGAENSVAYSNNSIAIPTNFTISVTTEILPVSMTKTSVDCTMYICGDST |
| | ECSNLLLQYGSFCTQLNRALTGIAVEQDKNTQEVFAQVKQIYKTPPIKDFGGFNFSQILPDPSKPSKRSFIEDLLFNKVTLAD |
| | AGFIKQYGDCLGDIAARDLICAQKFNGLTVLPPLLTDEMIAQYTSALLAGTITSGWTFGAGAALQIPFAMQMAYRFNGIGVTQ |
| | NVLYENQKLIANQFNSAIGKIQDSLSSTASALGKLQDVVNQNAQALNTLVKQLSSNFGAISSVLNDILSRLDKVEAEVQIDRL |
| | ITGRLQSLQTYVTQQLIRAAEIRASANLAATKMSECVLGQSKRVDFCGKGYHLMSFPQSAPHGVVFLHVTYVPAQEKNFTTAP |
| | AICHDGKAHFPREGVFVSNGTHWFVTQRNFYEPQIITTDNTFVSGNCDVVIGIVNNTVYDPLQPELDSFKEELDKYFKNHTSP |
| | DVDLGDISGINASVVNIQKEIDRLNEVAKNLNESLIDLQELGKYEQYIKWPWYIWLGFIAGLIAIVMVTIMLCCMTSCCSCLK |
| | GCCSCGSCCKFDEDDSEPVLKGVKLHYT |
| 404 | atgtttgtttttcttgttttattgccactagtctctagtcagtgtgttaatcttacaaccagaactcaattaccccctgcata |
| | cactaattctttcacacgtggtgtttattaccctgacaaagttttcagatcctcagttttacattcaactcaggacttgttct |
| | tacctttcttttccaatgttacttggttccatgctatacatgtctctgggaccaatggtactaagaggtttgataaccctgtc |
| | ctaccatttaatgatggtgtttattttgcttccactgagaagtctaacataataagaggctggatttttggtactactttaga |
| | ttcgaagacccagtccctacttattgttaataacgctactaatgttgttattaaagtctgtgaatttcaattttgtaatgatc |
| | catttttgggtgtttattaccacaaaaacaacaaaagttggatggaaagtgagttcagagtttattctagtgcgaataattgc |
| | acttttgaatatgtctctcagccttttcttatggaccttgaaggaaaacagggtaatttcaaaaatcttagggaatttgtgtt |
| | taagaatattgatggttattttaaaatatattctaagcacacgcctattaatttagtgcgtgatctccctcagggtttttcgg |
| | ctttagaaccattggtagatttgccaataggtattaacatcactaggtttcaaactttacttgctttacatagaagttatttg |
| | actcctggtgattcttcttcaggttggacagctggtgctgcagcttattatgtgggttatcttcaacctaggacttttctatt |
| | aaaatataatgaaaatggaaccattacagatgctgtagactgtgcacttgaccctctctcagaaacaaagtgtacgttgaaat |
| | ccttcactgtagaaaaaggaatctatcaaacttctaactttagagtccaaccaacagaatctattgttagatttcctaatatt |
| | acaaacttgtgcccttttggtgaagtttttaacgccaccagatttgcatctgtttatgcttggaacaggaagagaatcagcaa |
| | ctgtgttgctgattattctgtcctatataattccgcatcattttccacttttaagtgttatggagtgtctcctactaaattaa |
| | atgatctctgctttactaatgtctatgcagattcatttgtaattagaggtgatgaagtcagacaaatcgctccagggcaaact |
| | ggaaagattgctgattataattataaattaccagatgattttacaggctgcgttatagcttggaattctaacaatcttgattc |
| | taaggttggtggtaattataattacctgtatagattgtttaggaagtctaatctcaaaccttttgagagagatatttcaactg |
| | aaatctatcaggccggtagcacaccttgtaatggtgttgaaggttttaattgttactttcctttacaatcatatggtttccaa |
| | cccactaatggtgttggttaccaaccatacagagtagtagtactttcttttgaacttctacatgcaccagcaactgtttgtgg |
| | acctaaaaagtctactaatttggttaaaaacaaatgtgtcaatttcaacttcaatggtttaacaggcacaggtgttcttactg |
| | agtctaacaaaaagtttctgcctttccaacaatttggcagagacattgctgacactactgatgctgtccgtgatccacagaca |
| | cttgagattcttgacattacaccatgttcttttggtggtgtcagtgttataacaccaggaacaaatacttctaaccaggttgc |
| | tgttctttatcaggatgttaactgcacagaagtccctgttgctattcatgcagatcaacttactcctacttggcgtgtttatt |
| | ctacaggttctaatgtttttcaaacacgtgcaggctgtttaataggggctgaacatgtcaacaactcatatgagtgtgacata |
| | cccattggtgcaggtatatgcgctagttatcagactcagactaattctcctcgggggcacgtagtgtagctagtcaatccatc |
| | attgcctacactatgtcacttggtgcagaaaattcagttgcttactctaataactctattgccatacccacaaattttactat |
| | tagtgttaccacagaaattctaccagtgtctatgaccaagacatcagtagattgtacaatgtacatttgtggtgattcaactg |
| | aatgcagcaatcttttgttgcaatatggcagtttttgtacacaattaaaccgtgctttaactggaatagctgttgaacaagac |
| | aaaaacacccaagaagtttttgcacaagtcaaacaaatttacaaaacaccaccaattaaagattttggtggttttaatttttc |
| | acaaatattaccagatccatcaaaaccaagcaagaggtcatttattgaagatctacttttcaacaaagtgacacttgcagatg |
| | ctggcttcatcaaacaatatggtgattgccttggtgatattgctgctagagacctcatttgtgcacaaaagtttaacggcctt |
| | actgttttgccacctttgctcacagatgaaatgattgctcaatacacttctgcactgttagcgggtacaatcacttctggttg |
| | gacctttggtgcaggtgctgcattacaaataccatttgctatgcaaatggcttataggtttaatggtattggagttacacaga |
| | atgttctctatgagaaccaaaaattgattgccaaccaatttaatagtgctattggcaaaattcaagactcactttcttccaca |
| | gcaagtgcacttggaaaacttcaagatgtggtcaaccaaaatgcacaagctttaaacacgcttgttaaacaacttagctccaa |
| | ttttggtgcaatttcaagtgttttaaatgatatcctttcacgtcttgacaaagttgaggctgaagtgcaaattgataggttga |
| | tcacaggcagacttcaaagtttgcagacatatgtgactcaacaattaattagagctgcagaaatcagagcttctgctaatctt |
| | gctgctactaaaatgtcagagtgtgtacttggacaatcaaaaagagttgatttttgtggaaagggctatcatcttatgtcctt |
| | ccctcagtcagcacctcatggtgtagtcttcttgcatgtgacttatgtccctgcacaagaaaagaacttcacaactgctcctg |
| | ccatttgtcatgatggaaaagcacactttcctcgtgaaggtgtctttgtttcaaatggcacacactggtttgtaacacaaagg |
| | aatttttatgaaccacaaatcattactacagacaacacatttgtgtctggtaactgtgatgttgtaataggaattgtcaacaa |
| | cacagtttatgatcctttgcaacctgaattagactcattcaaggaggagttagataaatattttaagaatcatacatcaccag |
| | atgttgatttaggtgacatctctggcattaatgcttcagttgtaaacattcaaaaagaaattgaccgcctcaatgaggttgcc |
| | aagaatttaaatgaatctctcatcgatctccaagaacttggaaagtatgagcagtatataaaatggccatggtacatttggct |
| | aggttttatagctggcttgattgccatagtaatggtgacaattatgctttgctgtatgaccagttgctgtagttgtctcaagg |
| | gctgttgttcttgtggatcctgctgcaaatttgatgaagacgactctgagccagtgctcaaaggagtcaaattacattacaca |
| | taa |
| 405 | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaaaaaaaaaaaaaaaaaaaaaaaaaaaaaaaaaaaaaaaaaaaaaaaaaaaaaaaaaaaaaaaaaaaaaaaaaaaaa |
| | aaaaaaaa |

1.-42. (canceled)

43. A pharmaceutical formulation comprising
(i) an isolated antibody, or antigen-binding fragment thereof, which binds to the spike protein of a coronavirus (“CoV-S”), wherein the antibody, or antigen-binding fragment thereof, comprises a heavy chain variable region (VH) comprising a VH CDR1 comprising SEQ ID NO: 52, a VH CDR2 comprising SEQ ID NO:54, and a VH CDR3 comprising SEQ ID NO:56, and a light chain variable region (VL) comprising a VL CDR1 comprising SEQ ID NO:252, a VL CDR2 comprising SEQ ID NO:254, and a VL CDR3 comprising SEQ ID NO:256;
(ii) about 1−20 mM histidine buffer having a pH of about 5.0−6.0;
(iii) about 50−150 mM sucrose;
(iv) about 50−100 m arginine hydrochloride; and
(v) about 0.01−0.05% w/v polysorbate 80.

44. The pharmaceutical composition of claim 43, wherein the antibody, or antigen-binding fragment thereof, has a concentration of about 10−500 mg/mL, about 50−400 mg/mL, about 100−300 mg/mL, about 100−200 mg/mL, about 125−175 mg/mL, or about 130−170 mg/mL.

45. The pharmaceutical composition of claim 43, wherein the antibody, or antigen-binding fragment thereof, has a concentration of about 10 mg/mL, about 50 mg/mL, about 100 mg/mL, about 125 mg/mL, about 130 mg/mL, about 150 mg/mL, about 170 mg/mL, about 175 mg/mL, about 200 mg/mL, about 250 mg/mL, about 300 mg/mL, about 400 mg/mL, or about 500 mg/mL.

46. The pharmaceutical composition of claim 43, wherein the concentration of the antibody, or antigen-binding fragment thereof, is about 150 mg/mL.

47. The pharmaceutical formulation of claim 43, comprising
(i) about 150 mg/mL of the isolated antibody, or antigen-binding fragment thereof,
(ii) about 10 mM histidine or acetate buffer having a pH of about 5.4−5.6;
(iii) about 100 mM sucrose or mannitol;
(iv) about 75 mM arginine hydrochloride; and
(v) about 0.03% w/v polysorbate 80.

48. The pharmaceutical composition of claim 43, wherein the formulation is stable at about 2−8° C. for at least 1, 2, or 3 years.

49. The pharmaceutical composition of claim 43, wherein the formulation is stable at about 25° C., or about 40° C. for at least one, two, or four weeks.

50. The pharmaceutical composition of claim 43, wherein the formulation is stable at about <−30° C. for at least 3, 4 or 5 years.

51. A method of inducing an immune response against SARS-CoV, SARS-COV-2, or another coronavirus selected from the group consisting of MERS-COV, HCoV-HKUl, HCoV-OC43, HCOV-229E, and HCoV-NL63 in a human subject in need thereof, the method comprising administering the pharmaceutical formulation of claim 43.

52. A method of preventing infection of susceptible cells by SARS-COV, SARS-COV-2, or another coronavirus selected from the group consisting of MERS-CoV, HCoV-HKUl, HCoV-OC43, HCOV-229E, and HCoV-NL63 in a human subject in need thereof, the method comprising administering the pharmaceutical formulation of claim 43.

53. A method of treating a coronavirus infection or reducing a symptom of a coronavirus infection of a subject by SARS-COV, SARS-COV-2, or another coronavirus selected from the group consisting of MERS-COV, HCOV-HKUl, HCoV-OC43, HCoV-229E, and HCoV-NL63 in a human subject in need thereof, the method comprising administering the pharmaceutical formulation of claim 43.

54. (canceled)

55. The method of claim 53, wherein the symptom comprises at least one of bronchitis, pneumonia, respiratory failure, acute respiratory failure, organ failure, multi-organ system failure, pediatric inflammatory multisystem syndrome, acute respiratory distress syndrome, blood clot, a cardiac condition, myocardial injury, myocarditis, heart failure, cardiac arrest, acute myocardial infarction, dysrhythmia, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, a gastrointestinal complication, aspergillosis, increased susceptibility to infection with another virus or bacteria, and/or a pregnancy-related complication.

56. A method of decreasing the risk of mortality, hospitalization, mechanical ventilation, or a combination thereof, in a human subject infected by SARS-COV, SARS-COV-2, or another coronavirus selected from the group consisting of MERS-COV, HCoV-HKUl, HCoV-OC43, HCoV-229E, and HCoV-NL63, the method comprising administering the pharmaceutical formulation of claim 43.

57. (canceled)

58. The method of claim 53, wherein the subject has at least one risk factor which renders them more prone to a poor clinical outcome.

59. The method of claim 58, wherein the at least one risk factor is selected from the group consisting of: an old age selected from the group consisting of over 55, over 60 or over 65 years old; diabetes, a chronic respiratory condition, obesity, hypertension, a cardiac or cardiovascular condition, a chronic inflammatory or autoimmune condition, and an immune compromised status.

60. The method of claim 53, wherein the pharmaceutical formulation is administered intramuscularly, or intravenously, or subcutaneously.

61. The method of claim 53, wherein the pharmaceutical formulation is administered once or is administered yearly, monthly, every 2 months, every 3 months, every 4 months, every 5 months, every 6 months, every 7 months, every eight months, every 9 months, every 10 months, every 11 months, every 2 weeks, or weekly.

62. The pharmaceutical composition of claim 43, wherein the VH has at least 90% identity to SEQ ID NO:58 and the VL has at least 90% identity to SEQ ID NO:258.

63. The pharmaceutical composition of claim 43, wherein the VH has at least 95% identity to SEQ ID NO:58 and the VL has at least 95% identity to SEQ ID NO:258.

64. The pharmaceutical composition of claim 43, wherein the VH comprises SEQ ID NO: 58 and wherein the VL comprises of SEQ ID NO:258.
